(19) United States
(12) Patent Application Publication
(10) Pub. No.: US 2023/0235332 A1
(43) Pub. Date: Jul. 27, 2023

(54) TREATMENT OF NEUROLOGICAL DISEASES USING MODULATORS OF GENE TRANSCRIPTS

(71) Applicant: QURALIS CORPORATION, Cambridge, MA (US)

(72) Inventors: Daniel Elbaum, Newton, MA (US); Sandra Hinckley, Sudbury, MA (US)

(21) Appl. No.: 17/928,708

(22) PCT Filed: Jun. 3, 2021
(86) PCT No.: PCT/US2021/035603

§ 371 Date: Nov. 30, 2022

Related U.S. Application Data
(60) Provisional application No. 63/033,926, filed on Jun. 3, 2020.
(60) Provisional application No. 63/119,717, filed on Dec. 1, 2020.

(51) Int. Cl. C12N 15/113; A61K 45/06; A61P 25/28

(52) U.S. Cl. CPC C12N 15/1136; A61K 45/06; A61P 25/28; C12N 2310/3231; C12N 2310/321; C12N 2310/315; C12N 2310/311; C12N 2310/314; C12N 2310/312

(57) ABSTRACT

Disclosed herein are STMN2 oligonucleotides with one or more spacers. In various embodiments, STMN2 oligonucleotides with spacer(s) reduce STMN2 transcripts with cryptic exon and increase full length STMN2 transcripts, thereby imparting therapeutic efficacy against neurological diseases such as amyotrophic lateral sclerosis (ALS), frontotemporal dementia (FTD), or Alzheimer's disease (AD).

### FIELD OF THE DISCLOSURE

This application relates generally to methods of treating neurological diseases with antisense oligonucleotides, in particular, antisense oligonucleotides with one or more spacers that target a transcript.

### CROSS REFERENCE TO RELATED APPLICATIONS

This application claims the benefit of and priority to U.S. Provisional Patent Application No. 63/033,926 filed on Jun. 3, 2020 and U.S. Provisional Patent Application No. 63/119,717 filed on Dec. 1, 2020, the entire disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on May 28, 2021, is named QRL-006WO_SL.txt and is 510,394 bytes in size.

### BACKGROUND

Motor neuron diseases are a class of neurological diseases that result in the degeneration and death of motor neurons—those neurons which coordinate voluntary movement of muscles by the brain. Motor neuron diseases may be sporadic or inherited, and may affect upper motor neurons and/or lower motor neurons. Motor neuron diseases include amyotrophic lateral sclerosis, progressive bulbar palsy, pseudobulbar palsy, primary lateral sclerosis, progressive muscular atrophy, spinal muscular atrophy, and post-polio syndrome.

Amyotrophic lateral sclerosis (ALS) is a group of motor neuron diseases affecting about 15,000 individuals in the United States of America. ALS is characterized by degeneration and death of upper and lower motor neurons, resulting in loss of voluntary muscle control. Motor neuron death is accompanied by muscle fasciculation and atrophy. Early symptoms of ALS include muscle cramps, muscle spasticity, muscle weakness (for example, affecting an arm, a leg, neck, or diaphragm), slurred and nasal speech, and difficulty chewing or swallowing. Loss of strength and control over movements, including those necessary for speech, eating, and breathing, eventually occur. Disease progression may be accompanied by weight loss, malnourishment, anxiety, depression, increased risk of pneumonia, muscle cramps, neuropathy, and possibly dementia. Most individuals diagnosed with ALS die of respiratory failure within five years of the first appearance of symptoms. Currently, there is no effective treatment for ALS.

ALS occurs in individuals of all ages, but is most common in individuals between 55 to 75 years of age, with a slightly higher incidence in males. ALS can be characterized as sporadic or familial. Sporadic ALS appears to occur at random and accounts for more than 90% of all incidences of ALS. Familial ALS accounts for 5-10% of all incidences of ALS.

FTD refers to a spectrum of progressive neurodegenerative diseases caused by loss of neurons in frontal and temporal lobes of the brain. FTD is the third most common form of dementia (following Alzheimer's disease and dementia with Lewy bodies), and the second most common form of dementia in individuals below 65 years of age. FTD is estimated to affect 20,000 to 30,000 individuals in the United States of America. FTD is characterized by changes in behavior and personality, and language dysfunction. Forms of FTD include behavioral variant FTD (bvFTD), semantic variant primary progressive aphasia (svPPA), and nonfluent variant primary progressive aphasia (nfvPPA). ALS with FTD is characterized by symptoms associated with FTD, along with symptoms of ALS such as muscle weakness, atrophy, fasciculation, spasticity, speech impairment (dysarthria), and inability to swallow (dysphagia). Individuals usually succumb to FTD within 5 to 10 years, while ALS with FTD often results in death within 2 to 3 years of the first disease symptoms appearing.

Like ALS, there is no known cure for FTD, or ALS with FTD, nor a therapeutic known to prevent or retard either disease's progression.

Thus, there is a pressing need to identify compounds and/or compositions capable of preventing, ameliorating, and neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

RNA-binding protein transactive response DNA-binding protein 43 (TDP-43) is involved in fundamental RNA processing activities including RNA transcription, splicing, and transport. TDP-43 binds to thousands of pre-messenger RNA/mRNA targets, with high affinity for GU-rich sequences, including autoregulation of its own mRNA via binding to 3′ untranslated region. Reduction in TDP-43 from an otherwise normal adult nervous system alters the splicing or expression levels of more than 1,500 RNAs, including long intron-containing transcripts. See Melamed et al., Nat Neurosci. (2019), 22(2):180-190.

In affected neurons in most instances of ALS and approximately 45% of patients with FTD, cytoplasmic accumulation and nuclear loss of TDP-43 have been reported. See Melamed et al., Nat Neurosci. (2019), 22(2):180-190. Moreover, TDP-43 has been shown to regulate expression of the neuronal growth-associated factor Stathmin-2 (STMN2). See Melamed (2019); see also Klim et al., Nat Neurosci. (2019), 22(2):167-179. STMN2 encodes a protein necessary for normal motor neuron outgrowth and repair. See Melamed (2019); see also Klim (2019). TDP-43 disruption is shown to drive premature polyadenylation and aberrant splicing in intron 1 of stathmin-2 pre-mRNA, producing a non-functional mRNA. See Melamed (2019).

### SUMMARY

Described herein are oligonucleotides comprising one or more spacers and comprising a sequence that is between 85 and 98% complementary to an equal length portion of a STMN2 transcript. In one aspect, the present disclosure provides STMN2 oligonucleotides that target a STMN2 transcript (for example, a STMN2 transcript comprising a cryptic exon). In various embodiments, the oligonucleotides target a transcript for the treatment of neurological diseases, including motor neuron diseases, and/or neuropathies. For example, STMN2 oligonucleotides can be used to treat PD, ALS, FTD, and ALS with FTD.

In one aspect the present disclosure provides a compound comprising a modified oligonucleotide comprising a sequence that is between 85 and 98% complementary to an equal length portion of any one of SEQ ID NO: 1339 or SEQ ID NO: 1341, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer. In various embodiments, the oligonucleotide comprises a segment with at most 11 linked nucleosides. In various embodiments, the oligonucleotide comprises a segment with at most 10, 9, or 8 linked nucleosides. In various embodiments, the oligonucleotide comprises a segment with at most 7 linked nucleosides. In certain embodiments, the oligonucleotide comprises a segment with at most 6, 5, 4, 3, or 2 linked nucleosides. In certain embodiments, every segment of the oligonucleotide comprises at most 7 linked nucleosides.

In various embodiments, the oligonucleotide comprises a sequence that shares at least 85% identity with an equal length portion of any one of SEQ ID NOs: 1-466, SEQ ID NOs: 893-1338, SEQ ID NOs: 1342-1366, or SEQ ID NOs: 1392-1664. In various embodiments, the oligonucleotide comprises a sequence that shares at least 90% identity with an equal length portion of any one of SEQ ID NOs: 1-466, SEQ ID NOs: 893-1338, SEQ ID NOs: 1342-1366, or SEQ ID NOs: 1392-1664. In various embodiments, the oligonucleotide comprises a sequence that shares 95% identity with an equal length portion of any one of SEQ ID NOs: 1451-1664. In various embodiments, the oligonucleotide comprises a sequence that shares 100% identity with an equal length portion of any one of SEQ ID NOs: 1451-1664.

In various embodiments, the oligonucleotide comprises a segment with at most 11 linked nucleosides or at most 7 linked nucleosides, and wherein the oligonucleotide comprises a sequence that is between 85 and 98% complementary to an equal length portion within any one of positions 144-168, 173-197, 185-209, or 237-261 of SEQ ID NO: 1339. In various embodiments, the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 144-164, 144-166, 145-167, 146-166, 146-168, 147-165, or 148-168 of SEQ ID NO: 1339. In various embodiments, the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 173-191, 173-193, 173-195, 173-197, 175-195, 175-197, 177-197, or 179-197 of SEQ ID NO:

1339.

In various embodiments, the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 185-205, 187-209, 189-209, or 191-209 of SEQ ID NO: 1339. In various embodiments, the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 237-255, 237-259, 239-259, 239-261, 241-261, or 243-261 of SEQ ID NO: 1339. In various embodiments, the oligonucleotide comprises a segment with at most 6, 5, 4, 3, or 2 linked nucleosides, and wherein the oligonucleotide comprises a sequence that is between 85 and 98% complementary to an equal length portion of nucleobases within any one of positions 144-168, 173-197, 185-209, or 237-261 of SEQ ID NO: 1339.

In various embodiments, the oligonucleotide comprises a segment with at most 6, 5, 4, 3, or 2 linked nucleosides, and wherein the oligonucleotide comprises a sequence that is between 85 and 98% complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 187-209, 189-209, 191-209, 237-255, 237-259, 239-259, 239-261, 241-261, or 243-261 of SEQ ID NO: 1339. In various embodiments, the oligonucleotide comprises a segment with at most 11 linked nucleosides or at most 7 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, or SEQ ID NOs: 1392-1664. In various embodiments, the oligonucleotide comprises a segment with at most 6, 5, 4, 3, or 2 linked nucleosides, and wherein the oligonucleotide comprises a sequence that shares at least 85% identity with an equal length portion of any one of SEQ ID NOs: 36, 55, 144, 173, 177, 181, 185, 197, 203, 209, 215, 237, 244, 252, 380, 385, 390, 395, 400, 928, 947, 1036, 1065, 1069, 1073, 1077, 1089, 1095, 1101, 1107, 1129, 1136, 1144, 1272, 1277, 1282, 1287, or 1292. In various embodiments, the oligonucleotide comprises a segment with at most 6, 5, 4, 3, or 2 linked nucleosides, and wherein the oligonucleotide comprises a sequence that shares at least 90% identity with an equal length portion of any one of SEQ ID NOs: 36, 55, 144, 173, 177, 181, 185, 197, 203, 209, 215, 237, 244, 252, 380, 385, 390, 395, 400, 928, 947, 1036, 1065, 1069, 1073, 1077, 1089, 1095, 1101, 1107, 1129, 1136, 1144, 1272, 1277, 1282, 1287, or 1292.

In various embodiments, the oligonucleotide is at least 18, at least 19, at least 20, at least 21, at least 22, at least 23, at least 24, or at least 25 oligonucleotide units in length. In various embodiments, the oligonucleotide is at least 19 oligonucleotide units in length. In various embodiments, the spacer is a nucleoside-replacement group comprising a non-sugar substitute that is incapable of linking to a nucleotide base.

In various embodiments, the spacer is located between positions 10 and 15 of the oligonucleotide. In various embodiments, the spacer is located between positions 7 and 11 of the oligonucleotide. In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide. In various embodiments, the oligonucleotide further comprises a third spacer, wherein the third spacer is located between positions 21 and 24 of the oligonucleotide.

In various embodiments, the spacer is located between positions 2 and 5 of the oligonucleotide. In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides. In various embodiments, at least two of the three spacers are adjacent to a guanine nucleobase. In various embodiments, each of the at least two of the three spacers immediately precede a guanine nucleobase.

In various embodiments, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In certain embodiments, each of the first, second or third spacers is independently represented by Formula (X), wherein:

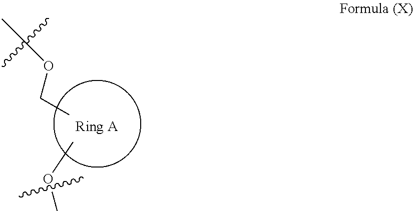

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and

the

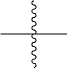

symbol represents the point of connection to an internucleoside linkage.

In various embodiments, each of the first, second or third spacers is independently represented by Formula (Xa), wherein:

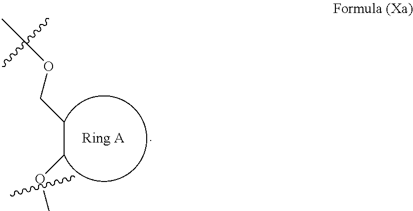

In some embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

In further embodiments, ring A is tetrahydrofuranyl.

In other embodiments, ring A is tetrahydropyranyl.

In various embodiments, each of the first, second or third spacers is independently represented by Formula I, wherein:

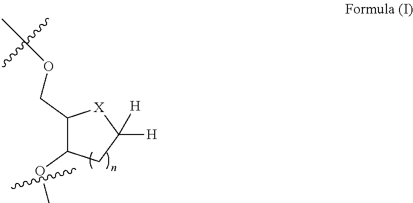

X is selected from —CH₂— and —O—; and

n is 0, 1, 2 or 3.

In various embodiments, each of the first, second or third spacers is independently represented by Formula I′, wherein:

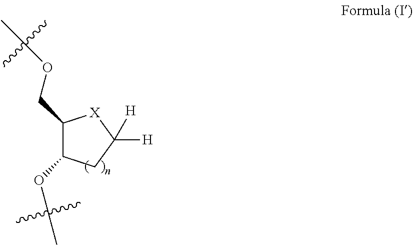

X is selected from —CH₂— and —O—; and

n is 0, 1, 2 or 3.

In various embodiments, each of the first, second or third spacers is independently represented by Formula (Ia), wherein:

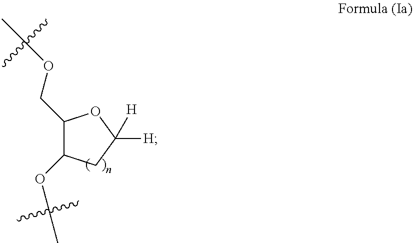

and

n is 0, 1, 2 or 3.

In various embodiments, each of the first, second or third spacers is independently represented by Formula (Ia′), wherein:

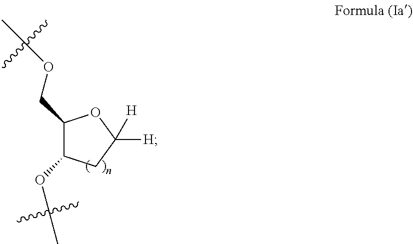

and

n is 0, 1, 2 or 3.

In certain embodiments, each of the first, second or third spacers is independently represented by Formula II, wherein:

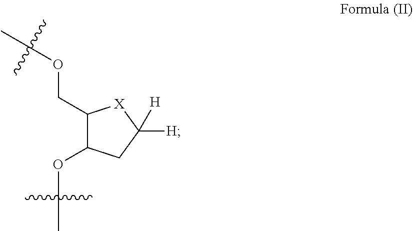

and

X is selected from —CH₂— and —O—.

In further embodiments, each of the first, second or third spacers is independently represented by Formula II′, wherein:

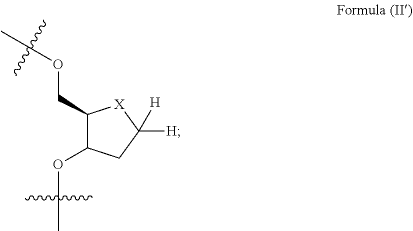

and

X is selected from —CH₂— and

In various embodiments, each of the first, second or third spacers is independently represented by Formula (IIa), wherein:

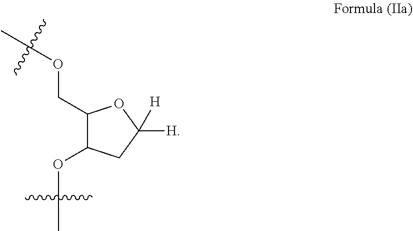

In further embodiments, each of the first, second or third spacers is independently represented by Formula (IIa′), wherein:

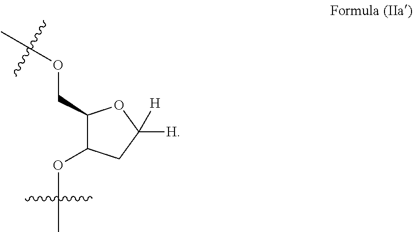

In various embodiments, each of the first, second or third spacers is independently represented by Formula III, wherein:

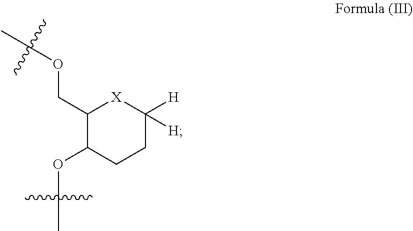

and

X is selected from —CH₂— and —O—.

In further embodiments, each of the first, second or third spacers is independently represented by Formula III′, wherein:

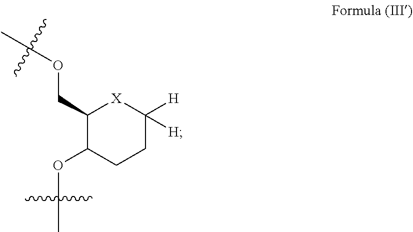

and

X is selected from —CH₂— and —O—.

In some embodiments, each of the first, second or third spacers is independently represented by Formula (IIIa), wherein:

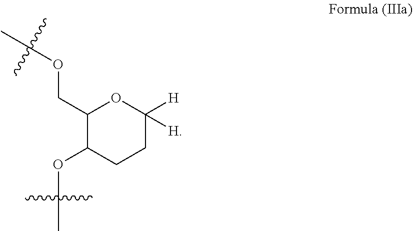

In further embodiments, each of the first, second or third spacers is independently represented by Formula (IIIa′), wherein:

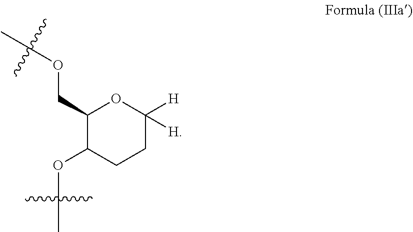

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%.

In various embodiments, the oligonucleotide is between 12 and 40 oligonucleotide units in length.

In various embodiments, at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, one or more nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond. In various embodiments, the oligonucleotide comprises a second spacer, wherein a base immediately preceding the second spacer is linked to a further preceding base through a phosphodiester bond.

In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds. In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds. In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds. In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers.

Additionally disclosed herein is a compound comprising an oligonucleotide comprising a nucleobase sequence that shares at least 90% identity to an equal length portion of any one of SEQ ID NOs: 1-466, SEQ ID NOs: 893-1338, SEQ ID NOs: 1342-1366, or SEQ ID NOs: 1392-1664. Additionally disclosed herein is an oligonucleotide comprising a nucleobase sequence that shares at least 90% identity to an equal length portion of any one of SEQ ID NOs: 1-466, SEQ ID NOs: 893-1338, SEQ ID NOs: 1342-1366, or SEQ ID NOs: 1392-1664. In various embodiments, the nucleobase sequence shares at least 95% identity to an equal length portion of any one of SEQ ID NOs: 1-466, SEQ ID NOs: 893-1338, SEQ ID NOs: 1342-1366, or SEQ ID NOs: 1392-1664. In various embodiments, the nucleobase sequence shares at least 100% identity to an equal length portion of any one of SEQ ID NOs: 1-466, SEQ ID NOs: 893-1338, SEQ ID NOs: 1342-1366, or SEQ ID NOs: 1392-1664. In various embodiments, the oligonucleotide is any one of a 19mer, 21mer, 23mer, or 25mer.

In various embodiments, an internucleoside linkage of the oligonucleotide is a modified internucleoside linkage. In various embodiments, the modified internucleoside linkage of the oligonucleotide is a phosphorothioate linkage. In various embodiments, all internucleoside linkages of the oligonucleotide are phosphorothioate linkages. In various embodiments, the phosphorothioate linkage is in one of a Rp configuration or a Sp configuration. In various embodiments, the oligonucleotide comprises at least one modified sugar moiety. In various embodiments, the modified sugar moiety is one of a 2′-OMe modified sugar moiety, bicyclic sugar moiety, 2′-O-(2-methoxyethyl) (MOE), 2′-deoxy-2′-fluoro nucleoside, 2′-fluoro-β-D-arabinonucleoside, locked nucleic acid (LNA), a tricyclic nucleic acid, constrained ethyl 2′-4′-bridged nucleic acid (cEt), S-cEt, tcDNA, hexitol nucleic acids (HNA), and tricyclic analog (e.g., tcDNA).

In various embodiments, the oligonucleotide exhibits at least a 30%, 40%, 50%, 60%, 70%, 80%, or 90% increase of full length STMN2 protein. In various embodiments, the oligonucleotide exhibits at least a 100% increase of full length STMN2 protein. In various embodiments, the oligonucleotide exhibits at least a 200% increase of full length STMN2 protein. In various embodiments, the oligonucleotide exhibits at least a 300% increase of full length STMN2 protein. In various embodiments, the oligonucleotide exhibits at least a 400% increase of full length STMN2 protein. In various embodiments, increase of the full length STMN2 protein is measured in comparison to a reduced level of full length STMN2 protein achieved using a TDP43 antisense oligonucleotide. In various embodiments, the oligonucleotide exhibits at least a 10%, 20%, 30%, 40%, 50%, 60%, 70%, 80%, 90%, or 100% rescue of full length STMN2 protein. In various embodiments, the oligonucleotide exhibits at least a 50%, 60%, 70%, 80%, or 90% reduction of a STMN2 transcript with a cryptic exon.

Additionally disclosed is a method of treating a neurological disease and/or a neuropathy in a patient in need thereof, the method comprising administering to the patient an oligonucleotide of any of the oligonucleotides disclosed above. In various embodiments, the neurological disease selected from the group consisting of: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)). In various embodiments, the neurological disease is ALS. In various embodiments, the neurological disease is FTD. In various embodiments, the neurological disease is ALS with FTD. In various embodiments, the neuropathy is chemotherapy induced neuropathy.

Additionally disclosed is a method of restoring axonal outgrowth and/or regeneration of a neuron, the method comprising exposing the motor neuron to an oligonucleotide of any of the oligonucleotides disclosed above. Additionally disclosed is a method of increasing, promoting, stabilizing, or maintaining STMN2 expression and/or function in a neuron, the method comprising exposing the cell to an oligonucleotide of any of the oligonucleotides disclosed above.

In various embodiments, the neuron is a neuron of a patient in need of treatment of a neurological disease and/or a neuropathy. In various embodiments, the neuropathy is chemotherapy induced neuropathy. In various embodiments, the exposing is performed in vivo or ex vivo. In various embodiments, the exposing comprises administering the oligonucleotide to a patient in need thereof. In various embodiments, the oligonucleotide is administered topically, parenterally, intrathecally, intrathalamically, intracisternally, orally, rectally, buccally, sublingually, vaginally, pulmonarily, intratracheally, intranasally, transdermally, or intraduodenally. In various embodiments, the oligonucleotide is administered orally. In various embodiments, a therapeutically effective amount of the oligonucleotide is administered intrathecally, intrathalamically or intracisternally. In various embodiments, the patient is a human.

Additionally disclosed herein is a pharmaceutical composition comprising the oligonucleotide of any one of the oligonucleotides disclosed above, or a pharmaceutically acceptable salt thereof, and a pharmaceutically acceptable excipient. In various embodiments, the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

Additionally disclosed herein is a method of treating a neurological disease or a neuropathy in a patient in need thereof, the method comprising administering to a patient in need thereof a therapeutically effective amount of a pharmaceutical composition disclosed above. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)). In various embodiments, the neurological disease is ALS. In various embodiments, the neurological disease is FTD. In various embodiments, the neurological disease is ALS with FTD. In various embodiments, the neuropathy is chemotherapy induced neuropathy. In various embodiments, the pharmaceutical composition is administered topically, parenterally, orally, pulmonarily, rectally, buccally, sublingually, vaginally, intratracheally, intranasally, intracisternally, intrathecally, intrathalamically, intravenously, intramuscularly, transdermally, or intraduodenally. In various embodiments, wherein the pharmaceutical composition is administered intrathecally, intrathalamically intracerebroventricularly, or intracisternally. In various embodiments, a therapeutically effective amount of the oligonucleotide is administered intrathecally, intrathalamically or intracisternally. In various embodiments, the patient is human.

Additionally disclosed herein is a method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating ALS in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating FTD in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating ALS with FTD in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, one or more nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond. In various embodiments, the oligonucleotide comprises a second spacer, wherein a base immediately preceding the second spacer is linked to a further preceding base through a phosphodiester bond.

In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds. In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds. In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds. In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers. In various embodiments, the oligonucleotide is any one of a 19mer, 21mer, 23mer, or 25mer.

In various embodiments, at least one (i.e., one or more) internucleoside linkage of the oligonucleotide is a phosphorothioate linkage. In various embodiments, all internucleoside linkages of the oligonucleotide are phosphorothioate linkages.

Additionally disclosed herein is an oligonucleotide and a pharmaceutically acceptable excipient, the oligonucleotide comprising a sequence that is between 85 and 98% complementary to an equal length portion of any one of SEQ ID NO: 1339 or SEQ ID NO: 1341, a sequence having 90% identity thereof, or to a 15 to 50 contiguous nucleobase portion thereof, wherein the oligonucleotide comprises a spacer and wherein the oligonucleotide is capable of increasing, restoring, or stabilizing expression of the STMN2 mRNA capable of translation of a functional STMN2 and/or activity and/or function of STMN2 protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

In various embodiments, the oligonucleotide comprises one or more chiral centers and/or double bonds. In various embodiments, the oligonucleotide exist as stereoisomers selected from geometric isomers, enantiomers, and diastereomers.

Additionally disclosed herein is a method of treating a neurological disease and/or a neuropathy in a patient in need thereof, the method comprising administering to a patient in need thereof a therapeutically effective amount of a pharmaceutical composition disclosed above, in combination with a second therapeutic agent. In various embodiments, the second therapeutic agent is selected from Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprosterone, KCNQ2/KCNQ3 openers (e.g., retigabine, XEN1101, QRL-101), anticonvulsants and psychostimulant agents, and/or a therapy (e.g., selected from breathing care, physical therapy, occupational therapy, speech therapy, nutritional support), for treating said neurologic disease.

Additionally disclosed herein is a method of treating a neurological disease and/or a neuropathy in a patient in need thereof, the method comprising administering to a patient in need thereof a therapeutically effective amount of a pharmaceutical composition disclosed above, wherein at least one (i.e., one or more) nucleoside linkage of the oligonucleotide is a non-natural linkage, wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

In various embodiments, the spacer is a nucleoside-replacement group comprising a non-sugar substitute that is incapable of linking to a nucleotide base. In various embodiments, the spacer is located between positions 10 and 15 of the oligonucleotide. In various embodiments, the spacer is located between positions 7 and 11 of the oligonucleotide. In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide.

In various embodiments, the oligonucleotide further comprises a third spacer, wherein the third spacer is located between positions 21 and 24 of the oligonucleotide. In various embodiments, the spacer is located between positions 2 and 5 of the oligonucleotide. In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides.

In various embodiments, at least two of the three spacers are adjacent to a guanine nucleobase. In various embodiments, each of the at least two of the three spacers immediately precede a guanine nucleobase.

In various embodiments, of the methods described herein, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In certain embodiments, each of the first, second or third spacers is independently represented by Formula (X), wherein:

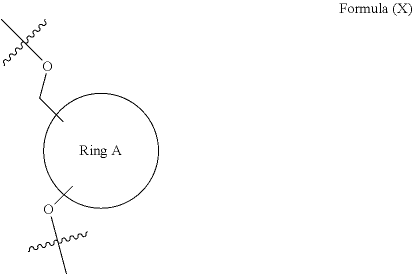

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and

the

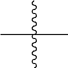

symbol represents the point of connection to an internucleoside linkage.

In various embodiments, each of the first, second or third spacers is independently represented by Formula (Xa), wherein:

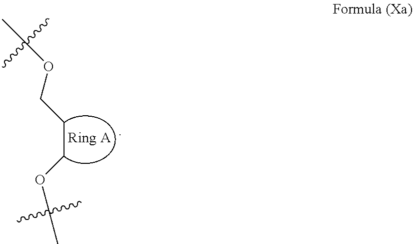 [SMILES: *OCCCO*]

In some embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

In further embodiments, ring A is tetrahydrofuranyl.

In other embodiments, ring A is tetrahydropyranyl.

In various embodiments, each of the first, second or third spacers is independently represented by Formula (I), wherein:

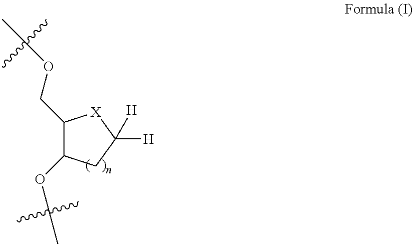

X is selected from —CH₂— and —O—; and

n is 0, 1, 2 or 3.

In various embodiments, the spacer or the second spacer is represented by Formula (I′), wherein:

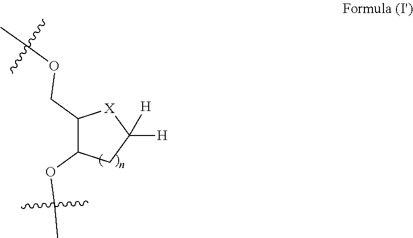

X is selected from —CH₂— and —O—; and

n is 0, 1, 2 or 3.

In various embodiments, each of the first, second or third spacers is independently represented by Formula (Ia), wherein:

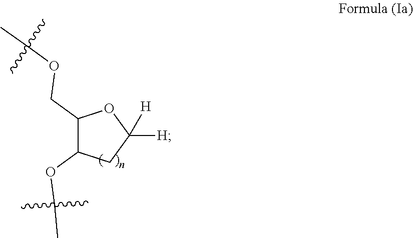

and

n is 0, 1, 2 or 3.

In various embodiments, each of the first, second or third spacers is independently represented by Formula (Ia′), wherein:

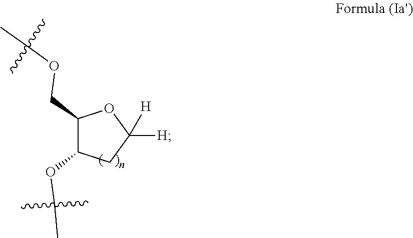

and

n is 0, 1, 2 or 3.

In certain embodiments, each of the first, second or third spacers is independently represented by Formula II, wherein:

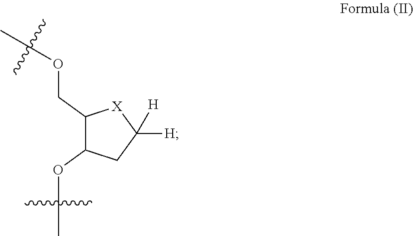

and

X is selected from —CH₂— and

In further embodiments, each of the first, second or third spacers is independently represented by Formula II′, wherein:

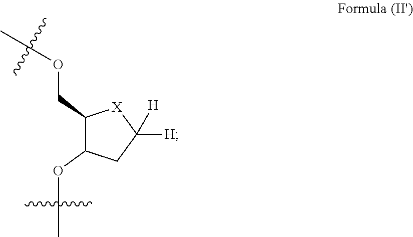

and

X is selected from —CH₂— and —O—.

In various embodiments, each of the first, second or third spacers is independently represented by Formula (IIa), wherein:

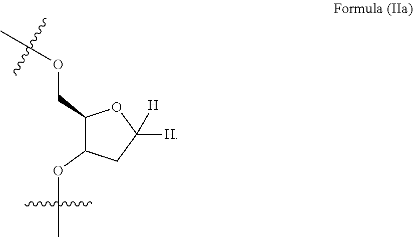

In further embodiments, each of the first, second or third spacers is independently represented by Formula (IIa′), wherein:

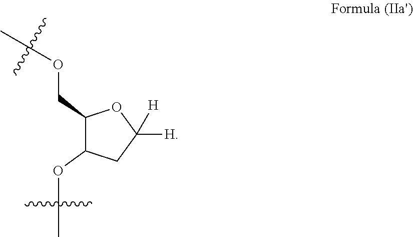

In various embodiments, each of the first, second or third spacers is independently represented by Formula III, wherein:

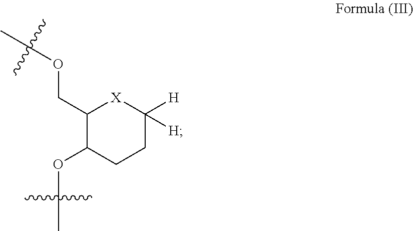

and

X is selected from —CH₂— and

In further embodiments, each of the first, second or third spacers is independently represented by Formula III′, wherein:

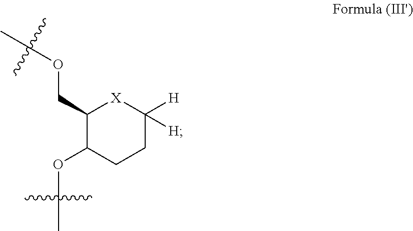

and

X is selected from —CH₂— and —O—.

In some embodiments, each of the first, second or third spacers is independently represented by Formula (IIIa), wherein:

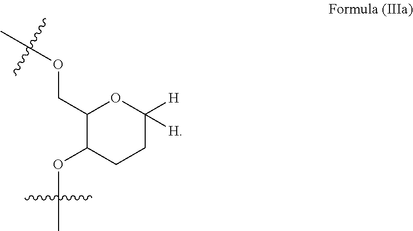

In further embodiments, each of the first, second or third spacers is independently represented by Formula (IIIa′), wherein:

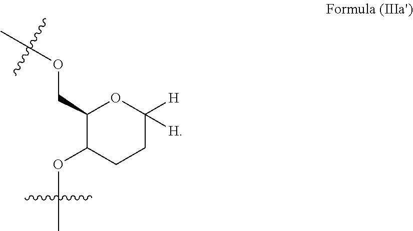

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%.

### BRIEF DESCRIPTION OF THE DRAWINGS

FIG. 1 is a schematic depiction of portions of the STMN2 transcript and STMN2 antisense oligonucleotides that are designed to target certain portions of the STMN2 transcript.

FIG. 2 is a bar graph showing the results of RT-qPCR analysis of TDP43 and STMN2 full-length mRNA levels in the presence of TDP43 antisense, and restoration of the full-length STMN2 transcript in the presence of 6 different STMN2 parent oligonucleotides (SEQ ID NO: 36, SEQ ID NO: 55, SEQ ID NO: 177, SEQ ID NO: 203, SEQ ID NO: 244, and SEQ ID NO: 395).

FIG. 3 is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 parent oligonucleotides (SEQ ID NO: 173, SEQ ID NO: 181, SEQ ID NO: 197, SEQ ID NO: 215, SEQ ID NO: 385, and SEQ ID NO: 400).

FIG. 4 is a bar graph showing the results of RT-qPCR analysis of STMN2 full-length mRNA levels in the presence of TDP43 antisense, and restoration of the full-length STMN2 transcript in the presence of 6 different STMN2 parent oligonucleotides (SEQ ID NO: 173, SEQ ID NO: 181, SEQ ID NO: 197, SEQ ID NO: 215, SEQ ID NO: 385, and SEQ ID NO: 400).

FIG. 5A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 parent oligonucleotides (SEQ ID NO: 185, SEQ ID NO: 209, SEQ ID NO: 237, SEQ ID NO: 252, SEQ ID NO: 380, and SEQ ID NO: 390).

FIG. 5B is a bar graph showing the results of RT-qPCR analysis of STMN2 full-length mRNA levels in the presence of TDP43 antisense, and restoration of the full-length STMN2 transcript in the presence of 6 different STMN2 parent oligonucleotides (SEQ ID NO: 185, SEQ ID NO: 209, SEQ ID NO: 237, SEQ ID NO: 252, SEQ ID NO: 380, and SEQ ID NO: 390).

FIG. 6A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 2 different STMN2 parent oligonucleotides (SEQ ID NO: 144 and SEQ ID NO: 237) over two duplicate experiments.

FIG. 6B is a bar graph showing the results of RT-qPCR analysis of STMN2 full-length mRNA levels in the presence of TDP43 antisense, and restoration of the full-length STMN2 transcript in the presence of 2 different STMN2 parent oligonucleotides (SEQ ID NO: 144 and SEQ ID NO: 237) over two duplicate experiments.

FIG. 7A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 parent oligonucleotides (SEQ ID NO: 36, SEQ ID NO: 173, SEQ ID NO: 177, SEQ ID NO: 181, and SEQ ID NO: 185).

FIG. 7B is a bar graph showing the results of RT-qPCR analysis of STMN2 full-length mRNA levels in the presence of TDP43 antisense, and restoration of the full-length STMN2 transcript in the presence of 5 different STMN2 parent oligonucleotides (SEQ ID NO: 36, SEQ ID NO: 173, SEQ ID NO: 177, SEQ ID NO: 181, and SEQ ID NO: 185).

FIG. 8A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 parent oligonucleotides (SEQ ID NO: 197, SEQ ID NO: 203, SEQ ID NO: 237, SEQ ID NO: 380, and SEQ ID NO: 395).

FIG. 8B is a bar graph showing the results of RT-qPCR analysis of STMN2 full-length mRNA levels in the presence of TDP43 antisense, and restoration of the full-length STMN2 transcript in the presence of 5 different STMN2 parent oligonucleotides (SEQ ID NO: 197, SEQ ID NO: 203, SEQ ID NO: 237, SEQ ID NO: 380, and SEQ ID NO: 395).

FIG. 9A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels in the presence of 3 different STMN2 parent oligonucleotides (SEQ ID NO: 144, SEQ ID NO: 173, and SEQ ID NO: 237).

FIG. 9B is a bar graph showing the results of RT-qPCR analysis of TDP43 and STMN2 full-length mRNA levels in the presence of TDP43 siRNA and TDP43 antisense, and restoration of the full-length STMN2 transcript in the presence of 3 different STMN2 parent oligonucleotides (SEQ ID NO: 144, SEQ ID NO: 173, and SEQ ID NO: 237).

FIG. 10A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 181 STMN2 parent oligonucleotide.

FIG. 10B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 181 STMN2 parent oligonucleotide.

FIG. 11A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 185 STMN2 parent oligonucleotide.

FIG. 11B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 185 STMN2 parent oligonucleotide.

FIG. 12A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 197 STMN2 parent oligonucleotide.

FIG. 12B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 197 STMN2 parent oligonucleotide.

FIG. 13A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 144 STMN2 parent oligonucleotide.

FIG. 13B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 144 STMN2 parent oligonucleotide.

FIG. 14A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 173 STMN2 parent oligonucleotide.

FIG. 14B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 173 STMN2 parent oligonucleotide.

FIG. 15A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 237 STMN2 parent oligonucleotide.

FIG. 15B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 237 STMN2 parent oligonucleotide.

FIG. 16 is a protein blot and quantified bar graph showing the normalized quantity of STMN2 full-length mRNA levels in the presence of TDP43 siRNA and TDP43 antisense, and restoration of the full-length STMN2 transcript for 2 different STMN2 parent oligonucleotides (SEQ ID NO: 173 and SEQ ID NO: 237).

FIG. 17A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 237 STMN2 parent oligonucleotide.

FIG. 17B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 237 STMN2 parent oligonucleotide.

FIG. 18A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 185 STMN2 parent oligonucleotide.

FIG. 18B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 185 STMN2 parent oligonucleotide.

FIG. 19A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 173 STMN2 parent oligonucleotide.

FIG. 19B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 173 STMN2 parent oligonucleotide.

FIG. 20A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 237 STMN2 parent oligonucleotide.

FIG. 20B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 237 STMN2 parent oligonucleotide.

FIG. 21A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 173 STMN2 parent oligonucleotide.

FIG. 21B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 173 STMN2 parent oligonucleotide.

FIG. 22A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 144 STMN2 parent oligonucleotide.

FIG. 22B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 144 STMN2 parent oligonucleotide.

FIG. 23 is a bar graph showing reversal of cryptic exon induction using SEQ ID NO: 237 STMN2 parent oligonucleotide even in view of increasing proteasome inhibition.

FIG. 24 shows the dose response curve illustrating increasing restoration of full length STMN2 transcript with increasing concentrations of STMN2 AON.

FIG. 25A shows a protein blot assay demonstrating the qualitative increase of full length STMN2 protein in response to higher concentrations of STMN2 AON.

FIG. 25B shows the quantitated levels of full length STMN2 protein normalized to GAPDH in response to different concentrations of STMN2 AON.

FIG. 26A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of STMN2 AONs including a SEQ ID NO: 144 AON, a SEQ ID NO: 144 AON with two spacers (SEQ ID NO: 1589), a SEQ ID NO: 173 AON, a SEQ ID NO: 173 with two spacers (SEQ ID NO: 1590), a SEQ ID NO: 237 AON, and a SEQ ID NO: 237 AON with two spacers (SEQ ID NO: 1591).

FIG. 26B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of STMN2 AONs including a SEQ ID NO: 144 AON, a SEQ ID NO: 144 AON with two spacers (SEQ ID NO: 1589), a SEQ ID NO: 173 AON, a SEQ ID NO: 173 with two spacers (SEQ ID NO: 1590), a SEQ ID NO: 237 AON, and a SEQ ID NO: 237 AON with two spacers (SEQ ID NO: 1591).

FIG. 27A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418.

FIG. 27B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418.

FIG. 28A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598.

FIG. 28B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598.

FIG. 29A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173 and SEQ ID NO: 1610.

FIG. 29B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173 and SEQ ID NO: 1610.

FIG. 30A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 185 and SEQ ID NO: 1635.

FIG. 30B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 185 and SEQ ID NO: 1635.

FIG. 31A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1347, SEQ ID NO: 1633, and SEQ ID NO: 1634.

FIG. 31B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 1347, SEQ ID NO: 1633, and SEQ ID NO: 1634.

FIG. 32A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 197, SEQ ID NO: 1617, SEQ ID NO: 1618, and SEQ ID NO: 1619.

FIG. 32B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 197, SEQ ID NO: 1617, SEQ ID NO: 1618, and SEQ ID NO: 1619.

FIG. 33A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 252, SEQ ID NO: 1650, SEQ ID NO: 1434, SEQ ID NO: 1651, and SEQ ID NO: 1620.

FIG. 33B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 252, SEQ ID NO: 1650, SEQ ID NO: 1434, SEQ ID NO: 1651, and SEQ ID NO: 1620.

FIG. 34A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1434 and SEQ ID NO: 1620.

FIG. 34B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 1434 and SEQ ID NO: 1620.

FIG. 35 is a bar graph showing normalized STMN2 protein levels following treatment with TDP43 antisense and restoration using STMN2 AONs including SEQ ID NO: 144, SEQ ID NO: 1589, SEQ ID NO: 173, SEQ ID NO: 1616, SEQ ID NO: 237, and SEQ ID NO: 1591.

### DETAILED DESCRIPTION

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

Disclosed herein are oligonucleotides capable of targeting a region of a transcript transcribed from a gene. In various embodiments, such oligonucleotides target a STMN2 transcript. Additionally disclosed herein are oligonucleotides, including antisense oligonucleotide sequences, and methods for treating neurological diseases, such as amyotrophic lateral sclerosis and frontotemporal dementia, and/or neuropathies such as chemotherapy induced neuropathy, using same. In one embodiment, the oligonucleotides target a cryptic exon sequence of STMN2 transcripts, thereby reducing levels of STMN2 transcripts with the cryptic exon sequence. Also disclosed are pharmaceutical compositions comprising STMN2 oligonucleotides that target a region of STMN2 transcripts that comprise a cryptic exon, for treating neurological diseases and/or neuropathies; and manufacture of medicaments containing a disclosed STMN2 oligonucleotide that targets a region of STMN2 transcripts that comprise a cryptic exon to be used in treating a neurological disease and/or neuropathy.

### Definitions

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, a STMN2 antisense oligonucleotide (AON), as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of STMN2 expression and/or activity is desired.

As used herein, “STMN2” (also known as Superior Cervical Ganglion-10 Protein, Stathmin-Like 2, SCGN10, SCG10, Neuronal Growth-Associated Protein, Neuron-Specific Growth-Associated Protein, or Protein SCG10 (Superior Cervical Ganglia NEAR Neural Specific 10) refers to the gene or gene products (e.g., protein or mRNA transcript (including pre-mRNA) encoded by the gene) identified by Entrez Gene ID No. 11075 and allelic variants thereof, as well as orthologs found in non-human species (e.g., non-human primates or mice).

The term “STMN2 transcript” refers to a STMN2 transcript comprising a cryptic exon. Such a STMN2 transcript comprising a cryptic exon can be a STMN2 pre-mRNA sequence or a STMN2 mature RNA sequence. The term “STMN2 transcript comprising a cryptic exon” refers to a STMN2 transcript that includes one or more cryptic exon sequences.

The term “STMN2 oligonucleotide,” “STMN2 antisense oligonucleotide,” or “STMN2 AON” refers to an oligonucleotide that is capable of increasing, restoring, or stabilizing full-length STMN2 activity e.g., full length STMN2 expression, for example, full length STMN2 mRNA and/or full length STMN2 protein expression. Generally, a STMN2 oligonucleotide reduces the level of mature STMN2 transcripts with a cryptic exon by targeting a STMN2 transcript comprising a cryptic exon. For example, the STMN2 oligonucleotide reduces the level of mature STMN2 transcripts with a cryptic exon by repressing premature polyadenylation of STMN2 pre-mRNA and/or increasing, restoring, or stabilizing activity or function of STMN2. In various embodiments, a STMN2 oligonucleotide comprises a sequence that is between 85 and 98% complementary to an equal length portion of a transcript comprising a sequence at least 90% identity to SEQ ID NO: 1339 or SEQ ID NO: 1341, or a contiguous 15 to 50 nucleobase portion of SEQ ID NO: 1339 or SEQ ID NO: 1341.

In various embodiments, STMN2 oligonucleotides are characterized by having one or more spacers, where each spacer divides up the STMN2 oligonucleotide into segments of linked nucleosides. In various embodiments, STMN2 oligonucleotides have two spacers. In one embodiment, STMN2 oligonucleotides have two segments of linked nucleosides separated by one spacer. In one embodiment, STMN2 oligonucleotides have three segments of linked nucleosides separated by two spacers. In such embodiments, STMN2 oligonucleotides have one segment with at most 7 linked nucleosides. For example, a STMN2 oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, STMN2 oligonucleotides have three spacers that divide the STMN2 oligonucleotide into four segments. In various embodiments, each of the four segments of the STMN2 oligonucleotide have at most 7 linked nucleosides.

As used herein, the term “STMN2 oligonucleotide” encompasses a “STMN2 parent oligonucleotide,” a “STMN2 oligonucleotide with one or more spacers” (e.g., STMN2 oligonucleotide with two spacers or a STMN2 oligonucleotide with three spacers), a “STMN2 oligonucleotide variant with one or more spacers.” Examples of STMN2 oligonucleotides include oligonucleotides comprising a sequence of any one of SEQ ID NOs: 1-466, SEQ ID NO: 893-1338, SEQ ID NOs: 1342-1366, and SEQ ID NOs: 1392-1664.

The term “STMN2 parent oligonucleotide” refers to an oligonucleotide that targets a STMN2 transcript with a cryptic exon and is capable of increasing, restoring, or stabilizing full-length STMN2 activity e.g., full length STMN2 expression, for example, full length STMN2 mRNA and/or full length STMN2 protein expression. STMN2 parent oligonucleotides do not include a spacer. Examples of STMN2 parent oligonucleotides include oligonucleotides comprising a sequence of any one of SEQ ID NOs: 1-446 and SEQ ID NOs: 893-1338. As described hereafter, STMN2 oligonucleotide with spacers and STMN2 oligonucleotide variants are described in relation to a corresponding STMN2 parent oligonucleotide.

The term “STMN2 oligonucleotide variant” refers to a STMN2 oligonucleotide that represents a modified version of a corresponding STMN2 parent oligonucleotide. For example, a STMN2 oligonucleotide variant represents a shortened version of a STMN2 parent oligonucleotide. In various embodiments, a STMN2 oligonucleotide variant is any one of a 15mer, 16mer, 17mer, 18mer 19mer, 20mer, 21mer, 22mer or 23mer. Examples of STMN2 oligonucleotide variants include oligonucleotides comprising a sequence of any one of SEQ ID NOs: 1342-1366 or SEQ ID NOs: 1392-1521. In various embodiments, STMN2 oligonucleotide variants comprise one or more spacers. Such STMN2 oligonucleotide variants comprise a sequence of any one of SEQ ID NOs: 1342-1366 and SEQ ID NOs: 1392-1416.

The term “oligonucleotide with one or more spacers” or “oligonucleotide comprising a spacer” refers to an oligonucleotide with at least one spacer. An oligonucleotide with one or more spacers can, in various embodiments, include one spacer, two spacers, three spacers, four spacer, five spacers, six spacers, seven spacers, eight spacers, nine spacers, or ten spacers. In various embodiments, an oligonucleotide comprising one or more spacers includes at least one segment with at most 7 linked nucleosides. For example, as described in a 5′ to 3′ direction, an oligonucleotide comprising a spacer can include a segment with 7 linked nucleosides, followed by a spacer, a second segment with 9 linked nucleosides, followed by a second spacer, and a third segment with 7 linked nucleosides. Here, the first segment of 7 linked nucleosides and the third segment of 7 linked nucleosides each represents segments with at most 7 linked nucleosides. As another example, an oligonucleotide comprising a spacer can include a segment with 10 linked nucleosides, followed by a spacer, a second segment with 10 linked nucleosides, followed by a second spacer, and a third segment with 3 linked nucleosides. Here, the third segment of 3 linked nucleosides represents the segment with at most 7 linked nucleosides. In various embodiments, an oligonucleotide with one or more spacers includes multiple segments with at most 7 linked nucleosides. In various embodiments, every segment of an oligonucleotide with one or more spacers has at most 7 linked nucleosides. For example, the oligonucleotide may be a 23mer and include two spacers that divide the 23mer into three separate segments of 7 linked nucleosides each. Therefore, each segment of the oligonucleotide has at most 7 linked nucleosides.

Generally, STMN2 oligonucleotides comprising one or more spacers are described in reference to a corresponding STMN2 parent oligonucleotide or a corresponding STMN2 oligonucleotide variant. Example STMN2 oligonucleotides comprising one or spacers include any of SEQ ID NOs: 1417-1420 and SEQ ID NOs: 1451-1664.

In the present specification, the term “therapeutically effective amount” means the amount of an oligonucleotide that will elicit the biological or medical response of a tissue, system, animal or human that is being sought by the researcher, veterinarian, medical doctor, or other clinician. In one embodiment, the oligonucleotide comprises a sequence that is between 85 and 98% complementary to an equal length portion of a transcript comprising a sequence at least 90% identity to SEQ ID NO: 1339 or SEQ ID NO: 1341, or a contiguous 15 to 50 nucleobase portion of SEQ ID NO: 1339 or SEQ ID NO: 1341. The oligonucleotide is administered in therapeutically effective amounts to treat and/or prevent a disease, condition, disorder, or state, for example, a neurological disease and/or a neuropathy. Alternatively, a therapeutically effective amount of an oligonucleotide is the quantity required to achieve a desired therapeutic and/or prophylactic effect, such as an amount which results in the prevention of or a decrease in the symptoms associated with a disease associated with reduced STMN2 activity in the motor neurons.

The phrase “a STMN2 oligonucleotide that targets a STMN2 transcript” refers to a STMN2 oligonucleotide that binds to a STMN2 transcript. Example regions of a STMN2 transcript are shown in Table 1, which depicts sequences corresponding to regions of branch points (e.g., branch point 1, 2, and 3) a 3′ splice acceptor region, an ESE binding region, TDP43 binding sites, a cryptic exon, and a Poly A region. In various embodiments, the oligonucleotide binds to a region of a STMN2 transcript with a cryptic exon, the region being located less than 75 nucleobases upstream or downstream to any of the branch points (e.g., branch point 1, 2, and 3) a 3′ splice acceptor region, an ESE binding region, TDP43 binding sites, a cryptic exon, and a Poly A region.

The term “pharmaceutically acceptable salt(s)” as used herein refers to salts of acidic or basic groups that may be present in a STMN2 oligonucleotide used in the present compositions. A STMN2 oligonucleotide included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. A STMN2 oligonucleotide included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, and lithium salts. Pharmaceutically acceptable salts of the disclosure include, for example, pharmaceutically acceptable salts of STMN2 oligonucleotides that include a sequence of any of SEQ ID NOs: 1-466, SEQ ID NO: 893-1338, SEQ ID NOs: 1342-1366, and SEQ ID NOs: 1392-1664.

A STMN2 oligonucleotide of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorous, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). In various embodiments, the STMN2 oligonucleotide can have a mixed configuration of phosphorothioate linkages. For example, the STMN2 oligonucleotide may have five phosphorothioate linkages in a Rp configuration, followed by fifteen phosphorothioate linkages in a Sp configuration, followed by five phosphorothioate linkages in a Rp configuration. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of a STMN2 oligonucleotide of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The STMN2 oligonucleotide disclosed herein can exist in solvated as well as unsolvated forms with pharmaceutically acceptable solvents such as water, ethanol, and the like, and it is intended that the invention embrace both solvated and unsolvated forms.

The disclosure also embraces isotopically labeled compounds of the invention (i.e., isotopically labeled STMN2 oligonucleotide) which are identical to those recited herein, except that one or more atoms are replaced by an atom having an atomic mass or mass number different from the atomic mass or mass number abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P, ³²P, ³³P, ³⁵S, ¹⁸F, and ³⁶Cl, respectively.

Certain isotopically labeled disclosed compounds (e.g., those labeled with ³H, ¹⁴ or ³⁵S) are useful in compound and/or substrate tissue distribution assays. Tritiated (i.e., ³H), carbon-14 (i.e., ¹⁴C), or ³⁵S methionine isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) is used interchangeably as “2′-O-methoxyethyl” in the present disclosure. A sugar moiety in a nucleoside modified with 2′-MOE is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “5-methyl cytosine” (5-MeC) means a cytosine modified with a methyl group attached to the 5 position. A 5-methyl cytosine (5-MeC) is a modified nucleobase.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cap structure” or “terminal cap moiety” means chemical modifications, which have been incorporated at either terminus of an antisense compound.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge. In some embodiments, cEt can be modified. In some embodiments, the cEt can be S-cEt (in an S-constrained ethyl 2′-4′-bridged nucleic acid). In some other embodiments, the cEt can be R-cEt.

As used herein, “internucleoside linkage” refers to the covalent linkage between adjacent nucleosides in an oligonucleotide. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence. As an example to the contrary, two nucleosides separated by a spacer are not contiguous.

As used herein, “locked nucleic acid” or “LNA” or “LNA nucleosides” means nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N®-2′) LNA and ® Oxyamino (4′-CH₂—N®—O-2′) LNA; wherein R is H, C₁-C₁₂ alkyl, or a protecting group (see U.S. Pat. No. 7,427,672, issued on Sep. 23, 2008).

As used herein, LNA compounds include, but are not limited to, compounds having at least one bridge between the 4′ and the 2′ position of the sugar wherein each of the bridges independently comprises 1 or from 2 to 4 linked groups independently selected from [C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₂-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. A-L-methyleneoxy (4′-CH₂—O-2′), an isomer of methyleneoxy (4′-CH₂—O-2′) LNA is also encompassed within the definition of LNA, as used herein.

As used herein, a “spacer” refers to a nucleoside-replacement group (e.g., a non-nucleoside group that replaces a nucleoside present in a STMN2 parent oligonucleotide). The spacer is characterized by the lack of a nucleotide base and by the replacement of the nucleoside sugar moiety with a non-sugar substitute. The non-sugar substitute group of a spacer lacks an aldehyde, ketone, acetal, ketal, hemiacetal or hemiketal group. The non-sugar substitute group of a spacer is thus capable of connecting to the 3′ and 5′ positions of the nucleosides adjacent to the spacer through an internucleoside linker as described herein, but not capable of forming a covalent bond with a nucleotide base (i.e., not capable of linking a nucleobase to another group, such as an internucleoside linkage, conjugate group, or terminal group in an oligonucleotide). Generally, a STMN2 oligonucleotide with a spacer is described in relation to a STMN2 parent oligonucleotide, wherein the spacer replaces a nucleoside of the STMN2 parent oligonucleotide. In all embodiments of the present disclosure, a spacer cannot hybridize to a nucleoside comprising a nucleobase at the corresponding position of a STMN2 transcript, within the numerical order of the length of the AON oligonucleotide (i.e., if the spacer is positioned after nucleoside 4 of an AON (i.e., at position 5 from the 5′-end), the spacer is not complementary to the nucleoside (A, C, G, or U) at the same corresponding position of the target STMN2 transcript)).

As used herein, “mismatch” or a “non-complementary group” refers to the case when a group (e.g., nucleobase) of a first nucleic acid is not capable of pairing with the corresponding group (e.g., nucleobase) of a second or target nucleic acid.

As used herein, “modified internucleoside linkage” refers to a substitution or any change from a naturally occurring internucleoside linkage (e.g., a phosphodiester internucleoside bond).

As used herein, “modified nucleobase” means any nucleobase other than adenine, cytosine, guanine, thymine, or uracil. Examples of a modified nucleobase include 5-methyl cytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, a “modified nucleoside” means a nucleoside having, independently, a modified sugar moiety and/or modified nucleobase. A universal base is a modified nucleobase that can pair with any one of the five unmodified nucleobases. Modified nucleosides include abasic nucleosides, which lack a nucleobase. However, modified nucleosides do not include spacers or other groups that are incapable of linking a nucleobase.

As used herein, “linked nucleosides” are nucleosides that are connected in a contiguous sequence (i.e., no additional nucleosides are presented between those that are linked). In various embodiments, an oligonucleotide may have different segments of linked nucleosides connected through a spacer. Here, the spacer (i.e., nucleoside replacement) is not considered a nucleoside and therefore, divides up the oligonucleotide into two segments of linked nucleosides. The oligonucleotide may have a first segment of Y linked nucleosides (e.g., Y nucleosides that are connected in a contiguous sequence), followed by a spacer, and then a second segment of Z linked nucleosides. Here, the Y and Z linked nucleosides is described in either the 5′ to 3′ direction or the 3′ to 5′ direction. In various embodiments, the first segment consists of 7 or fewer linked nucleosides (e.g., Y=7 or fewer) whereas the second segment comprises 8 or more linked nucleosides (e.g., Z=8 or more).

As used herein, “modified oligonucleotide” means an oligonucleotide comprising at least one (i.e., one or more) modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound.

As used herein, “natural sugar moiety” means a sugar moiety found in DNA (2′-H) or RNA (2′-OH).

As used herein, “naturally occurring internucleoside linkage” means a 3′ to 5′ phosphodiester linkage.

As used herein, “non-complementary nucleobases” refers to a pair of nucleobases that do not form hydrogen bonds with one another or otherwise support hybridization.

As used herein, “nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, non-coding RNA, small interfering ribonucleic acids (siRNA), short-hairpin RNA (shRNA), and microRNAs (miRNA).

As used herein, “nucleobase” means a heterocyclic moiety capable of base pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “nucleobase sequence” means the order of nucleobases independent of any sugar, linkage, and/or nucleobase modification.

As used herein, “nucleoside” refers to a nucleobase linked to a sugar. The term “nucleoside” also includes a “modified nucleoside” which has independently, a modified sugar moiety and/or modified nucleobase.

As used herein, “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by a phosphorodiamidate or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein, “oligonucleotide” means a polymer of one or more segments of linked nucleosides each of which can be modified or unmodified, independent one from another.

As used herein, “hotspot region” is a range of nucleobases on a target nucleic acid amenable to oligomeric compound-mediated modulation of the splicing of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding between complementary nucleobases.

As used herein, “increasing the amount of activity” refers to more transcriptional expression, more accurate splicing resulting in full length mature mRNA and/or protein expression, and/or more activity relative to the transcriptional expression or activity in an untreated or control sample.

### Antisense Therapeutics

Antisense therapeutics are a class of nucleic acid-based compounds that can be used to modulate a transcript, such as mRNA. In various embodiments, antisense therapeutics comprise one or more spacers and can be used to modulate a transcript that is transcribed from a gene, such as a STMN2 pre-mRNA comprising a cryptic exon.

Antisense therapeutics may be single- or double-stranded deoxyribonucleic acid (DNA)-based, ribonucleic acid (RNA)-based, or DNA/RNA chemical analogue compounds. In general, antisense therapeutics are designed to include a sequence that is complementary or nearly complementary to an mRNA or pre-mRNA sequence transcribed from a given gene in order to promote binding between the antisense therapeutic and the pre-mRNA or mRNA. In certain embodiments, antisense therapeutics act by binding to an mRNA or pre-mRNA, thereby inhibiting protein translation, altering pre-mRNA splicing into mature mRNA (e.g., by preventing appropriate proteins such as splicing activator proteins from binding), and/or causing destruction of mRNA. In certain embodiments, the antisense therapeutic sequence is complementary to a portion of a targeted gene's or mRNA's sense sequence. In certain embodiments, antisense therapeutics described herein are oligonucleotide-based compounds that include an oligonucleotide sequence complementary to a pre-mRNA sense, or a portion thereof, and one or more spacers. In certain embodiments, antisense therapeutics described herein can also be nucleotide chemical analog-based compounds.

In certain embodiments, an oligonucleotide, such as disclosed herein, may be an oligonucleotide sequence of 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, or 25 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, the oligonucleotides are 25 oligonucleotide units in length. In particular embodiments, the oligonucleotides are 23 oligonucleotide units in length. In particular embodiments, the oligonucleotides are 21 oligonucleotide units in length. In particular embodiments, the oligonucleotides are 19 oligonucleotide units in length. In various embodiments, the oligonucleotide is at least 18, at least 19, at least 20, at least 21, at least 22, at least 23, at least 24, or at least 25 oligonucleotide units in length. In various embodiments, the oligonucleotide is at least 18 oligonucleotide units in length. In various embodiments, the oligonucleotide is at least 19 oligonucleotide units in length. In various embodiments, the oligonucleotide is at least 20 oligonucleotide units in length. In various embodiments, the oligonucleotide is at least 21 oligonucleotide units in length. In various embodiments, the oligonucleotide is at least 22 oligonucleotide units in length. In various embodiments, the oligonucleotide is at least 23 oligonucleotide units in length. In various embodiments, the oligonucleotide is at least 24 oligonucleotide units in length. In various embodiments, the oligonucleotide is at least 25 oligonucleotide units in length.

In certain embodiments, AONs may include chemically modified nucleosides (for example, 2′-O-methylated nucleosides or 2′-O-(2-methoxyethyl) nucleosides) as well as modified internucleoside linkages (for example, phosphorothioate linkages). In certain embodiments, AONs described herein include oligonucleotide sequences that are complementary to RNA sequences, such as STMN2 mRNA sequences. In certain embodiments, AONs described herein can include chemically modified nucleosides and modified internucleoside linkages (for example, phosphorothioate linkages). In particular embodiments, AONs described herein include one or more spacers.

In various embodiments, the oligonucleotides comprise one or more spacers. In particular embodiments, the oligonucleotides comprise one spacer. In various embodiments, the oligonucleotides comprise two spacers. For example, the oligonucleotide includes 23 oligonucleotide units with 21 nucleobases and two nucleoside replacement groups (e.g., two spacers). Further embodiments of oligonucleotides with one spacer and oligonucleotides with two spacers are described herein.

In some embodiments, an antisense oligonucleotide can be, but is not limited to, inhibitors of a gene transcript (for example, shRNAs, siRNAs, PNAs, LNAs, 2′-O-methyl (2′Ome) antisense oligonucleotide (AON), 2′-O-(2-methoxyethyl) (MOE) AON, or morpholino oligomers (e.g., phosphorodiamidate morpholino (PMO))), or compositions that include such compounds. In some embodiments an oligonucleotide is an antisense oligonucleotide (AON) comprising 2′Ome (e.g., a AON comprising one or more 2′Ome modified sugar), MOE (e.g., a AON comprising one or more MOE modified sugar), peptide nucleic acids (e.g., a AON comprising one or more N-(2-aminoethyl)-glycine units linked by amide bonds or carbonyl methylene linkage as repeating units in place of a sugar-phosphate backbone), locked nucleic acids (e.g., a AON comprising one or more locked ribose, and can be a mixture of 2′-deoxy nucleotides or 2′Ome nucleotides), c-ET (e.g., a AON comprising one or more cET sugar), constrained methoxyethyl (cMOE) (e.g., a AON comprising one or more cMOE sugar), morpholino oligomer (e.g., a AON comprising a backbone comprising one or more PMO), deoxy-2′-fluoro nucleoside (e.g., a AON comprising one or more 2′-fluoro-(3-D-arabinonucleoside), tricyclo-DNAs (tcDNA) (e.g., a AON comprising one or more tcDNA modified sugar), 2′-0,4′-C-Ethylene-bridged nucleic acid (ENA) (e.g., a AON comprising one or more ENA modified sugar), or hexitol nucleic acids (HNA) (e.g., a AON comprising one or more HNA modified sugar). In some embodiments, a AON comprises one or more internucleoside linkage independently selected from a phosphorothioate linkage, phosphodiester linkage, phosphotriester linkage, methylphosphonate linkage, phosphoramidate linkage, a phosphoramidothioate linkage, a thiophosphorodiamidate linkage, phosphorodiamidate morpholino (PMO) (morpholino) linkage, PNA linkage, or any combination of phosphorothioate linkage, phosphodiester linkage, a phosphotriester linkage, methylphosphonate linkage, phosphoramidate linkage, a phosphoramidothioate linkage, thiophosphorodiamidate linkage, phosphorodiamidate morpholino (PMO) (morpholino) linkage, and PNA linkage. In some embodiments, a STMN2 AON comprises one or more phosphorothioate linkage, phosphodiester linkage, or a combination of phosphorothioate and phosphodiester linkages.

Peptide nucleic acids (PNAs) are short, artificially synthesized polymers with a structure that mimics DNA or RNA. PNAs include a backbone composed of repeating N-(2-aminoethyl)-glycine units linked by peptide bonds. In certain embodiments, PNAs described herein can be used as antisense therapeutics that bind to RNA sequences with high specificity and increase, restore, and/or stabilize levels (e.g., full length STMN2 mRNA or protein levels) and/or activity (e.g., biological activity, for example, STMN2 activity).

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. In certain embodiments, LNAs described herein can be used as antisense therapeutics that bind to RNA sequences with high specificity. For example, LNAs can bind to STMN2 pre-RNA and repress premature polyadenylation of STMN2 pre-mRNA, and increase, restore, and/or stabilize STMN2 levels (e.g., STMN2 mRNA or protein levels) and/or activity (e.g., biological activity, for example, STMN2 activity).

Morpholino oligomers are oligonucleotide compounds that include DNA bases attached to a backbone of methylenemorpholine rings linked through phosphorodiamidate groups. In certain embodiments, morpholino oligomers of the present invention can be designed to bind to specific pre-RNA sequence of interest. For example, morpholino oligomers bind to STMN2 pre-RNA thereby repressing premature polyadenylation of the pre-mRNA, and increase, restore, and/or stabilize STMN2 levels (e.g., STMN2 mRNA or protein levels) and/or activity (e.g., biological activity, for example, STMN2 activity). In certain embodiments, STMN2 morpholino oligomers described herein can be used as antisense therapeutics that bind to STMN2 pre-mRNA sequences with high specificity and repress premature polyadenylation of STMN2 pre-mRNA, and increase, restore, and/or stabilize STMN2 levels (e.g., STMN2 mRNA or protein levels) and/or activity (e.g., biological activity, for example, STMN2 activity). In certain embodiments, STMN2 morpholino oligomers described herein can also be used to bind STMN2 pre-mRNA sequences, altering STMN2 pre-mRNA splicing and STMN2 gene expression, and increase, restore, and/or stabilize STMN2 levels (e.g., STMN2 mRNA or protein levels) and/or activity (e.g., biological activity, for example, STMN2 activity).

STMN2 Oligonucleotides Complementary to STMN2 Transcript with a Cryptic Exon

In some embodiments, a STMN2 AON includes a sequence that is between 85 and 98% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In some embodiments, a STMN2 AON includes a sequence that is between 90-95% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 85% and 90% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341).

In various embodiments, a STMN2 AON comprises a sequence that shares at least 85% identity with an equal length portion of any one of SEQ ID NOs: 1-466, SEQ ID NOs: 893-1338, SEQ ID NOs: 1342-1366, or SEQ ID NOs: 1392-1664. In various embodiments, a STMN2 AON comprises a sequence that shares at least 90% identity with an equal length portion of any one of SEQ ID NOs: 1-466, SEQ ID NOs: 893-1338, SEQ ID NOs: 1342-1366, or SEQ ID NOs: 1392-1664.

In various embodiments, the region of the STMN2 transcript targeted by the STMN2 AON is the cryptic exon sequence. In various embodiments, the region of the STMN2 transcript targeted by the STMN2 AON is a sequence located upstream or downstream (e.g., 100 or 200 bases upstream or downstream) of the cryptic exon sequence. In some embodiments, the STMN2 AON comprises a spacer and has a segment having at most 7 linked nucleosides. In some embodiments, the STMN2 AON comprises a spacer and has a segment having at most 6, 5, 4, 3, or 2 linked nucleosides.

STMN2 AON binding specificity can be assessed via measurement of parameters such as dissociation constant, melting temperature ®, or other criteria such as changes in protein or RNA expression levels or other assays that measure STMN2 activity or expression.

In some embodiments, a STMN2 AON can include a non-duplexed oligonucleotide. In some embodiments, a STMN2 AON can include a duplex of two oligonucleotides where the first oligonucleotide includes a nucleobase sequence that is completely or almost completely complementary to a STMN2 pre-mRNA sequence and the second oligonucleotide includes a nucleobase sequence that is complementary to the nucleobase sequence of the first oligonucleotide.

In some embodiments, a STMN2 AON can target STMN2 pre-mRNAs that include a cryptic exon produced from STMN2 genes of one or more species. For example, a STMN2 AON can target a STMN2 pre-mRNA, which includes a cryptic exon, of a mammalian STMN2 gene, for example, a human (i.e., Homo sapiens) STMN2 gene. In particular embodiments, the STMN2 AON targets a human STMN2 pre-mRNA, which includes a cryptic exon. In some embodiments, the STMN2 AON includes a nucleobase sequence that is complementary to a nucleobase sequence of a STMN2 gene or a STMN2 pre-mRNA, which includes a cryptic exon, or a portion thereof.

STMN2 AONs described herein include antisense oligonucleotides comprising the oligonucleotide sequences listed in Table 1 below:

TABLE 1
STMN2 AON Sequences, in each one or more spacers described in the present
disclosure are incorporated for generation of an oligonucleotide of the present invention

| SEQ | | | SEQ | |
|---|---|---|---|---|
| ID | AON Sequence* | | ID | Target Sequence |
| NO: | (5′→3′) | Region | NO: | (5′→3′) |
| 1 | GGAGGGATACCTGTATATTACAAGT | | 447 | ACTTGTAATATACAGGTATCCCTCC |
| 2 | AGGAGGGATACCTGTATATTACAAG | | 448 | CTTGTAATATACAGGTATCCCTCCT |
| 3 | CAGGAGGGATACCTGTATATTACAA | | 449 | TTGTAATATACAGGTATCCCTCCTG |
| 4 | CCAGGAGGGATACCTGTATATTACA | | 450 | TGTAATATACAGGTATCCCTCCTGG |
| 5 | ACCAGGAGGGATACCTGTATATTAC | | 451 | GTAATATACAGGTATCCCTCCTGGT |
| 6 | TACCAGGAGGGATACCTGTATATTA | | 452 | TAATATACAGGTATCCCTCCTGGTA |
| 7 | TTACCAGGAGGGATACCTGTATATT | | 453 | AATATACAGGTATCCCTCCTGGTAA |
| 8 | CTTACCAGGAGGGATACCTGTATAT | | 454 | ATATACAGGTATCCCTCCTGGTAAG |
| 9 | GCTTACCAGGAGGGATACCTGTATA | | 455 | TATACAGGTATCCCTCCTGGTAAGC |
| 10 | AGCTTACCAGGAGGGATACCTGTAT | | 456 | ATACAGGTATCCCTCCTGGTAAGCT |
| 11 | GAGCTTACCAGGAGGGATACCTGTA | | 457 | TACAGGTATCCCTCCTGGTAAGCTC |
| 12 | AGAGCTTACCAGGAGGGATACCTGT | | 458 | ACAGGTATCCCTCCTGGTAAGCTCT |
| 13 | CAGAGCTTACCAGGAGGGATACCTG | | 459 | CAGGTATCCCTCCTGGTAAGCTCTG |
| 14 | CCAGAGCTTACCAGGAGGGATACCT | | 460 | AGGTATCCCTCCTGGTAAGCTCTGG |
| 15 | ACCAGAGCTTACCAGGAGGGATACC | | 461 | GGTATCCCTCCTGGTAAGCTCTGGT |
| 16 | TACCAGAGCTTACCAGGAGGGATAC | | 462 | GTATCCCTCCTGGTAAGCTCTGGTA |
| 17 | ATACCAGAGCTTACCAGGAGGGATA | | 463 | TATCCCTCCTGGTAAGCTCTGGTAT |
| 18 | AATACCAGAGCTTACCAGGAGGGAT | | 464 | ATCCCTCCTGGTAAGCTCTGGTATT |
| 19 | TAATACCAGAGCTTACCAGGAGGGA | | 465 | TCCCTCCTGGTAAGCTCTGGTATTA |
| 20 | ATAATACCAGAGCTTACCAGGAGGG | | 466 | CCCTCCTGGTAAGCTCTGGTATTAT |
| 21 | CATAATACCAGAGCTTACCAGGAGG | | 467 | CCTCCTGGTAAGCTCTGGTATTATG |
| 22 | ACATAATACCAGAGCTTACCAGGAG | | 468 | CTCCTGGTAAGCTCTGGTATTATGT |
| 23 | GACATAATACCAGAGCTTACCAGGA | | 469 | TCCTGGTAAGCTCTGGTATTATGTC |
| 24 | AGACATAATACCAGAGCTTACCAGG | | 470 | CCTGGTAAGCTCTGGTATTATGTCT |
| 25 | AAGACATAATACCAGAGCTTACCAG | | 471 | CTGGTAAGCTCTGGTATTATGTCTT |
| 26 | TAAGACATAATACCAGAGCTTACCA | | 472 | TGGTAAGCTCTGGTATTATGTCTTA |
| 27 | TTAAGACATAATACCAGAGCTTACC | | 473 | GGTAAGCTCTGGTATTATGTCTTAA |
| 28 | GTTAAGACATAATACCAGAGCTTAC | | 474 | GTAAGCTCTGGTATTATGTCTTAAC |
| 29 | TGTTAAGACATAATACCAGAGCTTA | | 475 | TAAGCTCTGGTATTATGTCTTAACA |
| 30 | ATGTTAAGACATAATACCAGAGCTT | branch | 476 | AAGCTCTGGTATTATGTCTTAACAT |
| | | point 1 | | |
| 31 | AATGTTAAGACATAATACCAGAGCT | branch | 477 | AGCTCTGGTATTATGTCTTAACATT |
| | | point 1 | | |
| 32 | AAATGTTAAGACATAATACCAGAGC | branch | 478 | GCTCTGGTATTATGTCTTAACATTT |
| | | point 1 | | |
| 33 | AAAATGTTAAGACATAATACCAGAG | branch | 479 | CTCTGGTATTATGTCTTAACATTTT |
| | | point 1 | | |
| 34 | AAAAATGTTAAGACATAATACCAGA | branch | 480 | TCTGGTATTATGTCTTAACATTTTT |
| | | point 1 | | |
| 35 | TAAAAATGTTAAGACATAATACCAG | branch | 481 | CTGGTATTATGTCTTAACATTTTTA |
| | | point 1 | | |
| 36 | TTAAAAATGTTAAGACATAATACCA | branch | 482 | TGGTATTATGTCTTAACATTTTTAA |
| | | point 1 | | |
| 37 | TTTAAAAATGTTAAGACATAATACC | branch | 483 | GGTATTATGTCTTAACATTTTTAAA |
| | | point 1 | | |
| 38 | ATTTAAAAATGTTAAGACATAATAC | branch | 484 | GTATTATGTCTTAACATTTTTAAAT |
| | | point 1 | | |
| 39 | GATTTAAAAATGTTAAGACATAATA | branch | 485 | TATTATGTCTTAACATTTTTAAATC |
| | | point 1 | | |
| 40 | AGATTTAAAAATGTTAAGACATAAT | branch | 486 | ATTATGTCTTAACATTTTTAAATCT |
| | | point 1 | | |
| 41 | TAGATTTAAAAATGTTAAGACATAA | branch | 487 | TTATGTCTTAACATTTTTAAATCTA |
| | | point 1 | | |
| 42 | ATAGATTTAAAAATGTTAAGACATA | branch | 488 | TATGTCTTAACATTTTTAAATCTAT |
| | | point 1 | | |
| 43 | CATAGATTTAAAAATGTTAAGACAT | branch | 489 | ATGTCTTAACATTTTTAAATCTATG |
| | | point 1 | | |
| 44 | CCATAGATTTAAAAATGTTAAGACA | branch | 490 | TGTCTTAACATTTTTAAATCTATGG |
| | | point 1 | | |
| 45 | ACCATAGATTTAAAAATGTTAAGAC | branch | 491 | GTCTTAACATTTTTAAATCTATGGT |
| | | point 1 | | |
| 46 | TACCATAGATTTAAAAATGTTAAGA | branch | 492 | TCTTAACATTTTTAAATCTATGGTA |
| | | point 1 | | |
| 47 | TTACCATAGATTTAAAAATGTTAAG | | 493 | CTTAACATTTTTAAATCTATGGTAA |
| 48 | ATTACCATAGATTTAAAAATGTTAA | | 494 | TTAACATTTTTAAATCTATGGTAAT |
| 49 | GATTACCATAGATTTAAAAATGTTA | | 495 | TAACATTTTTAAATCTATGGTAATC |
| 50 | AGATTACCATAGATTTAAAAATGTT | Branch | 496 | AACATTTTTAAATCTATGGTAATCT |
| | | point 2 | | |
| 51 | AAGATTACCATAGATTTAAAAATGT | Branch | 497 | ACATTTTTAAATCTATGGTAATCTT |
| | | point 2 | | |
| 52 | AAAGATTACCATAGATTTAAAAATG | Branch | 498 | CATTTTTAAATCTATGGTAATCTTT |
| | | point 2 | | |
| 53 | TAAAGATTACCATAGATTTAAAAAT | Branch | 499 | ATTTTTAAATCTATGGTAATCTTTA |
| | | point 2 | | |
| 54 | GTAAAGATTACCATAGATTTAAAAA | Branch | 500 | TTTTTAAATCTATGGTAATCTTTAC |
| | | point 2 | | |
| 55 | TGTAAAGATTACCATAGATTTAAAA | Branch | 501 | TTTTAAATCTATGGTAATCTTTACA |
| | | point 2 | | |
| 56 | TTGTAAAGATTACCATAGATTTAAA | Branch | 502 | TTTAAATCTATGGTAATCTTTACAA |
| | | point 2 | | |
| 57 | TTTGTAAAGATTACCATAGATTTAA | Branch | 503 | TTAAATCTATGGTAATCTTTACAAA |
| | | point 2 | | |
| 58 | TTTTGTAAAGATTACCATAGATTTA | Branch | 504 | TAAATCTATGGTAATCTTTACAAAA |
| | | point 2 | | |
| 59 | ATTTTGTAAAGATTACCATAGATTT | Branch | 505 | AAATCTATGGTAATCTTTACAAAAT |
| | | point 2 | | |
| 60 | TATTTTGTAAAGATTACCATAGATT | Branch | 506 | AATCTATGGTAATCTTTACAAAATA |
| | | point 2 | | |
| 61 | ATATTTTGTAAAGATTACCATAGAT | Branch | 507 | ATCTATGGTAATCTTTACAAAATAT |
| | | point 2 | | |
| 62 | AATATTTTGTAAAGATTACCATAGA | Branch | 508 | TCTATGGTAATCTTTACAAAATATT |
| | | point 2 | | |
| 63 | AAATATTTTGTAAAGATTACCATAG | Branch | 509 | CTATGGTAATCTTTACAAAATATTT |
| | | point 2 | | |
| 64 | AAAATATTTTGTAAAGATTACCATA | Branch | 510 | TATGGTAATCTTTACAAAATATTTT |
| | | point 2 | | |
| 65 | TAAAATATTTTGTAAAGATTACCAT | Branch | 511 | ATGGTAATCTTTACAAAATATTTTA |
| | | point 2 | | |
| 66 | GTAAAATATTTTGTAAAGATTACCA | Branch | 512 | TGGTAATCTTTACAAAATATTTTAC |
| | | point 2 | | |
| 67 | AGTAAAATATTTTGTAAAGATTACC | | 513 | GGTAATCTTTACAAAATATTTTACT |
| 68 | AAGTAAAATATTTTGTAAAGATTAC | | 514 | GTAATCTTTACAAAATATTTTACTT |
| 69 | GAAGTAAAATATTTTGTAAAGATTA | | 515 | TAATCTTTACAAAATATTTTACTTC |
| 70 | GGAAGTAAAATATTTTGTAAAGATT | | 516 | AATCTTTACAAAATATTTTACTTCC |
| 71 | CGGAAGTAAAATATTTTGTAAAGAT | | 517 | ATCTTTACAAAATATTTTACTTCCG |
| 72 | TCGGAAGTAAAATATTTTGTAAAGA | | 518 | TCTTTACAAAATATTTTACTTCCGA |
| 73 | TTCGGAAGTAAAATATTTTGTAAAG | | 519 | CTTTACAAAATATTTTACTTCCGAA |
| 74 | GTTCGGAAGTAAAATATTTTGTAAA | | 520 | TTTACAAAATATTTTACTTCCGAAC |
| 75 | AGTTCGGAAGTAAAATATTTTGTAA | | 521 | TTACAAAATATTTTACTTCCGAACT |
| 76 | GAGTTCGGAAGTAAAATATTTTGTA | | 522 | TACAAAATATTTTACTTCCGAACTC |
| 77 | TGAGTTCGGAAGTAAAATATTTTGT | | 523 | ACAAAATATTTTACTTCCGAACTCA |
| 78 | ATGAGTTCGGAAGTAAAATATTTTG | | 524 | CAAAATATTTTACTTCCGAACTCAT |
| 79 | TATGAGTTCGGAAGTAAAATATTTT | | 525 | AAAATATTTTACTTCCGAACTCATA |
| 80 | ATATGAGTTCGGAAGTAAAATATTT | | 526 | AAATATTTTACTTCCGAACTCATAT |
| 81 | TATATGAGTTCGGAAGTAAAATATT | | 527 | AATATTTTACTTCCGAACTCATATA |
| 82 | GTATATGAGTTCGGAAGTAAAATAT | | 528 | ATATTTTACTTCCGAACTCATATAC |
| 83 | GGTATATGAGTTCGGAAGTAAAATA | | 529 | TATTTTACTTCCGAACTCATATACC |
| 84 | AGGTATATGAGTTCGGAAGTAAAAT | | 530 | ATTTTACTTCCGAACTCATATACCT |
| 85 | CAGGTATATGAGTTCGGAAGTAAAA | | 531 | TTTTACTTCCGAACTCATATACCTG |
| 86 | CCAGGTATATGAGTTCGGAAGTAAA | | 532 | TTTACTTCCGAACTCATATACCTGG |
| 87 | CCCAGGTATATGAGTTCGGAAGTAA | | 533 | TTACTTCCGAACTCATATACCTGGG |
| 88 | CCCCAGGTATATGAGTTCGGAAGTA | | 534 | TACTTCCGAACTCATATACCTGGGG |
| 89 | TCCCCAGGTATATGAGTTCGGAAGT | | 535 | ACTTCCGAACTCATATACCTGGGGA |
| 90 | ATCCCCAGGTATATGAGTTCGGAAG | | 536 | CTTCCGAACTCATATACCTGGGGAT |
| 91 | AATCCCCAGGTATATGAGTTCGGAA | | 537 | TTCCGAACTCATATACCTGGGGATT |
| 92 | AAATCCCCAGGTATATGAGTTCGGA | | 538 | TCCGAACTCATATACCTGGGGATTT |
| 93 | AAAATCCCCAGGTATATGAGTTCGG | | 539 | CCGAACTCATATACCTGGGGATTTT |
| 94 | TAAAATCCCCAGGTATATGAGTTCG | | 540 | CGAACTCATATACCTGGGGATTTTA |
| 95 | ATAAAATCCCCAGGTATATGAGTTC | | 541 | GAACTCATATACCTGGGGATTTTAT |
| 96 | AATAAAATCCCCAGGTATATGAGTT | | 542 | AACTCATATACCTGGGGATTTTATT |
| 97 | TAATAAAATCCCCAGGTATATGAGT | | 543 | ACTCATATACCTGGGGATTTTATTA |
| 98 | GTAATAAAATCCCCAGGTATATGAG | | 544 | CTCATATACCTGGGGATTTTATTAC |
| 99 | AGTAATAAAATCCCCAGGTATATGA | | 545 | TCATATACCTGGGGATTTTATTACT |
| 100 | GAGTAATAAAATCCCCAGGTATATG | | 546 | CATATACCTGGGGATTTTATTACTC |
| 101 | AGAGTAATAAAATCCCCAGGTATAT | | 547 | ATATACCTGGGGATTTTATTACTCT |
| 102 | CAGAGTAATAAAATCCCCAGGTATA | | 548 | TATACCTGGGGATTTTATTACTCTG |
| 103 | CCAGAGTAATAAAATCCCCAGGTAT | | 549 | ATACCTGGGGATTTTATTACTCTGG |
| 104 | CCCAGAGTAATAAAATCCCCAGGTA | | 550 | TACCTGGGGATTTTATTACTCTGGG |
| 105 | TCCCAGAGTAATAAAATCCCCAGGT | | 551 | ACCTGGGGATTTTATTACTCTGGGA |
| 106 | TTCCCAGAGTAATAAAATCCCCAGG | | 552 | CCTGGGGATTTTATTACTCTGGGAA |
| 107 | ATTCCCAGAGTAATAAAATCCCCAG | | 553 | CTGGGGATTTTATTACTCTGGGAAT |
| 108 | AATTCCCAGAGTAATAAAATCCCCA | | 554 | TGGGGATTTTATTACTCTGGGAATT |
| 109 | TAATTCCCAGAGTAATAAAATCCCC | | 555 | GGGGATTTTATTACTCTGGGAATTA |
| 110 | ATAATTCCCAGAGTAATAAAATCCC | | 556 | GGGATTTTATTACTCTGGGAATTAT |
| ill | CATAATTCCCAGAGTAATAAAATCC | | 557 | GGATTTTATTACTCTGGGAATTATG |
| 112 | ACATAATTCCCAGAGTAATAAAATC | | 558 | GATTTTATTACTCTGGGAATTATGT |
| 113 | CACATAATTCCCAGAGTAATAAAAT | | 559 | ATTTTATTACTCTGGGAATTATGTG |
| 114 | ACACATAATTCCCAGAGTAATAAAA | | 560 | TTTTATTACTCTGGGAATTATGTGT |
| 115 | AACACATAATTCCCAGAGTAATAAA | | 561 | TTTATTACTCTGGGAATTATGTGTT |
| 116 | GAACACATAATTCCCAGAGTAATAA | | 562 | TTATTACTCTGGGAATTATGTGTTC |
| 117 | AGAACACATAATTCCCAGAGTAATA | | 563 | TATTACTCTGGGAATTATGTGTTCT |
| 118 | CAGAACACATAATTCCCAGAGTAAT | | 564 | ATTACTCTGGGAATTATGTGTTCTG |
| 119 | GCAGAACACATAATTCCCAGAGTAA | | 565 | TTACTCTGGGAATTATGTGTTCTGC |
| 120 | GGCAGAACACATAATTCCCAGAGTA | | 566 | TACTCTGGGAATTATGTGTTCTGCC |
| 121 | GGGCAGAACACATAATTCCCAGAGT | | 567 | ACTCTGGGAATTATGTGTTCTGCCC |
| 122 | GGGGCAGAACACATAATTCCCAGAG | | 568 | CTCTGGGAATTATGTGTTCTGCCCC |
| 123 | TGGGGCAGAACACATAATTCCCAGA | | 569 | TCTGGGAATTATGTGTTCTGCCCCA |
| 124 | ATGGGGCAGAACACATAATTCCCAG | | 570 | CTGGGAATTATGTGTTCTGCCCCAT |
| 125 | GATGGGGCAGAACACATAATTCCCA | | 571 | TGGGAATTATGTGTTCTGCCCCATC |
| 126 | TGATGGGGCAGAACACATAATTCCC | | 572 | GGGAATTATGTGTTCTGCCCCATCA |
| 127 | GTGATGGGGCAGAACACATAATTCC | | 573 | GGAATTATGTGTTCTGCCCCATCAC |
| 128 | AGTGATGGGGCAGAACACATAATTC | | 574 | GAATTATGTGTTCTGCCCCATCACT |
| 129 | GAGTGATGGGGCAGAACACATAATT | Branch | 575 | AATTATGTGTTCTGCCCCATCACTC |
| | | point 3 | | |
| 130 | AGAGTGATGGGGCAGAACACATAAT | Branch | 576 | ATTATGTGTTCTGCCCCATCACTCT |
| | | point 3 | | |
| 131 | GAGAGTGATGGGGCAGAACACATAA | Branch | 577 | TTATGTGTTCTGCCCCATCACTCTC |
| | | point 3 | | |
| 132 | AGAGAGTGATGGGGCAGAACACATA | Branch | 578 | TATGTGTTCTGCCCCATCACTCTCT |
| | | point 3 | | |
| 133 | GAGAGAGTGATGGGGCAGAACACAT | Branch | 579 | ATGTGTTCTGCCCCATCACTCTCTC |
| | | point 3 | | |
| 134 | AGAGAGAGTGATGGGGCAGAACACA | Branch | 580 | TGTGTTCTGCCCCATCACTCTCTCT |
| | | point 3 | | |
| 135 | AAGAGAGAGTGATGGGGCAGAACAC | Branch | 581 | GTGTTCTGCCCCATCACTCTCTCTT |
| | | point 3 | | |
| 136 | TAAGAGAGAGTGATGGGGCAGAACA | Branch | 582 | TGTTCTGCCCCATCACTCTCTCTTA |
| | | point 3 | | |
| 137 | TTAAGAGAGAGTGATGGGGCAGAAC | Branch | 583 | GTTCTGCCCCATCACTCTCTCTTAA |
| | | point 3 | | |
| 138 | ATTAAGAGAGAGTGATGGGGCAGAA | Branch | 584 | TTCTGCCCCATCACTCTCTCTTAAT |
| | | point 3 | | |
| 139 | AATTAAGAGAGAGTGATGGGGCAGA | Branch | 585 | TCTGCCCCATCACTCTCTCTTAATT |
| | | point 3 | | |
| 140 | CAATTAAGAGAGAGTGATGGGGCAG | Branch | 586 | CTGCCCCATCACTCTCTCTTAATTG |
| | | point 3 | | |
| 141 | CCAATTAAGAGAGAGTGATGGGGCA | Branch | 587 | TGCCCCATCACTCTCTCTTAATTGG |
| | | point 3 | | |
| 142 | TCCAATTAAGAGAGAGTGATGGGGC | Branch | 588 | GCCCCATCACTCTCTCTTAATTGGA |
| | | point 3 | | |
| 143 | ATCCAATTAAGAGAGAGTGATGGGG | Branch | 589 | CCCCATCACTCTCTCTTAATTGGAT |
| | | point 3 | | |
| 144 | AATCCAATTAAGAGAGAGTGATGGG | Branch | 590 | CCCATCACTCTCTCTTAATTGGATT |
| | | point 3 | | |
| 145 | AAATCCAATTAAGAGAGAGTGATGG | Branch | 591 | CCATCACTCTCTCTTAATTGGATTT |
| | | point 3 | | |
| 146 | AAAATCCAATTAAGAGAGAGTGATG | | 592 | CATCACTCTCTCTTAATTGGATTTT |
| 147 | AAAAATCCAATTAAGAGAGAGTGAT | | 593 | ATCACTCTCTCTTAATTGGATTTTT |
| 148 | TAAAAATCCAATTAAGAGAGAGTGA | | 594 | TCACTCTCTCTTAATTGGATTTTTA |
| 149 | TTAAAAATCCAATTAAGAGAGAGTG | | 595 | CACTCTCTCTTAATTGGATTTTTAA |
| 150 | TTTAAAAATCCAATTAAGAGAGAGT | | 596 | ACTCTCTCTTAATTGGATTTTTAAA |
| 151 | TTTTAAAAATCCAATTAAGAGAGAG | | 597 | CTCTCTCTTAATTGGATTTTTAAAA |
| 152 | ATTTTAAAAATCCAATTAAGAGAGA | | 598 | TCTCTCTTAATTGGATTTTTAAAAT |
| 153 | AATTTTAAAAATCCAATTAAGAGAG | | 599 | CTCTCTTAATTGGATTTTTAAAATT |
| 154 | TAATTTTAAAAATCCAATTAAGAGA | | 600 | TCTCTTAATTGGATTTTTAAAATTA |
| 155 | ATAATTTTAAAAATCCAATTAAGAG | | 601 | CTCTTAATTGGATTTTTAAAATTAT |
| 156 | TATAATTTTAAAAATCCAATTAAGA | | 602 | TCTTAATTGGATTTTTAAAATTATA |
| 157 | ATATAATTTTAAAAATCCAATTAAG | | 603 | CTTAATTGGATTTTTAAAATTATAT |
| 158 | AATATAATTTTAAAAATCCAATTAA | | 604 | TTAATTGGATTTTTAAAATTATATT |
| 159 | GAATATAATTTTAAAAATCCAATTA | | 605 | TAATTGGATTTTTAAAATTATATTC |
| 160 | TGAATATAATTTTAAAAATCCAATT | | 606 | AATTGGATTTTTAAAATTATATTCA |
| 161 | ATGAATATAATTTTAAAAATCCAAT | | 607 | ATTGGATTTTTAAAATTATATTCAT |
| 162 | TATGAATATAATTTTAAAAATCCAA | | 608 | TTGGATTTTTAAAATTATATTCATA |
| 163 | ATATGAATATAATTTTAAAAATCCA | | 609 | TGGATTTTTAAAATTATATTCATAT |
| 164 | AATATGAATATAATTTTAAAAATCC | | 610 | GGATTTTTAAAATTATATTCATATT |
| 165 | CAATATGAATATAATTTTAAAAATC | | 611 | GATTTTTAAAATTATATTCATATTG |
| 166 | GCAATATGAATATAATTTTAAAAAT | | 612 | ATTTTTAAAATTATATTCATATTGC |
| 167 | TGCAATATGAATATAATTTTAAAAA | | 613 | TTTTTAAAATTATATTCATATTGCA |
| 168 | CTGCAATATGAATATAATTTTAAAA | | 614 | TTTTAAAATTATATTCATATTGCAG |
| 169 | CCTGCAATATGAATATAATTTTAAA | | 615 | TTTAAAATTATATTCATATTGCAGG |
| 170 | TCCTGCAATATGAATATAATTTTAA | | 616 | TTAAAATTATATTCATATTGCAGGA |
| 171 | GTCCTGCAATATGAATATAATTTTA | Acceptor | 617 | TAAAATTATATTCATATTGCAGGAC |
| | | site | | |
| 172 | AGTCCTGCAATATGAATATAATTTT | Acceptor | 618 | AAAATTATATTCATATTGCAGGACT |
| | | site | | |
| 173 | GAGTCCTGCAATATGAATATAATTT | Acceptor | 619 | AAATTATATTCATATTGCAGGACTC |
| | | site | | |
| 174 | CGAGTCCTGCAATATGAATATAATT | Acceptor | 620 | AATTATATTCATATTGCAGGACTCG |
| | | site | | |
| 175 | CCGAGTCCTGCAATATGAATATAAT | Acceptor | 621 | ATTATATTCATATTGCAGGACTCGG |
| | | site | | |
| 176 | GCCGAGTCCTGCAATATGAATATAA | Acceptor | 622 | TTATATTCATATTGCAGGACTCGGC |
| | | site | | |
| 177 | TGCCGAGTCCTGCAATATGAATATA | Acceptor | 623 | TATATTCATATTGCAGGACTCGGCA |
| | | site | | |
| 178 | CTGCCGAGTCCTGCAATATGAATAT | Acceptor | 624 | ATATTCATATTGCAGGACTCGGCAG |
| | | site | | |
| 179 | TCTGCCGAGTCCTGCAATATGAATA | Acceptor | 625 | TATTCATATTGCAGGACTCGGCAGA |
| | | site | | |
| 180 | TTCTGCCGAGTCCTGCAATATGAAT | Acceptor | 626 | ATTCATATTGCAGGACTCGGCAGAA |
| | | site | | |
| 181 | CTTCTGCCGAGTCCTGCAATATGAA | Acceptor | 627 | TTCATATTGCAGGACTCGGCAGAAG |
| | | site | | |
| 182 | TCTTCTGCCGAGTCCTGCAATATGA | Acceptor | 628 | TCATATTGCAGGACTCGGCAGAAGA |
| | | site | | |
| 183 | GTCTTCTGCCGAGTCCTGCAATATG | Acceptor | 629 | CATATTGCAGGACTCGGCAGAAGAC |
| | | site | | |
| 184 | GGTCTTCTGCCGAGTCCTGCAATAT | Acceptor | 630 | ATATTGCAGGACTCGGCAGAAGACC |
| | | site | | |
| 185 | AGGTCTTCTGCCGAGTCCTGCAATA | Acceptor | 631 | TATTGCAGGACTCGGCAGAAGACCT |
| | | site | | |
| 186 | AAGGTCTTCTGCCGAGTCCTGCAAT | Acceptor | 632 | ATTGCAGGACTCGGCAGAAGACCTT |
| | | site | | |
| 187 | GAAGGTCTTCTGCCGAGTCCTGCAA | Acceptor | 633 | TTGCAGGACTCGGCAGAAGACCTTC |
| | | site | | |
| 188 | CGAAGGTCTTCTGCCGAGTCCTGCA | Acceptor | 634 | TGCAGGACTCGGCAGAAGACCTTCG |
| | | site | | |
| 189 | TCGAAGGTCTTCTGCCGAGTCCTGC | Acceptor | 635 | GCAGGACTCGGCAGAAGACCTTCGA |
| | | site | | |
| 190 | CTCGAAGGTCTTCTGCCGAGTCCTG | Acceptor | 636 | CAGGACTCGGCAGAAGACCTTCGAG |
| | | site | | |
| 191 | TCTCGAAGGTCTTCTGCCGAGTCCT | ESE | 637 | AGGACTCGGCAGAAGACCTTCGAGA |
| | | Binding | | |
| 192 | CTCTCGAAGGTCTTCTGCCGAGTCC | ESE | 638 | GGACTCGGCAGAAGACCTTCGAGAG |
| | | Binding | | |
| 193 | TCTCTCGAAGGTCTTCTGCCGAGTC | ESE | 639 | GACTCGGCAGAAGACCTTCGAGAGA |
| | | Binding | | |
| 194 | TTCTCTCGAAGGTCTTCTGCCGAGT | ESE | 640 | ACTCGGCAGAAGACCTTCGAGAGAA |
| | | Binding | | |
| 195 | TTTCTCTCGAAGGTCTTCTGCCGAG | ESE | 641 | CTCGGCAGAAGACCTTCGAGAGAAA |
| | | Binding | | |
| 196 | CTTTCTCTCGAAGGTCTTCTGCCGA | ESE | 642 | TCGGCAGAAGACCTTCGAGAGAAAG |
| | | Binding | | |
| 197 | CCTTTCTCTCGAAGGTCTTCTGCCG | ESE | 643 | CGGCAGAAGACCTTCGAGAGAAAGG |
| | | Binding | | |
| 198 | ACCTTTCTCTCGAAGGTCTTCTGCC | ESE | 644 | GGCAGAAGACCTTCGAGAGAAAGGT |
| | | Binding | | |
| 199 | TACCTTTCTCTCGAAGGTCTTCTGC | ESE | 645 | GCAGAAGACCTTCGAGAGAAAGGTA |
| | | Binding | | |
| 200 | CTACCTTTCTCTCGAAGGTCTTCTG | ESE | 646 | CAGAAGACCTTCGAGAGAAAGGTAG |
| | | Binding | | |
| 201 | TCTACCTTTCTCTCGAAGGTCTTCT | ESE | 647 | AGAAGACCTTCGAGAGAAAGGTAGA |
| | | Binding | | |
| 202 | TTCTACCTTTCTCTCGAAGGTCTTC | ESE | 648 | GAAGACCTTCGAGAGAAAGGTAGAA |
| | | Binding | | |
| 203 | TTTCTACCTTTCTCTCGAAGGTCTT | ESE | 649 | AAGACCTTCGAGAGAAAGGTAGAAA |
| | | Binding | | |
| 204 | TTTTCTACCTTTCTCTCGAAGGTCT | ESE | 650 | AGACCTTCGAGAGAAAGGTAGAAAA |
| | | Binding | | |
| 205 | ATTTTCTACCTTTCTCTCGAAGGTC | ESE | 651 | GACCTTCGAGAGAAAGGTAGAAAAT |
| | | Binding | | |
| 206 | TATTTTCTACCTTTCTCTCGAAGGT | ESE | 652 | ACCTTCGAGAGAAAGGTAGAAAATA |
| | | Binding | | |
| 207 | TTATTTTCTACCTTTCTCTCGAAGG | ESE | 653 | CCTTCGAGAGAAAGGTAGAAAATAA |
| | | Binding | | |
| 208 | CTTATTTTCTACCTTTCTCTCGAAG | ESE | 654 | CTTCGAGAGAAAGGTAGAAAATAAG |
| | | Binding | | |
| 209 | TCTTATTTTCTACCTTTCTCTCGAA | ESE | 655 | TTCGAGAGAAAGGTAGAAAATAAGA |
| | | Binding | | |
| 210 | TTCTTATTTTCTACCTTTCTCTCGA | ESE | 656 | TCGAGAGAAAGGTAGAAAATAAGAA |
| | | Binding | | |
| 211 | ATTCTTATTTTCTACCTTTCTCTCG | ESE | 657 | CGAGAGAAAGGTAGAAAATAAGAAT |
| | | Binding | | |
| 212 | AATTCTTATTTTCTACCTTTCTCTC | ESE | 658 | GAGAGAAAGGTAGAAAATAAGAATT |
| | | Binding | | |
| 213 | AAATTCTTATTTTCTACCTTTCTCT | ESE | 659 | AGAGAAAGGTAGAAAATAAGAATTT |
| | | Binding | | |
| 214 | CAAATTCTTATTTTCTACCTTTCTC | ESE | 660 | GAGAAAGGTAGAAAATAAGAATTTG |
| | | Binding | | |
| 215 | CCAAATTCTTATTTTCTACCTTTCT | ESE | 661 | AGAAAGGTAGAAAATAAGAATTTGG |
| | | Binding | | |
| 216 | GCCAAATTCTTATTTTCTACCTTTC | ESE | 662 | GAAAGGTAGAAAATAAGAATTTGGC |
| | | Binding | | |
| 217 | AGCCAAATTCTTATTTTCTACCTTT | ESE | 663 | AAAGGTAGAAAATAAGAATTTGGCT |
| | | Binding | | |
| 218 | GAGCCAAATTCTTATTTTCTACCTT | ESE | 664 | AAGGTAGAAAATAAGAATTTGGCTC |
| | | Binding | | |
| 219 | AGAGCCAAATTCTTATTTTCTACCT | ESE | 665 | AGGTAGAAAATAAGAATTTGGCTCT |
| | | Binding | | |
| 220 | GAGAGCCAAATTCTTATTTTCTACC | ESE | 666 | GGTAGAAAATAAGAATTTGGCTCTC |
| | | Binding | | |
| 221 | AGAGAGCCAAATTCTTATTTTCTAC | ESE | 667 | GTAGAAAATAAGAATTTGGCTCTCT |
| | | Binding | | |
| 222 | CAGAGAGCCAAATTCTTATTTTCTA | | 668 | TAGAAAATAAGAATTTGGCTCTCTG |
| 223 | ACAGAGAGCCAAATTCTTATTTTCT | | 669 | AGAAAATAAGAATTTGGCTCTCTGT |
| 224 | CACAGAGAGCCAAATTCTTATTTTC | | 670 | GAAAATAAGAATTTGGCTCTCTGTG |
| 225 | ACACAGAGAGCCAAATTCTTATTTT | | 671 | AAAATAAGAATTTGGCTCTCTGTGT |
| 226 | CACACAGAGAGCCAAATTCTTATTT | Overlaps | 672 | AAATAAGAATTTGGCTCTCTGTGTG |
| | | TDP-43 | | |
| | | site 1 | | |
| 227 | TCACACAGAGAGCCAAATTCTTATT | Overlaps | 673 | AATAAGAATTTGGCTCTCTGTGTGA |
| | | TDP-43 | | |
| | | site 1 | | |
| 228 | CTCACACAGAGAGCCAAATTCTTAT | Overlaps | 674 | ATAAGAATTTGGCTCTCTGTGTGAG |
| | | TDP-43 | | |
| | | site 1 | | |
| 229 | GCTCACACAGAGAGCCAAATTCTTA | Overlaps | 675 | TAAGAATTTGGCTCTCTGTGTGAGC |
| | | TDP-43 | | |
| | | site 1 | | |
| 230 | TGCTCACACAGAGAGCCAAATTCTT | Overlaps | 676 | AAGAATTTGGCTCTCTGTGTGAGCA |
| | | TDP-43 | | |
| | | site 1 | | |
| 231 | ATGCTCACACAGAGAGCCAAATTCT | Overlaps | 677 | AGAATTTGGCTCTCTGTGTGAGCAT |
| | | TDP-43 | | |
| | | site 1 | | |
| 232 | CATGCTCACACAGAGAGCCAAATTC | Overlaps | 678 | GAATTTGGCTCTCTGTGTGAGCATG |
| | | TDP-43 | | |
| | | site 1 | | |
| 233 | ACATGCTCACACAGAGAGCCAAATT | Overlaps | 679 | AATTTGGCTCTCTGTGTGAGCATGT |
| | | TDP-43 | | |
| | | site 1 | | |
| 234 | CACATGCTCACACAGAGAGCCAAAT | Overlaps | 680 | ATTTGGCTCTCTGTGTGAGCATGTG |
| | | TDP-43 | | |
| | | site 1 | | |
| 235 | ACACATGCTCACACAGAGAGCCAAA | Overlaps | 681 | TTTGGCTCTCTGTGTGAGCATGTGT |
| | | TDP-43 | | |
| | | site 1 | | |
| 236 | CACACATGCTCACACAGAGAGCCAA | Overlaps | 682 | TTGGCTCTCTGTGTGAGCATGTGTG |
| | | TDP-43 | | |
| | | site 1 & | | |
| | | 2 | | |
| 237 | GCACACATGCTCACACAGAGAGCCA | Overlaps | 683 | TGGCTCTCTGTGTGAGCATGTGTGC |
| | | TDP-43 | | |
| | | site 1 & | | |
| | | 2 | | |
| 238 | CGCACACATGCTCACACAGAGAGCC | Overlaps | 684 | GGCTCTCTGTGTGAGCATGTGTGCG |
| | | TDP-43 | | |
| | | site 1 & | | |
| | | 2 | | |
| 239 | ACGCACACATGCTCACACAGAGAGC | Overlaps | 685 | GCTCTCTGTGTGAGCATGTGTGCGT |
| | | TDP-43 | | |
| | | site 1 & | | |
| | | 2 | | |
| 240 | CACGCACACATGCTCACACAGAGAG | Overlaps | 686 | CTCTCTGTGTGAGCATGTGTGCGTG |
| | | TDP-43 | | |
| | | site 1 & | | |
| | | 2 | | |
| 241 | ACACGCACACATGCTCACACAGAGA | Overlaps | 687 | TCTCTGTGTGAGCATGTGTGCGTGT |
| | | TDP-43 | | |
| | | site 1 & | | |
| | | 2 | | |
| 242 | CACACGCACACATGCTCACACAGAG | Overlaps | 688 | CTCTGTGTGAGCATGTGTGCGTGTG |
| | | TDP-43 | | |
| | | site 1 & | | |
| | | 2 | | |
| 243 | ACACACGCACACATGCTCACACAGA | Overlaps | 689 | TCTGTGTGAGCATGTGTGCGTGTGT |
| | | TDP-43 | | |
| | | site 1 & | | |
| | | 2 | | |
| 244 | CACACACGCACACATGCTCACACAG | Overlaps | 690 | CTGTGTGAGCATGTGTGCGTGTGTG |
| | | TDP-43 | | |
| | | site 1 & | | |
| | | 2&3 | | |
| 245 | GCACACACGCACACATGCTCACACA | Overlaps | 691 | TGTGTGAGCATGTGTGCGTGTGTGC |
| | | TDP-43 | | |
| | | site 1 & | | |
| | | 2&3 | | |
| 246 | CGCACACACGCACACATGCTCACAC | Overlaps | 692 | GTGTGAGCATGTGTGCGTGTGTGCG |
| | | TDP-43 | | |
| | | site 2 & | | |
| | | 3 | | |
| 247 | TCGCACACACGCACACATGCTCACA | Overlaps | 693 | TGTGAGCATGTGTGCGTGTGTGCGA |
| | | TDP-43 | | |
| | | site 2 & | | |
| | | 3 | | |
| 248 | CTCGCACACACGCACACATGCTCAC | Overlaps | 694 | GTGAGCATGTGTGCGTGTGTGCGAG |
| | | TDP-43 | | |
| | | site 2 & | | |
| | | 3 | | |
| 249 | TCTCGCACACACGCACACATGCTCA | Overlaps | 695 | TGAGCATGTGTGCGTGTGTGCGAGA |
| | | TDP-43 | | |
| | | site 2 & | | |
| | | 3 | | |
| 250 | CTCTCGCACACACGCACACATGCTC | Overlaps | 696 | GAGCATGTGTGCGTGTGTGCGAGAG |
| | | TDP-43 | | |
| | | site 2 & | | |
| | | 3 | | |
| 251 | TCTCTCGCACACACGCACACATGCT | Overlaps | 697 | AGCATGTGTGCGTGTGTGCGAGAGA |
| | | TDP-43 | | |
| | | site 2 & | | |
| | | 3 | | |
| 252 | CTCTCTCGCACACACGCACACATGC | Overlaps | 698 | GCATGTGTGCGTGTGTGCGAGAGAG |
| | | TDP-43 | | |
| | | site 2 & | | |
| | | 3 | | |
| 253 | TCTCTCTCGCACACACGCACACATG | Overlaps | 699 | CATGTGTGCGTGTGTGCGAGAGAGA |
| | | TDP-43 | | |
| | | site 2 & | | |
| | | 3 | | |
| 254 | CTCTCTCTCGCACACACGCACACAT | Overlaps | 700 | ATGTGTGCGTGTGTGCGAGAGAGAG |
| | | TDP-43 | | |
| | | site 2 & | | |
| | | 3 | | |
| 255 | TCTCTCTCTCGCACACACGCACACA | Overlaps | 701 | TGTGTGCGTGTGTGCGAGAGAGAGA |
| | | TDP-43 | | |
| | | site 2 & | | |
| | | 3 | | |
| 256 | CTCTCTCTCTCGCACACACGCACAC | Overlaps | 702 | GTGTGCGTGTGTGCGAGAGAGAGAG |
| | | TDP-43 | | |
| | | site 3 | | |
| 257 | TCTCTCTCTCTCGCACACACGCACA | Overlaps | 703 | TGTGCGTGTGTGCGAGAGAGAGAGA |
| | | TDP-43 | | |
| | | site 3 | | |
| 258 | GTCTCTCTCTCTCGCACACACGCAC | Overlaps | 704 | GTGCGTGTGTGCGAGAGAGAGAGAC |
| | | TDP-43 | | |
| | | site 3 | | |
| 259 | TGTCTCTCTCTCTCGCACACACGCA | Overlaps | 705 | TGCGTGTGTGCGAGAGAGAGAGACA |
| | | TDP-43 | | |
| | | site 3 | | |
| 260 | CTGTCTCTCTCTCTCGCACACACGC | Overlaps | 706 | GCGTGTGTGCGAGAGAGAGAGACAG |
| | | TDP-43 | | |
| | | site 3 | | |
| 261 | TCTGTCTCTCTCTCTCGCACACACG | Overlaps | 707 | CGTGTGTGCGAGAGAGAGAGACAGA |
| | | TDP-43 | | |
| | | site 3 | | |
| 262 | GTCTGTCTCTCTCTCTCGCACACAC | Overlaps | 708 | GTGTGTGCGAGAGAGAGAGACAGAC |
| | | TDP-43 | | |
| | | site 3 | | |
| 263 | TGTCTGTCTCTCTCTCTCGCACACA | Overlaps | 709 | TGTGTGCGAGAGAGAGAGACAGACA |
| | | TDP-43 | | |
| | | site 3 | | |
| 264 | CTGTCTGTCTCTCTCTCTCGCACAC | | 710 | GTGTGCGAGAGAGAGAGACAGACAG |
| 265 | GCTGTCTGTCTCTCTCTCTCGCACA | | 711 | TGTGCGAGAGAGAGAGACAGACAGC |
| 266 | GGCTGTCTGTCTCTCTCTCTCGCAC | | 712 | GTGCGAGAGAGAGAGACAGACAGCC |
| 267 | AGGCTGTCTGTCTCTCTCTCTCGCA | | 713 | TGCGAGAGAGAGAGACAGACAGCCT |
| 268 | CAGGCTGTCTGTCTCTCTCTCTCGC | | 714 | GCGAGAGAGAGAGACAGACAGCCTG |
| 269 | GCAGGCTGTCTGTCTCTCTCTCTCG | | 715 | CGAGAGAGAGAGACAGACAGCCTGC |
| 270 | GGCAGGCTGTCTGTCTCTCTCTCTC | | 716 | GAGAGAGAGAGACAGACAGCCTGCC |
| 271 | AGGCAGGCTGTCTGTCTCTCTCTCT | | 717 | AGAGAGAGAGACAGACAGCCTGCCT |
| 272 | TAGGCAGGCTGTCTGTCTCTCTCTC | | 718 | GAGAGAGAGACAGACAGCCTGCCTA |
| 273 | TTAGGCAGGCTGTCTGTCTCTCTCT | | 719 | AGAGAGAGACAGACAGCCTGCCTAA |
| 274 | CTTAGGCAGGCTGTCTGTCTCTCTC | | 720 | GAGAGAGACAGACAGCCTGCCTAAG |
| 275 | TCTTAGGCAGGCTGTCTGTCTCTCT | | 721 | AGAGAGACAGACAGCCTGCCTAAGA |
| 276 | TTCTTAGGCAGGCTGTCTGTCTCTC | | 722 | GAGAGACAGACAGCCTGCCTAAGAA |
| 277 | CTTCTTAGGCAGGCTGTCTGTCTCT | | 723 | AGAGACAGACAGCCTGCCTAAGAAG |
| 278 | TCTTCTTAGGCAGGCTGTCTGTCTC | | 724 | GAGACAGACAGCCTGCCTAAGAAGA |
| 279 | TTCTTCTTAGGCAGGCTGTCTGTCT | | 725 | AGACAGACAGCCTGCCTAAGAAGAA |
| 280 | TTTCTTCTTAGGCAGGCTGTCTGTC | | 726 | GACAGACAGCCTGCCTAAGAAGAAA |
| 281 | ATTTCTTCTTAGGCAGGCTGTCTGT | | 727 | ACAGACAGCCTGCCTAAGAAGAAAT |
| 282 | CATTTCTTCTTAGGCAGGCTGTCTG | | 728 | CAGACAGCCTGCCTAAGAAGAAATG |
| 283 | TCATTTCTTCTTAGGCAGGCTGTCT | | 729 | AGACAGCCTGCCTAAGAAGAAATGA |
| 284 | TTCATTTCTTCTTAGGCAGGCTGTC | | 730 | GACAGCCTGCCTAAGAAGAAATGAA |
| 285 | ATTCATTTCTTCTTAGGCAGGCTGT | | 731 | ACAGCCTGCCTAAGAAGAAATGAAT |
| 286 | CATTCATTTCTTCTTAGGCAGGCTG | | 732 | CAGCCTGCCTAAGAAGAAATGAATG |
| 287 | ACATTCATTTCTTCTTAGGCAGGCT | | 733 | AGCCTGCCTAAGAAGAAATGAATGT |
| 288 | CACATTCATTTCTTCTTAGGCAGGC | | 734 | GCCTGCCTAAGAAGAAATGAATGTG |
| 289 | TCACATTCATTTCTTCTTAGGCAGG | | 735 | CCTGCCTAAGAAGAAATGAATGTGA |
| 290 | TTCACATTCATTTCTTCTTAGGCAG | | 736 | CTGCCTAAGAAGAAATGAATGTGAA |
| 291 | ATTCACATTCATTTCTTCTTAGGCA | | 737 | TGCCTAAGAAGAAATGAATGTGAAT |
| 292 | CATTCACATTCATTTCTTCTTAGGC | | 738 | GCCTAAGAAGAAATGAATGTGAATG |
| 293 | GCATTCACATTCATTTCTTCTTAGG | | 739 | CCTAAGAAGAAATGAATGTGAATGC |
| 294 | CGCATTCACATTCATTTCTTCTTAG | | 740 | CTAAGAAGAAATGAATGTGAATGCG |
| 295 | CCGCATTCACATTCATTTCTTCTTA | | 741 | TAAGAAGAAATGAATGTGAATGCGG |
| 296 | GCCGCATTCACATTCATTTCTTCTT | | 742 | AAGAAGAAATGAATGTGAATGCGGC |
| 297 | AGCCGCATTCACATTCATTTCTTCT | | 743 | AGAAGAAATGAATGTGAATGCGGCT |
| 298 | AAGCCGCATTCACATTCATTTCTTC | | 744 | GAAGAAATGAATGTGAATGCGGCTT |
| 299 | CAAGCCGCATTCACATTCATTTCTT | | 745 | AAGAAATGAATGTGAATGCGGCTTG |
| 300 | ACAAGCCGCATTCACATTCATTTCT | | 746 | AGAAATGAATGTGAATGCGGCTTGT |
| 301 | CACAAGCCGCATTCACATTCATTTC | | 747 | GAAATGAATGTGAATGCGGCTTGTG |
| 302 | CCACAAGCCGCATTCACATTCATTT | | 748 | AAATGAATGTGAATGCGGCTTGTGG |
| 303 | GCCACAAGCCGCATTCACATTCATT | | 749 | AATGAATGTGAATGCGGCTTGTGGC |
| 304 | TGCCACAAGCCGCATTCACATTCAT | | 750 | ATGAATGTGAATGCGGCTTGTGGCA |
| 305 | GTGCCACAAGCCGCATTCACATTCA | | 751 | TGAATGTGAATGCGGCTTGTGGCAC |
| 306 | TGTGCCACAAGCCGCATTCACATTC | | 752 | GAATGTGAATGCGGCTTGTGGCACA |
| 307 | CTGTGCCACAAGCCGCATTCACATT | | 753 | AATGTGAATGCGGCTTGTGGCACAG |
| 308 | ACTGTGCCACAAGCCGCATTCACAT | | 754 | ATGTGAATGCGGCTTGTGGCACAGT |
| 309 | AACTGTGCCACAAGCCGCATTCACA | | 755 | TGTGAATGCGGCTTGTGGCACAGTT |
| 310 | CAACTGTGCCACAAGCCGCATTCAC | | 756 | GTGAATGCGGCTTGTGGCACAGTTG |
| 311 | TCAACTGTGCCACAAGCCGCATTCA | | 757 | TGAATGCGGCTTGTGGCACAGTTGA |
| 312 | GTCAACTGTGCCACAAGCCGCATTC | | 758 | GAATGCGGCTTGTGGCACAGTTGAC |
| 313 | TGTCAACTGTGCCACAAGCCGCATT | | 759 | AATGCGGCTTGTGGCACAGTTGACA |
| 314 | TTGTCAACTGTGCCACAAGCCGCAT | | 760 | ATGCGGCTTGTGGCACAGTTGACAA |
| 315 | CTTGTCAACTGTGCCACAAGCCGCA | | 761 | TGCGGCTTGTGGCACAGTTGACAAG |
| 316 | CCTTGTCAACTGTGCCACAAGCCGC | | 762 | GCGGCTTGTGGCACAGTTGACAAGG |
| 317 | TCCTTGTCAACTGTGCCACAAGCCG | | 763 | CGGCTTGTGGCACAGTTGACAAGGA |
| 318 | ATCCTTGTCAACTGTGCCACAAGCC | | 764 | GGCTTGTGGCACAGTTGACAAGGAT |
| 319 | CATCCTTGTCAACTGTGCCACAAGC | | 765 | GCTTGTGGCACAGTTGACAAGGATG |
| 320 | TCATCCTTGTCAACTGTGCCACAAG | | 766 | CTTGTGGCACAGTTGACAAGGATGA |
| 321 | ATCATCCTTGTCAACTGTGCCACAA | | 767 | TTGTGGCACAGTTGACAAGGATGAT |
| 322 | TATCATCCTTGTCAACTGTGCCACA | | 768 | TGTGGCACAGTTGACAAGGATGATA |
| 323 | TTATCATCCTTGTCAACTGTGCCAC | | 769 | GTGGCACAGTTGACAAGGATGATAA |
| 324 | TTTATCATCCTTGTCAACTGTGCCA | | 770 | TGGCACAGTTGACAAGGATGATAAA |
| 325 | ATTTATCATCCTTGTCAACTGTGCC | | 771 | GGCACAGTTGACAAGGATGATAAAT |
| 326 | GATTTATCATCCTTGTCAACTGTGC | | 772 | GCACAGTTGACAAGGATGATAAATC |
| 327 | TGATTTATCATCCTTGTCAACTGTG | | 773 | CACAGTTGACAAGGATGATAAATCA |
| 328 | TTGATTTATCATCCTTGTCAACTGT | | 774 | ACAGTTGACAAGGATGATAAATCAA |
| 329 | ATTGATTTATCATCCTTGTCAACTG | | 775 | CAGTTGACAAGGATGATAAATCAAT |
| 330 | TATTGATTTATCATCCTTGTCAACT | | 776 | AGTTGACAAGGATGATAAATCAATA |
| 331 | TTATTGATTTATCATCCTTGTCAAC | | 777 | GTTGACAAGGATGATAAATCAATAA |
| 332 | ATTATTGATTTATCATCCTTGTCAA | | 778 | TTGACAAGGATGATAAATCAATAAT |
| 333 | CATTATTGATTTATCATCCTTGTCA | | 779 | TGACAAGGATGATAAATCAATAATG |
| 334 | GCATTATTGATTTATCATCCTTGTC | | 780 | GACAAGGATGATAAATCAATAATGC |
| 335 | TGCATTATTGATTTATCATCCTTGT | | 781 | ACAAGGATGATAAATCAATAATGCA |
| 336 | TTGCATTATTGATTTATCATCCTTG | | 782 | CAAGGATGATAAATCAATAATGCAA |
| 337 | CTTGCATTATTGATTTATCATCCTT | | 783 | AAGGATGATAAATCAATAATGCAAG |
| 338 | GCTTGCATTATTGATTTATCATCCT | | 784 | AGGATGATAAATCAATAATGCAAGC |
| 339 | AGCTTGCATTATTGATTTATCATCC | | 785 | GGATGATAAATCAATAATGCAAGCT |
| 340 | AAGCTTGCATTATTGATTTATCATC | | 786 | GATGATAAATCAATAATGCAAGCTT |
| 341 | TAAGCTTGCATTATTGATTTATCAT | | 787 | ATGATAAATCAATAATGCAAGCTTA |
| 342 | GTAAGCTTGCATTATTGATTTATCA | | 788 | TGATAAATCAATAATGCAAGCTTAC |
| 343 | AGTAAGCTTGCATTATTGATTTATC | | 789 | GATAAATCAATAATGCAAGCTTACT |
| 344 | TAGTAAGCTTGCATTATTGATTTAT | | 790 | ATAAATCAATAATGCAAGCTTACTA |
| 345 | ATAGTAAGCTTGCATTATTGATTTA | | 791 | TAAATCAATAATGCAAGCTTACTAT |
| 346 | GATAGTAAGCTTGCATTATTGATTT | | 792 | AAATCAATAATGCAAGCTTACTATC |
| 347 | TGATAGTAAGCTTGCATTATTGATT | | 793 | AATCAATAATGCAAGCTTACTATCA |
| 348 | ATGATAGTAAGCTTGCATTATTGAT | | 794 | ATCAATAATGCAAGCTTACTATCAT |
| 349 | AATGATAGTAAGCTTGCATTATTGA | | 795 | TCAATAATGCAAGCTTACTATCATT |
| 350 | AAATGATAGTAAGCTTGCATTATTG | | 796 | CAATAATGCAAGCTTACTATCATTT |
| 351 | TAAATGATAGTAAGCTTGCATTATT | | 797 | AATAATGCAAGCTTACTATCATTTA |
| 352 | ATAAATGATAGTAAGCTTGCATTAT | | 798 | ATAATGCAAGCTTACTATCATTTAT |
| 353 | CATAAATGATAGTAAGCTTGCATTA | | 799 | TAATGCAAGCTTACTATCATTTATG |
| 354 | TCATAAATGATAGTAAGCTTGCATT | | 800 | AATGCAAGCTTACTATCATTTATGA |
| 355 | TTCATAAATGATAGTAAGCTTGCAT | | 801 | ATGCAAGCTTACTATCATTTATGAA |
| 356 | ATTCATAAATGATAGTAAGCTTGCA | | 802 | TGCAAGCTTACTATCATTTATGAAT |
| 357 | TATTCATAAATGATAGTAAGCTTGC | | 803 | GCAAGCTTACTATCATTTATGAATA |
| 358 | CTATTCATAAATGATAGTAAGCTTG | | 804 | CAAGCTTACTATCATTTATGAATAG |
| 359 | GCTATTCATAAATGATAGTAAGCTT | | 805 | AAGCTTACTATCATTTATGAATAGC |
| 360 | TGCTATTCATAAATGATAGTAAGCT | | 806 | AGCTTACTATCATTTATGAATAGCA |
| 361 | TTGCTATTCATAAATGATAGTAAGC | | 807 | GCTTACTATCATTTATGAATAGCAA |
| 362 | ATTGCTATTCATAAATGATAGTAAG | | 808 | CTTACTATCATTTATGAATAGCAAT |
| 363 | TATTGCTATTCATAAATGATAGTAA | | 809 | TTACTATCATTTATGAATAGCAATA |
| 364 | GTATTGCTATTCATAAATGATAGTA | | 810 | TACTATCATTTATGAATAGCAATAC |
| 365 | AGTATTGCTATTCATAAATGATAGT | | 811 | ACTATCATTTATGAATAGCAATACT |
| 366 | CAGTATTGCTATTCATAAATGATAG | | 812 | CTATCATTTATGAATAGCAATACTG |
| 367 | TCAGTATTGCTATTCATAAATGATA | | 813 | TATCATTTATGAATAGCAATACTGA |
| 368 | TTCAGTATTGCTATTCATAAATGAT | | 814 | ATCATTTATGAATAGCAATACTGAA |
| 369 | CTTCAGTATTGCTATTCATAAATGA | | 815 | TCATTTATGAATAGCAATACTGAAG |
| 370 | TCTTCAGTATTGCTATTCATAAATG | | 816 | CATTTATGAATAGCAATACTGAAGA |
| 371 | TTCTTCAGTATTGCTATTCATAAAT | | 817 | ATTTATGAATAGCAATACTGAAGAA |
| 372 | TTTCTTCAGTATTGCTATTCATAAA | | 818 | TTTATGAATAGCAATACTGAAGAAA |
| 373 | ATTTCTTCAGTATTGCTATTCATAA | | 819 | TTATGAATAGCAATACTGAAGAAAT |
| 374 | AATTTCTTCAGTATTGCTATTCATA | | 820 | TATGAATAGCAATACTGAAGAAATT |
| 375 | TAATTTCTTCAGTATTGCTATTCAT | | 821 | ATGAATAGCAATACTGAAGAAATTA |
| 376 | TTAATTTCTTCAGTATTGCTATTCA | | 822 | TGAATAGCAATACTGAAGAAATTAA |
| 377 | TTTAATTTCTTCAGTATTGCTATTC | polyA | 823 | GAATAGCAATACTGAAGAAATTAAA |
| | | signal | | |
| 378 | TTTTAATTTCTTCAGTATTGCTATT | polyA | 824 | AATAGCAATACTGAAGAAATTAAAA |
| | | signal | | |
| 379 | GTTTTAATTTCTTCAGTATTGCTAT | polyA | 825 | ATAGCAATACTGAAGAAATTAAAAC |
| | | signal | | |
| 380 | TGTTTTAATTTCTTCAGTATTGCTA | polyA | 826 | TAGCAATACTGAAGAAATTAAAACA |
| | | signal | | |
| 381 | TTGTTTTAATTTCTTCAGTATTGCT | polyA | 827 | AGCAATACTGAAGAAATTAAAACAA |
| | | signal | | |
| 382 | TTTGTTTTAATTTCTTCAGTATTGC | polyA | 828 | GCAATACTGAAGAAATTAAAACAAA |
| | | signal | | |
| 383 | TTTTGTTTTAATTTCTTCAGTATTG | polyA | 829 | CAATACTGAAGAAATTAAAACAAAA |
| | | signal | | |
| 384 | CTTTTGTTTTAATTTCTTCAGTATT | polyA | 830 | AATACTGAAGAAATTAAAACAAAAG |
| | | signal | | |
| 385 | TCTTTTGTTTTAATTTCTTCAGTAT | polyA | 831 | ATACTGAAGAAATTAAAACAAAAGA |
| | | signal | | |
| 386 | ATCTTTTGTTTTAATTTCTTCAGTA | polyA | 832 | TACTGAAGAAATTAAAACAAAAGAT |
| | | signal | | |
| 387 | AATCTTTTGTTTTAATTTCTTCAGT | polyA | 833 | ACTGAAGAAATTAAAACAAAAGATT |
| | | signal | | |
| 388 | CAATCTTTTGTTTTAATTTCTTCAG | polyA | 834 | CTGAAGAAATTAAAACAAAAGATTG |
| | | signal | | |
| 389 | GCAATCTTTTGTTTTAATTTCTTCA | polyA | 835 | TGAAGAAATTAAAACAAAAGATTGC |
| | | signal | | |
| 390 | AGCAATCTTTTGTTTTAATTTCTTC | polyA | 836 | GAAGAAATTAAAACAAAAGATTGCT |
| | | signal | | |
| 391 | CAGCAATCTTTTGTTTTAATTTCTT | polyA | 837 | AAGAAATTAAAACAAAAGATTGCTG |
| | | signal | | |
| 392 | ACAGCAATCTTTTGTTTTAATTTCT | polyA | 838 | AGAAATTAAAACAAAAGATTGCTGT |
| | | signal | | |
| 393 | GACAGCAATCTTTTGTTTTAATTTC | polyA | 839 | GAAATTAAAACAAAAGATTGCTGTC |
| | | signal | | |
| 394 | AGACAGCAATCTTTTGTTTTAATTT | polyA | 840 | AAATTAAAACAAAAGATTGCTGTCT |
| | | signal | | |
| 395 | GAGACAGCAATCTTTTGTTTTAATT | polyA | 841 | AATTAAAACAAAAGATTGCTGTCTC |
| | | signal | | |
| | | and site | | |
| 396 | TGAGACAGCAATCTTTTGTTTTAAT | polyA | 842 | ATTAAAACAAAAGATTGCTGTCTCA |
| | | signal | | |
| | | and site | | |
| 397 | TTGAGACAGCAATCTTTTGTTTTAA | polyA | 843 | TTAAAACAAAAGATTGCTGTCTCAA |
| | | site | | |
| 398 | ATTGAGACAGCAATCTTTTGTTTTA | polyA | 844 | TAAAACAAAAGATTGCTGTCTCAAT |
| | | site | | |
| 399 | TATTGAGACAGCAATCTTTTGTTTT | polyA | 845 | AAAACAAAAGATTGCTGTCTCAATA |
| | | site | | |
| 400 | ATATTGAGACAGCAATCTTTTGTTT | polyA | 846 | AAACAAAAGATTGCTGTCTCAATAT |
| | | site | | |
| 401 | TATATTGAGACAGCAATCTTTTGTT | polyA | 847 | AACAAAAGATTGCTGTCTCAATATA |
| | | site | | |
| 402 | ATATATTGAGACAGCAATCTTTTGT | polyA | 848 | ACAAAAGATTGCTGTCTCAATATAT |
| | | site | | |
| 403 | GATATATTGAGACAGCAATCTTTTG | polyA | 849 | CAAAAGATTGCTGTCTCAATATATC |
| | | site | | |
| 404 | AGATATATTGAGACAGCAATCTTTT | polyA | 850 | AAAAGATTGCTGTCTCAATATATCT |
| | | site | | |
| 405 | AAGATATATTGAGACAGCAATCTTT | polyA | 851 | AAAGATTGCTGTCTCAATATATCTT |
| | | site | | |
| 406 | TAAGATATATTGAGACAGCAATCTT | polyA | 852 | AAGATTGCTGTCTCAATATATCTTA |
| | | site | | |
| 407 | ATAAGATATATTGAGACAGCAATCT | polyA | 853 | AGATTGCTGTCTCAATATATCTTAT |
| | | site | | |
| 408 | TATAAGATATATTGAGACAGCAATC | polyA | 854 | GATTGCTGTCTCAATATATCTTATA |
| | | site | | |
| 409 | ATATAAGATATATTGAGACAGCAAT | polyA | 855 | ATTGCTGTCTCAATATATCTTATAT |
| | | site | | |
| 410 | AATATAAGATATATTGAGACAGCAA | polyA | 856 | TTGCTGTCTCAATATATCTTATATT |
| | | site | | |
| 411 | AAATATAAGATATATTGAGACAGCA | polyA | 857 | TGCTGTCTCAATATATCTTATATTT |
| | | site | | |
| 412 | TAAATATAAGATATATTGAGACAGC | polyA | 858 | GCTGTCTCAATATATCTTATATTTA |
| | | site | | |
| 413 | ATAAATATAAGATATATTGAGACAG | | 859 | CTGTCTCAATATATCTTATATTTAT |
| 414 | AATAAATATAAGATATATTGAGACA | | 860 | TGTCTCAATATATCTTATATTTATT |
| 415 | TAATAAATATAAGATATATTGAGAC | | 861 | GTCTCAATATATCTTATATTTATTA |
| 416 | ATAATAAATATAAGATATATTGAGA | | 862 | TCTCAATATATCTTATATTTATTAT |
| 417 | AATAATAAATATAAGATATATTGAG | | 863 | CTCAATATATCTTATATTTATTATT |
| 418 | AAATAATAAATATAAGATATATTGA | | 864 | TCAATATATCTTATATTTATTATTT |
| 419 | TAAATAATAAATATAAGATATATTG | | 865 | CAATATATCTTATATTTATTATTTA |
| 420 | GTAAATAATAAATATAAGATATATT | | 866 | AATATATCTTATATTTATTATTTAC |
| 421 | GGTAAATAATAAATATAAGATATAT | | 867 | ATATATCTTATATTTATTATTTACC |
| 422 | TGGTAAATAATAAATATAAGATATA | | 868 | TATATCTTATATTTATTATTTACCA |
| 423 | TTGGTAAATAATAAATATAAGATAT | | 869 | ATATCTTATATTTATTATTTACCAA |
| 424 | TTTGGTAAATAATAAATATAAGATA | | 870 | TATCTTATATTTATTATTTACCAAA |
| 425 | ATTTGGTAAATAATAAATATAAGAT | | 871 | ATCTTATATTTATTATTTACCAAAT |
| 426 | AATTTGGTAAATAATAAATATAAGA | | 872 | TCTTATATTTATTATTTACCAAATT |
| 427 | TAATTTGGTAAATAATAAATATAAG | | 873 | CTTATATTTATTATTTACCAAATTA |
| 428 | ATAATTTGGTAAATAATAAATATAA | | 874 | TTATATTTATTATTTACCAAATTAT |
| 429 | AATAATTTGGTAAATAATAAATATA | | 875 | TATATTTATTATTTACCAAATTATT |
| 430 | GAATAATTTGGTAAATAATAAATAT | | 876 | ATATTTATTATTTACCAAATTATTC |
| 431 | AGAATAATTTGGTAAATAATAAATA | | 877 | TATTTATTATTTACCAAATTATTCT |
| 432 | TAGAATAATTTGGTAAATAATAAAT | | 878 | ATTTATTATTTACCAAATTATTCTA |
| 433 | TTAGAATAATTTGGTAAATAATAAA | | 879 | TTTATTATTTACCAAATTATTCTAA |
| 434 | CTTAGAATAATTTGGTAAATAATAA | | 880 | TTATTATTTACCAAATTATTCTAAG |
| 435 | TCTTAGAATAATTTGGTAAATAATA | | 881 | TATTATTTACCAAATTATTCTAAGA |
| 436 | CTCTTAGAATAATTTGGTAAATAAT | | 882 | ATTATTTACCAAATTATTCTAAGAG |
| 437 | ACTCTTAGAATAATTTGGTAAATAA | | 883 | TTATTTACCAAATTATTCTAAGAGT |
| 438 | TACTCTTAGAATAATTTGGTAAATA | | 884 | TATTTACCAAATTATTCTAAGAGTA |
| 439 | ATACTCTTAGAATAATTTGGTAAAT | | 885 | ATTTACCAAATTATTCTAAGAGTAT |
| 440 | AATACTCTTAGAATAATTTGGTAAA | | 886 | TTTACCAAATTATTCTAAGAGTATT |
| 441 | AAATACTCTTAGAATAATTTGGTAA | | 887 | TTACCAAATTATTCTAAGAGTATTT |
| 442 | GAAATACTCTTAGAATAATTTGGTA | | 888 | TACCAAATTATTCTAAGAGTATTTC |
| 443 | AGAAATACTCTTAGAATAATTTGGT | | 889 | ACCAAATTATTCTAAGAGTATTTCT |
| 444 | AAGAAATACTCTTAGAATAATTTGG | | 890 | CCAAATTATTCTAAGAGTATTTCTT |
| 445 | GAAGAAATACTCTTAGAATAATTTG | | 891 | CAAATTATTCTAAGAGTATTTCTTC |
| 446 | GGAAGAAATACTCTTAGAATAATTT | | 892 | AAATTATTCTAAGAGTATTTCTTCC |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 2 below identifies additional STMN2 AON sequences:

TABLE 2
Additional STMN2 AON Sequences (corresponding
to SEQ ID NOs: 1-446 but with thymine bases
replaced with uracil bases)

| SEQ | |
|---|---|
| ID | |
| NO: | AON Sequence* (5’ → 3’) |
| 893 | GGAGGGAUACCUGUAUAUUACAAGU |
| 894 | AGGAGGGAUACCUGUAUAUUACAAG |
| 895 | CAGGAGGGAUACCUGUAUAUUACAA |
| 896 | CCAGGAGGGAUACCUGUAUAUUACA |
| 897 | ACCAGGAGGGAUACCUGUAUAUUAC |
| 898 | UACCAGGAGGGAUACCUGUAUAUUA |
| 899 | UUACCAGGAGGGAUACCUGUAUAUU |
| 900 | CUUACCAGGAGGGAUACCUGUAUAU |
| 901 | GCUUACCAGGAGGGAUACCUGUAUA |
| 902 | AGCUUACCAGGAGGGAUACCUGUAU |
| 903 | GAGCUUACCAGGAGGGAUACCUGUA |
| 904 | AGAGCUUACCAGGAGGGAUACCUGU |
| 905 | CAGAGCUUACCAGGAGGGAUACCUG |
| 906 | CCAGAGCUUACCAGGAGGGAUACCU |
| 907 | ACCAGAGCUUACCAGGAGGGAUACC |
| 908 | UACCAGAGCUUACCAGGAGGGAUAC |
| 909 | AUACCAGAGCUUACCAGGAGGGAUA |
| 910 | AAUACCAGAGCUUACCAGGAGGGAU |
| 911 | UAAUACCAGAGCUUACCAGGAGGGA |
| 912 | AUAAUACCAGAGCUUACCAGGAGGG |
| 913 | CAUAAUACCAGAGCUUACCAGGAGG |
| 914 | ACAUAAUACCAGAGCUUACCAGGAG |
| 915 | GACAUAAUACCAGAGCUUACCAGGA |
| 916 | AGACAUAAUACCAGAGCUUACCAGG |
| 917 | AAGACAUAAUACCAGAGCUUACCAG |
| 918 | UAAGACAUAAUACCAGAGCUUACCA |
| 919 | UUAAGACAUAAUACCAGAGCUUACC |
| 920 | GUUAAGACAUAAUACCAGAGCUUAC |
| 921 | UGUUAAGACAUAAUACCAGAGCUUA |
| 922 | AUGUUAAGACAUAAUACCAGAGCUU |
| 923 | AAUGUUAAGACAUAAUACCAGAGCU |
| 924 | AAAUGUUAAGACAUAAUACCAGAGC |
| 925 | AAAAUGUUAAGACAUAAUACCAGAG |
| 926 | AAAAAUGUUAAGACAUAAUACCAGA |
| 927 | UAAAAAUGUUAAGACAUAAUACCAG |
| 928 | UUAAAAAUGUUAAGACAUAAUACCA |
| 929 | UUUAAAAAUGUUAAGACAUAAUACC |
| 930 | AUUUAAAAAUGUUAAGACAUAAUAC |
| 931 | GAUUUAAAAAUGUUAAGACAUAAUA |
| 932 | AGAUUUAAAAAUGUUAAGACAUAAU |
| 933 | UAGAUUUAAAAAUGUUAAGACAUAA |
| 934 | AUAGAUUUAAAAAUGUUAAGACAUA |
| 935 | CAUAGAUUUAAAAAUGUUAAGACAU |
| 936 | CCAUAGAUUUAAAAAUGUUAAGACA |
| 937 | ACCAUAGAUUUAAAAAUGUUAAGAC |
| 938 | UACCAUAGAUUUAAAAAUGUUAAGA |
| 939 | UUACCAUAGAUUUAAAAAUGUUAAG |
| 940 | AUUACCAUAGAUUUAAAAAUGUUAA |
| 941 | GAUUACCAUAGAUUUAAAAAUGUUA |
| 942 | AGAUUACCAUAGAUUUAAAAAUGUU |
| 943 | AAGAUUACCAUAGAUUUAAAAAUGU |
| 944 | AAAGAUUACCAUAGAUUUAAAAAUG |
| 945 | UAAAGAUUACCAUAGAUUUAAAAAU |
| 946 | GUAAAGAUUACCAUAGAUUUAAAAA |
| 947 | UGUAAAGAUUACCAUAGAUUUAAAA |
| 948 | UUGUAAAGAUUACCAUAGAUUUAAA |
| 949 | UUUGUAAAGAUUACCAUAGAUUUAA |
| 950 | UUUUGUAAAGAUUACCAUAGAUUUA |
| 951 | AUUUUGUAAAGAUUACCAUAGAUUU |
| 952 | UAUUUUGUAAAGAUUACCAUAGAUU |
| 953 | AUAUUUUGUAAAGAUUACCAUAGAU |
| 954 | AAUAUUUUGUAAAGAUUACCAUAGA |
| 955 | AAAUAUUUUGUAAAGAUUACCAUAG |
| 956 | AAAAUAUUUUGUAAAGAUUACCAUA |
| 957 | UAAAAUAUUUUGUAAAGAUUACCAU |
| 958 | GUAAAAUAUUUUGUAAAGAUUACCA |
| 959 | AGUAAAAUAUUUUGUAAAGAUUACC |
| 960 | AAGUAAAAUAUUUUGUAAAGAUUAC |
| 961 | GAAGUAAAAUAUUUUGUAAAGAUUA |
| 962 | GGAAGUAAAAUAUUUUGUAAAGAUU |
| 963 | CGGAAGUAAAAUAUUUUGUAAAGAU |
| 964 | UCGGAAGUAAAAUAUUUUGUAAAGA |
| 965 | UUCGGAAGUAAAAUAUUUUGUAAAG |
| 966 | GUUCGGAAGUAAAAUAUUUUGUAAA |
| 967 | AGUUCGGAAGUAAAAUAUUUUGUAA |
| 968 | GAGUUCGGAAGUAAAAUAUUUUGUA |
| 969 | UGAGUUCGGAAGUAAAAUAUUUUGU |
| 970 | AUGAGUUCGGAAGUAAAAUAUUUUG |
| 971 | UAUGAGUUCGGAAGUAAAAUAUUUU |
| 972 | AUAUGAGUUCGGAAGUAAAAUAUUU |
| 973 | UAUAUGAGUUCGGAAGUAAAAUAUU |
| 974 | GUAUAUGAGUUCGGAAGUAAAAUAU |
| 975 | GGUAUAUGAGUUCGGAAGUAAAAUA |
| 976 | AGGUAUAUGAGUUCGGAAGUAAAAU |
| 977 | CAGGUAUAUGAGUUCGGAAGUAAAA |
| 978 | CCAGGUAUAUGAGUUCGGAAGUAAA |
| 979 | CCCAGGUAUAUGAGUUCGGAAGUAA |
| 980 | CCCCAGGUAUAUGAGUUCGGAAGUA |
| 981 | UCCCCAGGUAUAUGAGUUCGGAAGU |
| 982 | AUCCCCAGGUAUAUGAGUUCGGAAG |
| 983 | AAUCCCCAGGUAUAUGAGUUCGGAA |
| 984 | AAAUCCCCAGGUAUAUGAGUUCGGA |
| 985 | AAAAUCCCCAGGUAUAUGAGUUCGG |
| 986 | UAAAAUCCCCAGGUAUAUGAGUUCG |
| 987 | AUAAAAUCCCCAGGUAUAUGAGUUC |
| 988 | AAUAAAAUCCCCAGGUAUAUGAGUU |
| 989 | UAAUAAAAUCCCCAGGUAUAUGAGU |
| 990 | GUAAUAAAAUCCCCAGGUAUAUGAG |
| 991 | AGUAAUAAAAUCCCCAGGUAUAUGA |
| 992 | GAGUAAUAAAAUCCCCAGGUAUAUG |
| 993 | AGAGUAAUAAAAUCCCCAGGUAUAU |
| 994 | CAGAGUAAUAAAAUCCCCAGGUAUA |
| 995 | CCAGAGUAAUAAAAUCCCCAGGUAU |
| 996 | CCCAGAGUAAUAAAAUCCCCAGGUA |
| 997 | UCCCAGAGUAAUAAAAUCCCCAGGU |
| 998 | UUCCCAGAGUAAUAAAAUCCCCAGG |
| 999 | AUUCCCAGAGUAAUAAAAUCCCCAG |
| 1000 | AAUUCCCAGAGUAAUAAAAUCCCCA |
| 1001 | UAAUUCCCAGAGUAAUAAAAUCCCC |
| 1002 | AUAAUUCCCAGAGUAAUAAAAUCCC |
| 1003 | CAUAAUUCCCAGAGUAAUAAAAUCC |
| 1004 | ACAUAAUUCCCAGAGUAAUAAAAUC |
| 1005 | CACAUAAUUCCCAGAGUAAUAAAAU |
| 1006 | ACACAUAAUUCCCAGAGUAAUAAAA |
| 1007 | AACACAUAAUUCCCAGAGUAAUAAA |
| 1008 | GAACACAUAAUUCCCAGAGUAAUAA |
| 1009 | AGAACACAUAAUUCCCAGAGUAAUA |
| 1010 | CAGAACACAUAAUUCCCAGAGUAAU |
| 1011 | GCAGAACACAUAAUUCCCAGAGUAA |
| 1012 | GGCAGAACACAUAAUUCCCAGAGUA |
| 1013 | GGGCAGAACACAUAAUUCCCAGAGU |
| 1014 | GGGGCAGAACACAUAAUUCCCAGAG |
| 1015 | UGGGGCAGAACACAUAAUUCCCAGA |
| 1016 | AUGGGGCAGAACACAUAAUUCCCAG |
| 1017 | GAUGGGGCAGAACACAUAAUUCCCA |
| 1018 | UGAUGGGGCAGAACACAUAAUUCCC |
| 1019 | GUGAUGGGGCAGAACACAUAAUUCC |
| 1020 | AGUGAUGGGGCAGAACACAUAAUUC |
| 1021 | GAGUGAUGGGGCAGAACACAUAAUU |
| 1022 | AGAGUGAUGGGGCAGAACACAUAAU |
| 1023 | GAGAGUGAUGGGGCAGAACACAUAA |
| 1024 | AGAGAGUGAUGGGGCAGAACACAUA |
| 1025 | GAGAGAGUGAUGGGGCAGAACACAU |
| 1026 | AGAGAGAGUGAUGGGGCAGAACACA |
| 1027 | AAGAGAGAGUGAUGGGGCAGAACAC |
| 1028 | UAAGAGAGAGUGAUGGGGCAGAACA |
| 1029 | UUAAGAGAGAGUGAUGGGGCAGAAC |
| 1030 | AUUAAGAGAGAGUGAUGGGGCAGAA |
| 1031 | AAUUAAGAGAGAGUGAUGGGGCAGA |
| 1032 | CAAUUAAGAGAGAGUGAUGGGGCAG |
| 1033 | CCAAUUAAGAGAGAGUGAUGGGGCA |
| 1034 | UCCAAUUAAGAGAGAGUGAUGGGGC |
| 1035 | AUCCAAUUAAGAGAGAGUGAUGGGG |
| 1036 | AAUCCAAUUAAGAGAGAGUGAUGGG |
| 1037 | AAAUCCAAUUAAGAGAGAGUGAUGG |
| 1038 | AAAAUCCAAUUAAGAGAGAGUGAUG |
| 1039 | AAAAAUCCAAUUAAGAGAGAGUGAU |
| 1040 | UAAAAAUCCAAUUAAGAGAGAGUGA |
| 1041 | UUAAAAAUCCAAUUAAGAGAGAGUG |
| 1042 | UUUAAAAAUCCAAUUAAGAGAGAGU |
| 1043 | UUUUAAAAAUCCAAUUAAGAGAGAG |
| 1044 | AUUUUAAAAAUCCAAUUAAGAGAGA |
| 1045 | AAUUUUAAAAAUCCAAUUAAGAGAG |
| 1046 | UAAUUUUAAAAAUCCAAUUAAGAGA |
| 1047 | AUAAUUUUAAAAAUCCAAUUAAGAG |
| 1048 | UAUAAUUUUAAAAAUCCAAUUAAGA |
| 1049 | AUAUAAUUUUAAAAAUCCAAUUAAG |
| 1050 | AAUAUAAUUUUAAAAAUCCAAUUAA |
| 1051 | GAAUAUAAUUUUAAAAAUCCAAUUA |
| 1052 | UGAAUAUAAUUUUAAAAAUCCAAUU |
| 1053 | AUGAAUAUAAUUUUAAAAAUCCAAU |
| 1054 | UAUGAAUAUAAUUUUAAAAAUCCAA |
| 1055 | AUAUGAAUAUAAUUUUAAAAAUCCA |
| 1056 | AAUAUGAAUAUAAUUUUAAAAAUCC |
| 1057 | CAAUAUGAAUAUAAUUUUAAAAAUC |
| 1058 | GCAAUAUGAAUAUAAUUUUAAAAAU |
| 1059 | UGCAAUAUGAAUAUAAUUUUAAAAA |
| 1060 | CUGCAAUAUGAAUAUAAUUUUAAAA |
| 1061 | CCUGCAAUAUGAAUAUAAUUUUAAA |
| 1062 | UCCUGCAAUAUGAAUAUAAUUUUAA |
| 1063 | GUCCUGCAAUAUGAAUAUAAUUUUA |
| 1064 | AGUCCUGCAAUAUGAAUAUAAUUUU |
| 1065 | GAGUCCUGCAAUAUGAAUAUAAUUU |
| 1066 | CGAGUCCUGCAAUAUGAAUAUAAUU |
| 1067 | CCGAGUCCUGCAAUAUGAAUAUAAU |
| 1068 | GCCGAGUCCUGCAAUAUGAAUAUAA |
| 1069 | UGCCGAGUCCUGCAAUAUGAAUAUA |
| 1070 | CUGCCGAGUCCUGCAAUAUGAAUAU |
| 1071 | UCUGCCGAGUCCUGCAAUAUGAAUA |
| 1072 | UUCUGCCGAGUCCUGCAAUAUGAAU |
| 1073 | CUUCUGCCGAGUCCUGCAAUAUGAA |
| 1074 | UCUUCUGCCGAGUCCUGCAAUAUGA |
| 1075 | GUCUUCUGCCGAGUCCUGCAAUAUG |
| 1076 | GGUCUUCUGCCGAGUCCUGCAAUAU |
| 1077 | AGGUCUUCUGCCGAGUCCUGCAAUA |
| 1078 | AAGGUCUUCUGCCGAGUCCUGCAAU |
| 1079 | GAAGGUCUUCUGCCGAGUCCUGCAA |
| 1080 | CGAAGGUCUUCUGCCGAGUCCUGCA |
| 1081 | UCGAAGGUCUUCUGCCGAGUCCUGC |
| 1082 | CUCGAAGGUCUUCUGCCGAGUCCUG |
| 1083 | UCUCGAAGGUCUUCUGCCGAGUCCU |
| 1084 | CUCUCGAAGGUCUUCUGCCGAGUCC |
| 1085 | UCUCUCGAAGGUCUUCUGCCGAGUC |
| 1086 | UUCUCUCGAAGGUCUUCUGCCGAGU |
| 1087 | UUUCUCUCGAAGGUCUUCUGCCGAG |
| 1088 | CUUUCUCUCGAAGGUCUUCUGCCGA |
| 1089 | CCUUUCUCUCGAAGGUCUUCUGCCG |
| 1090 | ACCUUUCUCUCGAAGGUCUUCUGCC |
| 1091 | UACCUUUCUCUCGAAGGUCUUCUGC |
| 1092 | CUACCUUUCUCUCGAAGGUCUUCUG |
| 1093 | UCUACCUUUCUCUCGAAGGUCUUCU |
| 1094 | UUCUACCUUUCUCUCGAAGGUCUUC |
| 1095 | UUUCUACCUUUCUCUCGAAGGUCUU |
| 1096 | UUUUCUACCUUUCUCUCGAAGGUCU |
| 1097 | AUUUUCUACCUUUCUCUCGAAGGUC |
| 1098 | UAUUUUCUACCUUUCUCUCGAAGGU |
| 1099 | UUAUUUUCUACCUUUCUCUCGAAGG |
| 1100 | CUUAUUUUCUACCUUUCUCUCGAAG |
| 1101 | UCUUAUUUUCUACCUUUCUCUCGAA |
| 1102 | UUCUUAUUUUCUACCUUUCUCUCGA |
| 1103 | AUUCUUAUUUUCUACCUUUCUCUCG |
| 1104 | AAUUCUUAUUUUCUACCUUUCUCUC |
| 1105 | AAAUUCUUAUUUUCUACCUUUCUCU |
| 1106 | CAAAUUCUUAUUUUCUACCUUUCUC |
| 1107 | CCAAAUUCUUAUUUUCUACCUUUCU |
| 1108 | GCCAAAUUCUUAUUUUCUACCUUUC |
| 1109 | AGCCAAAUUCUUAUUUUCUACCUUU |
| 1110 | GAGCCAAAUUCUUAUUUUCUACCUU |
| 1111 | AGAGCCAAAUUCUUAUUUUCUACCU |
| 1112 | GAGAGCCAAAUUCUUAUUUUCUACC |
| 1113 | AGAGAGCCAAAUUCUUAUUUUCUAC |
| 1114 | CAGAGAGCCAAAUUCUUAUUUUCUA |
| 1115 | ACAGAGAGCCAAAUUCUUAUUUUCU |
| 1116 | CACAGAGAGCCAAAUUCUUAUUUUC |
| 1117 | ACACAGAGAGCCAAAUUCUUAUUUU |
| 1118 | CACACAGAGAGCCAAAUUCUUAUUU |
| 1119 | UCACACAGAGAGCCAAAUUCUUAUU |
| 1120 | CUCACACAGAGAGCCAAAUUCUUAU |
| 1121 | GCUCACACAGAGAGCCAAAUUCUUA |
| 1122 | UGCUCACACAGAGAGCCAAAUUCUU |
| 1123 | AUGCUCACACAGAGAGCCAAAUUCU |
| 1124 | CAUGCUCACACAGAGAGCCAAAUUC |
| 1125 | ACAUGCUCACACAGAGAGCCAAAUU |
| 1126 | CACAUGCUCACACAGAGAGCCAAAU |
| 1127 | ACACAUGCUCACACAGAGAGCCAAA |
| 1128 | CACACAUGCUCACACAGAGAGCCAA |
| 1129 | GCACACAUGCUCACACAGAGAGCCA |
| 1130 | CGCACACAUGCUCACACAGAGAGCC |
| 1131 | ACGCACACAUGCUCACACAGAGAGC |
| 1132 | CACGCACACAUGCUCACACAGAGAG |
| 1133 | ACACGCACACAUGCUCACACAGAGA |
| 1134 | CACACGCACACAUGCUCACACAGAG |
| 1135 | ACACACGCACACAUGCUCACACAGA |
| 1136 | CACACACGCACACAUGCUCACACAG |
| 1137 | GCACACACGCACACAUGCUCACACA |
| 1138 | CGCACACACGCACACAUGCUCACAC |
| 1139 | UCGCACACACGCACACAUGCUCACA |
| 1140 | CUCGCACACACGCACACAUGCUCAC |
| 1141 | UCUCGCACACACGCACACAUGCUCA |
| 1142 | CUCUCGCACACACGCACACAUGCUC |
| 1143 | UCUCUCGCACACACGCACACAUGCU |
| 1144 | CUCUCUCGCACACACGCACACAUGC |
| 1145 | UCUCUCUCGCACACACGCACACAUG |
| 1146 | CUCUCUCUCGCACACACGCACACAU |
| 1147 | UCUCUCUCUCGCACACACGCACACA |
| 1148 | CUCUCUCUCUCGCACACACGCACAC |
| 1149 | UCUCUCUCUCUCGCACACACGCACA |
| 1150 | GUCUCUCUCUCUCGCACACACGCAC |
| 1151 | UGUCUCUCUCUCUCGCACACACGCA |
| 1152 | CUGUCUCUCUCUCUCGCACACACGC |
| 1153 | UCUGUCUCUCUCUCUCGCACACACG |
| 1154 | GUCUGUCUCUCUCUCUCGCACACAC |
| 1155 | UGUCUGUCUCUCUCUCUCGCACACA |
| 1156 | CUGUCUGUCUCUCUCUCUCGCACAC |
| 1157 | GCUGUCUGUCUCUCUCUCUCGCACA |
| 1158 | GGCUGUCUGUCUCUCUCUCUCGCAC |
| 1159 | AGGCUGUCUGUCUCUCUCUCUCGCA |
| 1160 | CAGGCUGUCUGUCUCUCUCUCUCGC |
| 1161 | GCAGGCUGUCUGUCUCUCUCUCUCG |
| 1162 | GGCAGGCUGUCUGUCUCUCUCUCUC |
| 1163 | AGGCAGGCUGUCUGUCUCUCUCUCU |
| 1164 | UAGGCAGGCUGUCUGUCUCUCUCUC |
| 1165 | UUAGGCAGGCUGUCUGUCUCUCUCU |
| 1166 | CUUAGGCAGGCUGUCUGUCUCUCUC |
| 1167 | UCUUAGGCAGGCUGUCUGUCUCUCU |
| 1168 | UUCUUAGGCAGGCUGUCUGUCUCUC |
| 1169 | CUUCUUAGGCAGGCUGUCUGUCUCU |
| 1170 | UCUUCUUAGGCAGGCUGUCUGUCUC |
| 1171 | UUCUUCUUAGGCAGGCUGUCUGUCU |
| 1172 | UUUCUUCUUAGGCAGGCUGUCUGUC |
| 1173 | AUUUCUUCUUAGGCAGGCUGUCUGU |
| 1174 | CAUUUCUUCUUAGGCAGGCUGUCUG |
| 1175 | UCAUUUCUUCUUAGGCAGGCUGUCU |
| 1176 | UUCAUUUCUUCUUAGGCAGGCUGUC |
| 1177 | AUUCAUUUCUUCUUAGGCAGGCUGU |
| 1178 | CAUUCAUUUCUUCUUAGGCAGGCUG |
| 1179 | ACAUUCAUUUCUUCUUAGGCAGGCU |
| 1180 | CACAUUCAUUUCUUCUUAGGCAGGC |
| 1181 | UCACAUUCAUUUCUUCUUAGGCAGG |
| 1182 | UUCACAUUCAUUUCUUCUUAGGCAG |
| 1183 | AUUCACAUUCAUUUCUUCUUAGGCA |
| 1184 | CAUUCACAUUCAUUUCUUCUUAGGC |
| 1185 | GCAUUCACAUUCAUUUCUUCUUAGG |
| 1186 | CGCAUUCACAUUCAUUUCUUCUUAG |
| 1187 | CCGCAUUCACAUUCAUUUCUUCUUA |
| 1188 | GCCGCAUUCACAUUCAUUUCUUCUU |
| 1189 | AGCCGCAUUCACAUUCAUUUCUUCU |
| 1190 | AAGCCGCAUUCACAUUCAUUUCUUC |
| 1191 | CAAGCCGCAUUCACAUUCAUUUCUU |
| 1192 | ACAAGCCGCAUUCACAUUCAUUUCU |
| 1193 | CACAAGCCGCAUUCACAUUCAUUUC |
| 1194 | CCACAAGCCGCAUUCACAUUCAUUU |
| 1195 | GCCACAAGCCGCAUUCACAUUCAUU |
| 1196 | UGCCACAAGCCGCAUUCACAUUCAU |
| 1197 | GUGCCACAAGCCGCAUUCACAUUCA |
| 1198 | UGUGCCACAAGCCGCAUUCACAUUC |
| 1199 | CUGUGCCACAAGCCGCAUUCACAUU |
| 1200 | ACUGUGCCACAAGCCGCAUUCACAU |
| 1201 | AACUGUGCCACAAGCCGCAUUCACA |
| 1202 | CAACUGUGCCACAAGCCGCAUUCAC |
| 1203 | UCAACUGUGCCACAAGCCGCAUUCA |
| 1204 | GUCAACUGUGCCACAAGCCGCAUUC |
| 1205 | UGUCAACUGUGCCACAAGCCGCAUU |
| 1206 | UUGUCAACUGUGCCACAAGCCGCAU |
| 1207 | CUUGUCAACUGUGCCACAAGCCGCA |
| 1208 | CCUUGUCAACUGUGCCACAAGCCGC |
| 1209 | UCCUUGUCAACUGUGCCACAAGCCG |
| 1210 | AUCCUUGUCAACUGUGCCACAAGCC |
| 1211 | CAUCCUUGUCAACUGUGCCACAAGC |
| 1212 | UCAUCCUUGUCAACUGUGCCACAAG |
| 1213 | AUCAUCCUUGUCAACUGUGCCACAA |
| 1214 | UAUCAUCCUUGUCAACUGUGCCACA |
| 1215 | UUAUCAUCCUUGUCAACUGUGCCAC |
| 1216 | UUUAUCAUCCUUGUCAACUGUGCCA |
| 1217 | AUUUAUCAUCCUUGUCAACUGUGCC |
| 1218 | GAUUUAUCAUCCUUGUCAACUGUGC |
| 1219 | UGAUUUAUCAUCCUUGUCAACUGUG |
| 1220 | UUGAUUUAUCAUCCUUGUCAACUGU |
| 1221 | AUUGAUUUAUCAUCCUUGUCAACUG |
| 1222 | UAUUGAUUUAUCAUCCUUGUCAACU |
| 1223 | UUAUUGAUUUAUCAUCCUUGUCAAC |
| 1224 | AUUAUUGAUUUAUCAUCCUUGUCAA |
| 1225 | CAUUAUUGAUUUAUCAUCCUUGUCA |
| 1226 | GCAUUAUUGAUUUAUCAUCCUUGUC |
| 1227 | UGCAUUAUUGAUUUAUCAUCCUUGU |
| 1228 | UUGCAUUAUUGAUUUAUCAUCCUUG |
| 1229 | CUUGCAUUAUUGAUUUAUCAUCCUU |
| 1230 | GCUUGCAUUAUUGAUUUAUCAUCCU |
| 1231 | AGCUUGCAUUAUUGAUUUAUCAUCC |
| 1232 | AAGCUUGCAUUAUUGAUUUAUCAUC |
| 1233 | UAAGCUUGCAUUAUUGAUUUAUCAU |
| 1234 | GUAAGCUUGCAUUAUUGAUUUAUCA |
| 1235 | AGUAAGCUUGCAUUAUUGAUUUAUC |
| 1236 | UAGUAAGCUUGCAUUAUUGAUUUAU |
| 1237 | AUAGUAAGCUUGCAUUAUUGAUUUA |
| 1238 | GAUAGUAAGCUUGCAUUAUUGAUUU |
| 1239 | UGAUAGUAAGCUUGCAUUAUUGAUU |
| 1240 | AUGAUAGUAAGCUUGCAUUAUUGAU |
| 1241 | AAUGAUAGUAAGCUUGCAUUAUUGA |
| 1242 | AAAUGAUAGUAAGCUUGCAUUAUUG |
| 1243 | UAAAUGAUAGUAAGCUUGCAUUAUU |
| 1244 | AUAAAUGAUAGUAAGCUUGCAUUAU |
| 1245 | CAUAAAUGAUAGUAAGCUUGCAUUA |
| 1246 | UCAUAAAUGAUAGUAAGCUUGCAUU |
| 1247 | UUCAUAAAUGAUAGUAAGCUUGCAU |
| 1248 | AUUCAUAAAUGAUAGUAAGCUUGCA |
| 1249 | UAUUCAUAAAUGAUAGUAAGCUUGC |
| 1250 | CUAUUCAUAAAUGAUAGUAAGCUUG |
| 1251 | GCUAUUCAUAAAUGAUAGUAAGCUU |
| 1252 | UGCUAUUCAUAAAUGAUAGUAAGCU |
| 1253 | UUGCUAUUCAUAAAUGAUAGUAAGC |
| 1254 | AUUGCUAUUCAUAAAUGAUAGUAAG |
| 1255 | UAUUGCUAUUCAUAAAUGAUAGUAA |
| 1256 | GUAUUGCUAUUCAUAAAUGAUAGUA |
| 1257 | AGUAUUGCUAUUCAUAAAUGAUAGU |
| 1258 | CAGUAUUGCUAUUCAUAAAUGAUAG |
| 1259 | UCAGUAUUGCUAUUCAUAAAUGAUA |
| 1260 | UUCAGUAUUGCUAUUCAUAAAUGAU |
| 1261 | CUUCAGUAUUGCUAUUCAUAAAUGA |
| 1262 | UCUUCAGUAUUGCUAUUCAUAAAUG |
| 1263 | UUCUUCAGUAUUGCUAUUCAUAAAU |
| 1264 | UUUCUUCAGUAUUGCUAUUCAUAAA |
| 1265 | AUUUCUUCAGUAUUGCUAUUCAUAA |
| 1266 | AAUUUCUUCAGUAUUGCUAUUCAUA |
| 1267 | UAAUUUCUUCAGUAUUGCUAUUCAU |
| 1268 | UUAAUUUCUUCAGUAUUGCUAUUCA |
| 1269 | UUUAAUUUCUUCAGUAUUGCUAUUC |
| 1270 | UUUUAAUUUCUUCAGUAUUGCUAUU |
| 1271 | GUUUUAAUUUCUUCAGUAUUGCUAU |
| 1272 | UGUUUUAAUUUCUUCAGUAUUGCUA |
| 1273 | UUGUUUUAAUUUCUUCAGUAUUGCU |
| 1274 | UUUGUUUUAAUUUCUUCAGUAUUGC |
| 1275 | UUUUGUUUUAAUUUCUUCAGUAUUG |
| 1276 | CUUUUGUUUUAAUUUCUUCAGUAUU |
| 1277 | UCUUUUGUUUUAAUUUCUUCAGUAU |
| 1278 | AUCUUUUGUUUUAAUUUCUUCAGUA |
| 1279 | AAUCUUUUGUUUUAAUUUCUUCAGU |
| 1280 | CAAUCUUUUGUUUUAAUUUCUUCAG |
| 1281 | GCAAUCUUUUGUUUUAAUUUCUUCA |
| 1282 | AGCAAUCUUUUGUUUUAAUUUCUUC |
| 1283 | CAGCAAUCUUUUGUUUUAAUUUCUU |
| 1284 | ACAGCAAUCUUUUGUUUUAAUUUCU |
| 1285 | GACAGCAAUCUUUUGUUUUAAUUUC |
| 1286 | AGACAGCAAUCUUUUGUUUUAAUUU |
| 1287 | GAGACAGCAAUCUUUUGUUUUAAUU |
| 1288 | UGAGACAGCAAUCUUUUGUUUUAAU |
| 1289 | UUGAGACAGCAAUCUUUUGUUUUAA |
| 1290 | AUUGAGACAGCAAUCUUUUGUUUUA |
| 1291 | UAUUGAGACAGCAAUCUUUUGUUUU |
| 1292 | AUAUUGAGACAGCAAUCUUUUGUUU |
| 1293 | UAUAUUGAGACAGCAAUCUUUUGUU |
| 1294 | AUAUAUUGAGACAGCAAUCUUUUGU |
| 1295 | GAUAUAUUGAGACAGCAAUCUUUUG |
| 1296 | AGAUAUAUUGAGACAGCAAUCUUUU |
| 1297 | AAGAUAUAUUGAGACAGCAAUCUUU |
| 1298 | UAAGAUAUAUUGAGACAGCAAUCUU |
| 1299 | AUAAGAUAUAUUGAGACAGCAAUCU |
| 1300 | UAUAAGAUAUAUUGAGACAGCAAUC |
| 1301 | AUAUAAGAUAUAUUGAGACAGCAAU |
| 1302 | AAUAUAAGAUAUAUUGAGACAGCAA |
| 1303 | AAAUAUAAGAUAUAUUGAGACAGCA |
| 1304 | UAAAUAUAAGAUAUAUUGAGACAGC |
| 1305 | AUAAAUAUAAGAUAUAUUGAGACAG |
| 1306 | AAUAAAUAUAAGAUAUAUUGAGACA |
| 1307 | UAAUAAAUAUAAGAUAUAUUGAGAC |
| 1308 | AUAAUAAAUAUAAGAUAUAUUGAGA |
| 1309 | AAUAAUAAAUAUAAGAUAUAUUGAG |
| 1310 | AAAUAAUAAAUAUAAGAUAUAUUGA |
| 1311 | UAAAUAAUAAAUAUAAGAUAUAUUG |
| 1312 | GUAAAUAAUAAAUAUAAGAUAUAUU |
| 1313 | GGUAAAUAAUAAAUAUAAGAUAUAU |
| 1314 | UGGUAAAUAAUAAAUAUAAGAUAUA |
| 1315 | UUGGUAAAUAAUAAAUAUAAGAUAU |
| 1316 | UUUGGUAAAUAAUAAAUAUAAGAUA |
| 1317 | AUUUGGUAAAUAAUAAAUAUAAGAU |
| 1318 | AAUUUGGUAAAUAAUAAAUAUAAGA |
| 1319 | UAAUUUGGUAAAUAAUAAAUAUAAG |
| 1320 | AUAAUUUGGUAAAUAAUAAAUAUAA |
| 1321 | AAUAAUUUGGUAAAUAAUAAAUAUA |
| 1322 | GAAUAAUUUGGUAAAUAAUAAAUAU |
| 1323 | AGAAUAAUUUGGUAAAUAAUAAAUA |
| 1324 | UAGAAUAAUUUGGUAAAUAAUAAAU |
| 1325 | UUAGAAUAAUUUGGUAAAUAAUAAA |
| 1326 | CUUAGAAUAAUUUGGUAAAUAAUAA |
| 1327 | UCUUAGAAUAAUUUGGUAAAUAAUA |
| 1328 | CUCUUAGAAUAAUUUGGUAAAUAAU |
| 1329 | ACUCUUAGAAUAAUUUGGUAAAUAA |
| 1330 | UACUCUUAGAAUAAUUUGGUAAAUA |
| 1331 | AUACUCUUAGAAUAAUUUGGUAAAU |
| 1332 | AAUACUCUUAGAAUAAUUUGGUAAA |
| 1333 | AAAUACUCUUAGAAUAAUUUGGUAA |
| 1334 | GAAAUACUCUUAGAAUAAUUUGGUA |
| 1335 | AGAAAUACUCUUAGAAUAAUUUGGU |
| 1336 | AAGAAAUACUCUUAGAAUAAUUUGG |
| 1337 | GAAGAAAUACUCUUAGAAUAAUUUG |
| 1338 | GGAAGAAAUACUCUUAGAAUAAUUU |

*At least one (i.e., one or more) nucleoside linkage of the oligonucleotide sequence is
independently selected from a phosphorothioate linkage, an alkyl phosphate linkage, a
phosphorodithioate linkage, a phosphotriester linkage, an alkylphosphonate linkage, a 3-
methoxypropyl phosphonate linkage, a methylphosphonate linkage, an aminoalkylphosphotriester
linkage, an alkylene phosphonate linkage, a phosphinate linkage, a phosphoramidate linkage, a
phosphoramidothioate linkage, a thiophosphorodiamidate linkage, a phosphorodiamidate (e.g.,
comprising a phosphorodiamidate morpholino (PMO), 3’ amino ribose, or 5’ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

Table 3 below identifies exemplary STMN2 AON sequences:

TABLE 3
Exemplary STMN2 AON Sequences, in each one or
more spacers described in the present disclo-
sure are incorporated for generation of an
oligonucleotide of the present invention

| SEQ ID NO: | Oligonucleotide sequence |
|---|---|
| (legacy ID*) | (5′ → 3′) |
| SEQ ID NO: 31 | AATGTTAAGACATAATACCAGAGCT |
| SEQ ID NO: 36 | TTAAAAATGTTAAGACATAATACCA |
| SEQ ID NO: 41 | TAGATTTAAAAATGTTAAGACATAA |
| SEQ ID NO: 46 | TACCATAGATTTAAAAATGTTAAGA |
| SEQ ID NO: 55 | TGTAAAGATTACCATAGATTTAAAA |
| SEQ ID NO: 144 | AATCCAATTAAGAGAGAGTGATGGG |
| SEQ ID NO: 146 | AAAATCCAATTAAGAGAGAGTGATG |
| SEQ ID NO: 150 | TTTAAAAATCCAATTAAGAGAGAGT |
| SEQ ID NO: 169 | CCTGCAATATGAATATAATTTTAAA |
| SEQ ID NO: 170 | TCCTGCAATATGAATATAATTTTAA |
| SEQ ID NO: 171 | GTCCTGCAATATGAATATAATTTTA |
| SEQ ID NO: 172 | AGTCCTGCAATATGAATATAATTTT |
| SEQ ID NO: 173 | GAGTCCTGCAATATGAATATAATTT |
| SEQ ID NO: 177 | TGCCGAGTCCTGCAATATGAATATA |
| SEQ ID NO: 181 | CTTCTGCCGAGTCCTGCAATATGAA |
| SEQ ID NO: 185 | AGGTCTTCTGCCGAGTCCTGCAATA |
| SEQ ID NO: 197 | CCTTTCTCTCGAAGGTCTTCTGCCG |
| SEQ ID NO: 203 | TTTCTACCTTTCTCTCGAAGGTCTT |
| SEQ ID NO: 209 | TCTTATTTTCTACCTTTCTCTCGAA |
| SEQ ID NO: 215 | CCAAATTCTTATTTTCTACCTTTCT |
| SEQ ID NO: 237 | GCACACATGCTCACACAGAGAGCCA |
| SEQ ID NO: 244 | CACACACGCACACATGCTCACACAG |
| SEQ ID NO: 249 | TCTCGCACACACGCACACATGCTCA |
| SEQ ID NO: 252 | CTCTCTCGCACACACGCACACATGC |
| SEQ ID NO: 380 | TGTTTTAATTTCTTCAGTATTGCTA |
| SEQ ID NO: 385 | TCTTTTGTTTTAATTTCTTCAGTAT |
| SEQ ID NO: 390 | AGCAATCTTTTGTTTTAATTTCTTC |
| SEQ ID NO: 395 | GAGACAGCAATCTTTTGTTTTAATT |
| SEQ ID NO: 400 | ATATTGAGACAGCAATCTTTTGTTT |

*At least one (i.e., one or more) nucleoside linkage of the oligonucleotide sequence is
independently selected from a phosphorothioate linkage, an alkyl phosphate linkage, a
phosphorodithioate linkage, a phosphotriester linkage, an alkylphosphonate linkage, a 3-
methoxypropyl phosphonate linkage, a methylphosphonate linkage, an aminoalkylphosphotriester
linkage, an alkylene phosphonate linkage, a phosphinate linkage, a phosphoramidate linkage, a
phosphoramidothioate linkage, a thiophosphorodiamidate linkage, a phosphorodiamidate (e.g.,
comprising a phosphorodiamidate morpholino (PMO), 3’ amino ribose, or 5’ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

In some embodiments, all internucleoside linkages of the STMN2 AON oligonucleotides listed in Table 3 are phosphorothioate linkages (except when a spacer is present, the linkage may or may not be a phosphorothioate linkage), and each of the linked nucleosides of the oligonucleotide are 2′-O-(2-methoxyethyl) (2′-MOE) nucleosides, and each “C” is replaced with a 5-MeC. In some embodiments, all internucleoside linkages of the STMN2 AON oligonucleotides listed in Table 3 are phosphorothioate linkages, and each of the linked nucleosides of the oligonucleotide are 2′-O-(2-methoxyethyl) (2′-MOE) nucleosides, and not all or none of the ‘C” is replaced with 5-MeC.

TABLE 4
Table 4 below identifies additional exemplary
STMN2 AON sequences:
Additional Exemplary STMN2 AON Sequences
(corresponding to AONs shown in Table 3
but with thymine bases replaced with
uracil bases)

| | Oligonucleotide sequence |
|---|---|
| SEQ ID NO | (5′ → 3′) |
| SEQ ID NO: 923 | AAUGUUAAGACAUAAUACCAGAGCU |
| SEQ ID NO: 928 | UUAAAAAUGUUAAGACAUAAUACCA |
| SEQ ID NO: 933 | UAGAUUUAAAAAUGUUAAGACAUAA |
| SEQ ID NO: 938 | UACCAUAGAUUUAAAAAUGUUAAGA |
| SEQ ID NO: 947 | UGUAAAGAUUACCAUAGAUUUAAAA |
| SEQ ID NO: 1036 | AAUCCAAUUAAGAGAGAGUGAUGGG |
| SEQ ID NO: 1038 | AAAAUCCAAUUAAGAGAGAGUGAUG |
| SEQ ID NO: 1042 | UUUAAAAAUCCAAUUAAGAGAGAGU |
| SEQ ID NO: 1061 | CCUGCAAUAUGAAUAUAAUUUUAAA |
| SEQ ID NO: 1062 | UCCUGCAAUAUGAAUAUAAUUUUAA |
| SEQ ID NO: 1063 | GUCCUGCAAUAUGAAUAUAAUUUUA |
| SEQ ID NO: 1064 | AGUCCUGCAAUAUGAAUAUAAUUUU |
| SEQ ID NO: 1065 | GAGUCCUGCAAUAUGAAUAUAAUUU |
| SEQ ID NO: 1077 | AGGUCUUCUGCCGAGUCCUGCAAUA |
| SEQ ID NO: 1089 | CCUUUCUCUCGAAGGUCUUCUGCCG |
| SEQ ID NO: 1095 | UUUCUACCUUUCUCUCGAAGGUCUU |
| SEQ ID NO: 1101 | UCUUAUUUUCUACCUUUCUCUCGAA |
| SEQ ID NO: 1107 | CCAAAUUCUUAUUUUCUACCUUUCU |
| SEQ ID NO: 1129 | GCACACAUGCUCACACAGAGAGCCA |
| SEQ ID NO: 1136 | CACACACGCACACAUGCUCACACAG |
| SEQ ID NO: 1141 | UCUCGCACACACGCACACAUGCUCA |
| SEQ ID NO: 1144 | CUCUCUCGCACACACGCACACAUGC |
| SEQ ID NO: 1272 | UGUUUUAAUUUCUUCAGUAUUGCUA |
| SEQ ID NO: 1277 | UCUUUUGUUUUAAUUUCUUCAGUAU |
| SEQ ID NO: 1282 | AGCAAUCUUUUGUUUUAAUUUCUUC |
| SEQ ID NO: 1287 | GAGACAGCAAUCUUUUGUUUUAAUU |
| SEQ ID NO: 1292 | AUAUUGAGACAGCAAUCUUUUGUUU |

STMN2 Transcript with a Cryptic Exon

In one embodiment, a STMN2 AON targets a region of a STMN2 transcript comprising a cryptic exon sequence, the STMN2 transcript comprising the sequence provided as SEQ ID NO: 1339.

(SEQ ID NO: 1339)
ACTTGTAATATACAGGTATCCCTCCTGGTAAGCTCTGGTATTATGTCTTA
ACATTTTTAAATCTATGGTAATCTTTACAAAATATTTTACTTCCGAACTC
ATATACCTGGGGATTTTATTACTCTGGGAATTATGTGTTCTGCCCCATCA
CTCTCTCTTAATTGGATTTTTAAAATTATATTCATATTGCAGGACTCGGC
AGAAGACCTTCGAGAGAAAGGTAGAAAATAAGAATTTGGCTCTCTGTGTG
AGCATGTGTGCGTGTGTGCGAGAGAGAGAGACAGACAGCCTGCCTAAGAA
GAAATGAATGTGAATGCGGCTTGTGGCACAGTTGACAAGGATGATAAATC
AATAATGCAAGCTTACTATCATTTATGAATAGCAATACTGAAGAAATTAA
AACAAAAGATTGCTGTCTCAATATATCTTATATTTATTATTTACCAAATT
ATTCTAAGAGTATTTCTTCC

A cryptic exon sequence within the STMN2 transcript is provided as SEQ ID NO: 1340.

(SEQ ID NO: 1340)
GACTCGGCAGAAGACCTTCGAGAGAAAGGTAGAAAATAAGAATTTGGCTC
TCTGTGTGAGCATGTGTGCGTGTGTGCGAGAGAGAGAGACAGACAGCCTG
CCTAAGAAGAAATGAATGTGAATGCGGCTTGTGGCACAGTTGACAAGGAT
GATAAATCAATAATGCAAGCTTACTATCATTTATGAATAGCAATACTGAA
GAAATTAAAACAAAAGATTGCTGTCTC
(Source: NCBI Reference Sequence: NC_000008.11).

In various embodiments, the STMN2 transcript with a cryptic exon shares between 90-100% identity with SEQ ID NO: 1339. In various embodiments, the STMN2 transcript with a cryptic exon shares at least 90%, 91%, 92%, 93%, 94%, 95%, 96%, 97%, 98%, or 99% identity with SEQ ID NO: 1341.

In one embodiment, a STMN2 transcript with a cryptic exon can comprise a pre-mRNA STMN2 transcript. In one embodiment, a STMN2 transcript with a cryptic exon can comprise the sequence provided as SEQ ID NO: 1341.

(SEQ ID NO: 1341)
AGCTCCTAGGAAGCTTCAGGGCTTAAAGCTCCACTCTACTTGGACTGTACTATCAGGC
CCCCAAAATGGGGGGAGCCGACAGGGAAGGACTGATTTCCATTTCAAACTGCATTCT
GGTACTTTGTACTCCAGCACCATTGGCCGATCAATATTTAATGCTTGGAGATTCTGAC
TCTGCGGGAGTCATGTCAGGGGACCTTGGGAGCCAATCTGCTTGAGCTTCTGAGTGA
TAATTATTCATGGGCTCCTGCCTCTTGCTCTTTCTCTAGCACGGTCCCACTCTGCAGAC
TCAGTGCCTTATTCAGTCTTCTCTCTCGCTCTCTCCGCTGCTGTAGCCGGACCCTTTGC
CTTCGCCACTGCTCAGCGTCTGCACATCCCTACAATGGCTAAAACAGCAATGGTAAG
GCACTGCGCCTCGTTCTCCGTCGGCTCTACCTGGAGCCCACCTCTCACCTCCTCTCTTG
AGCTCTAGAAGCATTCAGAGATATTTTATAAAGAAAAAGATGTTAATGGTAACACAG
GACCAGGAAGGACAGGGCAGTTCTGGGGGAGGTGGGAGGGCAGAGAAGAGGTCTAT
GGAAATCTAAAGCGAAGAATTTCTTTTAAAAGGTAGAAGCGGGTAAGTTGCCCTCCT
ATGGGTAGAGAATTTATTCTGTTTCCATATTTAAAATTAGGACTCAATCGTGAGGGGA
GGAAGCTACCTTAACTGTTTGCCTTAAATGGGCTTAAGGGACATTTTGGAAAGTGCTT
TATAACGACCTTTTTTTTTTTTATTTCTTCTCTAGTTTAAGAAGAAAATAGGAAAGGG
GTAAAGGGAAGGTGGGAGAAAGGAAAAAGAAAATTGCAAAGTCAAAGCGGTCCCAT
CCCGCTGTTTGAAAGATGGGTGGAGACGGGGGGAGGGGATGGAGAGAACTGGGCAC
ATTTTACGGTATTGTCTCGTCGAAGAAACCGCTAGTCCTGGGGTGCGGTGCAGGGAG
GTAAGACGGCGGGGGACAGGGTGGGGGTAGGACCTCCGCTCCTTTGTTTTAGGGCAA
GGGAGGGGAAGGAGAGAGGAAGTCGCGGAGGGCGTGGAGGGCGCGGGTGGGCAGC
TGCAGGGGCGGGGAAGCGCGCGGCAGGGAGGGGTGGAGGGACAGCGGCTTCGAAG
GCGCTGGGGTGGGGTTTCTTTGTGTGCGGACCAGCGGTCCCGGGGGGAGGCACCTGC
AGCGCTGGGCGCACAATGCGGACAGCCCCACCCAGTGCGGAACCGCGCAGCCCCGC
CCCCCCGCCCGGTGCTGCATCTTCATTCGAAAGGGGGTCGGGTGGGGAGCGCAGCGT
GACACCCAGGAGCCCAACCCTGCGGGGACAGCGGCGCCACGCCCCGCGCTCCCCGCT
CCCGACTCCCCGCCGCGGCTTCCAAGAGAGACCTGACCACTGACCCCGCCCTCCCCA
CGCTGGCCTCATTGTTCTGCTTTTAAGAGAGATGGGAAAAGTGGGTTAACATTTTTCT
TTTCGGAAGCAAATTACATAGAGTGTTTAGACATAGACACAGATAAAGGGTTCTTTG
AAGACCTTTGATCGTTTGCGGGAAAAGCTTCTAGAACCTAGACATGTGTATGTATAAT
AATAGAGATGACATGAAATCGTATATAAAGCAAAAGAGGTCAAAGTCTTAAGTTAA
GCCACGCGAAATTTCCGTTTTGTGGGTCAGACAGTGCCAAATATCGGCAATTTCATAA
GCTCAGAGAGACAAGACAGTGGAGACACAGGATGACCGGAAAAGATTCTGGATTCA
GGGCCTTCATCCGCAATTGGTCTTGTGCCTTGAGTGCCCACGGTTCTGGCGCTCAGTG
GCCCCGGGGTGAAAAGGCAGGGTGGGGCCTGGGGTCCTGTGGCAGCTGGAAGCACG
TGTCCCCCGGGACTTGGTTGCAGGATGCGGAGACAGGGAAAGCTGCCGAAAGGACTC
CATCTGCGCGGCTCCGCCCTGCCCTACCCTCCCCGCGGAGCCGGGGAGACCTCAGGC
TCCGAGACTGGCGGGGAAGAGGAATATGGGAGGGGCAGTTGAGCTGTATGCAGTCC
TGGAACCTCTTTTTTCAGCCCCGCAGTCCACAACGGCCCGAGCACCCCTTGATGTGCG
CAGACCCCCGGCGTGGCTCTCAGCCCCAGCACCGAGCCCCTCCCAGCCAAGCGGGTG
GCTCTGCAGAAAAGCTGGCTCGAGCCCCGCCCGGCCACACAAAGGCGCGGCCCCACC
CAGCCCGGGCGCGAGACCGCAGAGGTGACCCCCTTCCCAGGGATTCAGGGAGGGCT
GTCTCTTCTCGCCCACCCACGGTCCGCGGAGCTCGGGGCTTTTTTTCCCCCAGCCCAA
GCCCCCCGCCCACCCTCTGTTCTCTATGATTTTCCAGAATGGAGACCCCGCGAGGGGC
TTCTCTAAGGGAGACCCTCGCTCCTCCAGCGGGGCGCGGCTCGGCCCCACCCCTCCCA
GCTGAGGCCCAGAGCCGCCTACCGCTGGCCGGGTGGGGGCGCACGTGGCGACTGGGT
GTGTGGAGCGCAGCCAGCCCTGCAGAGCCCCGCGCCGCGCCCTGCGCTCCCCTCCCC
GGAGTTGGGCGCTCGCCCCCGCGGTGCAGCCGGGGAGACCGGTTTCTGCGCAGTGTC
CTGAGCTACCCCCGCTTTCCACAATTCGCAGTTCACTCGCACGTCCAGAAAGGTTCTG
AGAATGGGTGGTGGGGGCGATCTCGCCTCGCTTTCTGCACCCCTCAGAAAGGTTTCC
GCTGCAGGCTAGTGGCTGCAAACTCATCGTCATCATCAGTATTATTATCATTTCAAAT
CGTTGTTATTATTTAATGATTCAGTAGCCTTGTTTGTTCTCATTTGTTCAAAAGGGACG
TGGATTGCTCTTGGTTAAGGATTAACCCTTGTTGCGTTCGCTTTGCTTCCTCCTAATTG
CCCTCATCCCTTTCCCCCACAAAAAGGTAAATTTGTCTCCAGTTGTTCATTTTAAGTTA
TAAAGCAAATATATTTTTGCTTCCTGCCAGGATTATGTATGTTCATGTGGCTAAGATA
CATGTGCAAGTGCTTGCTAAGAGCAGGGTTTGTGTGCCAACGATTGCTGGAAAATTC
TCTGCAAAGAATTGTTTGTGGCTGCAATGGGTGAGAATACACATATATAATTGAGAT
GATCTTCAACATAAGGTTATATCTATAAATATATAAATATAGTTTATGCACAAAATTT
TAAGTTTTTTCCCCTGAAACTGTTCTTCCAACTGCTGATTCTTGATACAGCCTCAATCC
TACACAGATACATGGATCGTGAAATGGTAGCCGCCATCCAAATAAAAATCCCACCCC
AAATATGACAAACGCAAGCATCCTTTCTGGCCATAATTTAACTGCATTTGCAAATCAT
GAAAAAAACACTACTTCTGCAGTATTAAAATAATAGATTTTGAAATTAATTCCAATTT
CAAAGATAATTAATTATCAGGGCGAGTGCTTTTTTCCTGATTCATTAAACAATTATGT
ATTCAGCATGATTGTAAGAGGTGCATATAATATTCCCCATTATCTTTTCTAATGAAGT
GGGCACCTTCTGAATGGATATATAAGTAACTAGAAATGAAAAGCTGAGGATTTGGTC
AGAATTTCAGGATAAAACTGAAAGAAATGGCAGTAGTTTATCAATTAATCTCATGTA
TTTAGTTTATACCAGGTGAGTAAGCTGAGCCTGCAATAAACACTCTCTGTCCCAGTGT
AACACGTCGCAGGTAGCTAGAATGATAGGATAAATTAATAGACCTTGTGGTGTTTGT
CTATGCACGTTAAAATTCTCTGAGAGAAAGTATATTTTAAAATGATAATTAAGATTGG
ACATTTGTGCTATTAAAATCTACAACTTTAGTCAAAATTCACAATGGTTTTTTTTTACA
ATAATGTGACTTACAGATTTGTAGTAAATTATTCTATTCTAAAAGAGAAATGAGTGTT
TTTATTGTTACAGCTATTACCTCATTAATATTTTTAGCAAACTTTTATTTGTTGCATTG
AAAGCAGTTTTAATTACTTTGGGTTTTTATTTTTCAAATTACTAATGGATAGATGGTG
GAATAAGCATTTAATCATTTGGCACAATATGACTTCCATCAAATAGCTCATTCTCAGT
GATTAAAAAATGCTACAAGAGGCTACAATTTACTCAGATTCAGGAAATGTCCTTTCA
GAGTGCCATAAGGCTGATTCATATAATAAAATAGTTTTCTTCCCTATAATTTAAGATC
AAATAGTTACTTAGTTCTGTGAATACCTAGCAGTAGCTATCAAACAGAATTTTAAAGT
TAAATCTGTACAACTAACAATGAAGTGGAGGATGAATCGATACATATTGAATGGAAG
ACTTTGTCATTGATAAATTCAGGCCATCTTTAGGAAAATTCCGGATTTATCAATCACC
ATTATTTTTTACTTCAACTGAGTGTGACTGATCACATGCTCAGGCTACCTTGGTAGCT
CATTGCTCACAGGAGGCTGAAAAAAGCTGGCCTCCGAGCAGGAGGAAGCTCAGAGC
ACAAACCTAGGCCTGGGCGTGGCCACTGGGAGCTGCTGATAGCGAACCCCAGCTCAC
ACCAGTTTCTTTTTTGGTCGTGGGAAGAAAAACACATATTATCCTGTTGTCACAAGAT
CTGTGACCTTATATGAAAAAATGCTAGAATTTTTTCATTAAAAAAGAAAATACTGAA
CTAGCCAGTGACCCAGATGTTTTCAGAACCTAGACTGGTTCTGTCCATTGGAAAACCT
CGGTGTCTGCATTAACTTTTCACCACACTAGAGGGCAATCATGTTCTCTAAAAAAGCA
GATGATTGATGTAAACCTAGTTCCAAATATTAACTGTTTAATAAAATCTTTTCTTTTAC
CAGGAACATTCAAGTGTTTATTCAATAAGCTGATGCCATGCTTTACCCTAGTGGATGA
ACAGAGCTTGTACAATTTTCAAGGAGACAGGATGAAATGAGTGGTCATAATCTGAAA
GTAGATACACGCCCTGGTTAATTATTCCCTGATGGTTTTACTTCTCAGTTTTATTACAT
TGTTATTATAATACCATTTATGTTACTTCTGAGATTTTGTAGTGGATAAATAGTAGAA
AAATGTCAGTAGTAATAGCAAAGTTATTTAGCAGCCGAATATTTTAATGCTTAAAAA
TAAAGGAATAAATTAAAGAAAATCATTGTTTACTTCTTCATCGATTGAAATGTGCCCC
CTGTTCAGAGCACATCTGAATATCAGAGTCTCCACCTGCAGAGAACATGCAGCTTAG
CGAGTAAAACAGGCAGGTATGTGATACTGAGGAGGTGTACCAAAAACTGACTGCTGT
TATTTTTCCCATCTTCTAAGTCTGTCTTTCTTTTCCATTTAAAGATACCTTTTTAAACT
AATCCAATGTGATTTCAATCTAGTTTTATCAGATTTCAACAATTATTGAGCATCTCCTT
GTAGTGGTTTTCTGTTTATTAGAAAATCGATGTTAATTTTAACGAAGTAAGAAGAAAT
ATATAAGTATAAACTAATTTTGGGTATCATCAAAAGTGGATTTTTTAAATATGCATTG
ATAGAATTATTTTTTGATTACATTTTATGTAATTCTAATCCAGCTATAAAATATTTAAT
AGTGTCATATTACTGTGTTCCTCAAACTTTGATGTGCATATGAATTACCTTTGATTTTC
ATTAAAATGCAAATTCTGATTCAATACATCTGGCTTGAGGCAGACATTCTGTCTTCCG
AACAAGCTCCCAGATGATGCTGATTCTGACCACTAAACACATCAGTTTTAGGGATATT
AACTTGTAATATACAGGTATCCCTCCTGGTAAGCTCTGGTATTATGTCTTAACATTTTT
AAATCTATGGTAATCTTTACAAAATATTTTACTTCCGAACTCATATACCTGGGGATTT
TATTACTCTGGGAATTATGTGTTCTGCCCCATCACTCTCTCTTAATTGGATTTTTAAAA
TTATATTCATATTGCAGGACTCGGCAGAAGACCTTCGAGAGAAAGGTAGAAAATAAG
AATTTGGCTCTCTGTGTGAGCATGTGTGCGTGTGTGCGAGAGAGAGAGACAGACAGC
CTGCCTAAGAAGAAATGAATGTGAATGCGGCTTGTGGCACAGTTGACAAGGATGATA
AATCAATAATGCAAGCTTACTATCATTTATGAATAGCAATACTGAAGAAATTAAAAC
AAAAGATTGCTGTCTCAATATATCTTATATTTATTATTTACCAAATTATTCTAAGAGT
ATTTCTTCCTGAATACCATGTGAGAAAATTCTTAAGAATTTATTGAGTATGACTGTAT
ATTTGAAAAGAGTGTTTTCTTCTGCTTATCTAAGCCAATAAAGGATCTTCATTATTCA
ATTCTAACTTTCTAAGGAAGTCAACCTACAGATCAGAAAGAGGATCTTCAAGGAATA
GCATCAAAGACATAGTCAGGTCTCCCATGCAGTGACTGGCTGACCATGCAGCCATTA
CCACCTTTCTGGAAATATTATGCTGCAAAAATGATACAATACACGAAATATCTCAAA
TTAAAAAATATAACATTTCCCAAATAGGGCACTAAAAACATGATCCCAAATAAAACT
AGCTTCAGGGTTTGCAGAATATACTGTTACTCAACACAAAGTTGGACTAAGTCTCAA
AGTTAGCCATTCAGTTGTTGTTAACAGTTCATTTCAGGGTCTCTCAGAAGCTGGGAAA
CTTTCCATTTTTGCAATTTCTTGTACATTGAAGGAAAGGAAGACACACTTAAGACAGC
ATTACAAAAGTAATTCATGTTTTAAATGTTTAATTCTGGCAGTCGGGCAGGGCTCTCT
GTATAACCTCATTTGGAGATGACAAAAATCTAAACTTGAGGGCCTCGAGCCAATAAG
TCTTCCTATTTCTTTACTCAAACATTTTCCCGCAATGGTGCTTTCTTTCAACTGTTTTTC
TGGTGTATTCATAAATTCCAGATTCTCTATGGGAAGTAACTTTTATTGATTGATTTAA
CCCTTGTATAGCACATATAACATGCAAGGCATTGTTCTAAGAACTTTCCACATATTAA
CTGTGTTAATCACTTAATAATCCTAAGTAGGTTCTATTACAGATATGGAAACTGAGGC
ACAGAAAGTTGAAGTATCTTACTCAAGGTCACACAGTTAGTCAGATCCAGAATTTGG
GCCCAGGCCATCTGGCTTCGGAATCCATCTTTCACCGATTGCTGCTAGTCTCATATCT
GTTCCATGTTAGAGGTGAGCTCCCATTGCAGAGGTCACACCTGTGATATCACCATTTT
ATTTAAACAGACCAGAGATGGTCTTCTCCTTTCTGATCACAGACTCACCTTGAAGAGA
AAATACTTCCAAATTGATGCCTAGTTTTAATAGCTTACCTGGGGCTTATTCAAATAAT
TGCCATGATTTAGGCTTTGGGAGAAAGAGAGCTATGAGGCCGTGTGGGTTGTAACGT
ATGAGACACATGGCGTTCTGCAGGCTCAGCACAGCATCGATTTCTGGTGGGAACACA
CTCTGATGACCAGTTCCAGAAATAACATTGACTTAATCTCCTCAGTCCCATCATGGTT
AGCACATTTCAAAATGCCTCCTTAACTACTTCCATAGGCCAGAGATATTTAGTTTTAA
CATTTTGTTGAATAAAATAAATTTACACATTCACATTTAATATAACTATTAGATGTTA
TTTCAAGATTCTCTTCATATTACCATCAAAGCAGGCAGGCAGGCAGGAGAGAACTGT
AGGAAGGTTTTGAATCCCTTGTGAAACATTTTTAATTATCTTTTAATAAAGGAATCAG
GCCCTGTCATTTGTCAAGGAGACATTTGCAGTAGTAAAGCTTGTGTTTATAATATCCA
TTTTTATTAGTCATGATTAAAGATAACATTTGTGTACATTTGTTCTCACAAAACACTTT
TATATGAGTGTAAAGGTTAATTAATGCATTTCAGCCATCATTTTGCTGGTCATGTGGA
AATATAGCTTCTTTAGGAATTGTACTTAGAGTAGGAGCCACATATTATACTATAAAAC
CATAACAAAAATATTTTAAGTTTGTTCTCACTTGTTGTTGACCTCCAGAGTAAAATAT
TTAATACTCTGGAAAGTTATGGGTTTCAAAATTTATTTTATGGCAAGAAATAGATAAT
TACAGTTCTCATAGAGCACATTTAAAATAATTTATTTTTATAGGGCAAAAATATTGCC
TAGGACTGAATGATTTTTTTTTTTTTACAAAGATTGTAAAGCAACGCCTGCAAGAGTG
CCCATTTAGCAGTTATTCTTCTGGAATAATTGTATTTTGGATGTTGGAGTTCGCACATT
AACCATTAGTACAAGTACCCAATATAACAATAGATCATCAGGATAATAAATCTGTCC
ATCTTTTAGTTGTATGTCTTTATATCAGGATAAAGAGAATTGAGTGAAATTTATCTAA
ACCTAGTCCCACAAATACTTTTACAAGAGAGCATGTTAAAGTGTAAATTAAATTTTTA
TTAGCATTCTACTCTGTCTTTGGAAGTTTTTTTTCCTTATGAAATGCAGCCATAAAGTT
TAACTTCCATTAACAAAGCTGCTCACAGTAAACCTATTATAATAATAGTTTCCCAGTT
TGGGCTTCCTAGTGAGGAGCAACCTAACTCACACGAAACAACCCCAACTTATAATAT
ATTGACTGTTACAAAACTGAGACCAGAAAATCCCATCAAGATGGTACTGTTATCATTT
CCAGACTCTCGGGAAGAACATTAATCATCTCAGGCACTTTTAGGATAGACTTATTGCA
GCCTCCCTGGGAACTCTGCTTCAGAACATAATTATTTTTATTAATGCAGAGTTACTTTT
TATTTCCAACAAAAATATCTATTGTTATTATTTAAGTCTTACAGCTTTATCTGAGAAAT
TCCAATTAGCACCCTTCTCATAATAAATATTCAAACACATGAAAAATTACCAAAGTTG
TTCTAGTCTTTTAATGACATATTACATGATCCTGCACTCTTGTCACTTTAAAAATTATC
TTTTTATTATATTTCTGATGATTTTTTTCTTATATAGTTTTTTAAAAGGAGCAGGCAAG
CATAGAAGACTAAAAAATGTTCAAAAGAAAAATTAAATCGCATGATCTATCTATATG
GGACCTTGTCATTTTTAGAAAACATTCACCTGCTTCATCCTTTTGAATCTTCATATAAT
CCCTCTGAGATGGGCATACTATACAAGTTGTCTTATTTAAAGATTGGTAAATTTAAGC
TCAAATAATTTATTCAGTGGCAAGCCTCAGAGGCAGACTCGGAACACAGGTCTAATA
TATATTATATATATATTATAACATATAATATATATATTACATATAATAAAGTTGTGTA
TATTATTTACCTATCAAAATATTTATATGTAATATATAAATATGTTATATATCATGTAT
GTGCCTATTTCATACATATATACACATTCATGCAAAATAAGGTTTAGCACTCCCTCCA
CTGTCCTGTAATAAAACATGCACAGTGAGAATAGTCATACACGAGGCATATTTGTCTT
CAGTTTAAAGTCATTGATAGTCAGTGTCACTAACTAAAGTAAAATAGATTGGAGCAC
CAACTTTGTTCTGAAGCCTGTGCCAGGTATTATGAGAACAAAAATAAAAATGTTCCTC
ACCCTTGGTGGATTTAGTCTTTTGCAGAAAAAAAGATCCTGTACATGTCAGAAAGTTC
AATAGTAATAATGGTAATTTATAACTATAAATGGAAGTCACCATCTCACAATTTCACC
ATCTTAACAATTTTGTTAAACTGCCCTACAATATTACAAGATAGTACATAATGATACA
CTAGTAACATCAACTAGGAAGTACCAAGATCCACCAAAAGGCTGAAAAATTTAAATA
TTTAATGAGTCCATCAACCAATCTGGCCAGAGAATTCTTTAATTAAAATGCTTCCCAA
ATTTTACTGAGAATCAGCAGCGTTTGAGGAGCTAGCCTCCACCCCCAGAGGTTCTCAC
TCTATTAGGTCTGAAGCAGGTCCCATGGATTTGCATTTCTAACAAGCTCCCAGGTGGT
GCTGATGAGGCTGATTCAGAACCACACTTGGAGTAGACCTAAAACAGCAGTGACCTG
TAGGGTCCCCAAGCAGCAGGCCAGGACAGCATGTGAGTTACGTCCTCTGTGGAGCTC
TGCAACAAGGCGTCAAGAGGTCAGAGTCTAAGTCCCCATCAGCTCTGCCCTTCTCCA
CCAGTGCTGCTGGTGCTGCATGGAAGGAAGAGCCCAGAAGGGATTCTGAGTTTCAGT
CTTTACTCTTGCTGACGCACCTTGGTCAGGTCAATTTTCCTGTTTGTTCCTCTAATTCA
GCATCTGTAAAATAGCCATGTGAACTGCCTTGTCCATATCAGAGGGTCTTTTTCAGAC
TCAAGGAAAAAAACGTGAAAGTGATTAGTGTCTGTCAAGTAGTATATAAATGCAAGA
AGTTGAGTTTTTAAATTGTCATTAGATATAAATACCCATGTGCATGCATTTAGAATGA
GTAAAGAGGGAACAAGGAGCGCAATCAAAAACTGCGTCATTTGCTTTTTGAAAAATA
CTTTCTATGTAATGAAAAGTGAAATAAAATGTTAATTGAGTCCCTCTGACAACAGCAT
CAGACGTTTTGCAGTTCTTGTGATTAGAACCCACCTGGCCAGCCCTTCTTCCTCCTAA
AGAAGAGCCTTCTTCTTCTTAAATGAAGGTTGGCTCAGAAGAAGCAATTAACTCATTC
AACGTTTTGTTACAGTCAATCCACATCCAACTTTTCCCCAACTCAATCTGCTTTAAGG
GAAGGATGGTAAGTGGTGGCCCAAGATGGCAACCATCAAGCTTAGAGAATCTCTAGA
AGCAGGGGTGTCCCCAGCAAGTAGACACTGAAAATATGAGAGGGCTGATAAGCCAG
AGATAAAACTCAGTACTTACTTTGCTTCTAGTCCATGTCTACCCCTTTCTTGGCACCAC
CTTGACACTACCCTCTGAGTCCACCTTCCTGAGATGGTACAAACTCTGCTTAGACAAA
GCAGCCCATGTCCAAAGGTGTTAGGGCTCAGTTTAAAGCTGCCTTCAAAAGTTAAAA
CAGAAGTGTAAAGTTCTGTGCAATTAAAAATAATCAGCTTGTCTTGGAACTCAAACG
AATGTAAAATCCTATGAAAATTAAAAAGCAGTACCACAAGTTACCCCAAAAGTCCTT
AGGTCAGTAACTGTTCCTGTTACAGGTAAGAGAGAGCATGGATTAGAGGTGGGCGTG
GGTATCCAGTGGACATGGTTTTGAACCATGCTCCACTACTACTCACTATCTGAGAATT
CTTAAATTTATTAATCATTTCTATATTATAATTTTCTCAGTTATGAAATGGGAAAACA
ATACCTAAATCACATGGTTGTTAAGTAAGCAATTGATTGTTAAGCATTTGGTCATCAA
AAATATTAATCCCCTTCCCTGATTCCCTAGATAAATGATGAAAATACTAAATAAAAAT
AATAAAAATTTAAAGTGAACATCTCAATTCTTATACTTTGTTAATTTCTACATGTATT
ACAAATCTACTAGAAATTACTTGGAATTGAGGAAATGATTACTGCTTAATAATTCTTT
GTGGTAGAGGGAGAGTTGGTATCATATTTATGAGACAGCAGCCAATATAGTATATCT
CAAAGGAAAAAATCCATTCTACATAATGCCAGAATTTAATAGTTAAGCATTTTATCTA
GGTCACAGCACAATAAGCAAGATGGATAATTAAAATAAAAGTATATTTCTCTTGCAT
ATATTTCTCATTTCATGTTTCCCTATCATATTTTATATCTTACCTTACTTCAAATACATA
TATACCTTCAATAAAACTGAGCCTTCTTGCTTACCCAGGAAGTTTCATCATTCAGTAG
AAATAAAAGATGACTTTAGAAATATTAAAATACAAAAATCTACACTGAGGTCTTTTG
AATGCAGGAAAAAGAATTATATCACACACACACGTACACGCACGCATGCATACACAC
ACACAGAACCTCTCGTTCTTTCTTAACATCTTATCAATCCATCAGTTTCACTCCCACTC
CGTATCACCTGACTGTGCACAATATCTCATTGCCACCTCCCAGTCTTCTCCCTGCCTG
GCACCCTCCTGCTCTCCTGCTTCCACTTTAAACACCCTTCCTTCAGCTAGGTCTTTTCT
TTCAGGGATCCTCCCGTTGCTTTCTTATCTGGATCAATTTAGCCTTCCTCTTCTCCACC
CATTAGTGGATAAGCACGACAAAGACACTAGAGTCAAATAATACAAACAGAATATA
CCTTAGATGAGTATGGTGATGAAAAGGATATGGATACTTAGAGTTTAGCACTATTCTC
TCAGCCACTCAGGAAAGCAACGCCTTTACAATCAATAGTGTTTCAGGTACCAATCAA
TAATCTGTTATTGCTATTTTTAAAATCTATAAGGTATCAGTAAAATGTAATTACTAGA
GCAACAAAGATATCTTGTGAAATCAAATTAGTATTCATCCAGCAACTGAGTACAAAG
GTTTAAGGGAGGATAACTACCAATACCAAAACATTTTAAGCATTTTGTTTTGCCTCCT
AAATATCAAATCATGTAAATGTGTGGTACATAAATTAGGAATTATATTTATGACATA
GCTGCAGACATATTAAGAGAAATATGTGCTTATATTTACAAGTATAGTACAGTTCTTT
TTCATATTAGATACTGTTGATGATAATCTGCATATAAAAATGCTCAATATTTTTTCAC
ATTTATAAGCCATAAAATACAGCTAATAAAATGTGTTTCTACTTTCTCATAAACATGG
AATAGTGACAAACAAGGAGCTTTATATGAAAGCACCATTACAATTTAAACTCTCACA
AGGTCATAATATATTGCACTAAGCAGGAGAGTTCAGCTTATTTAAAAAAAAAAATAA
ACTCTAATGAGGTTCTGGAATGCAGAGCCAAAGCATAAAGATGGAAATAAAAGAAT
TGCATGTCTTCTGAACTGACTTGGTTGATGATTTTTTTAAAAAAGGTTTTGTGTCTTCT
GACTTGGTTGATGATTTTTTAAAAAAACGTTTTGTGGTAGAACAAATAAGGTAAATG
AAATTCAGTATTTAGGATGAAAAGTTTTTCTAATTTCAGGAACAACATTGAAGAAAT
ATTGAACTAAGCAGCTTTGAAAGAATCAGATTCCATTTGTTGAAATTTTTCTGAGAAT
GAATTTTTTTAAGACAGTGTACACAGTTGCAGTGTGTATTGGTTATGGATTGTGGCAA
GCTATATTACAACTTACCCAAGAAATAAGGAGGCTGGGCGTGGTGGCTCACACCTGT
AATCCCAGCACTTTGGGTGGCCGAGGCGGGCGGATCACGAGGTCAGGAGATCGAGA
CCATCCTGGCTAACACGGTGAAACCCCGTCTCTACTAAAAGTACAAAAAATTAGCCG
GGTGTGGTGGCGGGTGCCTGTAGTCCCAGCTACTCGGGAGGCTGAGGCAGGAGAATG
GCGTGAATCCGGGAGGGGGAGTTTGCAGTGAGCCGAGATTGTACCACTGCACTCCAG
CCTGGGCGACAGAGCGAGACTCCGTCTCAAAAAAAAAAAAAAAAAAAAAAAAAGA
AAGAAAGAAAGAAGGAAAAAAGTCACTTGAAAAGAATACTGGACTTTGTGTCCAGC
TTGCATAGCTGAAAAGAATAAAAACCTGTCCACTTAAACTCATTGCAAAAAGAAGAT
GTCACTCCTACAAATAGCAAAGAGTCATGAAATTATTCTATCCAGAAAAGTATACAT
TTCATCCCTTTGGATAAATTTTAGAAGTGAACTATGAATACATACGGTGAGGATAGCC
AGCTAAGAAGTCAAGAAGGATTTCTCAAATTTGCTGCTCAGAAAGATCATACTCTCC
ACAAAACAAATAATAGCAGGCTTTCCAAGTCAACCTTGAATCCAGCTTTCCTTTATCT
TTCCTTCTTGTGAACTTTCACTAGTTTACTATCTAACAATGAATTTGACGATAGCCAC
ATACCATCTTATAGCAATATTTGTTATCATATCCCTTGTTATTTATCATTCACCTGCTC
TGCTTGAGCCAGCTACAAGTCACATGTCCCACGCACTTTTTCCTGTTTGATTTTTTACA
GCACTTTGAGACATGTCTCATTATTCCTACTTGACAGGAAAGAAGCCATGGAAAGTT
GAGTGACTTGCTCCTGATCACAAATGCTGGCCAAGGAAGAGTCGAGTTTCAAATCTA
ATGATCTTTCCACTGCACTCTAGATTCCTCATTTTGAACTATTTTTTTATTTTTTGCACT
ATAGACTTTTTTCCACATTTTGAACTGTTTTTTATTTTTTGCACTATAGACTTTTCTCTT
ATACCCAACTATATTGATGACTTCTTTTAGGCTAGAAACTTGTTTCACTTACTTTCCCT
TTCTTCAGATTGCTGCAATATTGGCCAACATGTATTGGGTACTTACTGAGTCAAGTAC
TGTGATTGTGCCAAGTATCTTATAGGAGGATTATCATCCTCATTTTTACAGGTGAGAA
AGGAAAGGAGGTAAAGTCACACACAGCCAACAAAAATGGTAGCACCAGGATTTGAA
ACAAATCAGTCTGACCCAAGTTGACTTTGTTAACCACTGTATGCACAGTCTTCTTAGA
CATAGTAAGAGCTCTAATTGTGTTTGGTGATTTGATTATTATGACAAAGTAAGTAAGG
GAAGCAGGGAGAATTATAAGAAATAAGGCTCCACAACACTTGGCTATAGCAAAGCC
CCTTAAAACTTCAAAAGGTCACCCAAAGAATAAAGATCAGGCTGGGAGCAGTGGCTC
ACGCCTGTAATCCCAGCACTTTGGGAGGCCGAGGTGGGTGGATCACCTGAGTTCAGG
AGTTCGAGACCAGCCTGGACAACATGGTGAAACCCTGTCTCTACTAAAAATACAAAA
ATTAGCTGGATGTGGTGGTTGCCGCCTGTAATCCCAGCTACTTGGGAGGCTGAGGCA
GGGAGAATCGCTTGAACCCAGGAGGTGGAGGTTGCAGTGAGCCGAGATCATGCCACT
GCACTCCAGCCTGGGCAACAAGAGCAAAAAACTCTGACTCAAAAAAATAAATAAAT
CAATCAATAAAATAAAGATCAATTTGGAGAAATTAATGCTTATTAATAAGCAATGTC
TTGCACAGCACTTCAGTTTCTCAATACATTACCTAACTCAATCCTTACAACAACACCC
TATCCCCATTTTGTGGATAAATAAACTCATGTTCAGAAGGTTGAATAAATTATCTAAG
GTTAATAGTTCCTGACCTAGAGCTCAAATCTTCAGTTTCTATCATATTCTTGCCCTTAC
CCTGGGGTAGCTAACATTCACTCACTAGTATTGGAGCTAAAATAAGGGAGAGAACAT
ATAAATGAATACAAAGGAGACATTCACCTGCCTTCTCTTTCTCCTTACATAGAGAAGG
TTGATTATCTGCTATTGTGAAGTTTGCCTTTTGAAGGATAGAAATGAGAAGACTTTCT
TAAATTTTGCCTCTACGCCAAGAAATTAGAGTGGTACCACCAGTAGTTCCATTTTCAA
ACTATCACTGTAGCTAAAGCTATGTGGTAAGGGCCAAGGAAAAGAAGTATTCTTGCA
CTTCAAAATGCACTGAAATACCAGTCAGTAGCATAATATAAAGGAATTTAGTGGAGA
GAAGAGTTGACCTCAATCTGGCTCCAACATCTCGGCTCTTAACCCCTACCCTACACTT
GTTCTTCATGGGGAAGCTAATTGGGCCACTGGAAGATTCAGCAGCTACCATTTGCAG
CTGAGGGACAGCCCCTCCCTGCTTAGCAACCAATGGATATGCATTTATGGAACACCT
GCTAACTGCGACACACACTCCTATGTATGAGGGAAAATACAAAAAATGTTAAAGGAG
ATGCCTTCCCTTGCCCTCAGGAAACTTAAGTATAGTTGCAAAGAAATGATTAGCAGC
AAACGAAACCATGGAGAAGTAAGGGCTAAGGTCTGTGAAACAAGCCTAGAAAATAA
CCTTGTCCTTGAAAAACACAAAAAGAAAGAAAGAAAGAAAAGAAACTCCAAGGCCC
TTGTGAAGGAAACCATTAAGTTTGCTTCACTTCTGTGTTTAGGAAGACACAAACCCAG
TCTTAATGAACCTCAAGGCCACAACTACTGGAGACATTTAGGAATTGTCACCACATTC
TAATGTATATATCCTCTGTTTGGCCCTTCCTATTAATATTTTGTAAAATTTTTGAAGAT
ATGAGCAATGTTTAAAACCATGAATCCCCCTTTTTTTATAAGTAATATTTAGGCTGAA
TAAACAAGAGAAAATAGGACATAAAGGGGAGCCAACGTGTGCCTTCATTTATAATGT
ATTCCCAAGTTGTGAGTTTGGTTTATCAGCAATTTATCATGCCAAATTCCAAGTCATA
TTTATCTATGCAGATCAAACACTTGATTCTATTTTTGCCTTAATTTTTTTATTGGGTAT
GTTTATGACCAAGTCATATGGTATTTTCTGTGACAGATAAAATGCACAGGTTATTCCA
ATCTGGCTCAGCCAGTCATAGCAACATGTAGTCCTTCTCATGTCTTAAGAATGAGTAT
CAAGAATTCAAAGGGAGTTCCAGATGGCATCCAAAAAGCTTACAGTTTATGCATCAC
TTATTCTAACAGTAGAAAAAGAATATTTGAAGCCAAAAATAGACCTTGCATGTAGCA
TGTGGAAGAGTAGAAATTGCCCTGATAGTTAAACAATTTGAAATTCAAGACATTAAT
TTCTTTATGAAGCATTTGTCACATCATAGGTAATATTTTATGCCTATCATATATATACT
TATTATGAAATACAAAGAAATTATTCATTCTATCTAAGACTTTGTATCCTTTACCAAT
ATCTCTCCATTCTCCCACCTCCACCCTAGCCCCTGGAAACCACCCTTCTACTCTCTGCT
TCTATGAGTTCTTTTTTAGTGAGATCATGCAGTATTTGTCTTTCTGTTCCTGTCTTATTT
CACTTGACATAATGTCCTTCAGGCTTATCCATGTTGTCACAAATGACAGAATTTCCTT
CTTAAGGCTGAATAGTATTCCATTGTGTGTATGTAGCACATTTTCTTTATTAATTCATT
TGTTGATGGATACTCATATTGATTCCATATCTTGGGTCTTGTGAATAATGATGCAGTG
AACATAGGAGTGCAGATATCTTTTTGACATACTGATTCCACTTTGATGGGATATATAC
CCAGTAGTGGGACTGCTGGATCATCTAGTAGTTTTATTTTTTTTTATTTTTTATTTTTTT
TATTTTGAGACAGAGCCTTGCTATGTCGCCCAGGCTGGAGTACAGTGGTGCCATCTAG
GCTCACTGCAATCTCTGCCTCCTGGGTTCAAGCAATTTTCCTGCCTCAGCCTCCTGAG
TAGCTGGGATTACAGGCACGCACCACCATGCCCGGCTAATTTTTGTATGTTTAGTAGA
GACGGGGTTTCACCATGTCTCGAACTCCTGTCTTCAAGTGATCCGTCCACCTCAGACT
CCCAAAGTGCTGCGATTACAGGTGTGAGCCACCACGCCTGGCCTAGTAGTTCTGTTTT
TAATTTTTTGAGGAGCCTCCATACTGCTTTCCATAATGGCTCTAGGAATTTACATTCC
ACCAGCAGTGCACAAGGATTGCTTTTCTCCACATTCTGGCTAACCAGTCTCCTGTCTT
TTTGAGAACAGACATTTCAACACGTGTGAGATAATATCTCATTGTGGTTTTGATTTGC
ATTTCCCTGATGATTAGTGATCTTGTGCCTTTTTTCATATAACTGCTGGACATTAATAT
GCCTTCCTTTGAGAACTGTGTATACAGGAGAAAATAATCACTTCTCAGAGGAGCTTTC
ATTTCAAAATATCCGGGAAAAAAATAGAAAAAATGGAAAATTTATCCTAGAGTAAGT
TGTCTTTTATATTTTGACCCTGTTTGTGACATAAACTGGATGATACAAAACTGGAATG
CAAAGGCTTTAGGAGGATTACTTACTTACTTGTATATTGCTTTAGGTTGTTTGCAGAA
AATTATACTAATTGAAGTTCAGGCTATGATGTGATAAAATCTATGTCAGGAGATGAG
TCTACATGCAAAGTTTGAGGAAGTGACATTTGAGTTTCAAAACAAAAAAGCAATTTT
CAATGTCATATCTAGGTTAACCCAAAAGATTTCTTTCACCCTATTTAGCTGCCTCTAA
GATGGATGCTGAGGATAATTACACTGTAGAACAATAGGACGATGCTTCACACTCACC
TCACAGGCTCTGTTATTCCCACATACTGCCAGAGATACTCCAAAATAAAATCACTGCA
ACATCAGGCAGTTATAAACCTCAACGGTATTATTTTCTATTTATATACAGTATATTTT
ATATTTTACAAGTATAAAATAGAATATATTTATTCTATTCTCTTTGACACAAAGTGAC
CATAAGACATATTACTTAAGTATGACTAGCAAAGTCATGGGGCTTGTCATTCAGGAG
GAAACTCTTAACTAACTGTTCAGTTTTTGTTCACTGCACCATTTACATAAGCCAAACT
AATGCTTCACACTGTGCAAAACAATGCACAGTGTTGTGAATGAATGGCTAAAATAAA
ACTCTAATGAGTGGGGTTTGAAAAATGCAACTTTAGAAAACTGTTGAGAAAATGTTG
CACACTGCGCATTTTACAAAATTTCGTTGAAGGACACTGGATATTCTTTTTAGGATTA
TGGAGGGAAGCAAAATTTTGGCTCCTACATGCAGTTTTTGTGGCCTTTGCCTGAAATA
GTCATCTCCCATTAATTATTTAGATATCATTCATTTCCTAAGACAACATTTAGGGAGA
CTGCCTTAAGTACAATTTGTACACTACCCAGATAAGAATTCTTTTTGGTGAAACATCG
ATAAATATTACTTGGCAGTAACACCAAGTTAAAATATTTGTTTCACAGTCGACGTTAA
TAACTATTATAGATAAAGTGAATTTTATAAGACATACTCAGATCTAAAACAGCAATA
TGGAGCTCTTCAAATCCATTGAAACTTCATACCAGCCTACGGAAGTAGAGGTTTTTAT
GCAAACTCTTCAAGAAATATGCTCTGAACTTTTAATTCCTTAGATTGATAGAGGAATT
AAATCATGATATAACTAATAGGTTTGTGGTACAAATTGCTGCTGCTTAATCTGACTCT
GTGTCTTCCCAGTGTTCTATATGAATTAGATATTCCATTATCTAAAGACAATCAACCC
CATCCCACGGTGATAGCTCTAGGACTCCCTTTGAGTTCATTAAATCTGTATTCTCAGT
CTCCAAACTTCTGGTTAATTCAAACAGAAAAGTCAACTGGCCCATGAACTAAAATAA
AGTCATCTGAATTTTTTTTTTATTTTGCAGTGTGATAAAAGTCTCGCACTTTTTATTTC
TGAAAGTTTCTGCTTTCACTGAGAGCATAATAGGCTATCCACCCTTATGCAATCTTAC
ATACAAAGTCATAGTCAGGCTAAATTCAAAAACACATGTGAGATAGAAGTCAACGTT
TATTTTCTGGAGAAAAGCCACACATTACAACAAAGTGAACAATGAAGCTGGCATCCT
TATCACTGGTGACCAAAACATTTGTGACTCTGGACATTGGCCCCACAAATGCGATAA
ACATTCTGCATAGGAAGTGAGTTTTGCTAATTAAAAATGGATCCAAAATACTTTCTAC
TCTTCAGCCAAGAATTAAAAAGTAATAGGGAGGAATTGAAATCACTTGGGTGCTACA
TTGAGCCATTCTGGAGAAGCAATTCAGAGAATGTCATGGCAGCCTCAAATTGCTGCT
CAGGAGCATCCCAGCTTAGAAGATTGCAGGAAAGGAAGAGCAAAGTCATTCTTACAT
GAGAACTGTCCTTAACCAGATGAATAGACTCTCCATTTTTTACCCTGGCTTTGTCTCA
TTTAAGTCCCAACCAATCTAGCTATCATTTTAGGTTTTACTACCTGCTAGTATTTAGGA
GCTTAGGGGGATAAAAAAATCCCTCAATACTCAGAATTAGACTTGGTGATAAAAATC
TTGACACATAAACAGAATAAAGCGCTTTCATTACTCCTCTAAACCACAGTGTCATTTG
GTCTCTATCAAGGACTGTAAGAATTTCTTTCATCAGGGGAAAGAAAAAAAGGACAAG
AGCCTGCAAGATGTAGCGGAACTCTCATTAAACACAGCAGGAGCTTTAACTGGAATC
CAGAGTAAGGTGAGGTACCAGGTTACAACAATTTACTGCTTTTATTACAATTTTGATC
ACAAGGACTGATTCATGTCATCTAGTTTCTTTTCCTTGTCACTATCACTGGTGCTAAG
AATACATCAAATTGAAATTTAAGAGCCTCATATGTTTCTGTATAACCCAGTGATGGGT
TGTACTGCTTTGACCTTCTTAAATGTCCCTTTATTTCATTTGATATCCATTCCCATAGA
AAAACTATAATGCTTTGGTTGGTCAAAATATTAATCTTTCAAAACCTCCCTGGCTTAG
AAAACCAAATTTTTGTAGAGAGAGATGGGTAGAATCTAATTTTATTCTAAAGCAATT
AGCATTACATCATCACAGCAGAAATATCTAGAATATTACCTCATGTCAGTGATCTTCT
GATATGTTAAAAAGGGTATTTTAAAATCTGAGTTATTTCTTTTTCTTTTTAAAGTTACA
TCATTAATTACATACTCATCAACCAAAATATTTTATGCTCCAAATTTGAACCGATATA
GTATGTAAGAAGTGTTCAAAATGAAATTATTTTGGTCTATTTTGTCTTTGAAGAAGAT
CACAGGGATGGACCTCCCAAAAGGATTTTTAAATGGGATTACATATCTGACTTTTAA
AAAAAATTATCTGACCTTGAGTTATAGTGCCCCAAAGTAAGCAAAGTTCCAAACACA
CAGTATCATCAGAATTGAGTTAAAATTATCACCAGGGGCTTAATTTCTGAAATTAAA
AAGGAAATGTTATTTCCTTATGAAAAGAAAAGGAACCAAAAATGAACTTCAAGGTAG
CTGATTTCTGTCTATGTTAAGACTTAGGTAATGGGAGAAAGGGAAAAGGAAGGACAG
AATTAGGAGAGGAGCAGTGTTTAACAATTGCGGGTGCAAGACTCAAGTTTTTTAGAA
TCCATTAGCAGAGAACCCTATTTCTCCCATTAACTGCTGTCCTTTTAAATCCTGGCAC
CAGCTCTGAGGACTGCAGGGTCCATAGCTAGTGCCCCACTCTACCCAGTTTAAAGAC
ACCACTGCCTGGAAATGACAGGGGTTTTTTTCTTAAGGAAAGAGGTGCTTTCTGCCAC
GTATATATAAATTGGTAAGCTTCAAATAAAGTGCTTTTGTCCTTTCTGTCTATCAGAA
ACTGTGCAAATCGAATTGCTGTAAAACCAAGGGCAAGAGACATCAATCCTGCATTCT
ATAGCATCTGATTTTATCCTTTATCCCCAGGCACATTTCAAAAGGAAAAAAATGAGGT
TGCATTTAAATTGAGTATTTGGGACTTGCCAGGAAAACCTCCCGCTAGACTAATATGA
TTGCAGGGAAAACAAGAGAAAGGAAAAGTGGAGAGGGAGTGTGCTAACAGATCCTG
GGCCTCGTCAGCAGAGCCGTCCTGAGCACAAGGCCATGGTCAGACATCTGGTCCCGC
GAATGACGTTTTCTTTATGGTCATTAAGAACACCAGTGTGTCGGGACACAAACAAGT
ATTCCTTTCAGGGATTATGACACATTTTCTCCCAAAGTAGTATATTAATGACATTTCC
AGAGCATTCTTTACTATCTTTTATATGTGATCAGGAAGACTAATACATATCACTACTT
CTTTTACACACAGCATTAGCCAAAACTAAAGTGTCAAATACAATTTTGCCTAGGATG
AATAAACAGAAGAAATTTTTATGATACTGCACTATCAATTCCAAATTAAATAACAAC
AAAATGATAAGTGTTAAAATTCATATTAATGATTGTTCCCACACAAGCCGGAAAAAA
TCTTTCTAAGAAGTCTTTCATGAGTTAATCCCATCTTTCAAAGTGTTCAGTGGCTCCG
AATTCAGTTACTGTTTCCTATCAGTTCTTCTTTCATTAAGTCTCTTCCCTTTTTTTTCTC
TTTGCACTATTTCCCTTAGCCGGGTACATAATCTGCTGTGCTTTATTCATTTGTGTCTT
AAGTTTGTTTCCCGATGACATACCTTTCCAGCAACGCCATCTGGGGAGTTTGGGCAAC
TGTACCACGTTAGGAGGAAACCCTTCTTCACAGGAGAGTGTGCCTTTGCTGCAGGGA
AGGAATTAGGATTTGCTTGGACTGTGGTTGCAGCTGGCTTTTAAGGATCTCCTTAGAA
TGCAAGCAACTCATCAATGAGAATCTCTGCAATGGTTGTCACTGGGTAGAGTCATGC
TATGTGGGGTCATAGCCTTTGAAACAAATAACAGTAAAGATAAAAATGCTATTAAAG
GAATCACCACCCACAGAGGTTAACTGGGTTTTGTCCCCAGACCACCTCGAACAAGAA
AGAACATTTTTATCAGTCATTTTCTTAGTTTTAGCTGATAAAACAAAGTACCATAGAC
TAGGTGGCTTATAAACAACAGAAATTTATTTTTCACAGCTTTGGAAACTGGAAGTCTG
AGATCAGGCCGCCAGAATGATCAGATTCTAGTTAGGGCCTACTTTGCTTTTGCAGACT
GCCAACTTCTAGCTGCATTTTCATGTGGCAAAAGGAGATTGAGCTAGCTCTCTGGTCT
CTTCTTATAAGGACACTAATCCCATTCATGAAGGCTTCACCTTCATCATCTAATTACT
CTCCAAAGACCCCACCTCCAAATACTATCACATTGGGAATTAGATTTCAAATACAAA
TTTTGCGGGGACACAAATATTCAGTCCATAATAGTAATGATTACTCATTATACATAGG
GCTCTAAATGTGCTAGCTTCTGATAGTTTTTACACTCACTTCTCTTTATTAGCTTGTCA
AGCATAATTAGGGCAGTGGCCTTACTGAAAATTATTGAATTTAGTTTCCTAAGGACA
GATATTGAGGAGTTTTTTCTTCACTAAAAATTCACGTTCCGATACAGCTTTCATCTGTT
ACTACTTTGTGAGATGGAAAATCTTTTATTTTATTTTTATGTTTGGATTGACCCTTCTT
AATAAAGTCGGCATGTAATATGCTTCATGTGTTTCTAATATGTGCTTAATTTTGCAAA
ATGTTTTGCATACCAGAATGCATTTCTCTTCCAAAAAAGGTACCAGCCTACAAAACCT
TGCTGTTACTGTTTTCAATTAGTTCATGGAATTAAATGTATTAAATGTTTTATGCTCTG
GCAGAAATTATGATTCTCACTTAACTCCATATAAATCTGGATCTGCCTGGGCCTTTAT
AAGTGACACAATTTCATTAACTGAATAAACAAATGATACAAAGAAATTTGGTTTAGC
CTTCTAAAATTCCAAAGGCGTTCAACAAAATATCTCAGAATGGATGTTCCAGGACTTT
TATGGCACAGGACAACATGTATTGCTTATTTTAAGAAAATAAGCTAAATAGTGAGGG
GATTCTTTTAGCAGATCCTCAGGATGTGTTAGGTTGAATCATAGGCAAATGATATTTG
ATCATTGCACCTGTTAACACATTGAACCTCATCCTAAAATTGTAGAGCTAGAAGAAA
GCCTTCTGGCAGTTTTTAAATAGATTGATTTACTGCAATTTATCCAGAAGCTTCACCG
TTGTCACTGGCTACATGTGACTTTGGCCTCTGTGGGGCTATATCCTCATTTGTAAAATT
GGTGGTGAGGTAGGTGGACAGTTGACTAAATAATCTCTTAGAATAATTCTAGTATCT
GTGGATCTAAAGCATCCAGGGGTTGAATATGTTTCTTTCTGGCCAAGAAAAGATGCA
CCTGTCAATAATGCCCAAACTCATCTTCTGAGAATCCTCTTTCCCAAGATACCCACTC
TCCCTTGGGTTATATTATAGTAATGATCAGAAGCCCCTGCCAAGAAGAAACTGTTAA
CCTGGGAGGTCTATATTTTATTTCACAGCCATCTGTTTATACTTTCTCACAAGTTAGTG
CACAGTATACCCATCATTTTCTACCATTTTCCTTAATTTATTAATTTTACTAATTGCAT
AATTAACAAAAGTAAGAAGATTTTACCTCCTTATCCCCATCTGGTAGTTTGCAGATAC
TTGGCCTGATGACAACTGACAGTGATGAGATACTCACCAAGTTTACCAGGGCAGGAG
GCTTCCTAGAGAAAAAATGAGAAAATGAAATGGGGAAGGGGAGTGAAGGATTGAGG
AGGTGACAATCTGGACTCTTGCAACTGCATGGCAAGGTTGGCACACAAGCTGGGTTG
CAACGGAGGGAAGGAGATCCTTATCAGATGTAATCAGAGCTCAGATCGAGGGCTTTG
GTGTGTGTAGAAAGAGGGAGAGACAAAGAACTTAAAACAGAGCTGCCATTTGACCTT
GCAATCCCATTACTTGGTGTATACCCAAAGGAGAATAAATCATTCTATTAAAAAGAC
ACATGTGCTTGTATGTTCATGGCAGCACTATTCACAATAGCTAAGACATGGAATCAA
ACTAGGTGTCCATCTATGGCAGATTGGATAAAGAAAATGGGGTAAATATAAAGCATG
CAATACAACATGGCCATAAGAAAAAATGAAATCATGTCCTTTGCTGCAACATGGATG
CAGTTGGGACCCATAATCCTAAGTGAATTAACACAGGAACAGAAAACCAAATACAG
CATGTTCTCACTTATAAGTGGGAGCTAAACACTGAGCACACATGGACATAAATATGA
GAACAATAAACACTGTGGACTACTAGAGGGGGGAAGGAGAGAGGTTTGTAAAACTA
CCTATCAGGTGCTATGCTCAATACCTGGGTGATGGGATTTACACCCCAAACATCAGC
ATCATTTAATATTCCCATGTAAAAAGACTGCACATATACCCCTTGTATCTAAAATAAA
ACTTGAAATTAAAAAAAAAAGAAAGAAAGAAAGAGGCTGGAAATAGAGGCTCACAC
CTGTAATCCCAGCACTTTGGGTGGCCAAGGTGGGTGGATTGCTTGAGCCCGGGAATT
CAAGACCAGCCTGAGAAACCTGGTGAAACTCTGTCTGTACAAAAAATACAAAAATTA
TCCAGGCATGGTGGAGCGCACCTGTAGTCCCAGCTAATGGGGAGGCTGAGGGGGGA
ACATCACTTGAGCCCAGGAGGTGGAGGTTGCAGTGAGCTGGGATCACACCACTGCAC
TACAGCCTGGGTAACAGAGCAACTCTGTCTCAAAGAGAGAGAGGAAAGAAAAAAGA
AAAGATGGACAGATAAGAAAATGCACTTGGAGATTAAGAGAAAGCAGCAACATAGG
ACCCTGGATAATGTGTTTGCTTAATAACTATCCTGATGAGTTATCTGACTATTCCCAA
ATGAGTACGTGGCAATTCAGGCTGAACCATCAGAGTAGCCCTCCGGAATCTTACTTA
TGTACAATAGACCTGCATGCACATTTACTAGAATGAGCCTCTCTCTCTGGTAATCATG
TCTGCTTCCACTAATTCCATCTGTTTCCTCTCTCTCCCTCCTATCCTGCTAGATCTTAAT
TCCTTCGACCTTCCTTTGTTTTTCTAACTCCCTTTCTTTCTCTTGTTATTTAACCTGCTA
TACTATGCAATTGATCTCCTCTGCACTAAGGAACATGCACTTCAGAATTCTGTTGACA
TCTTGCATTCCTTTATATTTAGTGAAAGAATGCAAAGGAGTCTACCTGGCAATATTCA
CTCTGCAGGAGGCAATAATTATTATTCAAATTAAAGGAAGCAGTAAAGAGAAATTCA
GAAAAAATGAAATATACTAATCTTCAGCTTTTCATTTCAGCCTACAAGGAAAAAATG
AAGGAGCTGTCCATGCTGTCACTGATCTGCTCTTGCTTTTACCCGGAACCTCGCAACA
TCAACATCTATACTTACGATGGTGAGTAACCTAGGATAGACATACCCCTGCTAGCTA
GATCATTTGGAAAGGTTGACATATATTTGTTTCTTACAGCTCCTGATATAATTACATC
AATATTTTGTAGCTCTCACTATTGACTTGCCGTGTCTAGCTATTATGTCCAATTGATTA
CCTATTGCTGAAAACAGTTTGAATTTGGTGCTAATAACAACACATCAATGTCTGTTAA
GAAATGTGGATGGATTCTTATTAACAGCCACATCCAGCATATCAACATCCACAATAT
GTCTAAGGTCTTTCTTTGCAAATAATTTAATAGGCTAAGCCATAATTGGAGTAGATCA
TAATTTGTAAGAAAATGCTTTATACTTAGAAAACTCAAGAGAAAGAATCAACAACCA
TAATTGTTTTTGCTTTATTGTAGTCTTTATAAAGTTTCTATACTTTGTATATACATGTC
AACCAGCTAATGATAATAATAATTGGCTCAATAAATAAAACTGACTTACGACTGAGG
CCCTAGATAAAGAGGGTCTGAAAAGAAAAGCCTAAAGAATTAGCATGGCAATTAAC
ATGATTGAGGTGCAACTCTTTAGGTTTGATTTATCCTGATTCATTTTGCTTACTTTGGC
TCTGCCACAATCCACATGATCTTGGTCAAATAGATACTTGGATTCTCTAAGTCTCATT
TAACTCTAGCATCTTCCTCTTGGAGTTGTTGTGAGGTTTAAACGGTTTAATGTAAGTC
AAATATGCAAAACCAAGCCTAGCTCATTATATCACTCTACAATGATAGCTATCATTAT
CAACATCATCCTTACCTAATTCAGTCAATTTAACTAAAATATTTTATACAGTTCTATGT
ATCCTAGATATCCCTAAGGCATATTTTACTAACTCTCAGGCTCACAAATATTTTTCTTT
TCCATATATGTAAAGAAAGACATTAATGACAAAACAAACTGACCTTGTGGCAGTTAA
CCCCTTCTGCACCTTTAAAGCCTATTCAAGGACTCAAAGGCATTTACCTTCCAAAGTT
ATTCTATCGTAGCACAAAAATCATAAATGCTAATTAACTGTTCCATAAGGAAATGTCC
TCCATGTGAAAGGAATTCTGTCTCCAAACAAAACATTCATTAGAATGCAGGGCCAAT
GCCTACTTTGTACAAATTCATTCGGTCAGCAAATAAATTAGACAGACCTTTATTATTT
GCTAGATGTAGCTGTGAAGAAGGATCCAGCTATGTTTCTTATGAGACTAATGTCGAA
CTATGGGTTGTCACTGAGGATCCAGAGTTCCATAGGGCGTAGTCCTCACCTTCAAAG
AATTCAGGGCTTAGTAGAAGAGTCTTACACAAATGACTAGAATGTAGAACACAGAGT
GGTTAGGACAAAGGAGCCAGGGATGGTTTTTGCTGGGTTAGGGAATGAAAAAAGGG
GAAGAAAATATGTGAAGTTATGTGTGAGCTGATTCTTGAAATAAGCTGTTTTTATTTG
CCTGCGTTCTCTTATAATCCTTTTCCATAGGCTTCCATAATTTTTATTGAGCTGTATTT
AAAGTTGAATAGATAATTCAACATTTCTCGTAAACTGTGCTTCCTAAAAGAGTCCGTA
GAGAATTTCAAATTTCTGCAGTCTTTAACTTGACCTGGTATTTCTATGTTAGATAATA
ACGTGACTTGTTTATTGCAGGCAAACATTATAACAATAAATTATTATTATTGTTTACA
TTTGTAAGCACTAAGTATATGGCTTGTGCTTTGCATTCAGCATCCTTTATCATTTAATC
TTCACAACCACCTTAGAAGGAAGGTACTCTTTTTATTTCCATCTTTTAAATGAGGAAA
TAAAAGCATAAAGAAGTTAATTAACTTACCTAGTGTCACACAGCTATTAAGAGGGGC
TTACTATTTGGATGCAAATATAGGCAGTTCTAATTCCAGAGCCTCTAATCTAAGGCAT
TTAAAACCCCATCACCTTATCAAATAAGCTGTTTTTATTTGCCCGTGTTCTCTTATAAT
CCTTATCCATAGGTTTCCATAATTTTTATAAAATTGTATTTAAAATTTAAGTATAATCT
TGGATGCCATCAGGAAAATGAAAAACATTTTTACATTTGTGAAGGAAAAAGCCCACA
TCATTTCCAATATAGTTATTGAGTTAGTATTATCTAGACTATCTATTAGCAGCTAAGG
ATCTGAGGTCAAGGCCTGCCAGCCTGGCATTTTACTTGACCACAACCTCCATGTGCAC
TAACCAGGCTGCTAAAAGAACATTAACGGGAACATAACCTGCTGGCTTGGTTGCCAC
AATTTTAAAAAGACGTTAATAAATTAGAGAGCACTTAGAGGTTAGGAAATAATATGG
TGGTAAAGATCTAGAAACAGTGTCATTCTGGGGCACTTGAAGATGTTTAGCCTGGGG
GAACAACTTGAAATGGAACATAACTGTTTTCAAATACTTGAAAAATGGTGGTGCACC
ACAGAGAATGGCCTAATCATGGGTAGCTTCAGACTTCAAACAAGGATCAGTGGGCTA
AAACCAGAGAGATGGAGTTTGGGACTCAAAGAATGCTCATCTGAAATTGAGGGCTGA
CCAGCGAGGTTCTTTTAAAAATCATTGCATTTTACTAAATTGTGAGTTCTGTAATTAT
AAATGTCCTAGCAGGTGCTAGCTGTCATCTTTTCTATTATAAATTATACTATTTTATGT
TATAATTTGTATTATACAGGCTTAAAACATAAGGGTCTGATAATCTGCTTATCTTTAA
TACATAAGCCACTGATAGAAAATAAGTGGCTAACCATTCTTCAGTTCTTTTTTTAATT
GACAAAAATTGTATATGTTTGCGGTGTATGGCATATTTTGAAATATGTATACATTAGA
GAATGGCTAAGTGAAGCAAATTCACATATGCATTACCTCACACACCTGTCATTTATTT
GTGATGAGAACAAAAAATCTACTCTTTCAGTGATTTTCAAGAATACAGTACATTGTTA
TTAACAATAGTCAGCATGGTGTACAATAAGTCTTCTGCGGCCGGGCGTGGTGGCTCA
CGCCTATAATCCCAGCACTTTGGGAGGCCAAGGCTGGCAGATCACGAGGTCAGGAGT
TCGAGACCAGCCTGACCAACATGCTGAAACCTTGCCTCTACTAAAAATAGAAAAATT
AGCTGAGTGTGGTGGTAAGCGCCTGTAGTCCCAGCTACTCAGGAGGCTGAGGCAGGA
GAATTGCTTGAACCTGGGAGGCGGAGGTTGCAGTGAGTCGAGATAGTGCCACTGCAC
TCCAGCCTGGCAAAAGAGGGAAACTCCGTCTCAATAATAAGTCTCTTGCATTTGTTCT
TCCTGTTTAACTGAAATTATGTATTCTTTGATCAACATCTCCCCAGTCTCCACCCCTAA
CCCCTGGTAACCACAATTCTACTCTGCTTCCGTGAGTTCAACTTTATGAATAGTCCAC
ATGTAAGTGAGATCATGTGGTATTTGTCTTTCTGTGCCTAGCTTATTTCACTTAGCATA
GTGTCCTCCAGGTTCACCCATGTTGTCAAAAATGACAGGATTTCCCCCAACTTTTTTA
AGGCTGAACAGTATTCCATGTGTATGTGTATAAATTAGATTAGTAGATGTTGCCACTC
CCTCCTCCACCACAGTGGCTCTATCCCTGGCTCCTGGCTCCAGCCGAGTACACTAGAG
GAGGATATTCTAAACAGCAACAACACAGGAGCAAAGACATTACAATGGGGTGTTGTC
TTATTGCCCCCATTAGACTGTAAGCATCTTGAAGACAAGGACCCCCATCACAGAGTG
ATGTTGTCATCCCTGGAGTGGGCACTGTGCATGATTGATGACTGGAAGCAATGAACA
TACAGAAGGGCAAAACAGAAATCAGCAGGATGCTTTGCATTTCAGCATTGACTTTGC
CAAATATGCCCAACTGTTCAGGGAGTTACATTGGTTCTAACGAAGCTCCTGTGATTCC
TAAGCACAGGAATGGTGATAATATATATAATGGTGCATGCATATATACGCATATCTA
GATAATGATATCTCATTATATGTGAGAACTGAAGAACTCCGTTATGTTTCTCGTCTAA
CCAAAAAGGGCCTACAGCTACGATAATTTCCAAACAAATAAATCTGTGCTACTTGAT
TTTCATGCAAAGCTCATATTTGTTCAAAAGGAAAATAAAGCTTAATTTAAAATCAATT
TAGGCTATTTTTATCTAAGTATGCTTACCGTTATTCAACTCCCTGCAGATATTGTCAAA
TTTCTCAATATGGTAAATATTTATTCTGTTAAAATATATCCATAGTTACACTAAAGAC
AGAGAGGTCTTATATGTTCTAAACAACATAGAGCAAATGCTCATAAACAGCATTTTA
TTCCTATCTCCCGGAATAACAACGCTACTTCCAATTGCTGGAATCTAAATTATTAAAA
TAAACCCATGCTGCAAGCTTTGTATGCTTAACATTCTCAAATGTTCACTTTTCAGATA
TGGAAGTGAAGCAAATCAACAAACGTGCCTCTGGCCAGGCTTTTGAGCTGATCTTGA
AGCCACCATCTCCTATCTCAGAAGCCCCACGAACTTTAGCTTCTCCAAAGAAGAAAG
ACCTGTCCCTGGAGGAGATCCAGAAGAAACTGGAGGCTGCAGAGGAAAGAAGAAAG
GTAACTTTTTCCATAGGTTTTCCTTCTCTCTCTCCCTCCCCTGCTCCTCCCTCTCACACA
CTCGGGCACACATGCACGCACACACACACACACACACACACACACACACACACACA
CACACATACAGAGAGCAATGACAGCTGAACCTGTGCCATGCCAACATGTATAGGTTT
TCAGTAGACACAGAGCCAGGCTAGTTGGGGTAAAAACTGTAAGATAGATGCTAATTT
TAGGCTAGCCAAACCAGAGCTCTCAGAAATCCAAAGAGCTTCAGTGCTCTAGTGCCC
CTTCCCGTATATTGAATCCCCTTATTATAAAAGCCTCCCTTCCCTAGACCATCAGGCA
GAAGCACTGTAGAGAAAACACAGCCCTGGCGAACTCCAGTGGTGGGGAGGGGAAGA
AGTGCTGCTTCCTCCCTCTCAGGATCTGTGTCACCCCCTTTGTCAGGCGTGGTTTTCCT
TGGAATTACAAATTACCAGATCTTCCCTCCAAGATCTTTCCTGCCCAGGGTAAGGGCC
AAGAGCTTGCCCCTTTCCTCTTCAGAGTCCCACTGCCTGCCCTGGAAGTTGGTCCTTC
CAAGATCAGGACCTTCTCTGAGTTCTTTGAATATGTTCTTTATCTTTTTCTAAGACTTG
ATGGGGATTTTTCTCTTTTTGCCATTGGTCCCTGCTTATATTAAAGAGCTTTCCTTTTG
CCAAATCTTTACTTTTCCATAATCACATGGCTAAGAAGAGCCAAGGGTATTATTTGAG
AACACTTAGAAATCCTAGGGACTGTGTACACAAACAGAAGTTGTTTGAATGTGTCTG
TTCCAACCATGTGGTTATGGTAGTTAATCCCATCAAGGTACTCACGATCATCCAAAAA
TGGAATTCTTTTATGTAATTCATCCCCACATTGTATTTCCCAATATTTTTTATGATATA
ATTTTAGAATCAGGTAATCACTAAGAACATGTTCCCTGCACAGTTTTATGATGTTTTC
TCTAAAAAGTCAGCCAAAACTTTGGACACTTCTATGTTGGATAATTAAAAACAGAAT
GAAGATAATCCTCCTCCTAAAGATTGAATTCTCCAAGAGAGAATGCAGGACAAACAC
AGATGTGCTGTGTATAGTATATGTGCATATATACATGCATATATGTACACAAATATGT
GTATTATCAAATAATGAGGCTCAAACATTAGAAATCCTTAGATTAAATTTTCTAAACA
AGAAAACACTAATCTTTGTAGTTGAAAAAAAATCCTCCTATGATATGTAATATGCTG
ATCTCAATTTTCACCTAAGAGTGATGTTCTCCAAATGTCCGATGAGCATGTCATATAT
ATATATATGAATTTTTATATATATAATTACAATGGTAATTGGTATATAGAGATATCTA
TATTATAGATATATATAGCTATCTCTATATATTACATATACCAATTATAGATATAAAT
ATAACAATGGTAACTGGTGTATATGTGATGTGTATATATGTATATGTATACCATAATT
ATATATTAATATTGTATATATGCCATAATTATATATTAATATTGGTATATATACACCA
TGATTATATATTAATATTGGTGTGTGTATGTGTGTGTGTATATATATATATATATATAA
AATACTAGTTATCATTGTTCTAGATTTAAAAAACAGGAACCTGAGCTACTAACTCGAC
TATATATATATATATATATACAGGAAGTTGCTTTAAAACATTTTTATCAGCTTTTTTAT
TGTTATTTTTAGCTTTATTCTCATAGTAAAGCTAAAATAAATTATTCAACATTATCAA
AACTTTGCTGCCAGCAGATGTAAGCAATACCTAAAACAGTGGAGAGCATGTTGCACC
CAAAGCAGTTTAAGCTCTGACCCAAGCACTGGCATCTTATAGGCACTGGGTAGAGAT
AAGAGTCATAGGTCGACATATATTGAGATGCTATGACTTGATTAGAATATGGAGTCA
GTGACTGAGGTGAAATTAAAACTCAAACCACAATTCAACATCCTGATTTAGGATGTT
GCTGGTGTTTCTAGGTACTACACTTAATTTGAAAGAAATTATTGAGGATAAAAAAAG
AACTGGGATCAACAAAATTAACTAGGTGTTCTTATAAGAGTCCCTGAGGTTACTAATT
AATGAAACTGATAAAGCTCCTGCACCCTGACAGCAAGAAATTATCAATGATTATACA
TTTAAACAATTGAATTGAACTAGAAACTGGCCACATGGTTAAAAGACATTTACAAAT
GTAATCATCCAGTGTTATGATGCCCAGAAAAAAAAAATTCCTTAGAATGCTTTAAAA
GCCGTATTCCATCACCTTTCCAGTTATTTGTTAAACATTTTGTAATGCAAAAATAACC
ATATAGATTATGCCCTAGTGGTCGGGTTTTATTTTTAGTTTTTTATGGTTTTTTTTTGTT
AATGGTAGAGTTTTAATTAAAAGAAAATACAACTAATTAGCAGAAAGTGCCAACTTT
AAAAAATCACTAATTGATTTTATTCTATTGGGTTATACTGACTTAATTAGCACTAATT
TAAAGAACTATTAATTATCTTTAAAGAGTCTTTAGCAAGTGCATATATCTCAGTAATT
ATGTTAGTAAGGACATGCCTATAACCAAAACCCAACTCAACTAGTTAAAACAAAAAG
CAAATATGTGACTAAAAAGTCTAGGAGTGGCTACAGCATCAGGAACAGCTGGATCCA
GGGATCACAGTATTATCAGAAAACTTTCTTTCAGTGCCTGTCATCTCTTCCTGCATTTA
ACTGGTTTCATTATCAAGAAAGTTTAATTTCAATAGTCAGTTCCAAATTATTTTTCTCA
CAACTTAGCAACTCCAGCAGAAACAGAGCTTCTTTTTCCCAATAGTTTAACAAAAGTC
CCGAAATTGAGTCTCAATGGCCTGGCCTGGATCACAGGCCCAACCCAGAACCAATCA
TTATGGCCAAGAGGATGTAGTAGTTTGATATGCTAGCCTGAATCACATGCCCACCACT
GACCTGCAAAGGATTTTAGGTAAGATCCCTGGGGTAAGAATTGTGGAGGGGTAGTTC
CCCAGAAGAAAATCGAGGTGTTCTCACAAGAGGAAGGGGTAATGGATCTTAAATAA
ACAAAACTATAGATGTCCACATTTTCTATCTATAAATGTTTAGTGTTACTATAACAAT
TAGAATAATTATTTAGTTCATACACTATTCAATTTGTATCTCCCTTCTGTTGCCCTGTT
GCCGTTATTTTCTTACAGATAGAATGAAAAATATTAATCTAGGCAGCTCTGTGAAACA
GTACTGTCCAAGGAATATAACGTGAGCCAGGCCGGGTGTGGTGGCTCATGGCTATAA
TCCCAGCACTTTGGGACGCCGAGGCAGGTGGATCACCTGAGATCAGGAGTTCAAGAC
CAGCCTGGCCAACATGGCAAAACCCCATCTCTACTAAAAATACAAAAATTCGCAGGG
CATAGTGGCGAGTGCCTGTAATCCCAGCTACTGGGGAGGCTGAGGCAGAAGAATTGC
TTGAACCCAGGAGGTGGAGGTTGCAGTGAACCAAGATGGTACCATTGCACTCCAGCC
TGGATGACAGAGCAAGACTCCATCTCAAAAAAAAAAAAGAAAGAAATGTAATGGGA
GCCATATGTGTATTTTTAAATGTTCTAGAAGCCACATTTTTTAAAATAAAAGAAATAT
GAAATGAATTTTAGTAAAATATTCTTCACCCAATATATTCAAAACATTATTTCAATAT
GCATGTAATCAATATAGAAGTATTAATGAGCTGTTTCACATTATTTTATTCATACTAA
GTGTTTGAAATCCAGTGTGTATTTTACGTTTACAACTCATTTCAATTCATGTTAGACAT
ATTCCTAGTGCCTAGTAGCCAAAGGCAGCCAGTAGCACAGATACGGATATTAAAACA
GAAAACACCTAGTGAATAATGGGGAAATTTTAGGCCTAAGTTTTTAAAATCCATACC
AGATAATTATTCAGATTCAAATTTACTTTGTTTTTTCATATATATTCTTTAAAAATTAC
ATTAATATGGGAACTCAGAAAGTTCAAAAGAAATTTCCATTCTATGGTTTTAGTCTTT
ACATTGTCAGAACTAATGCAAGTGTGAAGTTTAGGATGTACTGTAAGTAATAGGATC
TTCTAAATCTCATGCCTTCTTCAGCTACCTACTCTGTTTCTATTTCAGTTCCTCACTGT
GGGGAGGGGACTTCTCTGAACCTAGGTTTCATCTCTCACTCTCGTTCATGGTAAACAG
GTTTTCCTTTGTGGCACCTAGCACAATTAGTAAGTAATTAGTATTTACTGGCATATTA
GTATATATATGCATATGTATTTATTTAACCCTATGTCTTCTACTAGATTATAAACTCCA
TGAAGATAGAACTTGTCTTTTGTTTAATAGTGCTTGGCAATAGTTATTACTGTAAACA
TTTTTTTTCTTTCTTATTCAACTCCTGTTAGTCATTGCCTGAGTACTACAAATGTTTTTA
AGTAAATTAATAAATAATAACTTTCAGGGCCAAATGTGAAAGCGGCAATATATAGCT
TGTTTTGATTTTTTATTCCACCCTCCCATCCTAAAACAATTATAGTCACTAAGTTTCCA
AATGACATCTGAAATTGCACTAAGGAAATCCTAGTCTGGGCAAAATCACTCAGTCAA
CAGATATTTATCAAGCACTTACTATTTGGCAGGCCCTGTTCTAGACACAGGGGATACT
CATCAAACTTACATTCCAGTGGGGGAGAAAGAGCTAATAAATACATACACAGCATAT
TAGATGATGCAAAATTAGCAGGACAAAGAGAACTGGGGGTGTGGGGGTGAAAGAAG
CTAATATTATATGTTATTATTACTATATATAATAATATAATTATTGGATAGTCAAAAA
AAAACCTCTTGAATAAGACATTTGAAAAGAAGCACAAAGGTAGCAAGGGAGTAGGG
CGGGCAGCTCTTCTCTGGGACCTGAACATTCAAAATGATGAGAGCAGCAGGTGCGGA
GGCCCTGAAATAGGAATGTATGAGGTGTGTTTGAGAAATAACATGGAGGCCAGCGTG
GCTGAAGCTGAGAGCAGGGGGAGAGTGGTAGCAACTGAAGTCAGAGGTCACAATTA
AGGACTTTGACTTCACATGAAATGGGAGATCATGAAGGATAATAAAGCCATTTCACT
ACTTTATGTGAATCACAGCATCTTTTTAAAGAAGTATCCTTTTTTAAAGGGGGAGATG
ACTAGAAAAATAAATAGTGTTAGATAAATAGAGAAAACAGGAAAACATTCTAGACT
AAGACAGTGATTCCAGAACTAAGGATCCACAGAGGCGAGAATGCAGAAAGTGTAGG
TTTCAGAGCAGTGGGTAGACTAAGGGTTTGGACTAGTGGATTTGGATAGGGAGTTGG
AGAGTAGCGAGGTGGGATTAGGGAGGGCTGTGAATGCCAGGTTAGTGTGCAAACTCC
ATTATATAAGCAGTAAGGAGTCACTACAGACTTTTCAAAAATACATACATGTTCCAC
CTGGCCCACGGGTTAGCAACATTTTCGTTGCCCTGGACCCATTTCCTTCCCAATAAGT
TACAGGTTTGTGAAGATTCTACCTAGCAAACATATTACTTTTAAATAACTATTAATAA
ATTATCTTACCATGATTATAATCAAAGGAATCTGTAATTGCTAATTATTTCTGATTATT
AAAAGATAAGCAGTATTGCACTAAATTGACATAATTCTAACTCAAAGTAAATATACA
GATAGACATGGCTATAGATGTGAAATATGATTTCTGTTAGGGCTTTTTAAATTTAAAA
AAACTTACGAGTTCTCCTCCCTCCCCCTACCCTTAATACCTTGAAGGCCTCTTTGTGG
GACTTCAGGGACCCCTTCAGGGAACTATGACCTAGGCTGTATTTGGGGGGCTTTCTGG
GTTTATAGCTGGAAGGCTGCCACAGAGGCATCGCCACTTGGGCTCAGATTCACTTTGT
GTTCAATGTTTTGGCAATGTCCCCACCTCCCCATTCCATCTGTTGACACTATTGCAGC
ACTGACCATCTGGTTACTAGGTTGGAGGATACTCCCTCGGGCTCCTTTGAACCAGAAT
TAGTGCTCCAGTGATTAGATAATAGAAGAAGCTTGTCATAAAAAGAATAAGCCCTTT
CCCTGCTTTTTCTCCATTCTTTGATTATCGCTGGTAGTCAGTGATGATCATCTCTATGA
GTCTATATCAATCTCATCAGGTCAGTTTGAACCTCATCTCTTGAAATCAAAGTTTCCA
TAATGCAACTGACCCACAAGGGTGAAATGACATGAATGCTTTAACCATCCATTTATC
ATTTATTCATTCATTCAACCAACATGTATTTAGCAAGAGGCAGCAGAGTTAGCATAAC
TATACATCCCAGTTGGCCCAGGACAACTCCAGCTAACTCTCGTTGTTTTGATACCATT
ATTAATTATTTCTCTTTACTCTCATAAGTGTTCCACTTTGGACAATCAATTACATGAGC
ATCCTTAGCAGGGCACAGTGTTTAAGGGCATCTTTAAAATATTGTCTTTAAGAACATG
TGGTTAAGAGAATGTCTGTGTTCAAATCCTGGTTCCACCACTTAAAAGCTGTGTGACC
TCAAGCAAGTGACTTAATCTCCGTATGTCCTCCTTTGTCAATCTGTAAAATGAGACTA
GTAATAGAACTTATGGAGTTAGTGTGAGAATTGGAAGGTTACTCTACAATAAAGACA
TATAACCAGCATGGTAAAAGGGTTAGCAATTACTATGTGAAGAAGCATCCAGTTTCT
GACCTCACAGAGATTATCTAGCAAACTCATGATTTTATAAAGAAAAGAAGTTTCTCA
TCAACAGAGACTGAAATGCTACCATACAATATACGTTGCTTTTTTTTTTTTTTTTTTTT
TGAGACGGAGTCTCGCTCTGCCACTCAGGCTCAGGCTGGAGTGCAGTGTTGCCACCTT
GGCTAATTGCAACCTCCACCTCCCAGGTTCAAGCAATTCTCCTGCCTCAGTCTCCCAA
GTAGCTGGGATTATAGGCACCCACCACCACACCCAGCTAATTTTTATATTTTTAGTAG
AGACAAGGTTTTGTCATGTTGGCCAGACTGGTCTCAAACTCCTGACCTCAGGTGATCC
ACCCACCTCAGCCTTCCGAAGTGCTGGCATTACAGGCATGAGCCACCATGCCCGGCC
AATATTTTTAAATATTATAAAATATTCTTTATCAAATTGCATAGAAGAAAAGACAGTT
TGATAGGTAATAGATATATAAATAGGTCAGGCCAACTAAAAGTGTCCTGAAAAAATT
AATATTGTGAAAACAAAAGGATTTTAATGACATTGATAAAATCTCACCCTAAAAGAG
ATTAAATTAAAAATCACCCTACTTGAACCAGTTCAGTGAGATTTCATTAGCATGCTCT
CATTACTGGCATAATCAGCTTCAAAGTCACTAAGCCTCTGAAAGGAAGATGTGTTGC
TTATTCTTAATAAAATGGCATAAAAGTAGATCATTAGTCACCAAACATGATAGACTT
ACCTTTTCCATTTGTTGGCATCTCACATTGTAGATGGCAATTAAAATGGAATCCAGGG
AAAGAGGGGGTGGTTTGTATAGCAATGGATTATGAAACAAAGTACTGGATTATTCAC
CGCTTGACATTCAGGAAACATTCTGCTCCTTACAGAATATGGCACGTGGGCCACAGA
ATCTTCCGTGTGCTACCTTCTCGGTGAAGAAGAGCACCCCCAAGTTTCTTTTCCTAGG
AGCTAACCACAGTAAACCCATTACACACTTTAGCAGAAGGGCTCATTCTAAAGGTCT
TAGGATTTTAATCATTTTAAATTTCCTGTTATGCTTCAGGCTCTTCAACACAAAGTGA
ATATTGTACTCTTTGGTTTTACATAATTATATTCAATTGTCATATTTCAACAGGACATT
ATTTGTGACTTTAGATGGGTCAATAATGATTTTCATTGTCAGCAGTAAAGTCAATAAT
TACAGACACATCACCTACCCTACTTGTGTAAAAGCATTTTTTGGTACTAGGAGATTTA
GTGTCTGATCAACGGTCCTGGATAGCAAGTAATATATCCCCCAAATAATGAAAAGTG
ACAAGAAAATAAATATGTTTACTTCAGAAATAAATGGAAAATTAGTGCTATCTAAAA
TGTAGTCTTAAGTCTCATCTGTGTACATAAAGTAAAATGAGTTTTATGTACTAGTTAC
TCAAATTTATCTTCCACTCCATTTGTATAGTAATTAAACTCTTACACTCAGTAATATAC
AAATTGGTAATTAACCTCTTTGCAAAATGTTAAAGTGTTCCTAAATGTACAATAAGTC
TCCTTTCCTGTCTCATTGTTTTTCGCTTCACGTACCTCTCATGTAATTATTTCAATGATT
GAGTTCAGTGTGAGGAGGTTTATGCCTAGAAAAGGTGCTCACCAATAACGTGCCTCA
GTTCCCATAATAGCAAGATCGAGAAGGTTCTTTAGTCTCCCGGAACGTCACGTTGAA
CATCTCAGTTCTATATTTTGCCTTGACATTTGCATTATATCAGCTGATCATTGTCTTGC
CCTAATTTTCCCTTTTAATATTTTAGTGACCTTCTATGTTAGGTACAGGTTATTTAGAA
GTGTTCCTCCAAGGCCAGATACTTTTTCCTTGAACAATTTATTTTTAACAACTTTTAGC
GATTTTCTCACTTCACCACCCTCCGTTTCATAAGTCCACGCAATCACAATTCCTTTCTG
CTAATCTGCACAGTCAAGATATAAAGTAAGAATACCTATTTGAACATGTAGTGAGAA
CTTTACTTCTCTGCCAAAAATGAAGGAAAATGCTGCCACTTTTGTATGTCACATGTTT
TTTATTCTACAGCCTCACTCACTTCATGTCATGTTTTAGTGCAGTTTTCTGGACTAACT
GCTTATTTTCTCATTGATTAAACTGCCTATTTGCTCATTGGAATTAGAGCCAATTTTTT
TCCTTGAGGGTCTGACTAGAAGATTAAACTATGTTCATGTGAGAATCAATTTCTACCT
AAGAAATGAGTTAGAGGAGTTATGGGCAGCAATATCTATCTGGATGCTACACTGTGA
AAAAGGAAGCGAGGTTATGCCTTTCTACCCCAATGGGGTAGCAGAGACCTCAGGAAC
TGAGGTAGATGCCCCCCTGGTTATTAGCGCCCCTGAATAATTTGTTCAAAAATTGACT
GCTGGACAGGTGTCGTGTTGCACGCCTGTAGTCCCAGCTGTGCAGGAGGCTGAGGCA
AGAGGATCTCTTGAGCCCAGGAATTTGAGGCTATAGTAAACTAAGGTCACACCACTA
TACTCCAGCCTGAGCAACAAAGCAAGACCCTGTCTCTAAATTTAAAAAAAAATATTG
AATGCTTATGAATAGAGACTAATATAGGAAGTCATAAGTATTTCCTTGGGATAGAAT
GCTTTCCACCATAATTGACTTGACATCCTGTATTTTTGTATGTGTGGACTTAAGTTTTA
AATATTTGAAACACAGACAATTATTAAGTCCTGCAAATGTGTGAGTTAATAGTGGAT
ATAACATTCCCTTCCAGGGTGTAAGAAAAGGTACCACAGAAGTGAGCAGCCCTGAAG
CACAGCCTGGCCTAGTTTGGCAGGTCTCTGTGAGTTAGCAGCAGACTCACGTGACCA
CACTCTGTACTGCCTTCTGTTTCTGTTTCACCCCATTAATTGTGCTAAAGAAATGCACT
TGACACCTATGCTGTGTAATCTCATTTAGCCCCAATAGCAACAAAAGTACTAACCCCA
TTAAATTGAGTCATTTCAAACTGAGCCAAATGTTGCACTCCAGTAAATGGAGTAGGC
ATTGGTTATAATGGGAATTCTCCATTATTCATAATGGAAACCACAGGAGTTTGTTCAT
GCAGATCAAATGTGTCCCACCAAGGCAAGAAGTATGGAAAAGTGGTGTTGCTGTATT
ACCTTGTAATTTCAAAGCCTTCCCGTCTGAATCTTATTTCCCTGCTGTTTCCTCTTGAC
TTTGGTTCTTTCACAAAGGAAAATTAAGAACACAAATATAAACATTAAGTTAAAACA
CAACTGAACAAAGTGCCAAACTTAATTGGAGCATCTGAAAATGAAACATTAGGCAGT
TGCAGTGGCCTCTTGATAATAATTCACAGTAACTCTCTGTAAGCTGATCCTGTCTGAA
GAGCAGCAGGCACAAGGCCCCTGGCCATGAAGTCCATCTCAAAGGGCCAGGCTCAG
CAAAGCAGGATGCAAACCCAGGCTTTCCAAATACCAGGTTGGGGCTCATGTCACTGT
GCCACAGGAGCTTCTGTAGAAAGGCTACTTGAAAAAAGTGGCCATTAAAAATCCAGG
TGGATCCTATCTAGGGCAGTGTTGGAAACACTGATCTATGGGAGGAGGAGCAGGAAG
GAATTGTTTAACCACTGAGCAGAAATGTTACATTGCTACCTGCCTTTAGCAGCTGTGG
CTGATGGGTACCAGTTGCTAAGAAGAGCATTACCTAACAGTGTATTAAGATAGAAAA
ATGATTTTAAAGCACGGCACTTAGAGAATGTTGAAGTTTTACTTTGCTTTATTTTGATT
TGTTTGGTTTGACTTTGTCTCCTGGAGCATCCTCCATGGATTTCTGTTCATTACAAGAG
AAACCTAGGGCTCTAACCCAATTCCTAATTCTTGGACACATTGCACCCTTGTTTTGTG
ATAATCCAGCCTTCTTCCTTGAGAAGGTTTGCTGGACTGGAGGTTACATGTATTGAAT
TTTCTAAAATGAAGGTGCAAAGCTGTCTCCTCTTATTTCTTTGTGGTGCTCACTTCACT
GTGAGATTTCCTATCAATACAGCCCAAGTCAGTGGGCATGCATGAGGTGGAGATGAG
GGAGTTAGGAAGGACTTGGACTCTCATCAACCATCAGGATCCCTGAATCCACTAACT
GTTCATAATCAAAGAAGTTTGAACAAATACTTCACACACATGAAATTGCCAAAATTT
TGCATTTGAGTTGTTATACCAGTAAGTCCAGTTGCCATCATCTCCTTGTCACAAGTGT
CTTAAATTTTGCTTTTGATAATAATGATTACCACTCATTCAGTACTAACTTACTTGATA
TTAGACACTGCATTAAATACCTTGCAAACATTATTTTGTTTGATCCTGACAACCATAT
GAGATAGGTACTATTCTTATCCATTACCAAAAAAATTAATTTCATGAAGACTTTTCCC
AGAGAGAGAAACTTTAAATATTTACACACACACCTCTCTCCCTGTAACAATTCCGTAG
TCCTGATAACAGCAAATAAGCAAAGTCTGTGTAGGATGCTTTACCAACAGTCCCACC
TAGAGGCAGGAGAGTGAACCAGCTAGAAAATATTTTATTCATATTTCTTCCAGAAAG
GCTCCATTGGAGTTTGAACTCAATTTATGTTATAATTTTCTTATTATTTTTGTATTGGT
TTTCCTGAAACCAATACAAAGTAAGAAAGCATTGGTTCCACTAAAAATGTCCTAAAA
CCAGCCAAGCACAGTGGCTCACACCTATAATCCCAGTACTTTGGGAGGCCGAGGCGG
GTGGATCACTTAAGCCAGGAGTTCAAGACTAGCCTGGCCAACATGACGAAACCCCAT
CTCTACTAAAAATACAAAAATTAGCAGGGTGTGGTAGCACACACCTGTAATCTCAGC
TACTCAGGAAGCTGAGACATGAGAATCGCTTGAACCTCAGAGGCAGAGATTACAGTG
AGCAGAGATCACGCCACTGTACTTCTGCCTGGGTGACAGAGCGAGACTCTATCTAAA
AAAAAATAAACACATAAATAGTAAAATGTCCTGAAACCATTATGGGGTTAAAGCAA
GAGGCAGGGCTGGTTCCCAGGATTTTCTGTCTAATCTCCAGTGAGCCACAGACCTATT
CCTGATCAACTTGAGAATAAACACATCAGTAAAGATGTGTAAGGCTGTCTGACTTTC
CCATTTCTGTAGAATTTTATTTGAAGAGAAGTTTCTCCTTTCTCCAGGCCCCATATTGT
TTATACAAAAAGACCTTTCCAGTAAATGTCCACAACCACTACCATCAACTAAAATGTT
TTCCCACTAATGCTTTCAATGGTAATCAGTATTTAACAGGGCACTTAGGATTATTTTTT
GATCAACCATTGTTTAGATATTCCCACTTATAATTACTCCTGTGAAGGATTGCCTCGG
GGCATCAGCTGATCCTGAGAAATTATCCAGAAGCCATGAGTGTGTAATAATTTAGTC
TTAAACCTAAATAGGTCAGTATTGGGTGGGACTTTTCTCAGCTGCATAATGGGGAGA
ATAAAAAGAATATGGAAAGAAGTTACGTAACACATCCTGGGTCACAAACAGAGGTA
AGACTTGAACACAGGCCTGACATCAAAGCCCATGCCAGTATGACTTACAAAAGGTAG
ACTGGACTACCTGCATTTGAGTCACTAGTGATGCTTATCACTGGGCCTCACCAAAGAA
CCTTGGAATCAGAATCTTTGGAGGTAGATGCCAGGCACCTGCATTGTTATCAAGTGCT
CCAGTGATTACCATTCACTGTACAGAGCCAAACAGACTCCTGATGCTGGAAGAAAAT
TACAGTGCTCAAAGTGCAGGGCAGGGTGTACATCTGGATCTAAATCACTGAGCAACC
ACAGGGTTTCAAGAGAGGGTCAAAACAAGGACTTTCTGCTCTCTGTGGCCAAGGGGA
CACTAAGTTTGCACTGTTCTCAGATCTCCAAAGAGACTTTGGTGTATGGGGGATAGG
GAGGGGGGAAGGGGGTGTGAAATAAAAGGAGAAAGTGAATTTGATTATTTGATTGA
TGAAAATTGAAAAGCTTATTGTAGGGCCTAGCCTACAGTTGATGAAAAAACAATGGA
TCAGGAAGAAGATCAGAACTTGTCTCAGTCCTCAACTGTTTTCCTCAGGCTTTGGTTG
AATATTGCCATCCTGTAATTCATTATAGCATTTTCTGTTGCATAAACGCTTAGCAACA
AAGCCTTTTTTTAAAAAAATTTGTAACTCCTCAATGAGGATTAAATGCTTCTTCTTCTA
AGACAGTCCGAAATATACTCACAGCTGAAAATTCAGCTAACCGCATTTCCCAACTAG
CCACATTCTATAGAAAACTCTAAGCCATGCAGATGAGTACAGACTTGACAATAGTGC
TCAAGGCTGGGAGTACTATTCATCTGAAAAGAATGCTCCCTCCAATTGGTGGGCCGTT
ATTCTGCTAGGTTTGTGTTTGGATAATTATAAGATGGCTATGTTTTTCTTCCCCAGTCT
CAGGAGGCCCAGGTGCTGAAACAATTGGCAGAGAAGAGGGAACACGAGCGAGAAGT
CCTTCAGAAGGCTTTGGAGGAGAACAACAACTTCAGCAAGATGGCGGAGGAAAAGC
TGATCCTGAAAATGGAACAAATTAAGGAAAACCGTGAGGCTAATCTAGCTGCTATTA
TTGAACGTCTGCAGGAAAAGGTAATCTCAGCAGAGTCCTGAGCAGATGGATATATTC
ATATGCAGCACAGCTGGGTGAACTTCCATATGCCTGAGCACAGAGACGAAGTCAAAA
TTTGCTGCAGGTGTGAGGACAACTAACTCCCATGGGCAGGGTCTCACAGTGTAGCAT
TGAGTTAGCAGGAGGTGCAACATGGTAGAGAAATGGGAATCCATCATGAAAGCTGG
AATTTTGTCAAATTTTCCCATGGTGAGTGGATTCAGGGAGGCTGATTCATGCTTTTGA
AATGTGTAAGACTTCTATACAAGCCTCACGAGGCAATCTGTAGGAAAAATGTTACAC
TGGAAATATTAATGTCTATATATTATATTGATATAAGTATAAATAACATTTGATTTAA
TATTTGTTTAATATATGACATTAAATATATATTTAATTAAAATATTAAATTAGAAAAA
TATATTTGCCAGAAAAGGCCAGGGTATTTATGAACACTGGTAAGCCCATTCTAGGGT
ATAATAGCATCACATGGGACCATAGCAAAGATTAGCTCATAGGGGATGTTTCATCCA
GTTCTGGTATCCTGGTGCCCTTCTCTTCAACAACCTAAACATATATTCATTCCCATGA
GTCAGGAGGAGCTGTGCTGGAGTTCTTCTGAAAAATGCTGTCTTTCACTTTTGTACTC
TCTATGCTGTCTCCCACCTATCCCCTCAAAAAACCTTTCCTTTGAAAATATACAGTAT
AGCTGTGAGTAGTTTAGCTGTGTCCGTTTCCAGAAATTGGAATAAGCATTGAGAAAT
GGGATGTTTGAGAAAGACGCCTCAATCCTTTTCTGAGCAGTCAGTCACCCTTCCCGCC
AGTAGCAAGTGCCTTTGTGTGATAGGCATTGGAGATGCAGAGCAAAACAGGAGTGTG
CCTGTCATCAGAGCCCTGAGAGTTTAATTAGATGAGCCTCCTGTTTTCTATTTCTCAG
AGTTTCATGTCTTCTGTTAGAGATGGCCCTTCTCATCTAAGGTTCAAAAAACCTTATC
CTGAAGTTCTGATGATTCTGTTTTCATTCTCAGTCTCTGACTGCAAATATCCAACTAG
AAACAAAGGAAATCAGGCATGAAAACTTTTAAAGATATAATTGCATGGAGATCTTCA
TTTGTGCTCGTGAGGAATTTTTGAAAGCATTGCTGGGGAAGGGTGTGTGGGCTCTGAT
GCAGCAGTAAGACACTGAGGCTCTCAGAGGTCCGTGGACGAGTACTGCTGACTTGGG
CAAGAACCGGAATAGTTACCTGATGCCTTATCCGAAACATGAAAGTTCGGATTAAAT
TTGTATTTATAAGCTAGTGTTTTTATACTCTCAGAACAATGTCATTGCGTTTCACCCAA
GTGAGTCAAGTCACGATTTGGAAGAGGCAACAGAATTTGGCTCTCTCCAGGTGATTT
ATGGCGGTATAGGAACACATGTTTTACTCAGATACAGGGGAGCAAAGTTCCATTTGC
TAAAGTTTACTCCCCTGACCTTCAACCAGTCAGTCTTCCTCCATCTGCCACCACTTTGC
ACTTCTCCAGAGAACTAAGGATGTTCCCGCTTGACCAGTGCTCATAACATGGACAGC
AGAGGGCCACTGTGTGATCTCTTTGAGATCACTGTGACTCAACCTTCTTCTCACATCC
TAGGCCCTAAAACAATTAAGTGAAGTTGCTAGGAACGGTACCTGCTGATCTTATTGC
AGCATTCTCAATTAGGCCTCAATGCAAGATTTATATCACTGGCAGTCCTGGAGCATTT
TTGTTTTTCAAATTACACATACCCAAACACACGGCATAGCCTCCTTTTTTGTTTGTTTG
TTTTTTTGAGATAGAGTCTCGCTGTGTCGCCCAGGCTGGAGTGCAGTGGCACGATCTC
AGCTCACTGCAACCTCTGCCTCCTGGGTTCAAGTGATTCTCATGTCTCAGCCTCCCAA
GTAGCTGAGATTACAGGCGTATACCACCACGCCCAGCTAATTTTTGTATTTTTAGTAG
AGACAGGGTTTTGCCGTGTTGGCCAAGCTGGTCTCAAACTCCTGACCTCAAGTGATCC
ACCCACCTCGGCCTCCCGAAGTGCTGGGATTACAGGTGTGAGCCACCGTGCCCAGCC
AGGGCATATCCTTCTTGATTTCAATTGTAAAATAGTTCAAAAATTTTCCATATTTTATC
TAATATTTCCAGAAGTGCTAGCTTTTAACGGACCATTTTTTTCCTCTGTGTGTTTTTTT
CTCTTCACCTAGCCCAGCCATGCTCAGCTCATTTTTGTACTCTTTCCACTCCCAACCAA
ATTTAGTGCCCTCCCCCATACATGCATACATGTACATCTGCACACACCACTTTTCCTGCA
AATAATCAACCCAAAGAGTGCTTAAAATTCCTGACATCAACCCACAGAATCTCCAAG
GATGGGACCCAGCATCCATACATTTTAAAAACTCTCCATATAGTTCCAATATGCAGCC
AGATTTGAGAACTAGTGGTTCGTAGCCTGTTCTGATTTAAATCTCAGCTCTCAGCATG
CTATCCCACGTCACATAATGCAGCCCAGAGAAATTCTAGGACCACATTTTTTTCTGGT
ATTTCATAGCTAATGAGGTGCTTTTCAAATCTAATAGGATCTTTGGCCAGTGTCAGTC
AAGATCTTTTATCTCCTCAATAAAAAGGAAATACCATATTTACTTTGATTTGATGTAT
ATCACATAGGTGGATTTAATACAAAATTGTGGTTTACATATTGTGAATGTGTATACTA
AAACTACTTTGCTTTTTCCTAAAATAAGACAAAGTTTTATATTGGAAGTAATATTTAG
CATTTTGTTTGAATGAAGTTACTCCTATTAAATTAGAAATTTAAAAGAGGGTCAGTAA
TAACAGTAAAGCCAAAAGGCATGACACTGCCAACGTAACATAAGCTGCTCTGAAATC
TACCATATCAAAAGATAATTATGCTGGGCATGGTGGCTCACACCTGTAATCCCAGCA
CTTTGGGAGGCCAAGGCAAGAGAATTGCTTGAAGCCAGGAGTTCGAGACCAGCCTGG
GAAATATAATGATACCTTGCCTCAAACAAAAATTCAAAAATTAGCCAGCAGTGGTGG
CACACTTGTAAAAATGCCTGTAGTCATAGCTACTTCAGAGGCTGAGATGAAAGGATT
GCTTGGGCCAAGGAGTTCGAGACTGCACTCCAACCTGGGAAATATTGTGCCACTGCA
CTCCAACCTGGGAAACAGAACAAGACCCTGTCTCTAAAATAAAAAGAAAAAAAAAG
ATGACCACTTCTGAAATGACACCTATCAATGAGTTAATCATTCAATGAATATGTATTG
AGTCCCTACTATATGCTTAGGAACCTTTGTAATATCATTACCAACCATGTCTTTCCCA
ATACAGACAATACAAAATTCAGCAATAAATAATATAGCACCAACAATTAGAGAATA
AGACAACATGTAGTATGGTCCAATATAGACAGTAAATACAAAGACACTGAATAATAT
CAGTAAAAGTAAATTCACATCAAGGTCACTACACCATGCGCCCACCCTTATGATAGC
CCTCACTGGCCCTATCAATTAAGCAAGAGACATGATACAACTCTGTGCAAGCTTTTCC
ACAATCTGCCTACCATTCAGCACTCAGTCGCTCTTCCCTTCAATTAAGAGAATTGAGC
ATTCAAGCATATTTTCACCATGATGCCCATAATGGTATCTTCAATGTCACTGACTGAT
AAATTCCCAGAAACCCCTCAGAGCCCCAGCCATGTTAGCTCAAAGCCTTTAGCTAAA
ACTGAAAGCCTAAAGCAAAAGCAGCCCTGGCTGCACTTCGGAATCTACTGGACAGCT
CTTTAAGGGATTCTGATTTAATGTCTGGAATAGGGCCAAGAACCTTGTATTATTTTAA
AGGCTCACTAGTAGGCTCTAATATTTAGCCGTGGTTGAGAACCACTGTGCTAAATGTT
TCTTAAATATGCTTTGTGATGTCATCATAAATTATATTTTAGTATTTTTTGTCTTTGTTG
CATAAGTGTTCTTTCTTCCTCCAAAGAAGAATGTTACACTCATTTCTTATTTCAGTTTC
CTGTTTTCATAGCACCTCATCTTAACACTCCAGGCTATTATATAGAAAAGAATCAAAT
GTGGAGAAGGCTGTGGGAGAAGGGATGCCTGTGCCACAAAGGCCTGCATTAGGCTG
ACCTATTGATGTCATATCCAGGACTCAAAAGACTAGTCTGTGGATTATGACTGGTGA
AGTTCAAAATGTTCTTATTCTTAGAGTGGTATGAGAAGTAGAAAGAGAGAGAAACAG
AGAAGGGGAGGAGAGGGGAAGAGAGGAAGATGAGAGAAAGGAAAGAGAGGGGGA
AACACCTGTTCTTGACATACAGGAATGATTCAAGACATTTTCTTCCTCCCCTGATGTG
TCCCTTTCTCCCCTAACGCACTATGCAGCATCCTGCAGAAAATTCACCACCTGACCCT
TTTAGAAACCCTGAGTAGTAGGAGCGCCAAATGACCCAATCAAGAATTGCAGTGAGA
CAGTTAGTTTTGAAAAATCAGTTAAAGCATGTATAATCATTTTAACAACAATACATCT
ATTCACTAAACATATAATTTTAATGTCAAATATTTACGTGTAAACATATTGACCAATC
TTTCGATGTAGTTGGGCCCAATACCTTTTCCAAAAATTGATCAGTTAATGGGGGTTCT
ATGGGGGTTTCTTTTCTTGCCATTATTCACACTTATGTCACATTAGCTATGATTTGCAG
TTTTAATTTCTTTAAAATTGAGTAGGGACTAAAGACATCTCCAAAAAGCCTGGATATA
GACTTTTTACAACTTTTCCATAGCTTTTATAGTTGACTCACCCAGTATCTACTAAATAC
TTCACTTTCTCACGTATTTCCAAAGGTTTCTCTCCACCCTCACAATTTTCCATTAATGT
AGTACTTAATTAAATTAGATAGTTAAATTTTCAAATGTGAATTGCTAAACAGGTGTGG
AAATACCATTGGCTATAATCAAGCATATAACACAACCATTTGAGAAGGAAAGTATGT
GGCAATATTAGGGAAGAGCCCTTTCCTCTCAAGCAATTCAGCATTTAGGAACCATCA
GACAGCAGGACGATGGAGGGAACAGAGAGGGTTAACATGGCAAGTTACTGAAGAGG
ACTTCTACTGAATCTTGTTGAATTCCCCACTTAATCCAGATTGTATCATATCTTCTTTC
TTTTGTAATTCTACCATATCATCTTAGTCAATGCCAAGACTTCTGAGCTCATAACATG
GTAACAAATACCAAAGGAGCTTTCAGTATCGTTTAGAAAGGAGAGAAGCAAGTAAC
CCAGACAAACTTGACAACTGCTTTCCCCTATCCAACCATGAAGTACAGTACTTAGGA
AATAAAAGAAATTGCTTCACTATAATTCATCATTTCACTTCTAATATCTAGAAAATGT
CAAATGAAAATATTATAGCCATATTTTAGTGGCAATAGTAGCACATAATATGATGCA
ACTTAAAATGATAAAAATATTTTCAGGGAATAAGATTCTGTGATTCTTTCCCTAAGAG
GTAATTTTGATAATATGTACCTGTTTTGTAAATGTCAATAGTCTTGGGGATACAGGTG
GTGTTTGGTTACATGGAAAAGTTCCTTAGTGGTGATTTCTGAGATTTTAGTGCACCCA
ATACCCAAGCAGTGTACACTGTACCCAATATGTAGTCTTTCATCCCTCGCCCCCACTC
CCAACCTTCCCCCACAAGTCTCTAAAGTCCATTATATCACTCTTATATCTTTGCATACT
CATAGCTTAGCTCCCACTTATGAGAACATATGATAGTTAGTGCTCAATTCCTGAGTTA
CTTCACTTAGAATAATGGCCTCCAGCTCCACCCAAGTTGCTGCAAAAGACACTATTTA
GTTCCTTTTTATGGCTGAGTAGTATTACATGGTGTATATATACCACATTTTATTTATCC
ACTTGTTGGTCAATGGACACTTAACATTAGTTCCATATCTTTGTAATTTCAAGTTGTGC
TGCTATAAGCATGCATGAGCCTGTGTCTTTTTCATATAATTACTTCTTTTCCTTTGGGT
AGATACCCAGCAGTGGGATTGCTGGATCAAATGATAGTTCTACTTTCAGTTCTTTATG
TTTTCCACAGTGGTCATACTAATTTACATTCCCATCAACAGTGTAAAGTGTTCCCTTTT
CATCACACCCATGCCAACACCTATTGTTTTTTGACTTTTTAATTACGGCCATTCTTGCA
GGAGTAAGGTGGTATTTCATTGTGGTTTTAATTTGCATTTCCCTGATGTTGACAATATT
TAACTCTTTAGTTATAGATTCCAGCTATTATCAATTTACACCTATTGCATTCTTCTCAT
CTTTTGTTTTCTTGTGATTCTGATGCACAAATATCATTTGTGCAACCACTTACTGTTGA
ACATGTCTGATGAACACTTACTATTGAACATGTCTGATGAATGAATAATGAAATAGG
AAAAGGGATTAAAACTAGCCTTTATTAATTGTTTGCTATAGGCCAGACATTTTTGGAT
GTACTATCACATTTCATCCAAACAACAACCTAAAAGAAAATACTGTGATTATCCCCAT
TTCACATCTAAGGAACCTGGTCTTTAGGAAGATTAAGTCATTTGGCCAAGATCACAA
GTAGACCACAGAGACTAGATTTGAATGCAAGTCTGTTTGACTCCAAACCTTTTTACTA
TCTGCCCATGACCCCTGATCACCAACATCTCAATGTATGAACATGTGCTTTCTTAGCT
CACACAACTCACTCCTGACCCCTTTTTTATATTGCAAGTGCATAGTCATTAGTAAAAA
GAAGGATTTTTGATGATACTGACCTCATCTTGAATTTAATTAGGCTCATATGACAGAA
TTCCATAGATGGAATTGACATCCTAGGTCATATAGTCCAAGTCCTTGTTTATATTTGA
TACCTAGTGAGATTAAAGGGACATTAAAAAGTAAAGAAAGGAAAGACCTCATATTTC
TTACCTTCCAGTAGAGAAATCTTTCTATGAAATCAGAGGAAAGAATTAGAGGACCAG
AATTTTTCCTAAAATCAACTTTCATACATCTTTTTTCATATAAAAGGCATAGCTGCAT
ACAATGCTAAAATATTGTATTACATTTCCTTTATATTGATGGGAGGAAGGGGGTAAAT
TGCAGAAAACATTGTAAATTTAGATATGCTTGGGCCTCTGACAGTGCCTAGCAAATA
TCAGGAGATCAATAATGAAATAAATATTATCAAAGAGTAGTCTTCTTGATGAACCTT
CTCTGAGTATCACAACTGCTTTAGGAACCTCTAGATTCAAGGTCTAGTAATTGCAAAC
AGTGAGCTGATAAGAAAAACAGACTGTATGGGAAATTACATGCTTCCTGCATGACTG
CCTTTTGTTCTCCCACATTTTGATATAAAGTCACATTAACAGTTCATGAGTAAATATTC
GATAATGTGAACGTAAAGTGTTCAAATAATAGAGTGACTAAAATGCCTGAAAACAAA
TAATTTTTAATTAGAAACTCATAATCATTTATTTTCTCTTTTTCCACATTATCTCAAGC
TCACAAATTATATTTATTCTTTCCTATGGCAAAATCCATTTTGTTAACACTAATTTTGA
GTTTAACAAGAAGTGTACTCCAAAGTAGCCTAATAATACTAATTATAATGTTTCCTGC
TATGTTATCAGTTTGAATTTATATGAATCTTTAGACTTGAGGCTTCTTTTTCCTAGCAT
AGTGATGGTCTGGGCTTTTTCTCAATTTTTGCCAGAGCTCAGCTCTCACTAATTAGTTT
CTTTCTGCATGAGAAAAAGATTTTGCTTCATCTTTTTCCTTATAATAGCAGAACAAAA
AGAAGAATCAGCTGCATCCATGCTAATTTCCCCTGTGACATTTCCAAACAGGATTTGA
TTTCTCTATGCATGCCTCTTTCCTTCTCTTCATGGTTTTTGAACATATACAAAAGCTCA
TTTAAACCAATTAAATAAAATTGTTTTTAATCTCTTTCTCTAGAGTCAACTTCCTGCTT
ACTCCAACTCTGTATCTTTGAAGGAAGTATAGGGTGGTCTATGCCTTTTTTCTCCCAG
AATCTACACTTGAAAAGACACATTTTTCCATGCAACTATAAAATGTTCTCCTCACTCA
ACATTGAAATTGTATAGCAGTGATTAAGAGAGTGAGCTGTAGAGCCAGGTTCCCTGG
GTTTAAATCCCACTTGTTAGTATCATGAAGATGGGCAAGTTACTTACCCTTCCTGTGT
TTCAGTTTCTTCATCTGCAAAATGGGGACAATAATAGAATGTCCACTATAAGATTATT
GTGAGGATTAAGGGAATTAATACAGGTAAAACGTGTACTGATGCAGGTCTGGTACAC
ATTAAGTGCCTAATAAATATTCAGTATTATGATATAAAGAACCCTATAAGTGTAGACT
CCTTGAGATTAATAGAGTTTAACGATAAGTTTTACTTTATAGCTGGTCAAGTTTATTT
CTTCTGAACTAAAAGAATCTATAGAGTCTCAATTTCTGGAGCTTCAGAGGGAAGGAG
AGAAGCAATGTAAGCAACATTCTACAGAAATATAAATAATACTACTAATAATTAGCA
TCTTAAAATTTCAATTCAATGAACATTTATTTAGCGCCTATGATATATGCAAGACAGT
TTGATTTTAGTCATCTGATGTATAGCCACATACTAAAAAATACTGATTTTAGTCATCT
GATGTATAGCCACATACTAAAAAATACTTCCTCCATCAGTTCCCTCCTCAGGAAGTTC
AGTTCCCAATCCCAGGCTAGTACCTTGGTTCCTTATGTAAATAAACATCCACCAATTA
CATGCTATCTGCAAAGCACTCTGCTAGGCCCTGCAAATGGAAAAAAAAATGATAAAA
CATAGTCCAGGCCCTCAATGAGCTTACAGTCAAATATAATAGAGGAGACAAGAACAG
AGAGGCTCATAATACAACTAGAATAAAATGACTGCCGAATAAAAGGAAAGATTTAT
GCAGGTGTTCAAATGGAAAGTGAGATAAGTTTGCAGGTTAGTCTTTGCAGTCTCATA
AAAATCTTTATGGAGAAAAGGACAATGGTCATAGGGCTTAAAGAGTAAGTTTATAAT
CCTGACCAGTGGAGATGAAAGACTAGCATTGAAAATTGCATGACAAGACAATTCCAT
TAAACTGAAACATCAAGTGTGTGTAGGAAAAGATGGGGGTTATGACTGGAAACGTCA
CTTGGACTGCAATTATGAAGGGCCTTGACAAACAGGTCAAGAGTTTAAGAAGCAGTA
TAGAAAGTCTTCGTCCTGGATCTAGCCCTCCCAGAGTGTCCATCAGGATTATAAAGTC
CTTAAAATATTAGTCAAAAGGAACGACATCATTAGAAATGATAGAGAAACAATAATG
TGATGTTTTATTACCTTTCTCTGGATTTATACTCTGATCCTAATATTCAAAACTATCTT
AATAACATGAACTTTTGGTCATAGTTTTAAACAAAAACAGTGTTAAATATATTTTTTA
AAACACAGTAAGTCTTGTAAGATCTTTTCTAACATGACATTTTGCAGGGCCCATATTT
TCCTTCTGAAATGGGAAAAATTCATAAAAGTAGACACCAAACTGGGTTACTTCTAGT
CAAGCGCATGGTACGCAAAGGACCAGACAAAAAGGGCCTGTGACATTTCTTCTTCCT
TTTGTGTTTTTTAGGAGAGGCATGCTGCGGAGGTGCGCAGGAACAAGGAACTCCAGG
TTGAACTGTCTGGCTGAAGCAAGGGAGGGTCTGGCACGCCCCACCAATAGTAAATCC
CCCTGCCTATATTATAATGGATCATGCGATATCAGGATGGGGAATGTATGACATGGTT
TAAAAAGAACTCATTATAAAAAAAAAAAAACAAAAAAAATCAAAAATTAAAAAAAA
TCAATGCGGTCTCTTTGCAGAATGTTTTGCTTGATGTTTAAAAAATACCTTGGATCTT
ATTTTGTAAATACTTACATTTTTGTTAAAAAATACAAGTATTGCATTATGCAAGTTAT
TTCATAATCTTACATGTCCTGTAACAGGCTTTTGATGTTGTGTCTTTCCACTCAAATGA
ATTTGCTAGGTCTGTTCTTTTTGAAGCTCCCCATGTCTAACTCCATTCCAAAAGAAAA
ATGAGGTCAGTAGACAGTCTATGGTGCTAGAAACCCACCATTGCCTAATGACCTAGA
AGGCTTTGTTGTCTCTGAGCTTGACTAAGACCATACCTAGATCACAGGTATTATGACT
CCACATGAACCTTCACATTTGTTCGCTCATAATCTACTTACTGCCTAAAAACTACAAA
ACCAGGCTAAGAAATACCACCAGTCATAGCATTTACTTCTGCTTCTCCTGGATTATGT
GCTACAAATGTGCTTTGGCTTTAGAAAGGGATGGATGAGAAGACAGACCTGAGACCA
ATCTGGGTAGAAGCAAAAAGTTGAACCTTTTAAAGTGCTGAACACAAATCCAAATTC
GAATGGTTCAAGCAGCCGTGAAATCGCTCTTCATAAAGTGGGCTTAATTCTCTAGTTT
AAGTTCTTTTGATGGAATGAATTAATTAATGTGTCAGGTGGCTTATTTGTGGATGCCA
TGATTGATGATGTTCATTTTAAGCTCTTACCTATAGTACAAGTACATGATGCTACTGA
ATATTTTTCCACTTGGAAACTGTGAGCTGGTTGTTGCATTAAAACACACATACAAACA
AAATCAAAAACACTGCGGACTTTCACTCAAGCTGGTCTTTCTTCCCCAGTGTAAGGCA
ATCCTGCCTACTAACAACACCAACAACAAAACACTCCATCTGTGAAGCTGACGCAGT
TAAGGGGGCTAGGCAGGGCATTTGTGCCAACTAAGAATCACCAGATACCCACCATAA
GTACCTATCGCAGTTTTGAAGTCGTTTCTCCCCAACTCCCAACTCCTGAAGGTTGCTG
CCTGCATATTTACTCTTCATTAGTGCTATTTTCCTGTATGTCATTGTGAGCAAGCTGTG
ATTAATAAAGAATTGGAGTTCTGTGAACTAATAAAGGTTTGGTCTGTT

### STMN2 Oligonucleotides Targeting Regions of the STMN2 Transcript

In various embodiments, STMN2 AON disclosed herein are complementary to specific regions of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1341. In some embodiments, a STMN2 AON comprises a sequence that is complementary to a specific region of the STMN2 transcript (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. In some embodiments, a STMN2 AON comprises a sequence that is between 85 and 98% complementary to a specific region of the STMN2 transcript. In some embodiments, a STMN2 AON comprises a sequence that is 90 to 95% complementary to a specific region of the STMN2 transcript.

In some embodiments, the STMN2 AON (e.g., STMN2 AON) has a segment that has, at most, 7 linked nucleosides. In some embodiments, the STMN2 AON has a segment that has, at most, 6, 5, 4, 3, or 2 linked nucleosides. The segments of the STMN2 AON may be separated from other segments of the STMN2 AON through a spacer. The segment of the STMN2 AON is complementary to a specific region of the STMN2 transcript (for example, a STMN2 transcript comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341.

In some embodiments, a STMN2 AON targets a specific portion of the STMN2 transcript, the specific portion of the STMN2 transcript comprising any one of positions 144-168, 173-197, 185-209, or 237-261 of SEQ ID NO: 1339. In some embodiments, a STMN2 AON targets a specific portion of the STMN2 transcript, the specific portion of the STMN2 transcript comprising any one of positions 121-144, 146-170, 150-170, 150-172, 150-170, 150-172, 150-174, 169-193, 170-194, 171-195, 172-196, 197-221, 249-273, 252-276, or 276-300. In some embodiments, a STMN2 AON targets a specific portion of the STMN2 transcript, the specific portion of the STMN2 transcript comprising any one of positions 144-164, 144-166, 145-167, 146-166, 146-168, 147-165, or 148-168 of SEQ ID NO: 1339. In some embodiments, a STMN2 AON targets a specific portion of the STMN2 transcript, the specific portion of the STMN2 transcript comprising any one of positions 173-191, 173-193, 173-195, 173-197, 175-195, 175-197, 177-197, or 179-197 of SEQ ID NO: 1339. In some embodiments, a STMN2 AON targets a specific portion of the STMN2 transcript, the specific portion of the STMN2 transcript comprising any one of positions 185-205, 187-209, 189-209, or 191-209 of SEQ ID NO: 1339. In some embodiments, a STMN2 AON targets a specific portion of the STMN2 transcript, the specific portion of the STMN2 transcript comprising any one of positions 237-255, 237-259, 239-259, 239-261, 241-261, or 243-261 of SEQ ID NO: 1339.

In some embodiments, a STMN2 AON targets a specific portion of the STMN2 transcript, the specific portion of the STMN2 transcript consisting of any one of positions 144-168, 173-197, 185-209, or 237-261 of SEQ ID NO: 1339. In some embodiments, a STMN2 AON targets a specific portion of the STMN2 transcript, the specific portion of the STMN2 transcript consisting of any one of positions 144-164, 144-166, 145-167, 146-166, 146-168, 147-165, or 148-168 of SEQ ID NO: 1339. In some embodiments, a STMN2 AON targets a specific portion of the STMN2 transcript, the specific portion of the STMN2 transcript consisting of any one of positions 173-191, 173-193, 173-195, 173-197, 175-195, 175-197, 177-197, or 179-197 of SEQ ID NO: 1339. In some embodiments, a STMN2 AON targets a specific portion of the STMN2 transcript, the specific portion of the STMN2 transcript consisting of any one of positions 185-205, 187-209, 189-209, or 191-209 of SEQ ID NO: 1339. In some embodiments, a STMN2 AON targets a specific portion of the STMN2 transcript, the specific portion of the STMN2 transcript consisting of any one of positions 237-255, 237-259, 239-259, 239-261, 241-261, or 243-261 of SEQ ID NO: 1339.

### STMN2 Oligonucleotide Variants

In various embodiments, STMN2 AONs include different variants, hereafter referred to as STMN2 AON variants. A STMN2 AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A STMN2 AON variant may be an oligonucleotide sequence complementary to a portion of a STMN2 pre-mRNA sequence or a STMN2 gene sequence.

In various embodiments, a STMN2 AON variant represents a modified version of a corresponding STMN2 parent oligonucleotide that includes a nucleobase sequence selected from any one of SEQ ID NOs: 1-446 or SEQ ID NOs: 893-1338. In some embodiments, a STMN2 AON variant includes a nucleobase sequence that represents a shortened version of a nucleobase sequence of a STMN2 AON selected from any one of SEQ ID NOs: 1-446 or SEQ ID NOs: 893-1338. As one example, if a STMN2 parent oligonucleotide includes a 25mer (e.g., 25 nucleotide bases in length) a variant (e.g., a STMN2 variant) may include a shorter version (e.g., 15mer, 17mer, 19mer, 21mer, or 23mer) of the 25mer STMN2 parent oligonucleotide. In one embodiment, a nucleobase sequence of a STMN2 AON variant differs from a corresponding nucleobase sequence of a STMN2 parent oligonucleotide in that 1, 2, 3, 4, 5, or 6 nucleotide bases are removed from one or both of the 3′ and 5′ ends of the nucleobase sequence of the STMN2 parent oligonucleotide. In one embodiment, the corresponding STMN2 AON variant may include a 23mer where two nucleotide bases were removed from one of the 3′ or 5′ end of a 25mer included in the STMN2 parent oligonucleotide. In one embodiment, the corresponding STMN2 AON variant may include a 23mer where one nucleotide base is removed from each of the 3′ and 5′ ends of the 25mer included in the STMN2 parent oligonucleotide. In one embodiment, the corresponding STMN2 AON variant may include a 21mer where two nucleotide bases are removed from each of the 3′ and 5′ ends of the 25mer included in the STMN2 parent oligonucleotide. In one embodiment, the corresponding STMN2 AON variant may include a 21mer where four nucleotide bases are removed from either the 3′ or 5′ end of the 25mer included in the STMN2 parent oligonucleotide. In one embodiment, the corresponding STMN2 AON variant may include a 19mer where three nucleotide bases are removed from each of the 3′ and 5′ ends of the 25mer included in the STMN2 parent oligonucleotide. In one embodiment, the corresponding STMN2 AON variant may include a 19mer where six nucleotide bases are removed from either the 3′ or 5′ end of the 25mer included in the STMN2 parent oligonucleotide.

Example sequences of STMN2 AON variants are shown below in Tables 5A and 5B.

TABLE 5A
STMN2 Oligonucleotide Variant Sequences

| SEQ | |
|---|---|
| ID | |
| NO: | |
| | AON Sequence* (5′ → 3′) |
| 1342 | ATCCAATTAAGAGAGAGTGATGG |
| 1343 | AATCCAATTAAGAGAGAGTGATG |
| 1344 | TCCAATTAAGAGAGAGTGATGGG |
| 1345 | GAGTCCTGCAATATGAATATAAT |
| 1346 | GTCCTGCAATATGAATATAATTT |
| 1347 | GTCTTCTGCCGAGTCCTGCAATA |
| 1348 | GCACACATGCTCACACAGAGAGC |
| 1349 | ACACATGCTCACACAGAGAGCCA |
| 1350 | TCCAATTAAGAGAGAGTGATG |
| 1351 | AATCCAATTAAGAGAGAGTGA |
| 1352 | CAATTAAGAGAGAGTGATGGG |
| 1353 | GTCCTGCAATATGAATATAAT |
| 1354 | GAGTCCTGCAATATGAATATA |
| 1355 | CCTGCAATATGAATATAATTT |
| 1356 | AGGTCTTCTGCCGAGTCCTGC |
| 1357 | CTTCTGCCGAGTCCTGCAATA |
| 1358 | ACACATGCTCACACAGAGAGC |
| 1359 | GCACACATGCTCACACAGAGA |
| 1360 | ACATGCTCACACAGAGAGCCA |
| 1361 | CCAATTAAGAGAGAGTGAT |
| 1362 | GAGTCCTGCAATATGAATA |
| 1363 | TGCAATATGAATATAATTT |
| 1364 | TCTGCCGAGTCCTGCAATA |
| 1365 | GCACACATGCTCACACAGA |
| 1366 | ATGCTCACACAGAGAGCCA |
| | Target Sequence (5′ → 3′) |
| 1367 | CCATCACTCTCTCTTAATTGGAT |
| 1368 | CATCACTCTCTCTTAATTGGATT |
| 1369 | CCCATCACTCTCTCTTAATTGGA |
| 1370 | ATTATATTCATATTGCAGGACTC |
| 1371 | AAATTATATTCATATTGCAGGAC |
| 1372 | TATTGCAGGACTCGGCAGAAGAC |
| 1373 | GCTCTCTGTGTGAGCATGTGTGC |
| 1374 | TGGCTCTCTGTGTGAGCATGTGT |
| 1375 | CATCACTCTCTCTTAATTGGA |
| 1376 | TCACTCTCTCTTAATTGGATT |
| 1377 | CCCATCACTCTCTCTTAATTG |
| 1378 | ATTATATTCATATTGCAGGAC |
| 1379 | TATATTCATATTGCAGGACTC |
| 1380 | AAATTATATTCATATTGCAGG |
| 1381 | GCAGGACTCGGCAGAAGACCT |
| 1382 | TATTGCAGGACTCGGCAGAAG |
| 1383 | GCTCTCTGTGTGAGCATGTGT |
| 1384 | TCTCTGTGTGAGCATGTGTGC |
| 1385 | TGGCTCTCTGTGTGAGCATGT |
| 1386 | ATCACTCTCTCTTAATTGG |
| 1387 | TATTCATATTGCAGGACTC |
| 1388 | AAATTATATTCATATTGCA |
| 1389 | TATTGCAGGACTCGGCAGA |
| 1390 | TCTGTGTGAGCATGTGTGC |
| 1391 | TGGCTCTCTGTGTGAGCAT |

* At least one nucleoside linkage of the nucleobase sequence is selected from a phosphorothioate linkage, an alkyl phosphate linkage, a phosphorodithioate linkage, a phosphotriester linkage,
an alkylphosphonate linkage, a 3-methoxypropyl phosphonate linkage, a methylphosphonate linkage, an aminoalkylphosphotriester linkage, an alkylene phosphonate linkage, a phosphinate linkage,
a phosphoramidate linkage, a phosphoramidothioate linkage, a thiophosphorodiamidate linkage, a phosphorodiamidate (e.g., comprising a phosphorodiamidate morpholino (PMO), 3’ amino ribose, or
5’ amino ribose) linkage, an aminoalkylphosphoramidate linkage, a thiophosphoramidate linkage, a thionoalkylphosphonate linkage, a thionoalkylphosphotriester linkage, a thiophosphate
linkage, a selenophosphate linkage, and a boranophosphate linkage.

TABLE 5B
Additional STMN2 Oligonucleotide Variant
Sequences

| SEQ ID NO: | AON Sequence* (5′ → 3′) |
|---|---|
| 1421 | CCTGCAATATGAATATAATTTTA |
| 1422 | TGCAATATGAATATAATTTTAAA |
| 1423 | CTGCAATATGAATATAATTTTAA |
| 1424 | TGCAATATGAATATAATTTTA |
| 1425 | TCCTGCAATATGAATATAATTTT |
| 1426 | CTGCAATATGAATATAATTTT |
| 1427 | AGTCCTGCAATATGAATATAATT |
| 1428 | TCCTGCAATATGAATATAATT |
| 1429 | TTTCTCTCGAAGGTCTTCTGCCG |
| 1430 | CCTTTCTCTCGAAGGTCTTCTGC |
| 1431 | CTTTCTCTCGAAGGTCTTCTGCC |
| 1432 | CTCTCGCACACACGCACACATGC |
| 1433 | CTCTCTCGCACACACGCACACAT |
| 1434 | TCTCTCGCACACACGCACACATG |
| 1435 | CTCTCGCACACACGCACACAT |

* At least one nucleoside linkage of the nucleobase sequence is selected from a phosphorothioate linkage, an alkyl phosphate linkage, a phosphorodithioate linkage, a phosphotriester linkage,
an alkylphosphonate linkage, a 3-methoxypropyl phosphonate linkage, a methylphosphonate linkage, an aminoalkylphosphotriester linkage, an alkylene phosphonate linkage, a phosphinate linkage,
a phosphoramidate linkage, a phosphoramidothioate linkage, a thiophosphorodiamidate linkage, a phosphorodiamidate (e.g., comprising a phosphorodiamidate morpholino (PMO), 3’ amino ribose,
or 5’ amino ribose) linkage, an aminoalkylphosphoramidate linkage, a thiophosphoramidate linkage, a thionoalkylphosphonate linkage, a thionoalkylphosphotriester linkage, a thiophosphate
linkage, a selenophosphate linkage, and a boranophosphate linkage.

Table 6 below identifies additional variants of STMN2 AON sequences:

TABLE 6
Additional STMN2 Oligonucleotide
Variant Sequences

| SEQ | |
|---|---|
| ID | |
| NO: | AON Sequence* (5′ → 3′) |
| 1392 | AUCCAAUUAAGAGAGAGUGAUGG |
| 1393 | AAUCCAAUUAAGAGAGAGUGAUG |
| 1394 | UCCAAUUAAGAGAGAGUGAUGGG |
| 1395 | GAGUCCUGCAAUAUGAAUAUAAU |
| 1396 | GUCCUGCAAUAUGAAUAUAAUUU |
| 1397 | GUCUUCUGCCGAGUCCUGCAAUA |
| 1398 | GCACACAUGCUCACACAGAGAGC |
| 1399 | ACACAUGCUCACACAGAGAGCCA |
| 1400 | UCCAAUUAAGAGAGAGUGAUG |
| 1401 | AAUCCAAUUAAGAGAGAGUGA |
| 1402 | CAAUUAAGAGAGAGUGAUGGG |
| 1403 | GUCCUGCAAUAUGAAUAUAAU |
| 1404 | GAGUCCUGCAAUAUGAAUAUA |
| 1405 | CCUGCAAUAUGAAUAUAAUUU |
| 1406 | AGGUCUUCUGCCGAGUCCUGC |
| 1407 | CUUCUGCCGAGUCCUGCAAUA |
| 1408 | ACACAUGCUCACACAGAGAGC |
| 1409 | GCACACAUGCUCACACAGAGA |
| 1410 | ACAUGCUCACACAGAGAGCCA |
| 1411 | CCAAUUAAGAGAGAGUGAU |
| 1412 | GAGUCCUGCAAUAUGAAUA |
| 1413 | UGCAAUAUGAAUAUAAUUU |
| 1414 | UCUGCCGAGUCCUGCAAUA |
| 1415 | GCACACAUGCUCACACAGA |
| 1416 | AUGCUCACACAGAGAGCCA |
| 1436 | CCUGCAAUAUGAAUAUAAUUUUA |
| 1437 | UGCAAUAUGAAUAUAAUUUUAAA |
| 1438 | CUGCAAUAUGAAUAUAAUUUUAA |
| 1439 | UGCAAUAUGAAUAUAAUUUUA |
| 1440 | UCCUGCAAUAUGAAUAUAAUUUU |
| 1441 | CUGCAAUAUGAAUAUAAUUUU |
| 1442 | AGUCCUGCAAUAUGAAUAUAAUU |
| 1443 | UCCUGCAAUAUGAAUAUAAUU |
| 1444 | UUUCUCUCGAAGGUCUUCUGCCG |
| 1445 | CCUUUCUCUCGAAGGUCUUCUGC |
| 1446 | CUUUCUCUCGAAGGUCUUCUGCC |
| 1447 | CUCUCGCACACACGCACACAUGC |
| 1448 | CUCUCUCGCACACACGCACACAU |
| 1449 | UCUCUCGCACACACGCACACAUG |
| 1450 | CUCUCGCACACACGCACACAU |

* At least one nucleoside linkage of the nucleobase sequence is selected from a phosphorothioate linkage, an alkyl phosphate linkage, a phosphorodithioate linkage, a phosphotriester linkage,
an alkylphosphonate linkage, a 3-methoxypropyl phosphonate linkage, a methylphosphonate linkage, an aminoalkylphosphotriester linkage, an alkylene phosphonate linkage, a phosphinate linkage,
a phosphoramidate linkage, a phosphoramidothioate linkage, a thiophosphorodiamidate linkage, a phosphorodiamidate (e.g., comprising a phosphorodiamidate morpholino (PMO), 3’ amino ribose,
or 5’ amino ribose) linkage, an aminoalkylphosphoramidate linkage, a thiophosphoramidate linkage, a thionoalkylphosphonate linkage, a thionoalkylphosphotriester linkage, a thiophosphate
linkage, a selenophosphate linkage, and a boranophosphate linkage.

Antisense Oligonucleotides with One or More Spacers

In various embodiments, antisense oligonucleotides comprise one or more spacers. In particular embodiments, an antisense oligonucleotide includes one spacer. In particular embodiments, an antisense oligonucleotide includes two spacers. In particular embodiments, an antisense oligonucleotide includes three spacers. Generally, a spacer refers to a nucleoside-replacement group lacking a nucleotide base and wherein the nucleoside sugar moiety is replaced by a non-sugar substitute group. The non-sugar substitute group is not capable of linking to a nucleobase, but is capable of linking with the 3′ and 5′ positions of nucleosides adjacent to the spacer through an internucleoside linking group.

In certain embodiments, an oligonucleotide with one or more spacers, such as disclosed herein, may be an oligonucleotide with 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, or 25 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, oligonucleotides with one or more spacers are 25 oligonucleotide units in length. In particular embodiments, the oligonucleotides with one or more spacers are 23 oligonucleotide units in length. In particular embodiments, the oligonucleotides with one or more spacers are 21 oligonucleotide units in length. In particular embodiments, the oligonucleotides with one or more spacers are 19 oligonucleotide units in length. In various embodiments, the oligonucleotides with one or more spacers are at least 18, at least 19, at least 20, at least 21, at least 22, at least 23, at least 24, or at least 25 oligonucleotide units in length. In various embodiments, the oligonucleotides with one or more spacers are at least 18 oligonucleotide units in length. In various embodiments, the oligonucleotides with one or more spacers are at least 19 oligonucleotide units in length. In various embodiments, the oligonucleotides with one or more spacers are at least 20 oligonucleotide units in length. In various embodiments, the oligonucleotides with one or more spacers are at least 21 oligonucleotide units in length. In various embodiments, the oligonucleotides with one or more spacers are at least 22 oligonucleotide units in length. In various embodiments, the oligonucleotides with one or more spacers are at least 23 oligonucleotide units in length. In various embodiments, the oligonucleotides with one or more spacers are at least 24 oligonucleotide units in length. In various embodiments, the oligonucleotides with one or more spacers are at least 25 oligonucleotide units in length.

In various embodiments, a STMN2 AON comprises a sequence that shares at least 80% identity with an equal length portion of any one of SEQ ID NOs: 1451-1664. In various embodiments, a STMN2 AON comprises a sequence that shares at least 85% identity with an equal length portion of any one of SEQ ID NOs: 1451-1664. In various embodiments, a STMN2 AON comprises a sequence that shares at least 90% identity with an equal length portion of any one of SEQ ID NOs: 1451-1664. In various embodiments, a STMN2 AON comprises a sequence that shares at least 95% identity with an equal length portion of any one of SEQ ID NOs: 1451-1664. In various embodiments, a STMN2 AON comprises a sequence that shares 100% identity with an equal length portion of any one of SEQ ID NOs: 1451-1664.

In some embodiments, the spacer is of Formula (X):

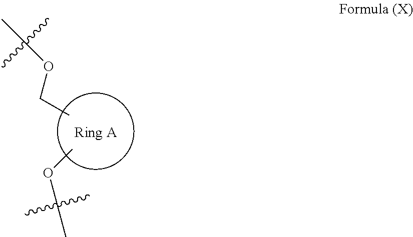

wherein ring A is as defined herein.

In some embodiments, the spacer is of Formula (Xa):

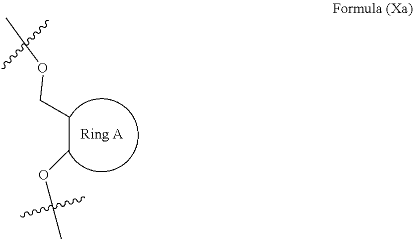 [SMILES: *OCCCO*]

wherein ring A is as defined herein and the —CH₂—O— group is on a ring A atom adjacent to the —O— group.

As generally defined herein, ring A of formulae (X) and (Xa), is an optionally substituted 4-8 member monocyclic cycloalkyl group (e.g. ring A is cyclobutyl, cyclopentyl, cyclohexyl, cycloheptyl, cyclooctyl) or a 4-8 member monocyclic heterocyclyl group, wherein the heterocyclyl group contains 1 or 2 heteroatoms selected from O, S and N (e.g. ring A is oxetanyl, tetrahydrofuranyl, tetrahydropyranyl, 1,4-dioxanyl, pyrrolidinyl, piperidinyl, piperazinyl, morpholinyl, azepanyl). In some embodiments, ring A is tetrahydrofuranyl. In some embodiments, ring A is tetrahydropyranyl. In some embodiments, ring A is pyrrolidinyl. In some embodiments, ring A is cyclopentyl. In some embodiments, the monocyclic cycloalkyl or monocyclic heterocyclyl is not further substituted. In some embodiments, the cycloalkyl or heterocyclyl is further substituted with 0, 1, 2 or 3 substituents selected from halo (e.g., —F, —Cl), -Ome, -Oet —O(CH₂)Ome, —O(CH₂)₂Ome and CN. In some embodiments, the spacer is represented by Formula (I), wherein:

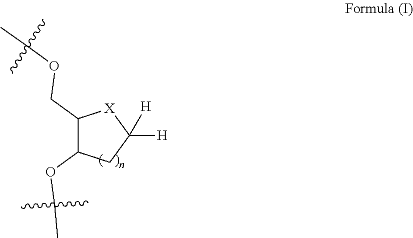

X is selected from —CH₂— and —O—; and

n is 0, 1, 2 or 3.

In some embodiments, the spacer is represented by Formula (I′), wherein:

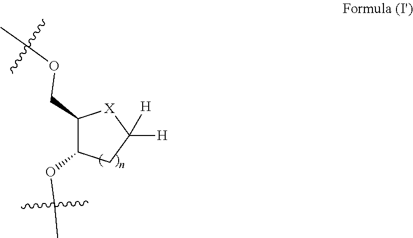

X is selected from —CH₂— and —O—; and

n is 0, 1, 2 or 3.

In some embodiments, the spacer is represented by Formula (Ia), wherein:

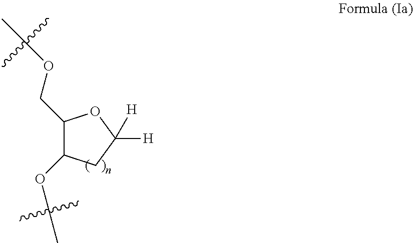

and n is 0, 1, 2 or 3.

In some embodiments, the spacer is represented by Formula (Ia′), wherein:

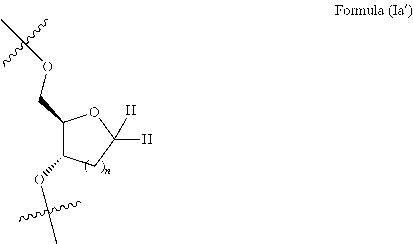

and n is 0, 1, 2 or 3.

As generally defined herein, X is selected from —CH₂— and —O—. In some embodiments, X is —CH₂—. In other embodiments, X is —O—.

As generally defined herein, n is 0, 1, 2 or 3. In some embodiments, n is 0. In some embodiments, n is 1 or 2. In some embodiments, n is 1. In other embodiments, n is 2. In certain embodiments, n is 3.

In some embodiments, the spacer is represented by Formula (II), wherein:

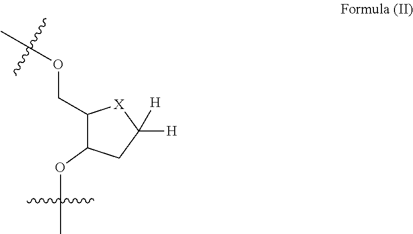

X is selected from —CH₂— and

In some embodiments, the spacer is represented by Formula (II′), wherein:

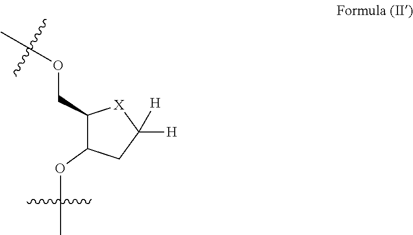

X is selected from —CH₂— and —O.

In some embodiments, the spacer is represented by Formula (Iia), wherein:

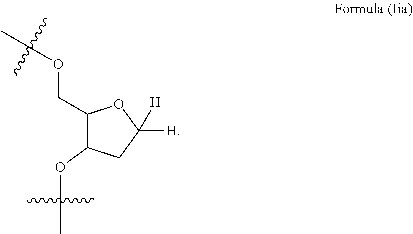

In some embodiments, the spacer is represented by Formula (Iia′), wherein:

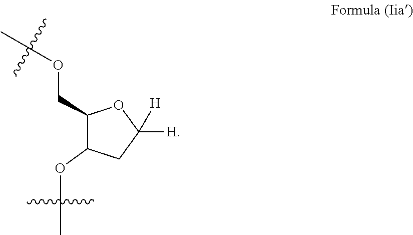

In some embodiments, the spacer is represented by Formula (III), wherein:

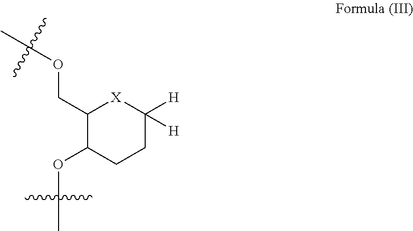

X is selected from —CH₂— and —O—.

In some embodiments, the spacer is represented by Formula (III′), wherein:

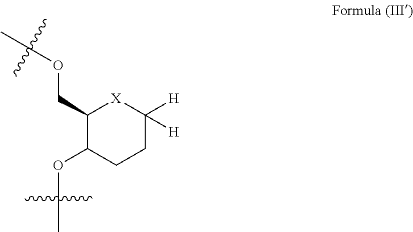

X is selected from —CH₂— and —O.

In some embodiments, the spacer is represented by Formula (IIIa), wherein:

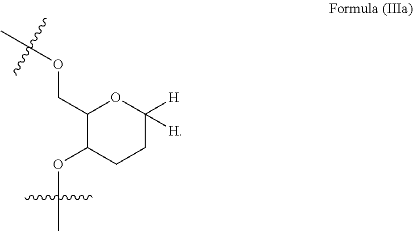

In some embodiments, the spacer is represented by Formula (IIIa′), wherein:

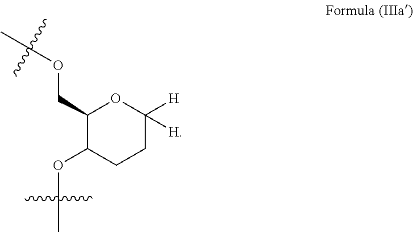

In some embodiments, the open positions of Formulae (I), (I′), (Ia), (Ia′), (II), (II′), (Iia), (Iia′), (III), (III′), (IIIa) and (IIIa′) (i.e., the positions not specifically depicted as bearing exclusively hydrogen atoms, including the —CH₂— group of X) are further substituted with 0-3 substituents selected from halo (e.g., —F, —Cl), -Ome, -Oet —O(CH₂)Ome, —O(CH₂)₂Ome and CN. In some embodiments, Formulae (I), (I′), (Ia), (Ia′), (II), (II′), (Iia), (Iia′), (III), (III′), (IIIa) and (IIIa′) are not further substituted.

As described further below, a STMN2 oligonucleotide with one or more spacers is described in reference to a corresponding STMN2 parent oligonucleotide. In various embodiments, a STMN2 oligonucleotide with a spacer differs from a STMN2 parent oligonucleotide in that the spacer replaces a nucleoside in the STMN2 parent oligonucleotide. As used hereafter, the “position” of the STMN2 oligonucleotide refers to a particular location as counted from the 5′ end of the STMN2 oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the STMN2 parent oligonucleotide. In particular embodiments, a spacer replaces a nucleoside at one of positions 7, 8, 11, 14, 16, 19, or 22 of the STMN2 parent oligonucleotide.

In various embodiments, a STMN2 oligonucleotide includes one spacer that replaces a nucleoside in the STMN2 parent oligonucleotide (e.g., one spacer replaces one nucleoside of the STMN2 parent oligonucleotide). In particular embodiments, the spacer replaces a nucleoside between positions 9 and 15 of the STMN2 parent oligonucleotide. In particular embodiments, the spacer replaces a nucleoside between positions 9 and 12 of the STMN2 parent oligonucleotide. In particular embodiments, the spacer replaces a nucleoside at position 10 of the STMN2 parent oligonucleotide. In particular embodiments, the spacer replaces a nucleoside at position 11 of the STMN2 parent oligonucleotide. In particular embodiments, the spacer replaces a nucleoside at position 12 of the STMN2 parent oligonucleotide. In particular embodiments, the spacer replaces a nucleoside between positions 12 and 16 of the STMN2 parent oligonucleotide. In particular embodiments, the spacer replaces a nucleoside at position 15 of the STMN2 parent oligonucleotide.

In various embodiments, a STMN2 oligonucleotide including one spacer has 2 segments, where at least one of the 2 segments has at most 11 linked nucleosides. For example, the STMN2 oligonucleotide may be 23 oligonucleotide units in length, and the spacer can be located at position 12. Therefore, the STMN2 oligonucleotide has 2 segments divided by the spacer, where both of the 2 segments are 11 nucleobases in length. In various embodiments, a STMN2 oligonucleotide including one spacer has 2 segments, where at least one of the 2 segments has at most 10 linked nucleosides. For example, the STMN2 oligonucleotide may be 21 oligonucleotide units in length, and the spacer can be located at position 11. Therefore, the STMN2 oligonucleotide has 2 segments divided by the spacer, where both of the 2 segments are 10 nucleobases in length. As another example, the STMN2 oligonucleotide may be 25 oligonucleotide units in length, and the spacer can be located at position 15. Therefore, the STMN2 oligonucleotide has 2 segments divided by the spacer, where one of the 2 segments is 14 nucleobases in length and the second of the 2 segments is 10 nucleobases in length.

In various embodiments, a STMN2 oligonucleotide includes two spacers that each replace a nucleoside in the STMN2 parent oligonucleotide (e.g., two spacers replace two separate nucleosides of the STMN2 parent oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the STMN2 parent oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the STMN2 parent oligonucleotide. In particular embodiments, the second spacer replaces a nucleoside between positions 14 and 22 of the STMN2 parent oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the STMN2 parent oligonucleotide.

In preferred embodiments, the first spacer replaces a nucleoside at position 7 of the STMN2 parent oligonucleotide and the second spacer replaces a nucleoside at position 14 of the STMN2 parent oligonucleotide. In preferred embodiments, the first spacer replaces a nucleoside at position 8 of the STMN2 parent oligonucleotide and the second spacer replaces a nucleoside at position 16 of the STMN2 parent oligonucleotide. In preferred embodiments, the first spacer replaces a nucleoside at position 11 of the STMN2 parent oligonucleotide and the second spacer replaces a nucleoside at position 22 of the STMN2 parent oligonucleotide. In preferred embodiments, the first spacer replaces a nucleoside at position 9 of the STMN2 parent oligonucleotide and the second spacer replaces a nucleoside at position 19 of the STMN2 parent oligonucleotide.

In various embodiments, a STMN2 oligonucleotide includes three spacers that each replace a nucleoside in the STMN2 parent oligonucleotide (e.g., three spacers replace three separate nucleosides of the STMN2 parent oligonucleotide). In particular embodiments, the first spacer replaces a nucleoside between positions 7 and 11 of the STMN2 parent oligonucleotide. In particular embodiments, the second spacer replaces a nucleoside between positions 14 and 22 of the STMN2 parent oligonucleotide. In particular embodiments, the third spacer replaces a nucleoside between positions 21 and 24 of the STMN2 parent oligonucleotide. In some embodiments, the first spacer replaces a nucleoside between positions 2 and 5 of the STMN2 parent oligonucleotide. In particular embodiments, the second spacer replaces a nucleoside between positions 8 and 12 of the STMN2 parent oligonucleotide. In particular embodiments, the third spacer replaces a nucleoside between positions 18 and 22 of the STMN2 parent oligonucleotide.

In various embodiments, the three spacers in a STMN2 oligonucleotide are positioned such that each of the four segments of the STMN2 oligonucleotide are at most 7 linked nucleosides in length. For example, a STMN2 oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides).). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the STMN2 oligonucleotide with one or more spacers has a particular GC content. As used herein, GC content (or guanine-cytosine content) is the percentage of nitrogenous bases in the oligonucleotide that are either guanine (G) or cytosine ®. In various embodiments, the STMN2 oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the STMN2 oligonucleotide with one or more spacers has at least 30% GC content. In particular embodiments, the STMN2 oligonucleotide with one or more spacers has at least 40% GC content. In various embodiments, the one or more spacers are positioned in the STMN2 oligonucleotide to maximize GC content. For example, instead of selecting a guanine or cytosine for replacement by a spacer in the STMN2 oligonucleotide, a thymine or adenine can be selected for replacement by a spacer.

In various embodiments, a STMN2 oligonucleotide with spacers is designed such that 1) each segment of the STMN2 oligonucleotide has at most 7 linked nucleosides and 2) at least two, three, or four spacers are positioned adjacent to a guanine group. In some embodiments, a STMN2 oligonucleotide with spacers is designed such that 1) each segment of the STMN2 oligonucleotide has at most 7 linked nucleosides and 2) each of two spacers precede a guanine group.

In various embodiments, the inclusion of one or more spacers in the STMN2 oligonucleotide does not decrease the effectiveness of the STMN2 oligonucleotide with the spacers in restoring full length STMN2 protein or full length STMN2 mRNA in comparison to the effect of a corresponding STMN2 parent oligonucleotide. In various embodiments, the inclusion of one or more spacers in the STMN2 oligonucleotide increases the effectiveness of the STMN2 oligonucleotide with the spacers in restoring full length STMN2 protein or full length STMN2 mRNA in comparison to the effect of a corresponding STMN2 parent oligonucleotide. In various embodiments, the inclusion of one or more spacers in the STMN2 oligonucleotide does not decrease the effectiveness of the STMN2 oligonucleotide with the spacers in reducing quantity of STMN2 transcripts with a cryptic exon in comparison to the effect of a corresponding STMN2 parent oligonucleotide. In various embodiments, the inclusion of one or more spacers in the STMN2 oligonucleotide increases the effectiveness of the STMN2 oligonucleotide with the spacers in reducing quantity of STMN2 transcripts with a cryptic exon in comparison to the effect of a corresponding STMN2 parent oligonucleotide.

Tables 7A, 7B, 8, and 9 document example STMN2 oligonucleotides with one or more spacers and their relation to corresponding STMN2 parent oligonucleotides. Each STMN2 oligonucleotide is assigned a sequence name. As used hereafter, the nomenclature of the sequence name is expressed as “X_spA” (for a STMN2 AON with one spacer), “X_spA_spB” (for a STMN2 AON with two spacers), or “X_spA_spB_spC” (for a STMN2 AON with three spacers). Here, “X” refers to the length of the STMN2 AON, “A” refers to the position in the STMN2 AON where the first spacer is located, “B” refers to the position in the STMN2 AON where the second spacer is located, and if present, “C” refers to the position in the STMN2 AON where the third spacer is located.

In various embodiments, STMN2 oligonucleotides include one spacer. In various embodiments, the STMN2 oligonucleotides are oligonucleotide variants, such as any one of a 23mer, 21mer, or 19mer. In various embodiments, the inclusion of a spacer divides up the STMN2 oligonucleotide into two separate segments, where at least one of the segments is at most 11 linked nucleosides in length. In various embodiments, the inclusion of a spacer divides up the STMN2 oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide. Example STMN2 AONs with one spacer are documented below in Table 7A.

TABLE 7A
Identification of STMN2 AONs with one spacer.
Here, each STMN2 AON has 2 segments, where at
least one of the segments has at most 11
linked nucleosides.

| | | | Sequence* |
|---|---|---|---|
| | | | (where X |
| | | | indicates |
| | | | a nucleo- |
| | | | side of |
| | | | the STMN2 |
| | | | parent |
| | | | oligonu- |
| | | | cleotide |
| | | | and S_{y} |
| | | | indicates |
| | | | presence |
| | | Se- | of |
| | | quence | a Spacer |
| | Relation | ID | where y |
| | to STMN2 | Number | denotes |
| | oligonu- | (SEQ | the |
| Sequence | cleotide | ID | position) |
| name | variant | NO) | (5′ → 3′) |
| STMN2 parent | N/A | 1522 | XXXXXXXXXXXXXX |
| oligonucleo- | | | XXXXXXXXXXX |
| tide | | | (25mer) |
| STMN2 Oligo- | Nucleo- | 1523 | XXXXXXXXXXXXXX |
| nucleotide | side at | | S₁₅XXXXXXXXXX |
| (25mer) with | position | | |
| Spacer at | 15 of | | |
| position 15 | 25mer is | | |
| (STMN2 | substi- | | |
| AON 25_sp15) | tuted with | | |
| | a spacer | | |
| STMN2 oligo- | N/A | 1524 | XXXXXXXXXXXXXX |
| nucleotide | | | XXXXXXXXX |
| variant | | | (23mer) |
| (23mer) | | | |
| STMN2 Oligo- | Nucleo- | 1525 | XXXXXXXXXXX |
| nucleotide | side at | | S₁₂XXXXXXXXXXX |
| (23mer) with | position | | |
| Spacer at | 12 of | | |
| position 12 | 23mer is | | |
| (STMN2 | substi- | | |
| AON 23_sp12) | tuted with | | |
| | a spacer | | |
| STMN2 oligo- | N/A | 1526 | XXXXXXXXXXXXXX |
| nucleotide | | | XXXXXXX |
| variant | | | (21mer) |
| (21mer) | | | |
| STMN2 Oligo- | Nucleo- | 1527 | XXXXXXXXXX |
| nucleotide | side at | | S₁₁XXXXXXXXXX |
| (21mer) with | position | | |
| Spacer at | 11 of | | |
| position 11 | 21mer is | | |
| (STMN2 | substi- | | |
| AON 21_sp11) | tuted with | | |
| | a spacer | | |
| STMN2 oligo- | N/A | 1528 | XXXXXXXXXXXXXX |
| nucleotide | | | XXXXX |
| variant | | | (19mer) |
| (19mer) | | | |
| STMN2 Oligo- | Nucleo- | 1529 | XXXXXXXXX |
| nucleotide | side at | | S₁₀XXXXXXXXX |
| (19mer) with | position | | |
| Spacer at | 10 of | | |
| position 10 | 19mer is | | |
| (STMN2 | substi- | | |
| AON 19_sp10) | tuted with | | |
| | a spacer | | |

* At least one nucleoside linkage of the nucleobase sequence is selected from a phosphorothioate linkage, an alkyl phosphate linkage, a phosphorodithioate linkage, a phosphotriester linkage,
an alkylphosphonate linkage, a 3-methoxypropyl phosphonate linkage, a methylphosphonate linkage, an aminoalkylphosphotriester linkage, an alkylene phosphonate linkage, a phosphinate linkage,
a phosphoramidate linkage, a phosphoramidothioate linkage, a phosphorodiamidate (e.g, comprising a phosphorodiamidate morpholino (PMO), 3’ amino ribose, or 5’ amino ribose) linkage, an
aminoalkylphosphoramidate linkage, a thiophosphoramidate linkage, a thionoalkylphosphonate linkage, a thionoalkylphosphotriester linkage, a thiophosphate linkage, a
selenophosphate linkage, and a boranophosphate linkage.

In various embodiments, STMN2 oligonucleotides include two spacers. In various embodiments, the inclusion of a spacer divides up the STMN2 oligonucleotide into three separate segments, where at least one of the segments is at most 7 linked nucleosides in length. Example STMN2 AONs with two spacers are documented below in Table 7B.

TABLE 7B
Identification of STMN2 AONs with two spacers. Here, each
STMN2 AON has 3 segments, where at least one of the
segments has at most 7 linked nucleosides.

| | | | Sequence* (where X |
|---|---|---|---|
| | | | indicates a nucleoside |
| | | Sequence | of the STMN2 parent |
| | | ID | oligonucleotide and S_{y} |
| | Relation to | Number | indicates presence of |
| | STMN2 parent | (SEQ ID | a Spacer where y denotes |
| Sequence name | oligonucleotide | NO) | the position) (5′ → 3′) |
| STMN2 parent | N/A | 1530 | XXXXXXXXXXXXXXXXXXXXXXXXX |
| oligonucleotide | | | |
| STMN2 | Nucleosides at | 1531 | XXXXXXXXXXS₁₁XXXXXXXXXXS₂₂XXX |
| Oligonucleotide | positions 11 and | | |
| with Spacers at | 22 are each | | |
| positions 11 and 22 | substituted with | | |
| (STMN2 AON | a spacer | | |
| 25_sp11sp22) | | | |
| STMN2 | Nucleosides at | 1532 | XXXXXXS₇XXXXXXS₁₄XXXXXXXXXXX |
| Oligonucleotide | positions 7 and | | |
| with Spacers at | 14 are each | | |
| positions 7 and 14 | substituted with | | |
| (STMN2 AON | a spacer | | |
| 25_sp7sp14) | | | |
| STMN2 | Nucleosides at | 1533 | XXXXXXXS₈XXXXXXXXXXS₁₉XXXXXX |
| Oligonucleotide | positions 8 and | | |
| with Spacers at | 19 are | | |
| positions 8 and 19 | substituted with | | |
| (STMN2 AON | spacers | | |
| 25_sp8sp19) | | | |
| STMN2 | Nucleosides at | 1534 | XXXXXXXS₈XXXXXS₁₄XXXXXXXXXXX |
| Oligonucleotide | positions 8 and | | |
| with Spacers at | 14 are | | |
| positions 8 and 14 | substituted with | | |
| (STMN2 AON | spacers | | |
| 25_sp8sp14) | | | |
| STMN2 | Nucleosides at | 1535 | XXXXXXXXS₉XXXXS₁₄XXXXXXXXXXX |
| Oligonucleotide | positions 9 and | | |
| with Spacers at | 14 are | | |
| positions 9 and 14 | substituted with | | |
| (STMN2 AON | spacers | | |
| 25_sp9sp14) | | | |
| STMN2 | Nucleosides at | 1536 | XXXXXXXXXS₁₀XXXS₁₄XXXXXXXXXXX |
| Oligonucleotide | positions 10 and | | |
| with Spacers at | 14 are | | |
| positions 10 and 14 | substituted with | | |
| (STMN2 AON | spacers | | |
| 25_sp10spM) | | | |
| STMN2 | Nucleosides at | 1537 | XXXXXXXXXXS₁₁XXS₁₄XXXXXXXXXXX |
| Oligonucleotide | positions 11 and | | |
| with Spacers at | 14 are | | |
| positions 11 and 14 | substituted with | | |
| (STMN2 AON | spacers | | |
| 25_sp11sp14) | | | |
| STMN2 | Nucleosides at | 1538 | XXXXXXXS₈XXXXXXS₁₅XXXXXXXXXX |
| Oligonucleotide | positions 8 and | | |
| with Spacers at | 15 are | | |
| positions 8 and 15 | substituted with | | |
| (STMN2 AON | spacers | | |
| 25_sp8sp15) | | | |
| STMN2 | Nucleosides at | 1539 | XXXXXXXXS₉XXXXXS₁₅XXXXXXXXXX |
| Oligonucleotide | positions 9 and | | |
| with Spacers at | 15 are | | |
| positions 9 and 15 | substituted with | | |
| (STMN2 AON | spacers | | |
| 25_sp9sp15) | | | |
| STMN2 | Nucleosides at | 1540 | XXXXXXXXXS₁₀XXXXS₁₅XXXXXXXXXX |
| Oligonucleotide | positions 10 and | | |
| with Spacers at | 15 are | | |
| positions 10 and 15 | substituted with | | |
| (STMN2 AON | spacers | | |
| 25_sp10sp15) | | | |
| STMN2 | Nucleosides at | 1541 | XXXXXXXXXXS₁₁XXXS₁₅XXXXXXXXXX |
| Oligonucleotide | positions 11 and | | |
| with Spacers at | 15 are | | |
| positions 11 and 15 | substituted with | | |
| (STMN2 AON | spacers | | |
| 25_sp11sp15) | | | |
| STMN2 | Nucleosides at | 1542 | XXXXXXXS₈XXXXXXXS₁₆XXXXXXXXX |
| Oligonucleotide | positions 8 and | | |
| with Spacers at | 16 are | | |
| positions 8 and 16 | substituted with | | |
| (STMN2 AON | spacers | | |
| 25_sp8sp16) | | | |
| STMN2 | Nucleosides at | 1543 | XXXXXXXXS₉XXXXXXS₁₆XXXXXXXXX |
| Oligonucleotide | positions 9 and | | |
| with Spacers at | 16 are | | |
| positions 9 and 16 | substituted with | | |
| (STMN2 AON | spacers | | |
| 25_sp9sp16) | | | |
| STMN2 | Nucleosides at | 1544 | XXXXXXXXXS₁₀XXXXXS₁₆XXXXXXXXX |
| Oligonucleotide | positions 10 and | | |
| with Spacers at | 16 are | | |
| positions 10 and 16 | substituted with | | |
| (STMN2 AON | spacers | | |
| 25_sp10sp16) | | | |
| STMN2 | Nucleosides at | 1545 | XXXXXXXXXXS₁₁XXXXS₁₆XXXXXXXXX |
| Oligonucleotide | positions 11 and | | |
| with Spacers at | 16 are | | |
| positions 11 and 16 | substituted with | | |
| (STMN2 AON | spacers | | |
| 25_sp11sp16) | | | |
| STMN2 | Nucleosides at | 1546 | XXXXXXXS₈XXXXXXXXS₁₇XXXXXXXX |
| Oligonucleotide | positions 8 and | | |
| with Spacers at | 17 are | | |
| positions 8 and 17 | substituted with | | |
| (STMN2 AON | spacers | | |
| 25_sp8sp17) | | | |
| STMN2 | Nucleosides at | 1547 | XXXXXXXXS₉XXXXXXXS₁₇XXXXXXXX |
| Oligonucleotide | positions 9 and | | |
| with Spacers at | 17 are | | |
| positions 9 and 17 | substituted with | | |
| (STMN2 AON | spacers | | |
| 25_sp9sp17) | | | |
| STMN2 | Nucleosides at | 1548 | XXXXXXXXXS₁₀XXXXXXS₁₇XXXXXXXX |
| Oligonucleotide | positions 10 and | | |
| with Spacers at | 17 are | | |
| positions 10 and 17 | substituted with | | |
| (STMN2 AON | spacers | | |
| 25_sp10sp17) | | | |
| STMN2 | Nucleosides at | 1549 | XXXXXXXXXXS₁₁XXXXXS₁₇XXXXXXXX |
| Oligonucleotide | positions 11 and | | |
| with Spacers at | 17 are | | |
| positions 11 and 17 | substituted with | | |
| (STMN2 AON | spacers | | |
| 25_sp11sp17) | | | |
| STMN2 | Nucleosides at | 1550 | XXXXXXXS₈XXXXXXXXXS₁₈XXXXXXX |
| Oligonucleotide | positions 8 and | | |
| with Spacers at | 18 are | | |
| positions 8 and 18 | substituted with | | |
| (STMN2 ATON | spacers | | |
| 25_sp8sp18) | | | |
| STMN2 | Nucleosides at | 1551 | XXXXXXXXS₉XXXXXXXXS₁₈XXXXXXX |
| Oligonucleotide | positions 9 and | | |
| with Spacers at | 18 are | | |
| positions 9 and 18 | substituted with | | |
| (STMN2 AON | spacers | | |
| 25_sp9sp18) | | | |
| STMN2 | Nucleosides at | 1552 | XXXXXXXXXS₁₀XXXXXXXS₁₈XXXXXXX |
| Oligonucleotide | positions 10 and | | |
| with Spacers at | 18 are | | |
| positions 10 and 18 | substituted with | | |
| (STMN2 AON | spacers | | |
| 25_sp10sp18) | | | |
| STMN2 | Nucleosides at | 1553 | XXXXXXXXXXS₁₁XXXXXXS₁₈XXXXXXX |
| Oligonucleotide | positions 11 and | | |
| with Spacers at | 18 are | | |
| positions 11 and 18 | substituted with | | |
| (STMN2 AON | spacers | | |
| 25_sp11sp18) | | | |
| STMN2 | Nucleosides at | 1554 | XXXXXXXXS₉XXXXXXXXXS₁₉XXXXXX |
| Oligonucleotide | positions 9 and | | |
| with Spacers at | 19 are | | |
| positions 9 and 19 | substituted with | | |
| (STMN2 AON | spacers | | |
| 25_sp9sp19) | | | |
| STMN2 | Nucleosides at | 1555 | XXXXXXXXXS₁₀XXXXXXXXS₁₉XXXXXX |
| Oligonucleotide | positions 10 and | | |
| with Spacers at | 19 are | | |
| positions 10 and 19 | substituted with | | |
| (STMN2 AON | spacers | | |
| 25_sp10sp19) | | | |
| STMN2 | Nucleosides at | 1556 | XXXXXXXXXXS₁₁XXXXXXXS₁₉XXXXXX |
| Oligonucleotide | positions 11 and | | |
| with Spacers at | 19 are | | |
| positions 11 and 19 | substituted with | | |
| (STMN2 AON | spacers | | |
| 25_sp11sp19) | | | |
| STMN2 | Nucleosides at | 1557 | XXXXXXXXS₉XXXXXXXXXXS₂₀XXXXX |
| Oligonucleotide | positions 9 and | | |
| with Spacers at | 20 are | | |
| positions 9 and 20 | substituted with | | |
| (STMN2 AON | spacers | | |
| 25_sp9sp20) | | | |
| STMN2 | Nucleosides at | 1558 | XXXXXXXXXS₁₀XXXXXXXXXS₂₀XXXXX |
| Oligonucleotide | positions 10 and | | |
| with Spacers at | 20 are | | |
| positions 10 and 20 | substituted with | | |
| (STMN2 AON | spacers | | |
| 25_sp10sp20) | | | |
| STMN2 | Nucleosides at | 1559 | XXXXXXXXXXS₁₁XXXXXXXXS₂₀XXXXX |
| Oligonucleotide | positions 11 and | | |
| with Spacers at | 20 are | | |
| positions 11 and 20 | substituted with | | |
| (STMN2 AON | spacers | | |
| 25_sp11sp20) | | | |
| STMN2 | Nucleosides at | 1560 | XXXXXXXXXS_{1O}XXXXXXXXXXS₂₁XXXX |
| Oligonucleotide | positions 10 and | | |
| with Spacers at | 21 are | | |
| positions 10 and 21 | substituted with | | |
| (STMN2 AON | spacers | | |
| 25_sp10sp21) | | | |
| STMN2 | Nucleosides at | 1561 | XXXXXXXXXXS₁₁XXXXXXXXXS₂₁XXXX |
| Oligonucleotide | positions 11 and | | |
| with Spacers at | 21 are | | |
| positions 11 and 21 | substituted with | | |
| (STMN2 AON | spacers | | |
| 25_sp11sp21) | | | |
| STMN2 | Nucleosides at | 1562 | XXXS₄XXXXXXXXXXS₁₅XXXXXXXXXX |
| Oligonucleotide | positions 4 and | | |
| with Spacers at | 15 are | | |
| positions 4 and 15 | substituted with | | |
| (STMN2 AON | spacers | | |
| 25_sp4sp15) | | | |
| STMN2 | Nucleosides at | 1563 | XXXXXXS₇XXXXXXXXXXXS₁₉XXXXXX |
| Oligonucleotide | positions 7 and | | |
| with Spacers at | 19 are | | |
| positions 7 and 19 | substituted with | | |
| (STMN2 AON | spacers | | |
| 25_sp7sp19) | | | |
| STMN2 | Nucleosides at | 1564 | XXXXXXS₇XXXXXXXXXXS₁₈XXXXXXX |
| Oligonucleotide | positions 7 and | | |
| with Spacers at | 18 are | | |
| positions 7 and 18 | substituted with | | |
| (STMN2 AON | spacers | | |
| 25_sp7sp18) | | | |
| STMN2 | Nucleosides at | 1565 | XXXXXXXXS₉XXXXXXXXXXXS₂₁XXXX |
| Oligonucleotide | positions 9 and | | |
| with Spacers at | 21 are | | |
| positions 9 and 21 | substituted with | | |
| (STMN2 AON | spacers | | |
| 25_sp9sp21) | | | |

* At least one nucleoside linkage of the nucleobase sequence is selected from a phosphorothioate linkage, an alkyl phosphate linkage, a phosphorodithioate linkage, a phosphotriester linkage,
an alkylphosphonate linkage, a 3-methoxypropyl phosphonate linkage, a methylphosphonate linkage, an aminoalkylphosphotriester linkage, an alkylene phosphonate linkage, a phosphinate linkage,
a phosphoramidate linkage, a phosphoramidothioate linkage, a phosphorodiamidate (e.g., comprising a phosphorodiamidate morpholino (PMO), 3’ amino ribose, or 5’ amino ribose) linkage, an
aminoalkylphosphoramidate linkage, a thiophosphoramidate linkage, a thionoalkylphosphonate linkage, a thionoalkylphosphotriester linkage, a thiophosphate linkage, a
selenophosphate linkage, and a boranophosphate linkage.

In various embodiments, STMN2 oligonucleotides include three spacers. The inclusion of three spacers divides up the STMN2 oligonucleotide into four separate segments. In various embodiments, the three spacers are located at different positions of the STMN2 oligonucleotide such that each of the segments of the STMN2 oligonucleotide are at most 7 linked nucleosides in length. Example STMN2 AONs with three spacers are documented below in Table 8.

TABLE 8
Identification of STMN2 AONs or AON variants with three spacers. Here, each
STMN2 AON has 4 segments, where each segment has at most 7 linked nucleosides.

| | | | Sequence* (where X indicates |
|---|---|---|---|
| | Relation | Sequence | a nucleoside of the STMN2 |
| | to STMN2 | ID | parent oligonucleotide and |
| | parent | Number | S_{y} indicates presence of a |
| | oligonu- | (SEQ | Spacer where y denotes the |
| Sequence name | cleotide | ID NO) | position) (5′ → 3′) |
| STMN2 | Nucleosides | 1566 | XXXXXXXS₈XXXXXXXS₁₆XXXXXXXS₂₄X |
| Oligonucleotide | at positions 8 | | |
| with Spacers at | and 16 and 24 | | |
| positions 8 and | are substituted | | |
| 16 and 24 (STMN2 | with spacers | | |
| AON | | | |
| 25 sp8sp16sp24) | | | |
| STMN2 | Nucleosides | 1567 | XXXXXXXS₈XXXXXXXS₁₆XXXXXXS₂₃XX |
| Oligonucleotide | at positions 8 | | |
| with Spacers at | and 16 and 23 | | |
| positions 8 and | are substituted | | |
| 16 and 23 (STMN2 | with spacers | | |
| AON | | | |
| 25 sp8sp16sp23) | | | |
| STMN2 | Nucleosides | 1568 | XS₂XXXXXXXS₁₀XXXXXXXS₁₈XXXXXXX |
| Oligonucleotide | at positions 2 | | |
| with Spacers at | and 10 and 18 | | |
| positions 2 and | are substituted | | |
| 10 and 18 (STMN2 | with spacers | | |
| AON | | | |
| 25 sp8sp16sp23) | | | |
| STMN2 | Nucleosides | 1569 | XXS₃XXXXXXS₁₀XXXXXXXS₁₈XXXXXXX |
| Oligonucleotide | at positions 3 | | |
| with Spacers at | and 10 and 18 | | |
| positions 3 and | are substituted | | |
| 10 and 18 (STMN2 | with spacers | | |
| AON | | | |
| 25 sp8sp16sp23) | | | |
| STMN2 | Nucleosides | 1570 | XXXS₄XXXXXXXS₁₂XXXXXXS₁₉XXXXXX |
| Oligonucleotide | at positions 4 | | |
| with Spacers at | and 12 and 19 | | |
| positions 4 and | are substituted | | |
| 12 and 19 (STMN2 | with spacers | | |
| AON | | | |
| 25 sp4sp12sp19) | | | |
| STMN2 | Nucleosides | 1571 | XXXXXXXS₈XXXXS₁₃XXXXS₁₈XXXXXXX |
| Oligonucleotide | at positions 8 | | |
| with Spacers at | and 13 and 18 | | |
| positions 8 and | are substituted | | |
| 13 and 18 (STMN2 | with spacers | | |
| AON | | | |
| 25 sp8sp13sp18) | | | |
| STMN2 | Nucleosides | 1572 | XXXXS₅XXXXXXXS₁₃XXXXXXXS₂₁XXXX |
| Oligonucleotide | at positions 5 | | |
| with Spacers at | and 13 and 21 | | |
| positions 5 and | are substituted | | |
| 13 and 21 (STMN2 | with spacers | | |
| AON | | | |
| 25 sp5sp13sp21) | | | |
| STMN2 | Nucleosides | 1573 | XXXXXXS₇XXXXXS₁₃XXXXXS₁₉XXXXXX |
| Oligonucleotide | at positions 7 | | |
| with Spacers at | and 13 and 19 | | |
| positions 7 and | are substituted | | |
| 13 and 19 (STMN2 | with spacers | | |
| AON | | | |
| 25 sp7sp13sp19) | | | |
| STMN2 | Nucleosides | 1574 | XXXXXS₆XXXXXXS₁₃XXXXXXS₂₀XXXXX |
| Oligonucleotide | at positions 6 | | |
| with Spacers at | and 13 and 20 | | |
| positions 6 and | are substituted | | |
| 13 and 20 (STMN2 | with spacers | | |
| AON | | | |
| 25 sp6sp13sp20) | | | |
| STMN2 | Nucleosides | 1575 | XXXXXXXS₈XXS₁₁XXXXXXXS₁₉XXXXXX |
| Oligonucleotide | at positions 8 | | |
| with Spacers at | and 11 and 19 | | |
| positions 8 and | are substituted | | |
| 11 and 19 (STMN2 | with spacers | | |
| AON | | | |
| 25 sp8sp11sp19) | | | |
| STMN2 | Nucleosides | 1576 | XXXXXXXS₈XXS₁₁XXXXS₁₆XXXXXXX |
| Oligonucleotide | at positions 8 | | |
| with Spacers at | and 11 and 16 | | |
| positions 8 and | are substituted | | |
| 11 and 16 (STMN2 | with spacers | | |
| AON | | | |
| 23 sp8sp11sp16) | | | |
| STMN2 | Nucleosides | 1577 | XXXXXXS₇XXXXXXS₁₄XXXXXXXS₂₂XXX |
| Oligonucleotide | at positions 7 | | |
| with Spacers at | and 14 and 22 | | |
| positions 7 and | are substituted | | |
| 14 and 22 (STMN2 | with spacers | | |
| AON | | | |
| 23 sp7sp14sp22) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage,
a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate merpholino (PMO),
3’ amino ribose, or 5’ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate
linkage.

In various embodiments, STMN2 AONs with one or more spacers are reduced in length in comparison to the STMN2 AONs described above in Tables 7B and 8. For example, such STMN2 AONs may be STMN2 oligonucleotide variants with one or more spacers. In various embodiments, the STMN2 oligonucleotide variants with one or more spacers are 23mers, 21mers, or 19mers. In various embodiments, STMN2 oligonucleotide variants include two spacers such that the STMN2 oligonucleotide variant includes three segments that are divided up by the two spacers. In various embodiments, at least one of the three segments has at most 7 linked nucleosides. In various embodiments, each of the three segments has at most 7 linked nucleosides. Example STMN2 oligonucleotide variants with one or more spacers are shown below in Table 9.

TABLE 9
STMN2 AON variants with two spacers. Here, each STMN2 AON variant has 3
segments, where each segment has at most 7 linked nucleosides.

| | Relation | Sequence | Sequence* (where X indicates a |
|---|---|---|---|
| | to STMN2 | ID | nucleoside of the STMN2 oligonu- |
| | oligonu- | Number | cleotide variant and S_{y} indicates |
| Sequence | cleotide | (SEQ ID | presence of a Spacer where y |
| name | variant | NO) | denotes the position) (5′ → 3′) |
| STMN2 | N/A | 1578 | XXXXXXXXXXXXXXXXXXXXXXX (23mer) |
| oligonucleotide | | | |
| variant (23mer) | | | |
| STMN2 Variant | Nucleosides at | 1579 | XXXXXXXS₈XXXXXXXS₁₆XXXXXXX |
| Oligonucleotide | positions 8 and | | |
| (23mer) with | 16 are | | |
| Spacers at | substituted with | | |
| positions 8 and | spacers | | |
| 16 (STMN2 | | | |
| AON variant | | | |
| 23 sp8sp16) | | | |
| STMN2 | N/A | 1580 | XXXXXXXXXXXXXXXXXXXXX |
| oligonucleotide | | | (21mer) |
| variant (21mer) | | | |
| STMN2 Variant | Nucleosides at | 1581 | XXXXS₅XXXXXXS₁₂XXXXXXXXX |
| Oligonucleotide | positions 5 and | | |
| (21mer) with | 12 are | | |
| Spacers at | substituted with | | |
| positions 5 and | spacers | | |
| 12 (STMN2 | | | |
| AON variant | | | |
| 21 sp5sp12) | | | |
| STMN2 Variant | Nucleosides at | 1582 | XXXXXXXS₈XXXXXXXS₁₆XXXXX |
| Oligonucleotide | positions 8 and | | |
| (21mer) with | 16 are | | |
| Spacers at | substituted with | | |
| positions 8 and | spacers | | |
| 16 (STMN2 | | | |
| AON variant | | | |
| 21 sp8sp16) | | | |
| STMN2 Variant | Nucleosides at | 1583 | XXXXXS₆XXXXXXXS₁₄XXXXXXX |
| Oligonucleotide | positions 6 and | | |
| (21mer) with | 14 are | | |
| Spacers at | substituted with | | |
| positions 6 and | spacers | | |
| 14 (STMN2 | | | |
| AON variant | | | |
| 21 sp6sp14) | | | |
| STMN2 Variant | Nucleosides at | 1584 | XXXXXXXS₈XXXXXS₁₄XXXXXXX |
| Oligonucleotide | positions 8 and | | |
| (21mer) with | 14 are | | |
| Spacers at | substituted with | | |
| positions 8 and | spacers | | |
| 14 (STMN2 | | | |
| AON variant | | | |
| 21 sp8sp14) | | | |
| STMN2 Variant | Nucleosides at | 1585 | XXXXXS₆XXXXXXXXXXXXXS₂₀X |
| Oligonucleotide | positions 6 and | | |
| (21mer) with | 20 are | | |
| Spacers at | substituted with | | |
| positions 8 and | spacers | | |
| 14 (STMN2 | | | |
| AON variant | | | |
| 21 sp8sp14) | | | |
| STMN2 | N/A | 1586 | XXXXXXXXXXXXXXXXXXX |
| oligonucleotide | | | (19mer) |
| variant (19mer) | | | |
| STMN2 Variant | Nucleosides at | 1587 | XXXXS₅XXXXXXS₁₂XXXXXXX |
| Oligonucleotide | positions 5 and | | |
| (19mer) with | 12 are | | |
| Spacers at | substituted with | | |
| positions 5 and | spacers | | |
| 12 (STMN2 | | | |
| AON variant | | | |
| 19 sp5sp12) | | | |
| STMN2 Variant | Nucleosides at | 1588 | XXXXXXXS₈XXXXXXS₁₅XXXX |
| Oligonucleotide | positions 8 and | | |
| (19mer) with | 15 are | | |
| Spacers at | substituted with | | |
| positions 8 and | spacers | | |
| 15 (STMN2 | | | |
| AON variant | | | |
| 19 sp8sp15) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage,
a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate merpholino (PMO),
3’ amino ribose, or 5’ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate
linkage.

### Performance of STMN2 Oligonucleotides

Generally, STMN2 oligonucleotides and/or STMN2 parent oligonucleotides (e.g., STMN2 oligonucleotides with sequences of any of SEQ ID NOs: 1-466, SEQ ID NO: 893-1338, SEQ ID NOs: 1342-1366, and SEQ ID NOs: 1392-1664) target STMN2 transcripts (for example, a STMN2 pre-mRNA comprising a cryptic exon) comprising a sequence that shares at least 90% 15 (e.g., 90%, 91%, 92%, 93%, 94%, 95%, 96%, 97%, 98%, 99%, or 100%) identity to SEQ ID NO: 1339 or SEQ ID NO: 1341 in order to increase, restore, rescue, or stabilize levels of expression of STMN2 mRNA that is capable of translation to produce a functional STMN2 protein (e.g., full length STMN2). In various embodiments, STMN2 AONs can exhibit at least a 20%, 30%, 40%, 50%, 60%, 70%, 80%, or 90% increase of full length STMN2 protein. In various embodiments, STMN2 AONs can exhibit at least a 100%, 200%, 300%, or 400% increase of full length STMN2 protein. In some embodiments, the percent increase of the full length STMN2 protein is an increase in comparison to a reduced level of full length STMN2 protein achieved using a TDP43 antisense oligonucleotide. For example, a TDP43 antisense oligonucleotide can be used to deplete full length STMN2 protein followed by increase of the full length STMN2 protein using a STMN2 AON.

In some embodiments, STMN2 AONs can exhibit at least a 10%, 20%, 30%, 40%, 50%, 60%, 70%, 80%, 90%, or 100% rescue of full length STMN2 protein. In some embodiments, the percent rescue of full length STMN2 refers to the % of full length STMN2 following depletion using a TDP43 antisense oligonucleotide and a treatment using STMN2 AONs in comparison to a negative control (e.g., cells that did not undergo depletion or treatment or cells that were treated with a vehicle solution).

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a STMN2 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a STMN2 nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N®, or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′-O CH₂ CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ)—, where each Rₗ, Rₘ and Rₙ is, independently, H or substituted or unsubstituted C₁-C₁₀ alkyl.

Additional examples of modified sugar moieties include a 2′-Ome modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—Ng-O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—O—, —Si(Rₐ)₂—, —S(═O)ₓ—, and —N(Rₐ)—;

wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙO—, —C(RₐR_{b})—N®—O— or —C(RₐR_{b})—O—N®-. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N®-2′ and 4′-CH₂—N®—O-2′- wherein each R is, independently, H, a protecting group or C₁-C₁₂ alkyl, each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group; and R is H, C₁-C₁₂ alkyl, or a protecting group (see U.S. Pat. No. 7,427,672, issued on Sep. 23, 2008).

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′-CH₂—O-2′) BNA, β-D-methyleneoxy (4′-CH₂—O-2′) BNA, ethyleneoxy (4′-(CH₂)₂—O-2) BNA, aminooxy (4′-CH₂—O—N®-2′) BNA, 130yrrolid (4′-CH₂—N®—O-2′) BNA, methyl(methyleneoxy) (4′-CH(CH₃)—O-2′) BNA, methylene-thio (4′-CH₂—S-2′) BNA, methylene-amino (4′-CH₂—N®-2′) BNA, methyl carbocyclic (4′-CH₂—CH(CH₃)-2′) BNA, and propylene carbocyclic (4′-(CH₂)₃-2′) BNA; wherein R is H, C₁-C₁₂ alkyl, or a protecting group (see U.S. Pat. No. 7,427,672, issued on Sep. 23, 2008).

The present disclosure provide, in some embodiments, methods for treating, ameliorating, or preventing a neurological disease and/or a neuropathy further include methods of administering, to a patient, a pharmaceutically acceptable composition, for example, a pharmaceutically acceptable formulation that includes one or more STMN2 oligonucleotides. STMN2 oligonucleotides can increase, restore, or stabilize STMN2 activity, for example, STMN2 activity, and/or levels of STMN2 expression, for example, STMN2 mRNA and/or protein expression.

The present disclosure also provides pharmaceutical compositions comprising a STMN2 oligonucleotide formulated together with one or more pharmaceutically or cosmetically acceptable excipients. These formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular STMN2 oligonucleotide being used.

The present disclosure also provides a pharmaceutical composition comprising a STMN2 oligonucleotide or a pharmaceutically acceptable salt thereof (for example, a STMN2 AON that includes a sequence of any of SEQ ID NOs: 1-466, SEQ ID NO: 893-1338, SEQ ID NOs: 1342-1366, and SEQ ID NOs: 1392-1664).

The present disclosure also provides methods that include the use of pharmaceutical compositions comprising a STMN2 AON is formulated together with one or more pharmaceutically acceptable excipients. Exemplary compositions provided herein include compositions comprising a STMN2 AON, and one or more pharmaceutically acceptable excipients. Formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

### Additional Chemically Modified STMN2 Oligonucleotides

STMN2 AONs described herein, can include chemically modified nucleosides, including modified ribonucleosides and modified deoxyribonucleosides. Chemically modified nucleosides include, but are not limited to, uracil, uracine, uridine, 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, and 2′-O-(2-methoxyethyl)thymidine. In certain embodiments, mixed modalities, e.g., a combination of a STMN2 peptide nucleic acid (PNA) and a STMN2 locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA), and Fluoro Cyclohexenyl nucleic acid (F-CeNA) modifications. Chemically modified nucleosides that can be included in STMN2 AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

STMN2 AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, STMN2 AONs described herein can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. STMN2 AONs described herein can include one or more chemically modified bases, including a 5-methylpyrimidine, for example, 5-methylcytosine, and/or a 5-methylpurine, for example, 5-methylguanine. Chemically modified bases can further include pseudo-uridine or 5′methoxyuridine. STMN2 AONs described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

STMN2 AONs described herein can include a phosphate backbone where one or more of the oligonucleoside linkages is a phosphate linkage. STMN2 AONs described herein may include a modified oligonucleotide backbone, where one or more of the nucleoside linkages of the sequence is selected from the group consisting of a phosphorothioate linkage, a phosphorodithioate linkage, a phosphotriester linkage, an alkylphosphonate linkage, a 3-methoxypropyl phosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of STMN2 AONs described herein, at least one (i.e., one or more) internucleoside linkage of the oligonucleotide is a phosphorothioate linkage. For example, in some embodiments of STMN2 AONs described herein, one, two, three, or more internucleoside linkages of the oligonucleotide is a phosphorothioate linkage. In preferred embodiments of STMN2 AONs described herein, all internucleoside linkages of the oligonucleotide are phosphorothioate linkages. Thus, in some embodiments, all of the nucleotide linkages of a STMN2 AON of any of SEQ ID NOs: 1-466, SEQ ID NO: 893-1338, SEQ ID NOs: 1342-1366, and SEQ ID NOs: 1392-1664 are phosphorothioate linkages. In some embodiments, one or more of the nucleotide linkages of a STMN2 AON of any of SEQ ID NOs: 1-466, SEQ ID NO: 893-1338, SEQ ID NOs: 1342-1366, and SEQ ID NOs: 1392-1664 are phosphorothioate linkages.

In various embodiments, nucleotide linkages of STMN2 AON described herein such as any of SEQ ID NOs: 1-466, SEQ ID NO: 893-1338, SEQ ID NOs: 1342-1366, and SEQ ID NOs: 1392-1664 include a mix of phosphodiester and phosphorothioate linkages.

In some embodiments, nucleoside linkages linking a base at position 3 of a STMN2 AON described herein are phosphodiester bonds. For example, the base at position 3 may be linked to each adjacent base (e.g., preceding base and succeeding base) through a phosphodiester bond. An example 25mer STMN2 AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a STMN2 AON described herein is a phosphodiester bond. For example, the base at position 3 may be linked to either the preceding base or the succeeding base through a phosphodiester bond. An example 25mer STMN2 AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer STMN2 AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a STMN2 AON described herein being a phosphodiester bond, the STMN2 AON further includes two spacers. The two spacers can be positioned in the STMN2 AON such that the STMN2 AON includes a segment with at most 7 linked nucleosides. An example 25mer STMN2 AON with two spacers and with a phosphodiester bond linking the base at position 3 to a preceding base can be denoted as:

XxoDS₁XXXXXXXXXS₂XXXXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer STMN2 AON with two spacers and with a phosphodiester bond linking the base at position 3 to a succeeding base can be denoted as:

XXDoXXXXXXXS₁XXXXXXXXXS₂XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a STMN2 AON described herein are phosphodiester bonds. For example, the base at position 4 may be linked to each adjacent base (e.g., preceding base and succeeding base) through a phosphodiester bond. An example 25mer STMN2 AON with phosphodiester bonds linking the base at position 4 can be denoted as:

XXXoDoXXXXXXXXXXXXXXXXXXXXX

where “o” represents a phosphodiester bond and “D” represents the base at position 4. Any nucleobase in the AON can be a nucleobase analog.

In some embodiments, one of the nucleoside linkages linking a base at position 4 of a STMN2 AON described herein is a phosphodiester bond. For example, the base at position 4 may be linked to either the preceding base or the succeeding base through a phosphodiester bond. An example 25mer STMN2 AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer STMN2 AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a STMN2 AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer STMN2 AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the STMN2 AON are linked through phosphorothioate bonds. Any nucleobase in the AON can be a nucleobase analog.

In various embodiments, STMN2 AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the STMN2 AON are each linked to their respective adjacent bases through phosphodiester bonds. As described herein, the spacer can be located at various positions in the STMN2 AON and therefore, the 2 bases immediately preceding the spacer can vary within the STMN2 AON depending on where the spacer is situated.

In various embodiments, the STMN2 AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the STMN2 AON have Y number of bases immediately preceding the spacers that are linked through phosphodiester bonds. In various embodiments, all other bases of the STMN2 AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the STMN2 AON are each linked to their respective adjacent bases through phosphodiester bonds. To provide an example, such a STMN2 AON (e.g., 25mer) can be denoted as:

XXXXXXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the STMN2 AON and therefore, the bases immediately preceding or immediately succeeding the spacer can vary within the STMN2 AON depending on where the spacer is situated.

In various embodiments, the STMN2 AON may include more than one spacer. In some embodiments, only one of the spacers has Y number of bases immediately preceding the spacer and Z number of bases immediately succeeding the spacer that are linked through phosphodiester bonds. In such embodiments, the other spacers of the STMN2 AON are linked to respective preceding and succeeding bases through phosphorothioate bonds. To provide an example, such a STMN2 AON (e.g., 25mer) can be denoted as:

XXXXoDoS₁oEoXXXXXXXXXXXS₂XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a STMN2 AON (e.g., 25mer) can be denoted as:

XXXXXS₁XXXXXXXXXXXoDoS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a STMN2 AON includes a first spacer that is linked to the immediately preceding base through a phosphodiester bond, which can be denoted as:

XXXXXXoS₁XXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a STMN2 AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” represents a phosphodiester bond. Any nucleobase in the AON can be a nucleobase analog.

In various embodiments, the STMN2 AON may be a AON variant (e.g., a 23mer, a 21mer, or a 19mer) where one of the spacers is linked to the immediately preceding base through a phosphodiester bond. For example, the STMN2 AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the STMN2 AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” represents a phosphodiester bond. Any nucleobase in the AON can be a nucleobase analog.

In some embodiments, the STMN2 AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer STMN2 AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer STMN2 AON can be denoted as:

XXXXXS₁XXXXEoDoS₂XXXXXXX

where “S₁” represents a first spacer, “S₂” represents a second spacer, “o” represents a phosphodiester bond, “D” represents the base immediately preceding S₂ and “E” represents the base immediately preceding “D.” Any nucleobase in the AON can be a nucleobase analog.

In some embodiments, the STMN2 AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer STMN2 AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer STMN2 AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer S₁ is linked to an immediately preceding base through a phosphodiester bond. Any nucleobase in the AON can be a nucleobase analog.

In some embodiments, one of the spacers is linked to the immediately succeeding base through a phosphodiester bond. For example, a STMN2 AON includes a first spacer that is linked to the immediately succeeding base through a phosphodiester bond, which can be denoted as:

XXXXXXS₁oXXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a STMN2 AON includes a second spacer that is linked to the immediately succeeding base through a phosphodiester bond, which can be denoted as:

XXXXXXS₁XXXXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the STMN2 AON may be a AON variant (e.g., a 23mer, a 21mer, or a 19mer) where one of the spacers is linked to the immediately succeeding base through a phosphodiester bond. For example, the STMN2 AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the STMN2 AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the STMN2 AON have Y number of bases immediately preceding the spacers and Z number of bases immediately succeeding the spacers that are linked through phosphodiester bonds. An example of such a STMN2 AON (e.g., 25mer) can be denoted as:

XXXXoDoS₁oEoXXXXXXXXXXoFoS₂oHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the STMN2 AON are linked through phosphorothioate bonds. Any nucleobase in the AON can be a nucleobase analog.

In various STMN2 AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the STMN2 AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a STMN2 AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a STMN2 AON (e.g., 23mer) can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the STMN2 AON are linked through phosphorothioate bonds. Any nucleobase in the AON can be a nucleobase analog.

Table 10 below further depicts examples of STMN2 AON with a mix of phosphodiester and phosphorothioate linkages. In particular, Table 10 depicts examples of STMN2 AONs including spacers and a mix of phosphodiester and phosphorothioate linkages. Any nucleobase in the AON can be a nucleobase analog.

TABLE 10
Example STMN2 AONs with a mixture of phosphodiester and
phosphorothioate bonds.

| | AON Sequence* (5′ → 3′), where “o” | |
|---|---|---|
| SEQ | represents a phosphodiester bond, and | |
| ID | where “S” indicates a spacer. All other | Bases linked with |
| NO: | linkages are phosphorothioate bonds. | phosphodiester bonds |
| 173 | GAGTCCTGCAATATGAATATAATTT | N/A |
| 1451 | GAoGoSoCCTGCAATATSAATATAATTT | Bases at positions 3 and 4 |
| 1452 | GAoGoToCCTGCASTATGAATATSATTT | Bases at positions 3 and 4 |
| 1453 | GAoGoToCCSGCAATATGAATSTAATTT | Bases at positions 3 and 4 |
| 1454 | GAoGoToCCSGCAATATGAASATAATTT | Bases at positions 3 and 4 |
| 1455 | GToCoCoTGCSATATGAASATAAT | Bases at positions 3 and 4 |
| 1456 | GToCoCoTSCAATATGSATATAAT | Bases at positions 3 and 4 |
| 1457 | GToCoCoTGCSATATGSATATAAT | Bases at positions 3 and 4 |
| 1458 | GAGSCCTGCAAToAoToSAATATAATTT | 2 bases preceding a spacer |
| 1459 | GAGTCCTGCASTATGAAToAoToSATTT | 2 bases preceding a spacer |
| 1460 | GAGTCCSGCAATATGAoAoToSTAATTT | 2 bases preceding a spacer |
| 1461 | GAGTCCSGCAATATGoAoAoSATAATTT | 2 bases preceding a spacer |
| 1462 | GTCCTGCSATATGoAoAoSATAAT | 2 bases preceding a spacer |
| 1463 | GTCCTSCAATAoToGoSATATAAT | 2 bases preceding a spacer |
| 1464 | GTCCTGCSATAoToGoSATATAAT | 2 bases preceding a spacer |
| 1465 | GAoGoSoCoCTGCAATATSAATATAATTT | 1 base preceding and 1 base |
| | | after a spacer |
| 1466 | GAGTCCTGCoAoSoToATGAATATSATTT | 1 base preceding and 1 base |
| | | after a spacer |
| 1467 | GAGTCoCoSoGoCAATATGAATSTAATTT | 1 base preceding and 1 base |
| | | after a spacer |
| 1468 | GAGTCoCoSoGoCAATATGAASATAATTT | 1 base preceding and 1 base |
| | | after a spacer |
| 1469 | GTCCTGoCoSoAoTATGAASATAAT | 1 base preceding and 1 base |
| | | after a spacer |
| 1470 | GTCCoToSoCoAATATGSATATAAT | 1 base preceding and 1 base |
| | | after a spacer |
| 1471 | GTCCTGoCoSoAoTATGSATATAAT | 1 base preceding and 1 base |
| | | after a spacer |
| 1472 | GAGSCCTGCAATAoToSoAoATATAATTT | 1 base preceding and 1 base |
| | | after a spacer |
| 1473 | GAGTCCTGCASTATGAATAoToSoAoTTT | 1 base preceding and 1 base |
| | | after a spacer |
| 1474 | GAGTCCSGCAATATGAAoToSoToAATTT | 1 base preceding and 1 base |
| | | after a spacer |
| 1475 | GAGTCCSGCAATATGAoAoSoAoTAATTT | 1 base preceding and 1 base |
| | | after a spacer |
| 1476 | GTCCTGCSATATGAoAoSoAoTAAT | 1 base preceding and 1 base |
| | | after a spacer |
| 1477 | GTCCTSCAATAToGoSoAoTATAAT | 1 base preceding and 1 base |
| | | after a spacer |
| 1478 | GTCCTGCSATAToGoSoAoTATAAT | 1 base preceding and 1 base |
| | | after a spacer |
| 1479 | GAoGoSoCoCTGCAATAoToSoAoATATAATTT | 1 base preceding AND 1 |
| | | base after EACH spacer |
| 1480 | GAGTCCTGCoAoSoToATGAATAoToSoAoTTT | 1 base preceding AND 1 |
| | | base after EACH spacer |
| 1481 | GAGTCoCoSoGoCAATATGAAoToSoToAATTT | 1 base preceding AND 1 |
| | | base after EACH spacer |
| 1482 | GAGTCoCoSoGoCAATATGAoAoSoAoTAATTT | 1 base preceding AND 1 |
| | | base after EACH spacer |
| 1483 | GTCCTGoCoSoAoTATGAoAoSoAoTAAT | 1 base preceding AND 1 |
| | | base after EACH spacer |
| 1484 | GTCCoToSoCoAATAToGoSoAoTATAAT | 1 base preceding AND 1 |
| | | base after EACH spacer |
| 1485 | GTCCTGoCoSoAoTAToGoSoAoTATAAT | 1 base preceding AND 1 |
| | | base after EACH spacer |
| 197 | CCTTTCTCTCGAAGGTCTTCTGCCG | N/A |
| 1430 | CCTTTCTCTCGAAGGTCTTCTGC | N/A |
| 1431 | CTTTCTCTCGAAGGTCTTCTGCC | N/A |
| 1486 | CCoToToTCTCTCGAAGGTCTTCTGCCG | Bases at positions 3 and 4 |
| 1487 | CCoToToTCTCSCGAAGGTCTTCSGCCG | Bases at positions 3 and 4 |
| 1488 | CToToToCTCSCGAAGGTSTTCTGCC | Bases at positions 3 and 4 |
| 1489 | TToToCoTCTSGAAGGTCSTCTGCCG | Bases at positions 3 and 4 |
| 1490 | TToToCoTCTCGAAGGTCTTCTGCCG | Bases at positions 3 and 4 |
| 1491 | CCoToToTCTCTCGAAGGTCTTCTGC | Bases at positions 3 and 4 |
| 1492 | CCTTTCoToCoSCGAAGGTCTTCSGCCG | 2 bases preceding a spacer |
| 1493 | CTTTCoToCoSCGAAGGTSTTCTGCC | 2 bases preceding a spacer |
| 1494 | TTTCToCoToSGAAGGTCSTCTGCCG | 2 bases preceding a spacer |
| 1495 | CCTTTCTCSCGAAGGTCToToCoSGCCG | 2 bases preceding a spacer |
| 1496 | CTTTCTCSCGAAGoGoToSTTCTGCC | 2 bases preceding a spacer |
| 1497 | TTTCTCTSGAAGGoToCoSTCTGCCG | 2 bases preceding a spacer |
| 1498 | CCTTTCToCoSoCoGAAGGTCTTCSGCCG | 1 base preceding and 1 base |
| | | after a spacer |
| 1499 | CTTTCToCoSoCoGAAGGTSTTCTGCC | 1 base preceding and 1 base |
| | | after a spacer |
| 1500 | TTTCTCoToSoGoAAGGTCSTCTGCCG | 1 base preceding and 1 base |
| | | after a spacer |
| 1501 | CCTTTCTCSCGAAGGTCTToCoSoGoCCG | 1 base preceding and 1 base |
| | | after a spacer |
| 1502 | CTTTCTCSCGAAGGoToSoToTCTGCC | 1 base preceding and 1 base |
| | | after a spacer |
| 1503 | TTTCTCTSGAAGGToCoSoToCTGCCG | 1 base preceding and 1 base |
| | | after a spacer |
| 1504 | CCTTTCToCoSoCoGAAGGTCTToCoSoGoCCG | 1 base preceding AND 1 |
| | | base after EACH spacer |
| 1505 | CTTTCToCoSoCoGAAGGoToSoToTCTGCC | 1 base preceding AND 1 |
| | | base after EACH spacer |
| 1506 | TTTCTCoToSoGoAAGGToCoSoToCTGCCG | 1 base preceding AND 1 |
| | | base after EACH spacer |
| 1507 | CCTTTCTCSCGAAoGoGoToCoTTCSGCCG | Range of 4 bases between |
| | | two spacers |
| 1508 | CTTTCTCSCGAoAoGoGTSTTCTGCC | Range of 2 bases between |
| | | two spacers |
| 1509 | TTTCTCTSGAAoGoGoTCSTCTGCCG | Range of 2 bases between |
| | | two spacers |
| 1510 | GAoGSCCTGCAATATSAATATAATTT | Base 3 linked to preceding |
| | | base through phosphodiester |
| | | linkage |
| 1511 | GAGoTCCTGCASTATGAATATSATTT | Base 3 linked to preceding |
| | | base through phosphodiester |
| | | linkage |
| 1512 | GAGTCCoSGCAATATGAATSTAATTT | First spacer linked to |
| | | preceding base through |
| | | phosphodiester linkage |
| 1513 | GAGTCCSoGCAATATGAASATAATTT | First spacer linked to |
| | | succeeding base through |
| | | phosphodiester linkage |
| 1514 | GTCCTGCSoATATGAASATAAT | First spacer linked to |
| | | succeeding base through |
| | | phosphodiester linkage |
| 1515 | GTCCTGCoSATATGSATATAAT | First spacer linked to |
| | | preceding base through |
| | | phosphodiester linkage |
| 1516 | GTCCoToSCAATATGSATATAAT | 1 base preceding a first |
| | | spacer linked through |
| | | phosphodiester linkage |
| 1517 | GAGTCCSGCAATATGAAToSTAATTT | Second spacer linked to |
| | | preceding base through |
| | | phosphodiester linkage |
| 1518 | GAGTCCSGCAATATGAASoATAATTT | Second spacer linked to |
| | | succeeding base through |
| | | phosphodiester linkage |
| 1519 | GTCCTGCSATATGAAoSATAAT | Second spacer linked to |
| | | preceding base through |
| | | phosphodiester linkage |
| 1520 | GTCCTGCSATATGSoATATAAT | First spacer linked to |
| | | succeeding base through |
| | | phosphodiester linkage |
| 1521 | GTCCoTSCAATATGoSATATAAT | 1 base preceding a first |
| | | spacer linked through |
| | | phosphodiester linkage and |
| | | second spacer linked to |
| | | preceding base through |
| | | phosphodiester linkage |

In some embodiments, a disclosed STMN2 AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

STMN2 AONs may include at least one modified sugar. For example, the sugar moiety of at least one nucleotide constituting the oligonucleotide is a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-Ome modified sugar moiety, bicyclic sugar moiety, 2′-O-(2-methoxyethyl) (2′MOE or MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, STMN2 AONs comprise 2′Ome (e.g., a STMN2 AON comprising one or more 2′Ome modified sugar), 2′MOE or MOE (e.g., a STMN2 AON comprising one or more 2′MOE modified sugar), PNA (e.g., a STMN2 AON comprising one or more N-(2-aminoethyl)-glycine units linked by amide bonds or carbonyl methylene linkage as repeating units in place of a sugar-phosphate backbone), LNA (e.g., a STMN2 AON comprising one or more locked ribose, and can be a mixture of 2′-deoxy nucleotides or 2′Ome nucleotides), c-ET (e.g., a STMN2 AON comprising one or more cET sugar), cMOE (e.g., a STMN2 AON comprising one or more cMOE sugar), morpholino oligomer (e.g., a STMN2 AON comprising a backbone comprising one or more PMO), deoxy-2′-fluoro nucleoside (e.g., a STMN2 AON comprising one or more 2′-fluoro-β-D-arabinonucleoside), tcDNA (e.g., a STMN2 AON comprising one or more tcDNA modified sugar), ENA (e.g., a STMN2 AON comprising one or more ENA modified sugar), or HNA (e.g., a STMN2 AON comprising one or more HNA modified sugar). In some embodiments, a STMN2 AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a STMN2 AON comprises one or more phosphorothioate linkage, phosphodiester linkage, or a combination of phosphorothioate and phosphodiester linkages.

In some embodiments, STMN2 AONs with a sequence of any one of SEQ ID NOs: 1-466, SEQ ID NO: 893-1338, SEQ ID NOs: 1342-1366, and SEQ ID NOs: 1392-1664 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a STMN2 AON with a sequence of any one of SEQ ID NOs: 1-466, SEQ ID NO: 893-1338, SEQ ID NOs: 1342-1366, and SEQ ID NOs: 1392-1664 is a chirally controlled oligonucleotide comprising a plurality of oligonucleotides of at least one type, wherein each type is defined by: 1) base sequence; 2) pattern of backbone linkages; 3) pattern of backbone chiral centers; and 4) pattern of backbone X-moieties (—X-L-R¹); wherein: the oligonucleotides of the at least one type comprise one or more phosphorothioate triester internucleotidic linkages and one or more phosphate diester linkage; the oligonucleotides of the at least one type comprise at least two consecutive modified internucleotidic linkages; and oligonucleotides of the at least one oligonucleotide type comprise one or more modified internucleotidic linkages independently having the structure of:

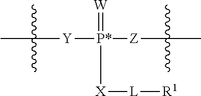

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl; and each

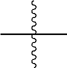

independently represents a connection to a nucleoside. In some embodiments, a STMN2 AON with a sequence of any one of SEQ ID NOs: 1-466, SEQ ID NO: 893-1338, SEQ ID NOs: 1342-1366, and SEQ ID NOs: 1392-1664 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-Ome, phosphorothioate linkages, lipid conjugation, etc.), as described in U.S. Pat. No. 10,450,568.

### Motor Neuron Diseases

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

### Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDBP, FUS, ANG, ATXN2, CHCHD10, CHMP2B, DCTN1, ErbB4, FIG4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

### Frontotemporal Dementia

Frontotemporal dementia (FTD) is a form of dementia that affects the frontal and temporal lobes of the brain. FTD includes frontotemporal lobar degeneration (FTLD). It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior, and primary progressive aphasia (PPA), which affects language skills, speaking, writing and comprehension. FTD is associated with tau protein accumulation (Pick bodies) and altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDBP, FUS, ITM2B, CHCHD10, SQSTM1, PSEN1, PSEN2, CTSF, CYP27A1, TBK1 and TBP.

Amyotrophic Lateral Sclerosis with Frontotemporal Dementia

Amyotrophic lateral sclerosis with frontotemporal dementia (ALS with FTD) is a clinical syndrome in which FTD and ALS occur in the same individual. Interestingly, mutations in C₉orf72 are the most common cause of familial forms of ALS and FTD. Additionally, mutations in TBK1, VCP, SQSTM1, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins. TBK1 mutations are associated with ALS, FTD, and ALS with FTD.

### Limbic-Predominant Age-Related TDP-43 Encephalopathy (LATE)

Limbic-predominant age-related TDP-43 encephalopathy (LATE) is characterized by accumulation of misfolded TDP-43 protein in the brain, specifically in the limbic system. LATE is a neurological disorder that typically manifests in older patients (e.g., greater than 80 years old). LATE can be a diagnosis for dementia and LATE often mimics the symptoms of Alzheimer's Disease including memory loss, confusion, and mood changes.

### Methods of Treatment

Further (for example, amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, corticobasal degeneration (CBD), and Limbic-predominant age-related TDP-43 encephalopathy (LATE) in a patient in need thereof comprising administering a STMN2 AON. In some embodiments, provided herein are methods for treatment of a neurological disease in a patient in need thereof, comprising administering a disclosed STMN2 AON. In some embodiments of the disclosure, an effective amount of a disclosed STMN2 oligonucleotide may be administered to a patient in need thereof to treat a neurological disease, and/or to increase, restore, or stabilize expression of STMN2 mRNA that is capable of translation to produce a functional STMN2 protein, thereby increase, restore, or stabilize STMN2 activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed STMN2 AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed STMN2 AON. The methods include for example, treating a subject at risk of developing a neurological disease; e.g., administering to the subject an effective amount of a disclosed STMN2 AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a STMN2 AON disclosed herein. For example, a method of preventing or treating a neurological disease is provided comprising administering to a patient in need thereof a STMN2 AON disclosed herein.

Patients treated using an above method may experience an increase, restoration of, or stabilization of STMN2 mRNA expression, which is capable of translation to produce a functional STMN2 protein, of at least about 5%, 10%, 20%, 30%, 40% or even 50%, thereby increase, restore, or stabilize STMN2 activity and/or function in a target cell (for example, a motor neuron) after administering a STMN2 oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a STMN2 oligonucleotide may be on, e.g., at least a daily basis. The STMN2 oligonucleotide may be administered orally. In some embodiments, the STMN2 oligonucleotide is administered intrathecally, intrathalamically, or intracisternally. For example, in an embodiment described herein, a STMN2 oligonucleotide is administered intrathecally, intrathalamically or intracisternally about every 3 months. The delay or amelioration of clinical manifestation of a neurological disease in a patient as a consequence of administering a STMN2 oligonucleotide disclosed here may be at least e.g., 6 months, 1 year, 18 months or even 2 years or more as compared to a patient who is not administered a STMN2 oligonucleotide, such as one disclosed herein.

STMN2 oligonucleotides can be used alone or in combination with each other whereby at least two STMN2 oligonucleotides are used together in a single composition or as part of a treatment regimen. STMN2 oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, and SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, and SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, and SEQ ID NOs: 1392-1664, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Treatment and Evaluation

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. A patient may be an individual diagnosed with a high risk of developing a neurological disease, someone who has been diagnosed with a neurological disease, someone who previously suffered from a neurological disease, or an individual evaluated for symptoms or indications of a neurological disease, for example, any of the signs or symptoms associated with neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed STMN2 oligonucleotide is the amount of the STMN2 oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed STMN2 oligonucleotide.

Efficacy of treatment may also be evaluated at the tissue or cellular level, for example, by means of obtaining a tissue biopsy (e.g., a brain, spinal, muscle, motor neuron tissue biopsy, or olfactory neurosphere cell biopsy) and evaluating gross tissue or cell morphology or staining properties. Biochemical assays that examine protein or RNA expression may also be used to evaluate efficacy of treatment. For instance, one may evaluate levels of a protein or gene product indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or dePCR), qPCR etc.) reaction. One may also evaluate the presence or level of expression of useful biomarkers (e.g., neurofilament light (NEFL), neurofilament heavy (NEFH), TDP-43 or p75 extracellular domain (p75^{ECD})) found in spinal cord fluid, cerebrospinal fluid, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. One may also evaluate the presence or level of expression of useful biomarkers found in the plasma, neuronal extracellular vesicles/exosomes. Additional measurements of efficacy may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in C9ORF72-associated amyotrophic lateral sclerosis (c9ALS) patients. CSF pNFH as a prognostic biomarker for clinical trials, which will increase the likelihood of successfully developing a treatment for c9ALS.

In evaluating efficacy of treatment, suitable controls may be chosen to ensure a valid assessment. For instance, one can compare symptoms evaluated in a patient with a neurological disease following administration of a disclosed STMN2 oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed STMN2 oligonucleotide with those of tissue from the same patient or from an individual not diagnosed with the neurological disease or from the same patient prior to administration of the STMN2 oligonucleotide. Additionally, one may compare blood, plasma, serum, cell, urine, lymphatic fluid, spinal cord fluid, cerebrospinal fluid, or fecal samples following administration of the STMN2 oligonucleotide with comparable samples from an individual not diagnosed with the neurological disease or from the same patient prior to administration of the STMN2 oligonucleotide. In some embodiments one may compare extracellular vesicles (for example CSF exosomes), following administration of the STMN2 oligonucleotide with extracellular vesicles from an individual not diagnosed with the neurological disease or from the same patient prior to administration of the STMN2 oligonucleotide.

Validation of STMN2 oligonucleotides may be determined by direct or indirect assessment of STMN2 expression levels or activity. For instance, biochemical assays that measure STMN2 protein or RNA expression may be used to evaluate overall effect on STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. For instance, one may measure STMN2 protein levels in cells or tissue by Western blot to evaluate overall STMN2 levels. One may also measure STMN2 mRNA levels by means of Northern blot or quantitative polymerase chain reaction to determine overall effect on STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. One may also evaluate STMN2 protein levels or levels of another protein indicative of STMN2 signaling activity in dissociated cells, non-dissociated tissue, extracellular vesicles (for example, CSF exosomes), blood, serum, or fecal matter via immunocytochemical or immunohistochemical methods.

Modulation of expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate the modulation of expression of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. Modulation of expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), 157yrrolidiny, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length STMN2 transcripts in cells of a patient suffering from a neurological disease. Full length STMN2 transcripts may be restored in any cell in which STMN2 expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### Pharmaceutical Compositions and Routes of Administration

The present disclosure also provides methods for treating a neurological disease via administration of a pharmaceutical composition comprising a disclosed STMN2 oligonucleotide. In another aspect, the disclosure provides a pharmaceutical composition for use in treating a neurological disease. The pharmaceutical composition may be comprised of a disclosed STMN2 oligonucleotide, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, described herein are pharmaceutical compositions comprising a disclosed STMN2 oligonucleotide, and a pharmaceutically acceptable carrier. In another aspect, the disclosure provides use of a disclosed STMN2 oligonucleotide in the manufacture of a medicament for treating a neurological disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In one embodiment, a disclosed STMN2 oligonucleotide and any pharmaceutical composition thereof may be administered by one or several routes, including topically, intrathecally, intrathalamically, intracisternally, parenterally, orally, rectally, buccally, sublingually, vaginally, pulmonarily, intratracheally, intranasally, transdermally, or intraduodenally. The term parenteral as used herein includes subcutaneous injections, intrapancreatic administration, intravenous, intracisternal, intracerebroventricular, intrathecal, intrathalamic, intramuscular, intraperitoneal, intrasternal injection or infusion techniques. For example, a disclosed STMN2 oligonucleotide may be administered subcutaneously to a subject. In another example, a disclosed STMN2 oligonucleotide may be administered orally to a subject. In another example, a disclosed STMN2 oligonucleotide may be administered directly to the nervous system, or specific regions or cells of the nervous system (e.g., the brain, brain stem, lower motor neurons, spinal cord, upper motor neurons) via parenteral administration, for example, a disclosed STMN2 oligonucleotide may be administered intrathecally, intrathalamically or intracisternally.

In various embodiments, a STMN2 oligonucleotide, for example a STMN2 AON, can be exposed to calcium-containing buffers prior to administration. Such calcium-containing buffers can mitigate toxicity adverse effects of the STMN2 oligonucleotide. Further details of exposing an example antisense oligonucleotide to calcium-containing buffers is described in Moazami, et al., Quantifying and Mitigating Motor Phenotypes Induced by Antisense Oligonucleotides in the Central Nervous System, bioRxiv 2021.02.14.431096, which is hereby incorporated by reference in its entirety.

In some embodiments, a STMN2 oligonucleotide, for example a STMN2 AON, can be encapsulated in a nanoparticle coating. It is believed that nanoparticle encapsulation prevents AON degradation and enhances cellular uptake. For example, in some embodiments a STMN2 oligonucleotide is encapsulated in a coating of a cationic polymer, for example, a synthetic polymer (e.g., poly-L-lysine, polyamidoamine, a poly((3-amino ester), and polyethyleneimine) or a naturally occurring polymer (e.g., chitosan and a protamine). In some embodiments, a STMN2 oligonucleotide is encapsulated in a lipid or lipid-like material, for example, a cationic lipid, a cationic lipid-like material, or an ionizable lipid that is positively charged only at an acidic pH. For example, in some embodiments, a STMN2 oligonucleotide is encapsulated in a lipid nanoparticle that includes hydrophobic moieties, e.g., cholesterol and/or a polyethylene glycol (PEG) lipid

Pharmaceutical compositions containing a disclosed STMN2 oligonucleotide, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18^{th} ed. (Mack Publishing Company, 1990).

Pharmaceutical formulations, in some embodiments, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intracisternal, intracerebroventricular, intramuscular, subcutaneous, intrathecal, intrathalamic, intralesional, or intraperitoneal routes. The preparation of an aqueous composition, such as an aqueous pharmaceutical composition containing a disclosed STMN2 oligonucleotide, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including normal saline, artificial cerebrospinal fluid, sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed STMN2 antisense oligonucleotide may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed STMN2 antisense oligonucleotide in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed oligonucleotide to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium 161yrrolidiny, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

### Oral Administration

In some embodiments, contemplated herein are compositions suitable for oral delivery of a disclosed STMN2 oligonucleotide, e.g., tablets that include an enteric coating, e.g., a gastro-resistant coating, such that the compositions may deliver a STMN2 oligonucleotide to, e.g., the gastrointestinal tract of a patient.

For example, a tablet for oral administration is provided that comprises granules (e.g., is at least partially formed from granules) that include a disclosed STMN2 oligonucleotide, e.g., a STMN2 oligonucleotide represented by any SEQ ID NOs: 1-466, SEQ ID NO: 893-1338, SEQ ID NOs: 1342-1366, and SEQ ID NOs: 1392-1664 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed STMN2 oligonucleotide, e.g., a STMN2 oligonucleotide represented by any of SEQ ID NOs: 1-466, SEQ ID NO: 893-1338, SEQ ID NOs: 1342-1366, and SEQ ID NOs: 1392-1664 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and a pharmaceutically acceptable salt. In some embodiments, contemplated pharmaceutical formulations include an intra-granular phase that includes a disclosed STMN2 oligonucleotide, e.g., a STMN2 oligonucleotide represented by any of SEQ ID NOs: 1-466, SEQ ID NO: 893-1338, SEQ ID NOs: 1342-1366, and SEQ ID NOs: 1392-1664 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and a pharmaceutically acceptable filler. For example, a disclosed STMN2 oligonucleotide and a filler may be blended together, optionally, with other excipients, and formed into granules. In some embodiments, the intragranular phase may be formed using wet granulation, e.g., a liquid (e.g., water) is added to the blended STMN2 oligonucleotide and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed STMN2 oligonucleotide and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE, Eudragit® grades. In some embodiments, an enteric coating may comprise about 5% to about 10%, about 5% to about 20%, 8% to about 15%, about 8% to about 20%, about 10% to about 20%, or about 12% to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g., about 0.5% to about 10%, or about 1% to about 20%, by weight of a disclosed STMN2 oligonucleotide or a pharmaceutically acceptable salt thereof. Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed STMN2 oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g., about 2% to about 4% sodium starch glycolate by weight.

In another contemplated embodiment, a pharmaceutical tablet formulation for oral administration of a disclosed STMN2 oligonucleotide comprises an intra-granular phase, wherein the intra-granular phase includes a disclosed STMN2 AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed STMN2 AON, e.g., a disclosed STMN2 AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed STMN2 AON, e.g., a disclosed STMN2 AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g., when tested in a USP/EP Type 2 apparatus (paddle) at 100 rpm and 37° C. in a phosphate buffer with a pH of 7.2, of about 50% to about 100% of the STMN2 oligonucleotide releasing after about 120 minutes to about 240 minutes, for example after 180 minutes. In another embodiment, a contemplated tablet may have a dissolution profile, e.g., when tested in a USP/EP Type 2 apparatus (paddle) at 100 rpm and 37° C. in diluted HCl with a pH of 1.0, where substantially none of the STMN2 oligonucleotide is released after 120 minutes. A contemplated tablet, in another embodiment, may have a dissolution profile, e.g., when tested in USP/EP Type 2 apparatus (paddle) at 100 rpm and 37° C. in a phosphate buffer with a pH of 6.6, of about 10% to about 30%, or not more than about 50% of the STMN2 oligonucleotide releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

### Dosage and Frequency of Administration

The dosage or amounts described below refer either to the oligonucleotide or a pharmaceutically acceptable salt thereof.

In some embodiments, methods described herein include administering at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of a STMN2 antisense oligonucleotide e.g., a STMN2 oligonucleotide. In some embodiments, methods include administering from 10 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a STMN2 antisense oligonucleotide.

In some embodiments, methods described herein include administering formulations that include about 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed STMN2 oligonucleotide. In some embodiments, a formulation may include about 40 mg, 80 mg, or 160 mg of a disclosed STMN2 oligonucleotide. In some embodiments, a formulation may include at least 100 μg of a disclosed STMN2 oligonucleotide. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed STMN2 oligonucleotide. The amount administered will depend on variables such as the type and extent of disease or indication to be treated, the overall health and size of the patient, the in vivo potency of the STMN2 oligonucleotide, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once every 4 weeks, once every 5 weeks, once every 6 weeks, once every 7 weeks, once every 8 weeks, once every 9 weeks, once every 10 weeks, once every 11 weeks, or once every 12 weeks. In some embodiments, dosing is once a month to every three months.

### Combination Therapies

In various embodiments, a STMN2 AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

Example additional therapies include any of Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprosterone, KCNQ2/KCNQ3 openers (e.g., retigabine, XEN1101, or QRL-101), anticonvulsants and psychostimulant agents. Additional therapies can further include breathing care, physical therapy, occupational therapy, speech therapy, and nutritional support. In various embodiments, an additional therapy can be a second antisense oligonucleotide. As an example, the second antisense oligonucleotide may target a STMN2 transcript (e.g., STMN2 pre-mRNA, mature STMN2 mRNA) to modulate the expression levels of full length STMN2 protein.

In various embodiments, the disclosed oligonucleotide and the one or more additional therapies can be conjugated to one another and provided in a conjugated form. Further description regarding conjugates involving the disclosed oligonucleotide is described below. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

### Conjugates

In certain embodiments, provided herein are oligomeric compounds, which comprise an oligonucleotide (e.g., STMN2 oligonucleotide) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Conjugate Groups

In certain embodiments, a STMN2 AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et Molecular Therapy Nucleic Acids, 2015, 4, e220; and Nishina et al., Molecular Therapy, 2008, 16, 734-740), or a GalNAc cluster (e.g., WO2014/179620).

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratine).

In certain embodiments, a conjugate moiety comprises an active drug substance, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

### Conjugate Linkers

Conjugate moieties are attached to a STMN2 AON through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbon chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the STMN2 AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

### Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside. In various embodiments, terminal groups comprise one or more spacers.

### Diagnostic Methods

The disclosure also provides a method of diagnosing a patient with a neurological disease that relies upon detecting levels of STMN2 expression signal in one or more biological samples of a patient. As used herein, the term “STMN2 expression signal” can refer to any indication of STMN2 gene expression, or gene or gene product activity. STMN2 gene products include RNA (e.g., mRNA), peptides, and proteins. Indices of STMN2 gene expression that can be assessed include, but are not limited to, STMN2 gene or chromatin state, STMN2 gene interaction with cellular components that regulate gene expression, STMN2 gene product expression levels (e.g., expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, or interaction of STMN2 RNA or protein with transcriptional, translational, or post-translational processing machinery.

Detection of STMN2 expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, extracellular vesicles (for example, CSF exosomes), or cells of a patient. Detection may be achieved by measuring expression signal of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Methods of detection include assays that measure levels of STMN2 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Modifications in General

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as ® or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool.

### EXAMPLES

The disclosure is further illustrated by the following examples. The examples are provided for illustrative purposes only, and are not to be construed as limiting the scope or content of the disclosure in any way.

### Example 1: Design and Selection of STMN2 Oligonucleotides

STMN2 AONs oligonucleotides that target a STMN2 transcript including a cryptic exon are designed and tested to identify STMN2 AONs capable of reducing quantity of STMN2 transcripts that comprise a cryptic exon. Such STMN2 AONs include STMN2 parent oligonucleotides represented by any of SEQ ID NOs: 1-446 or SEQ ID NOs: 893-1338. The STMN2 parent oligonucleotides are 25 nucleosides in length. Each of the nucleosides of the STMN2 parent oligonucleotides are modified nucleosides with 2′MOE sugar moieties, and each “C” is replaced with a 5-MeC. Additionally, each of the internucleoside linkages between the nucleosides of the STMN2 oligonucleotides are phosphorothioate internucleoside linkages.

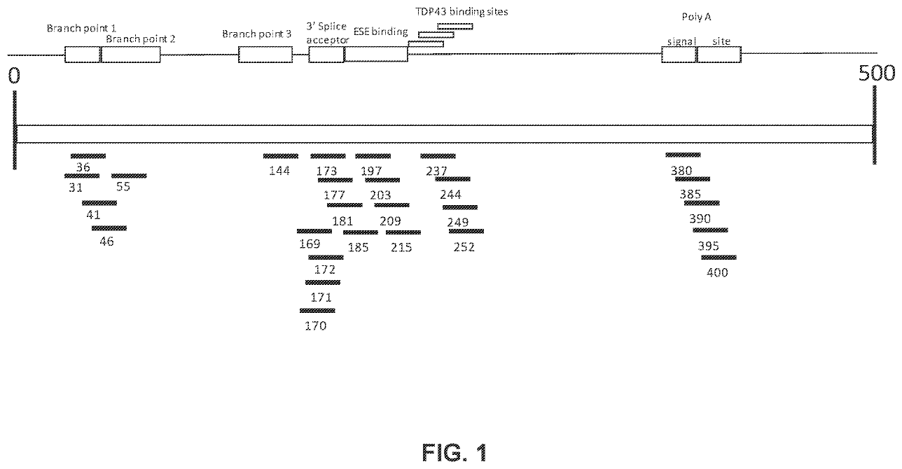

FIG. 1 is a depiction of portions of the STMN2 transcript and STMN2 parent oligonucleotides that are designed to target certain portions of the STMN2 transcript including a cryptic exon. Specifically, regions of the STMN2 transcript include branch points (e.g., branch point 1, 2, and 3) a 3′ splice acceptor region, an ESE binding region, TDP43 binding sites, and a Poly A region. STMN2 oligonucleotides, are identified according to the position of the STMN2 transcript that the STMN2 oligonucleotide corresponds to. For example, FIG. 1 depicts a STMN2 oligonucleotide that targets positions 36 to 60 of the STMN2 transcript including a cryptic exon, which includes a branch point 1. Similarly, a different STMN2 oligonucleotide targets positions 144 to 178 of the STMN2 transcript including a cryptic exon, which includes a branch point 3. Other STMN2 oligonucleotides can be designed using any of the sequences disclosed above.

Generally, the length of the STMN2 antisense oligonucleotides are 25 nucleotide bases in length. However, variants of the STMN2 antisense oligonucleotides were also designed with varying lengths (e.g., 23mers, 21mers, or 19mers). Examples of these variant STMN2 antisense oligonucleotides were designed to include the sequences of SEQ ID NOs: 1342-1366 or SEQ ID NOs: 1392-1521.

### Example 2: Methods for Evaluating STMN2 Antisense Oligonucleotides

STMN2 antisense oligonucleotides were evaluated in SY5Y cells and human motor neurons (hMN). The cells were plated in 6-well or 96-well plates and cultured to 80% confluency. Antisense oligonucleotide (AON) to TDP43 was transfected with RNAiMax (Thermo Fisher Scientific, Waltham, Mass., USA) to express the cryptic exon, thus preventing transcription of full-length STMN2 (STMN2-FL) product. Vehicle was treated with RNAiMax alone. Positive controls included cells that were treated with TDP43 siRNA alone (“siRNA TDP43”) and/or TDP43 AON alone (“AON TDP43” or “TDP43 AON”). siRNA TDP43 was purchased as ON-TARGETplus Human TARDBP (23435) siRNA-SMARTpool (#L-012394-00-0005) from Horizon/Dharmacon. TARDBP (23435) siRNA includes four individual siRNAs that targets four separate sequences:

| (SEQ ID NO: 1665) | |
|---|---|
| 1) Target sequence 1: | GCUCAAGCAUGGAUUCUAA |
| (SEQ ID NO: 1666) | |
| 2) Target sequence 2: | CAAUCAAGGUAGUAAUAUG |
| (SEQ ID NO: 1667) | |
| 3) Target sequence 3: | GGGCUUCGCUACAGGAAUC |
| (SEQ ID NO: 1668) | |
| 4) Target sequence 4: | CAGGGUGGAUUUGGUAAUA |

TDP43 AON is a gapmer oligonucleotide and has the following sequence and chemistry:

5′A*A*G*G*C*T*T*C*A*T*A*T*T*G*T*A*C*T*T*T 3′ (SEQ ID NO: 1669)

where * phosphorothioate, underlined=DNA, other=2A10E RNA; each “C” is 5-MeC.

To evaluate STMN2 AON ability to restore STMN2-FL, antisense oligonucleotides to STMN2 were co-incubated with TDP43 AON in RNAiMax. After 96 hours, transcript levels (e.g., STMN2 full length transcript, STMN2 transcript with cryptic exon, or TDP43 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting STMN2 transcripts with cryptic exon using the following primer sequences: 1) Forward primer: 5′-CTCAGTGCCTTATTCAGTCTTCTC-3′ (SEQ ID NO: 1670), 2) Reverse primer: 5′-TCTTCTGCCGAGTCCCATTT-3′ (SEQ ID NO: 1671) and 3) Probe: 5′-/56-FAM/TCAGCGTCTGCACATCCCTACAAT/3BHQ_1/-3′ (SEQ ID NO: 1672). RT-qPCR was performed for detecting full length STMN2 transcripts using the following primer sequences: 1) Forward primer: 5′-CCACGAACTTTAGCTTCTCCA-3′ (SEQ ID NO: 1673), 2) Reverse primer: 5′-GCCAATTGTTTCAGCACCTG-3′ (SEQ ID NO: 1674), and 3) Probe: 5′-/56-FAM/ACTTTCTTCTTTCCTCTGCAGCCTCC/3BHQ_1/-3′ (SEQ ID NO: 1675).

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

STMN2-FL or STMN2 cryptic signal (Ct) was normalized to GAPDH (deltaCt). To visualize the quantitative changes (e.g., % increase of STMN-FL), the normalized STMN2-FL signal was further normalized to the vehicle (treated with RNAiMax alone, deltadeltaCt). Relative quantity of transcript level was calculated using the equation RQ=2^{-deltadeltaCt} and is used to describe the treatment condition comparison to normal, healthy levels (1.0).

Percent decrease of STMN2 with cryptic exon expression was calculated using the equation of:

$$100 - ( \frac{\begin{matrix}Mean relative quantity of STMN2 with \\ cryptic exon in response to STMN2 AON\end{matrix}}{\begin{matrix}Mean relative quantity of STMN2 with \\ cryptic exon in response to TDP43 AON\end{matrix}} * 100 )$$

The percent increase of full length STMN2 mRNA transcript was calculated using the equation of:

$$( ( \frac{\begin{matrix}Mean relative quantity of FL STMN2 \\ transcript in response to STMN2 AON\end{matrix}}{\begin{matrix}Mean relative quantity of FL STMN2 \\ transcript in response to TDP43 AON\end{matrix}} ) * 100 ) - 100$$

STMN2 antisense oligonucleotides were also evaluated in human motor neurons for potency in reducing cryptic exon and increasing STMN2 full length transcript. iCell human motor neurons (Cellular Dynamics International) were plated at 15×10³ cells/well in a 96-well plate for RT-qPCR RNA quantification or 3×10⁵ cells/well in a 6-well plate for western blot protein quantification according to manufacturer's instructions. Neurons were transfected with TDP43 AON and/or STMN2 AON using endoporter (GeneTools, LLC.) or treated with endoporter alone. Treatment conditions were tested in biological triplicate (qRT-PCR) or duplicate (western blot) wells. The same TDP43 AON described above is used here for evaluating human motor neurons. TDP43 AON is a gapmer oligonucleotide and has the following sequence and chemistry:

(SEQ ID NO: 1669)
5’ A*A*G*G*C*T*T*C*A*T*A*T*T*G*T*A*C*T*T*T 3’

where *=phosphorothioate, underlined=DNA, other=2′MOE RNA.

After 72 hours, antisense oligonucleotides and endoporter were washed out and replaced with fresh media. After 72 additional hours, RNA was collected from the 96-well plates for RT-qPCR or protein collected from the 6-well plates for western blot. RNA was isolated, cDNA generated and multiplexed RT-qPCR assay performed with taqman probes for STMN2 cryptic exon, STMN2 full length transcript and reference GAPDH quantification. The same primers for detecting GAPDH, STMN2 transcript with cryptic exon, and full length STMN2, as described above in reference to SY5Y cells, were applied here for conducting RT-qPCR for human motor neurons. For protein quantification, the soluble portion of the protein collection was denatured and separated by SDS-PAGE, transferred to polyvinylidene difluoride membranes and probed with antibodies against GAPDH (Proteintech, 60004-1-1g), TDP-43 (Proteintech, 10782-2-AP), and Stathmin-2 (ThermoFisher, PA5-23049).

### Example 3: STMN2 Parent Oligonucleotides and Oligonucleotide Variants Restore Full Length STMN2 and Reduce STMN2 Transcripts with a Cryptic Exon

STMN2 parent oligonucleotides and oligonucleotide variants are tested for their ability to increase or restore full-length STMN2 mRNA (i.e., mRNA from which full-length STMN2 is translated) levels in TDP43 silenced cells. In some cases, STMN2 oligonucleotides are tested for their ability to reduce STMN2 transcripts with a cryptic exon. As described further below, the quantified percentage increase/restoration of STMN2-FL and/or percentage reduction of STMN2 transcripts with cryptic exon is described in reference to levels of STMN-FL and/or STMN2 transcripts with cryptic exon in a control group (e.g., cells treated with 500 nM TDP43 AON).

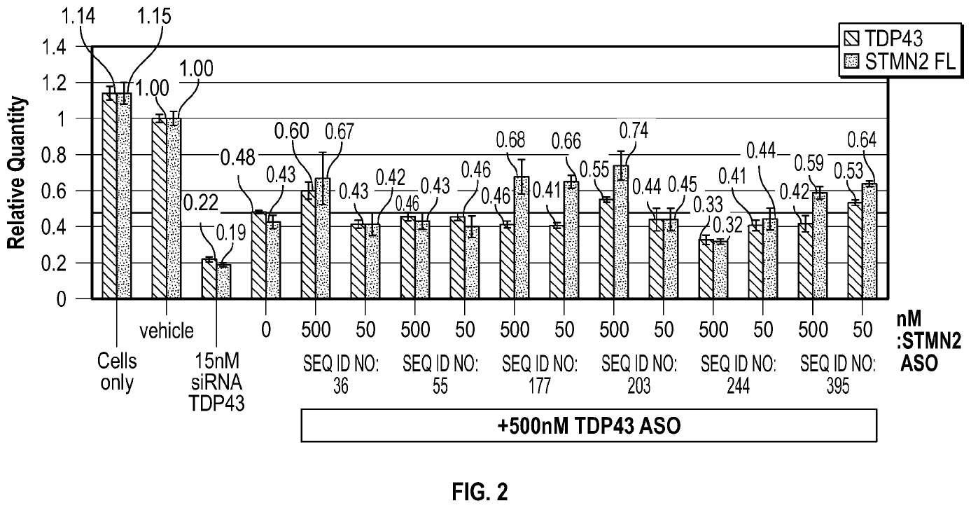

Referring to FIG. 2, TDP43 transcript was decreased by around 52% and STMN2-FL was decreased by around 57% when treated with 500 nM TDP43 AON. A 500 nM treatment of a STMN2 parent AON with SEQ ID NO: 36 increased TDP43 levels by 25% and increased STMN-FL levels by 55% (rescued to 67%). A 50 nM and a 500 nM treatment of a STMN2 parent oligonucleotide with SEQ ID NO: 177 increased STMN-FL levels by 58% (rescued to 66%) and 53% (rescued to 68%) respectively. A 500 nM treatment of a STMN2 parent oligonucleotide with SEQ ID NO: 203 increased TDP43 levels by 15% and STMN-FL levels by 72% (rescued to 74%). A 50 nM and a 500 nM treatment of a STMN2 parent oligonucleotide with SEQ ID NO: 395 increased STMN-FL levels by 49% (rescued to 64%) and 37% (rescued to 59%) respectively.

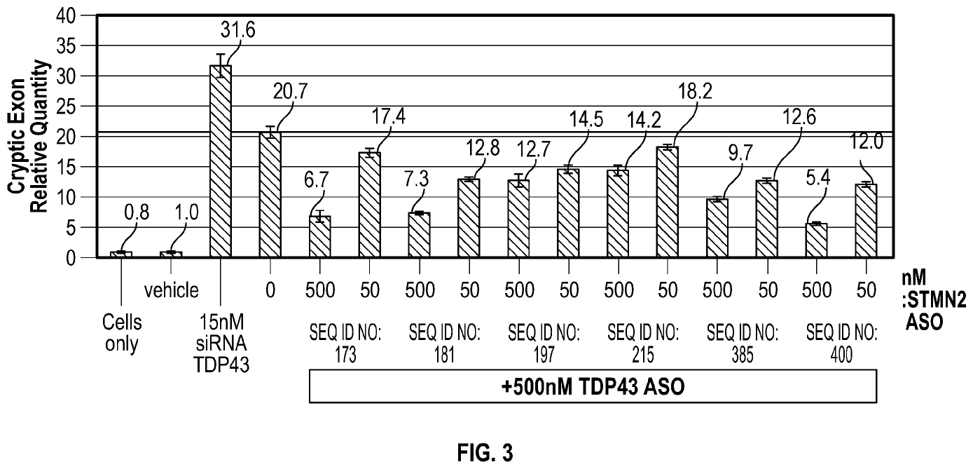

Referring to FIG. 3, the quantity of STMN2 transcript with cryptic exon was increased more than 20-fold when treated with 500 nM TDP43 AON. A 500 nM treatment of a STMN2 parent oligonucleotide with SEQ ID NO: 173 reduced STMN2 transcript with cryptic exon levels by 68%. A 500 nM treatment of a STMN2 parent oligonucleotide with SEQ ID NO: 181 reduced STMN2 transcript with cryptic exon levels by 65%. A 500 nM treatment of a STMN2 parent oligonucleotide with SEQ ID NO: 197 reduced STMN2 transcript with cryptic exon levels by 39%. A 500 nM treatment of a STMN2 parent oligonucleotide with SEQ ID NO: 215 reduced STMN2 transcript with cryptic exon levels by 31%. A 500 nM treatment of a STMN2 parent oligonucleotide with SEQ ID NO: 385 reduced STMN2 transcript with cryptic exon levels by 53%. A 500 nM treatment of a STMN2 parent oligonucleotide with SEQ ID NO: 400 reduced STMN2 transcript with cryptic exon levels by 74%.

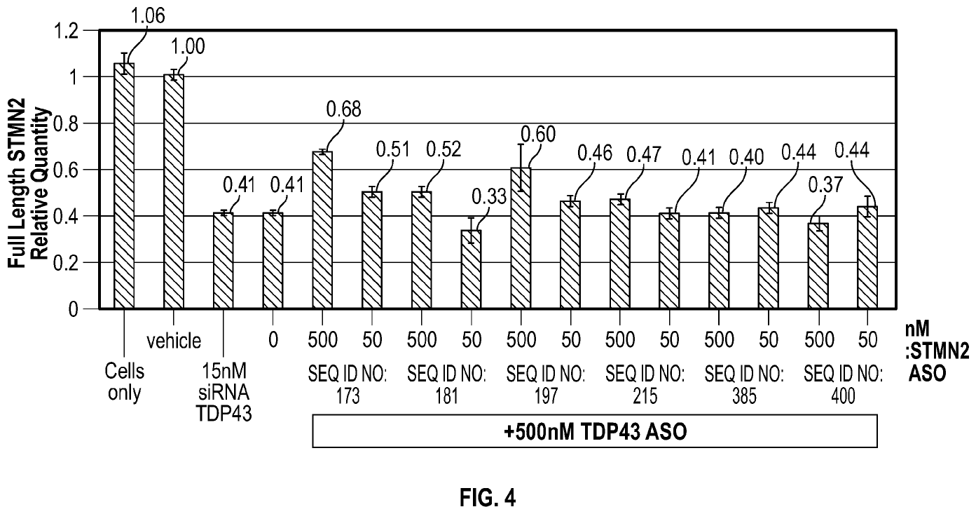

Referring to FIG. 4, STMN2-FL was decreased by around 59% when treated with 500 nM TDP43 AON. A 500 nM treatment of a STMN2 parent oligonucleotide with SEQ ID NO: 173 increased STMN-FL levels by 66% (rescued to 68%). A 500 nM treatment of a STMN2 parent oligonucleotide with SEQ ID NO: 197 increased STMN-FL levels by 46% (rescued to 60%).

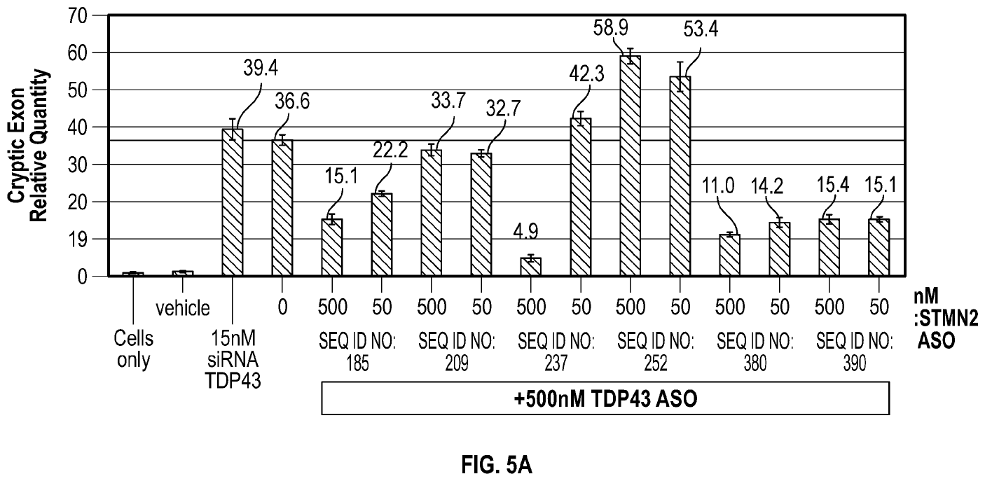

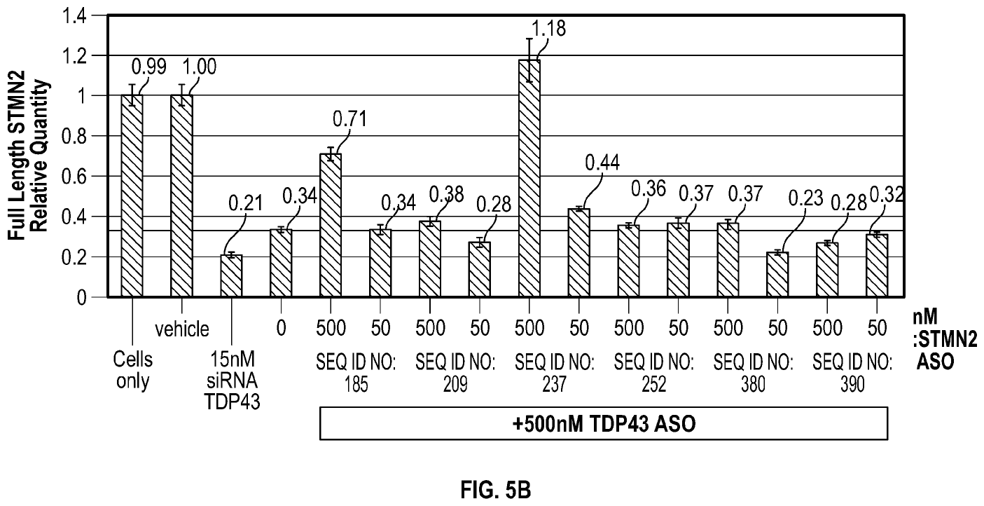

Referring to FIG. 5A, the quantity of STMN2 transcript with cryptic exon was increased more than 36-fold when treated with 500 nM TDP43 AON. A 500 nM treatment of a STMN2 parent oligonucleotide with SEQ ID NO: 185 reduced STMN2 transcript with cryptic exon levels by 58%. A 500 nM treatment of a STMN2 parent oligonucleotide with SEQ ID NO: 237 reduced STMN2 transcript with cryptic exon levels by 87%. A 500 nM treatment of a STMN2 parent oligonucleotide with SEQ ID NO: 380 reduced STMN2 transcript with cryptic exon levels by 70%. A 500 nM treatment of a STMN2 parent oligonucleotide with SEQ ID NO: 390 reduced STMN2 transcript with cryptic exon levels by 58%.

Referring to FIG. 5B, STMN2-FL was decreased by 66% when treated with 500 nM TDP43 AON. A 500 nM treatment of a STMN2 parent oligonucleotide with SEQ ID NO: 185 increased STMN-FL levels by 109% (rescued to 71%). A 500 nM treatment of a STMN2 parent oligonucleotide with SEQ ID NO: 237 increased STMN-FL levels by 247% (rescued to 118%).

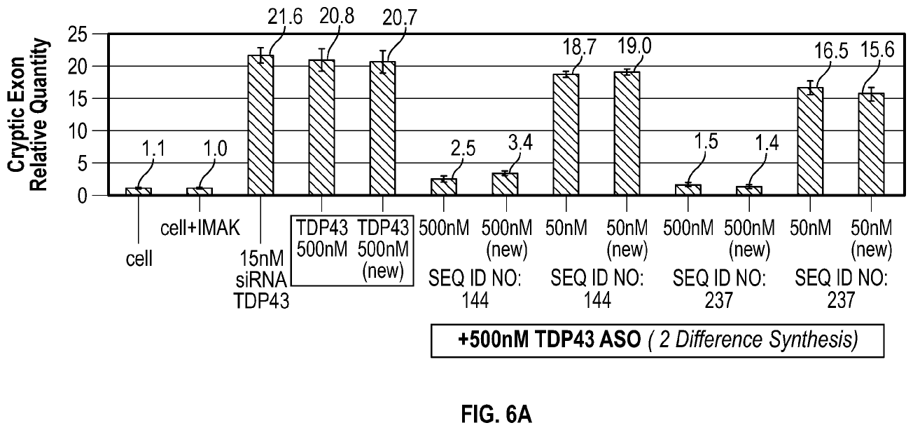

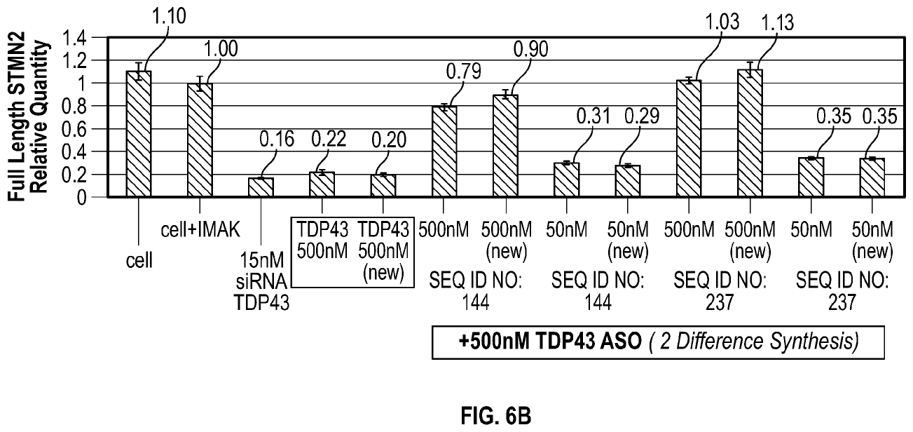

Referring to FIG. 6A, the quantity of STMN2 transcript with cryptic exon was increased more than 20-fold when treated with 500 nM TDP43 AON (two different syntheses). A 500 nM treatment of a STMN2 parent oligonucleotide with SEQ ID NO: 144 reduced STMN2 transcript with cryptic exon levels by 83 to 88%. A 500 nM treatment of a STMN2 parent oligonucleotide with SEQ ID NO: 237 reduced STMN2 transcript with cryptic exon levels by 92 to 93%.

Referring to FIG. 6B, STMN2-FL was decreased by about 80% when treated with 500 nM TDP43 AON. A 500 nM treatment of a STMN2 parent oligonucleotide with SEQ ID NO: 144 increased STMN-FL levels by 276% to 329% (rescued to 79% to 90%). A 500 nM treatment of a STMN2 parent oligonucleotide with SEQ ID NO: 237 increased STMN-FL levels by 390% to 438% (rescued to 103% to 113%).

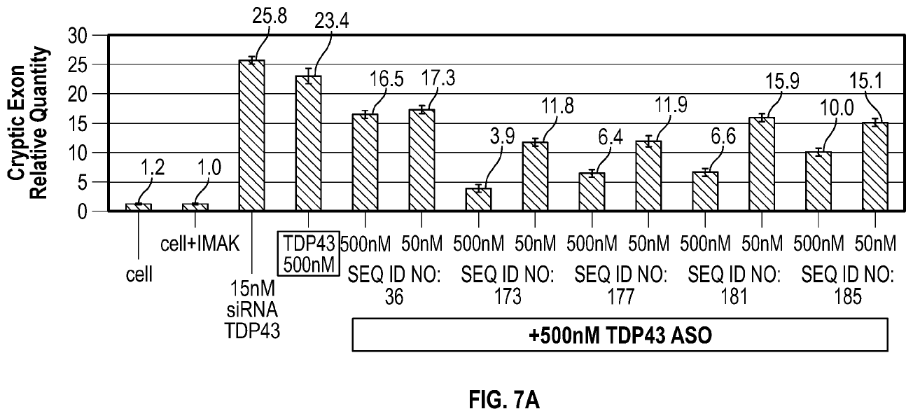

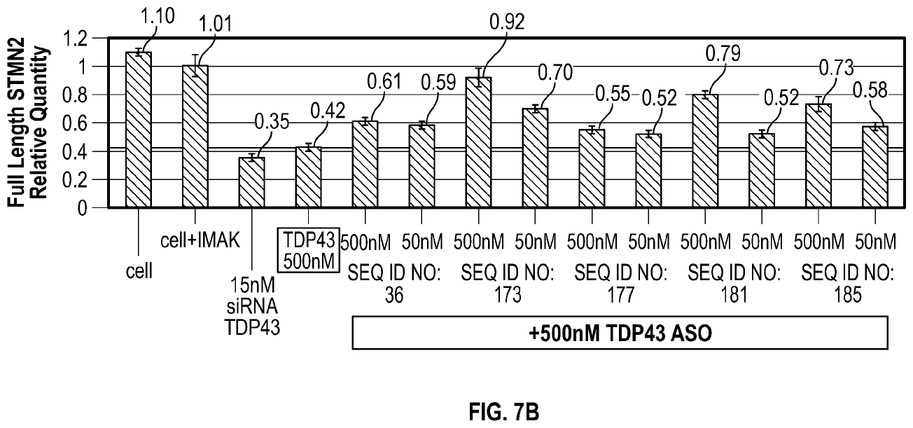

Referring to FIG. 7A, the quantity of STMN2 transcript with cryptic exon was increased more than 23-fold when treated with 500 nM TDP43 AON. A 500 nM treatment of a STMN2 parent oligonucleotide with SEQ ID NO: 173 reduced STMN2 transcript with cryptic exon levels by 83%. A 500 nM treatment of a STMN2 parent oligonucleotide with SEQ ID NO: 177 reduced STMN2 transcript with cryptic exon levels by 83%. A 500 nM treatment of a STMN2 parent oligonucleotide with SEQ ID NO: 181 reduced STMN2 transcript with cryptic exon levels by 72%.

Referring to FIG. 7B, STMN2-FL was decreased by about 58% when treated with 500 nM TDP43 AON. A 500 nM treatment of a STMN2 parent oligonucleotide with SEQ ID NO: 173 increased STMN-FL levels by 119% (rescued to 92%). A 500 nM treatment of a STMN2 parent oligonucleotide with SEQ ID NO: 181 increased STMN-FL levels by 88% (rescued to 79%). A 500 nM treatment of a STMN2 parent oligonucleotide with SEQ ID NO: 185 increased STMN-FL levels by 74% (rescued to 73%).

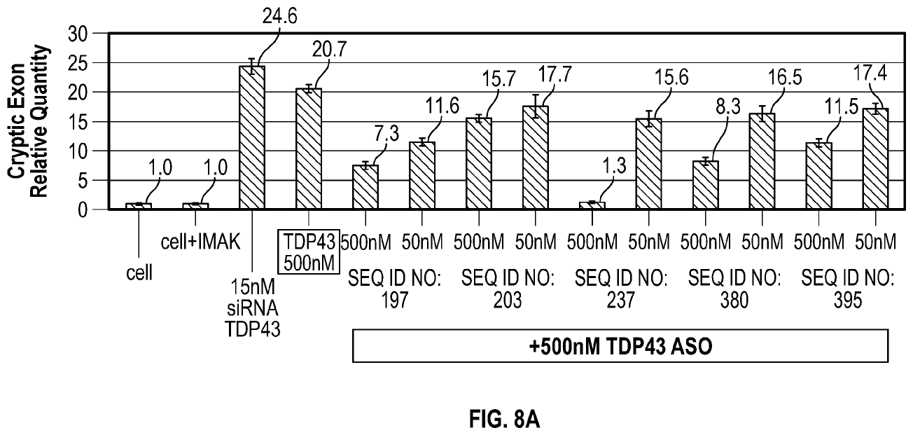

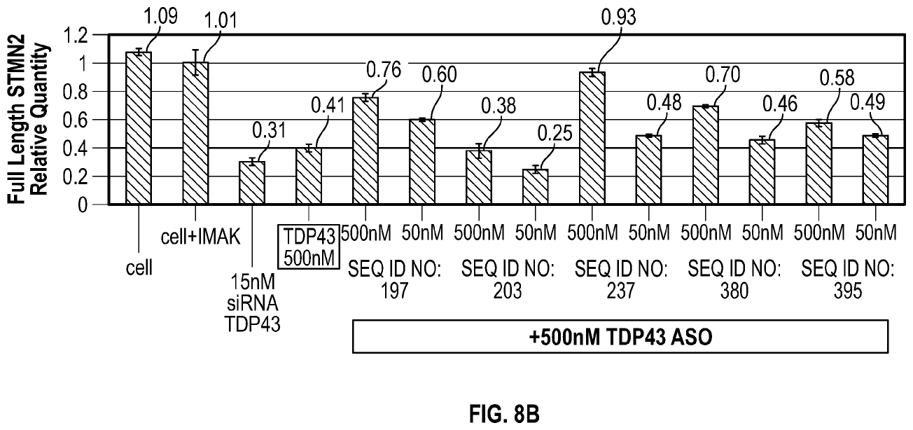

Referring to FIG. 8A, the quantity of STMN2 transcript with cryptic exon was increased more than 20-fold when treated with 500 nM TDP43 AON. A 500 nM treatment of a STMN2 parent oligonucleotide with SEQ ID NO: 197 reduced STMN2 transcript with cryptic exon levels by 65%. A 500 nM treatment of a STMN2 parent oligonucleotide with SEQ ID NO: 237 reduced STMN2 transcript with cryptic exon levels by 94%.

Referring to FIG. 8B, STMN2-FL was decreased by 59% when treated with 500 nM TDP43 AON. A 500 nM treatment of a STMN2 parent oligonucleotide with SEQ ID NO: 197 increased STMN-FL levels by 85% (rescued to 76%). A 500 nM treatment of a STMN2 parent oligonucleotide with SEQ ID NO: 237 increased STMN-FL levels by 127% (rescued to 93%). A 500 nM treatment of a STMN2 parent oligonucleotide with SEQ ID NO: 380 increased STMN-FL levels by 71% (rescued to 70%).

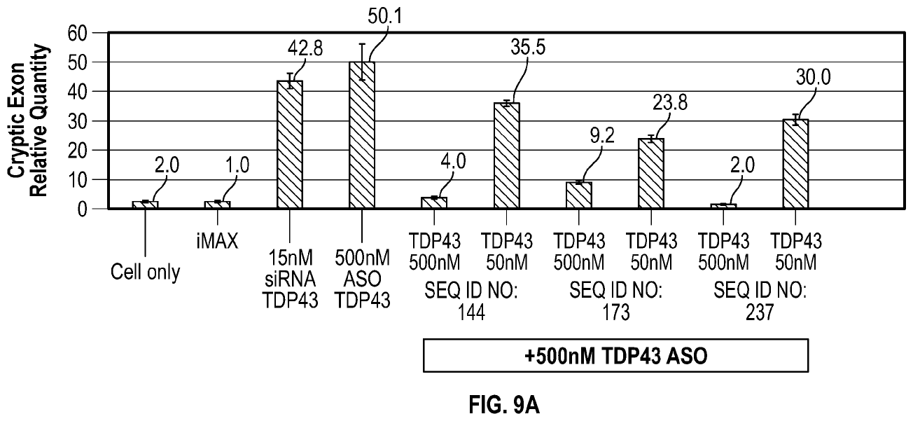

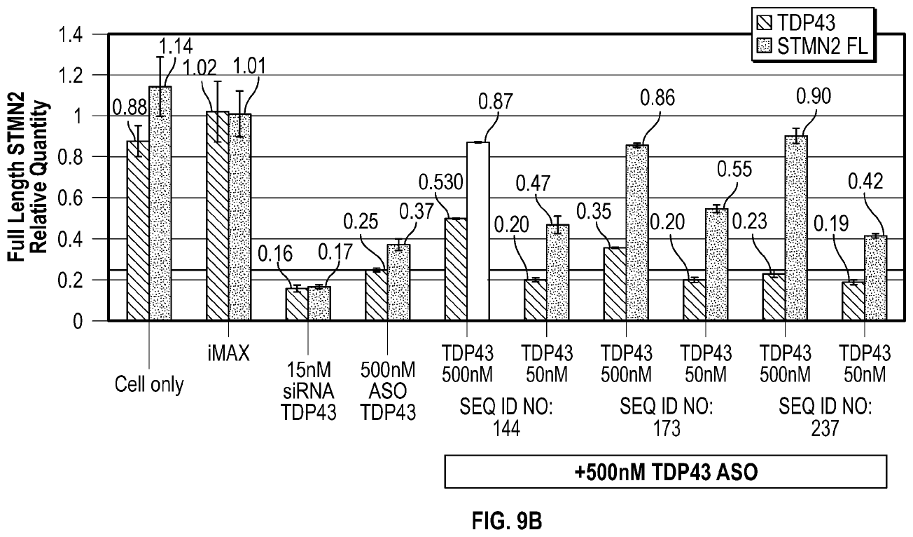

Referring to FIG. 9A, the quantity of STMN2 transcript with cryptic exon was increased more than 50-fold when treated with 500 nM TDP43 AON. A 500 nM treatment of a STMN2 parent oligonucleotide with SEQ ID NO: 144 reduced STMN2 transcript with cryptic exon levels by 92%. A 500 nM treatment of a STMN2 parent oligonucleotide with SEQ ID NO: 173 reduced STMN2 transcript with cryptic exon levels by 82%. A 500 nM treatment of a STMN2 parent oligonucleotide with SEQ ID NO: 237 reduced STMN2 transcript with cryptic exon levels by 96%.

Referring to FIG. 9B, STMN2-FL was decreased by 67% when treated with 500 nM TDP43 AON. A 500 nM treatment of a STMN2 parent oligonucleotide with SEQ ID NO: 144 increased STMN-FL levels by 135% (rescued to 87%). A 500 nM treatment of a STMN2 parent oligonucleotide with SEQ ID NO: 173 increased STMN-FL levels by 132% (rescued to 86%). A 500 nM treatment of a STMN2 parent oligonucleotide with SEQ ID NO: 237 increased STMN-FL levels by 143% (rescued to 90%).

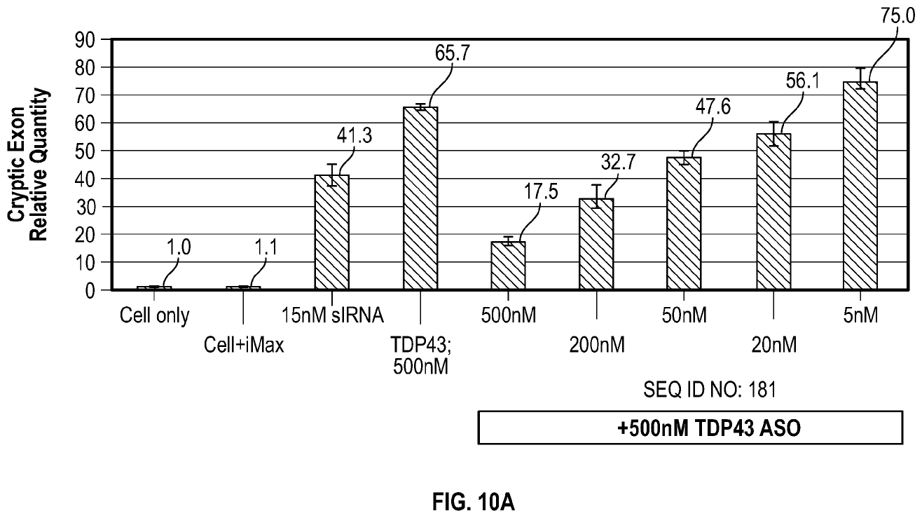

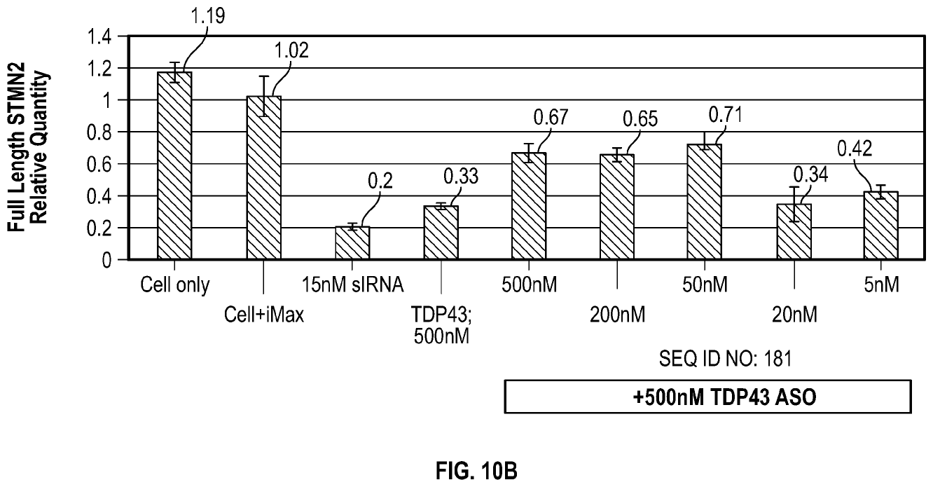

Referring to FIG. 10A, the quantity of STMN2 transcript with cryptic exon was increased more than 65-fold when treated with 500 nM TDP43 AON. A 200 nM treatment of a STMN2 parent oligonucleotide with SEQ ID NO: 181 reduced STMN2 transcript with cryptic exon levels by 50%. A 500 nM treatment of a STMN2 parent oligonucleotide with SEQ ID NO: 181 reduced STMN2 transcript with cryptic exon levels by 73%. Referring to FIG. 10B, STMN2-FL was decreased by 67% when treated with 500 nM TDP43 AON. A 50 nM treatment of a STMN2 parent oligonucleotide with SEQ ID NO: 181 increased STMN-FL levels by 115% (rescued to 71%). A 200 nM treatment of a STMN2 parent oligonucleotide with SEQ ID NO: 181 increased STMN-FL levels by 97% (rescued to 65%). A 500 nM treatment of a STMN2 parent oligonucleotide with SEQ ID NO: 181 increased STMN-FL levels by 94% (rescued to 64%).

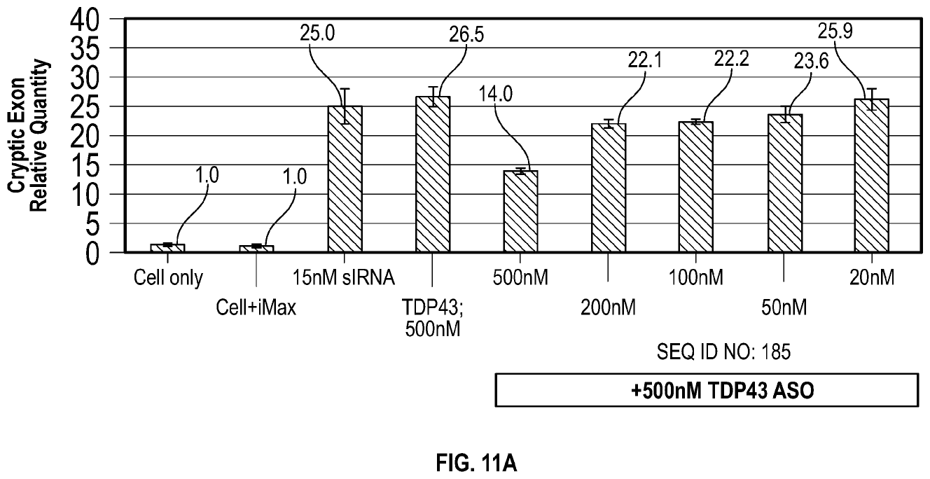

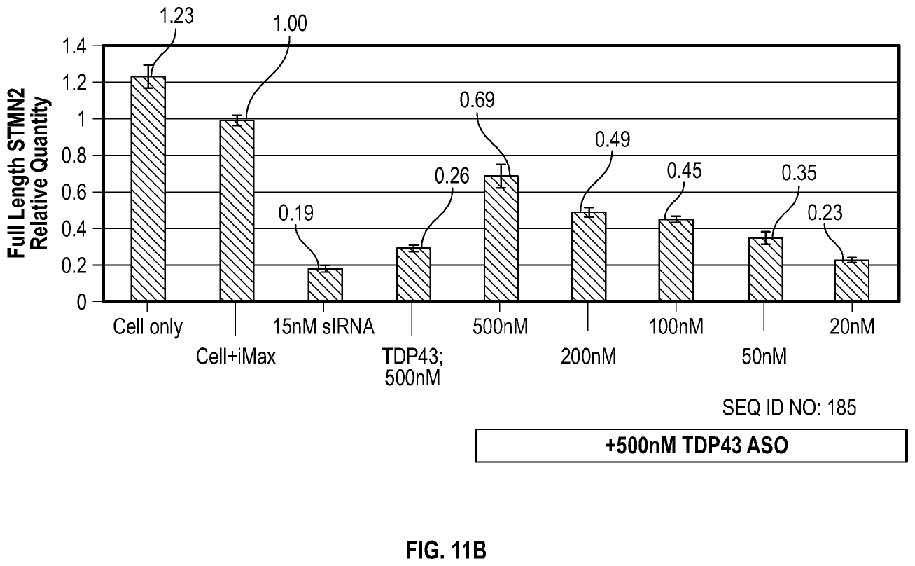

Referring to FIG. 11A, the quantity of STMN2 transcript with cryptic exon was increased more than 26-fold when treated with 500 nM TDP43 AON. A 500 nM treatment of a STMN2 parent oligonucleotide with SEQ ID NO: 185 reduced STMN2 transcript with cryptic exon levels by 47%. Referring to FIG. 11B, STMN2-FL was decreased by 74% when treated with 500 nM TDP43 AON. A 50 nM treatment of a STMN2 parent oligonucleotide with SEQ ID NO: 185 increased STMN-FL levels by 73% (rescued to 45%). A 200 nM treatment of a STMN2 parent oligonucleotide with SEQ ID NO: 185 increased STMN-FL levels by 246% (rescued to 90%). A 500 nM treatment of a STMN2 parent oligonucleotide with SEQ ID NO: 185 increased STMN-FL levels by 165% (rescued to 69%).

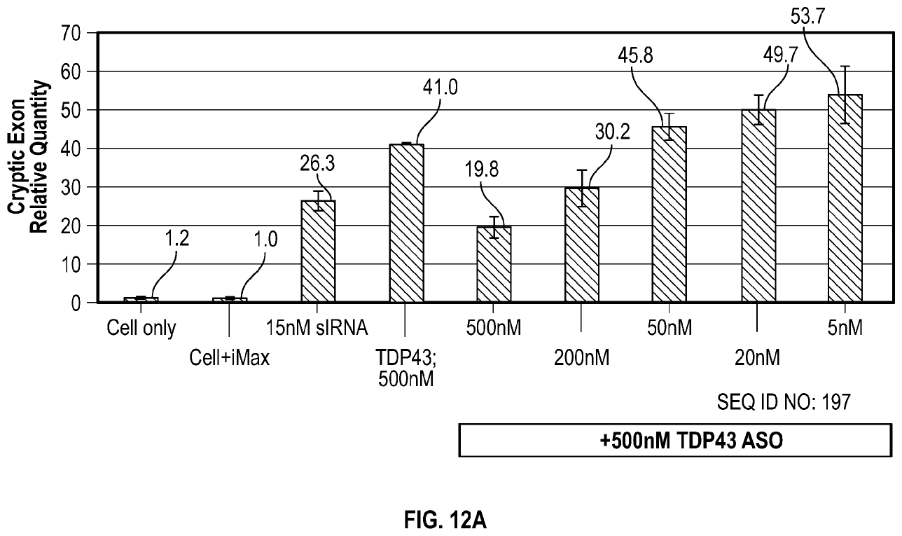

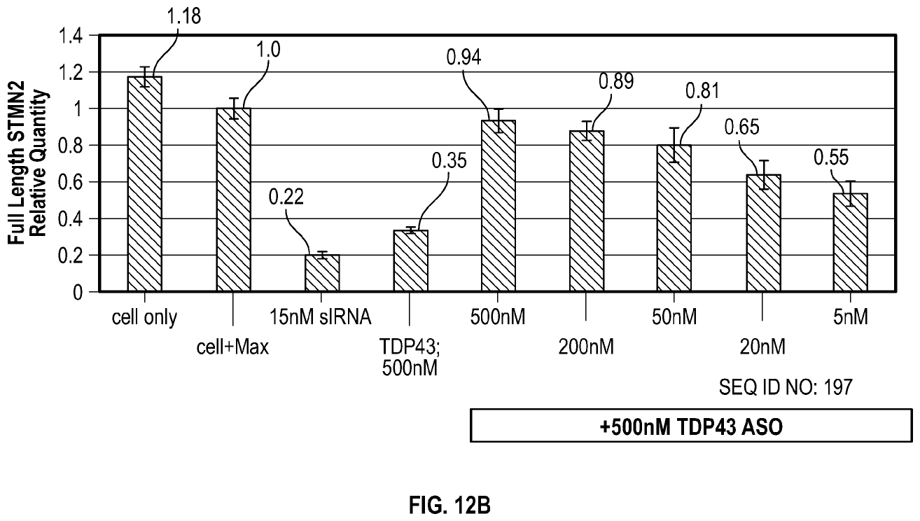

Referring to FIG. 12A, the quantity of STMN2 transcript with cryptic exon was increased more than 41-fold when treated with 500 nM TDP43 AON. A 500 nM treatment of a STMN2 parent oligonucleotide with SEQ ID NO: 197 reduced STMN2 transcript with cryptic exon levels by 51%. Referring to FIG. 12B, STMN2-FL was decreased by 65% when treated with 500 nM TDP43 AON. A 20 nM treatment of a STMN2 parent oligonucleotide with SEQ ID NO: 197 increased STMN-FL levels by 86% (rescued to 65%). A 50 nM treatment of a STMN2 parent oligonucleotide with SEQ ID NO: 197 increased STMN-FL levels by 131% (rescued to 81%). A 200 nM treatment of a STMN2 parent oligonucleotide with SEQ ID NO: 197 increased STMN-FL levels by 154% (rescued to 89%). A 500 nM treatment of a STMN2 parent oligonucleotide with SEQ ID NO: 197 increased STMN-FL levels by 169% (rescued to 94%).

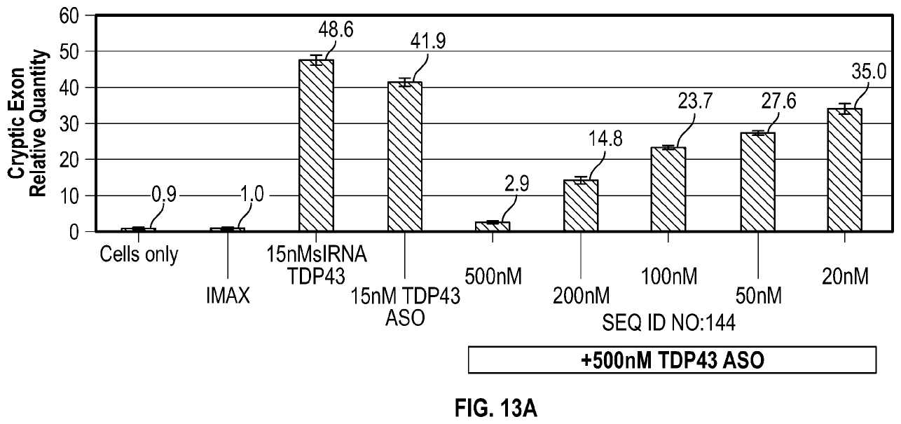

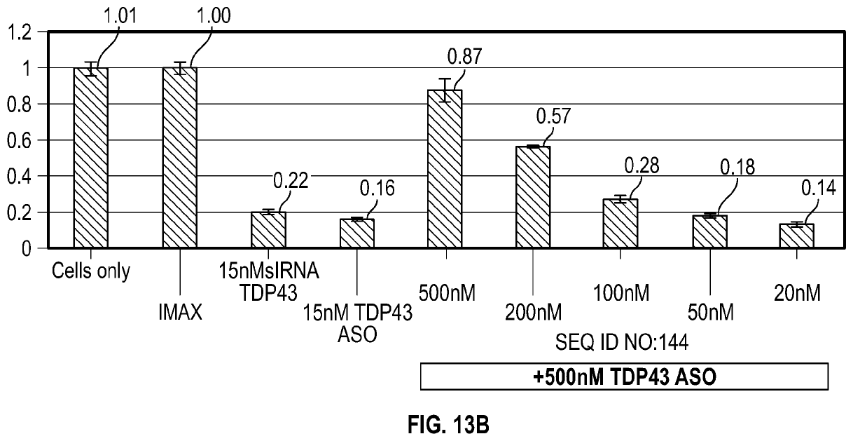

Referring to FIG. 13A, the quantity of STMN2 transcript with cryptic exon was increased more than 41-fold when treated with 500 nM TDP43 AON. A 500 nM treatment of a STMN2 parent oligonucleotide with SEQ ID NO: 144 reduced STMN2 transcript with cryptic exon levels by 93%. Referring to FIG. 13B, STMN2-FL was decreased by 84% when treated with 500 nM TDP43 AON. A 50 nM treatment of a STMN2 parent oligonucleotide with SEQ ID NO: 144 increased STMN-FL levels by 75% (rescued to 28%). A 200 nM treatment of a STMN2 parent oligonucleotide with SEQ ID NO: 144 increased STMN-FL levels by 260% (rescued to 57%). A 500 nM treatment of a STMN2 parent oligonucleotide with SEQ ID NO: 144 increased STMN-FL levels by 444% (rescued to 87%).

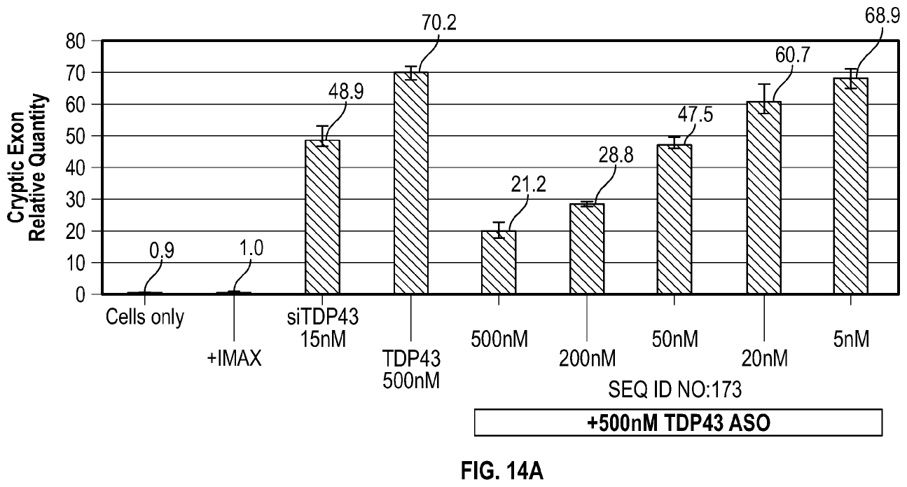

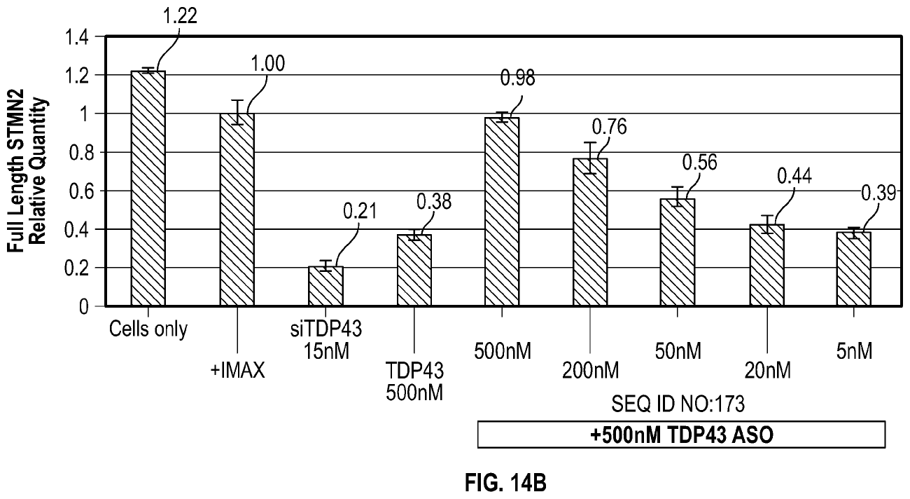

Referring to FIG. 14A, the quantity of STMN2 transcript with cryptic exon was increased more than 24-fold when treated with 500 nM TDP43 AON. A 200 nM treatment of a STMN2 parent oligonucleotide with SEQ ID NO: 173 reduced STMN2 transcript with cryptic exon levels by 59%. A 500 nM treatment of a STMN2 parent oligonucleotide with SEQ ID NO: 173 reduced STMN2 transcript with cryptic exon levels by 70%. Referring to FIG. 14B, STMN2-FL was decreased by 62% when treated with 500 nM TDP43 AON. A 200 nM treatment of a STMN2 parent oligonucleotide with SEQ ID NO: 173 increased STMN-FL levels by 100% (rescued to 76%). A 500 nM treatment of a STMN2 parent oligonucleotide with SEQ ID NO: 173 increased STMN-FL levels by 158% (rescued to 98%).

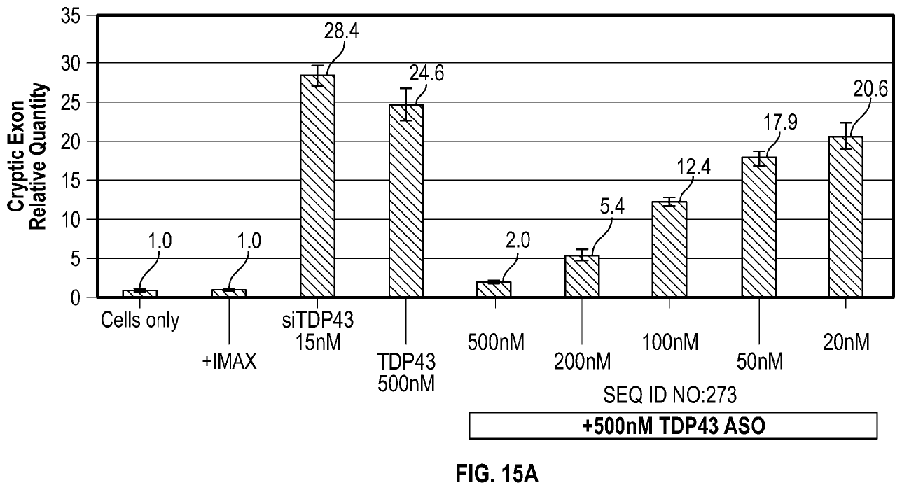

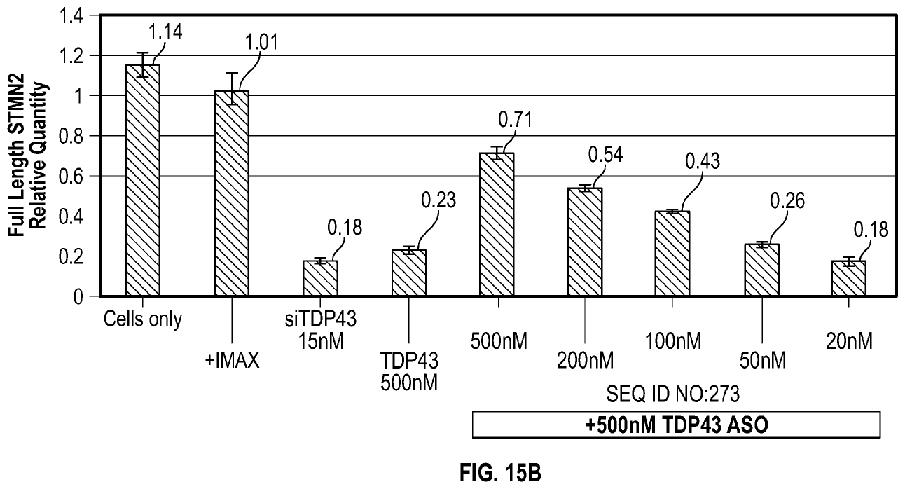

Referring to FIG. 15A, the quantity of STMN2 transcript with cryptic exon was increased more than 70-fold when treated with 500 nM TDP43 AON. A 200 nM treatment of a STMN2 parent oligonucleotide with SEQ ID NO: 237 reduced STMN2 transcript with cryptic exon levels by 78%. A 500 nM treatment of a STMN2 parent oligonucleotide with SEQ ID NO: 237 reduced STMN2 transcript with cryptic exon levels by 92%. Referring to FIG. 15B, STMN2-FL was decreased by 77% when treated with 500 nM TDP43 AON. A 50 nM treatment of a STMN2 parent oligonucleotide with SEQ ID NO: 237 increased STMN-FL levels by 87% (rescued to 43%). A 200 nM treatment of a STMN2 parent oligonucleotide with SEQ ID NO: 237 increased STMN-FL levels by 135% (rescued to 54%). A 500 nM treatment of a STMN2 parent oligonucleotide with SEQ ID NO: 237 increased STMN-FL levels by 209% (rescued to 71%).

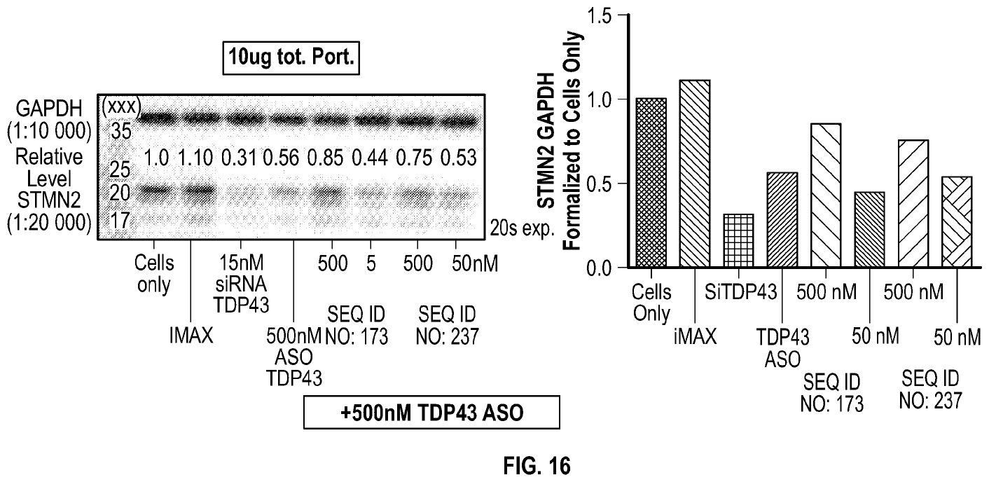

Referring to FIG. 16, STMN2 protein levels were decreased by 44% when treated with 500 nM TDP43 AON. A 500 nM treatment of a STMN2 parent oligonucleotide with SEQ ID NO: 173 increased STMN protein levels by 52%. A 500 nM treatment of a STMN2 parent oligonucleotide with SEQ ID NO: 237 increased STMN protein levels by 34%.

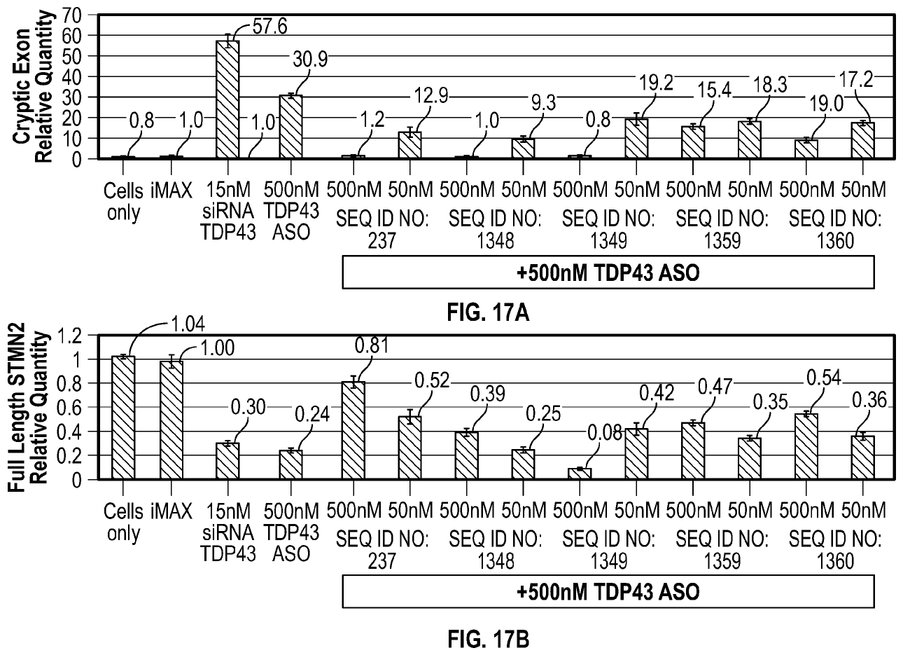

Referring to FIG. 17A, the quantity of STMN2 transcript with cryptic exon was increased more than 30-fold when treated with 500 nM TDP43 AON. A 500 nM treatment of a STMN2 parent oligonucleotide with SEQ ID NO: 237 reduced STMN2 transcript with cryptic exon levels by 96%. A 500 nM treatment of a STMN2 oligonucleotide variant with SEQ ID NO: 1348 reduced STMN2 transcript with cryptic exon levels by 97%. A 500 nM treatment of a STMN2 oligonucleotide variant with SEQ ID NO: 1349 reduced STMN2 transcript with cryptic exon levels by 97%. A 500 nM treatment of a STMN2 oligonucleotide variant with SEQ ID NO: 1360 reduced STMN2 transcript with cryptic exon levels by 71%.

Referring to FIG. 17B, STMN2-FL was decreased by 76% when treated with 500 nM TDP43 AON. A 500 nM treatment of a STMN2 parent oligonucleotide with SEQ ID NO: 237 increased STMN-FL levels by 238% (rescued to 81%). A 500 nM treatment of a STMN2 oligonucleotide variant with SEQ ID NO: 1348 increased STMN-FL levels by 63% (rescued to 39%). A 500 nM treatment of a STMN2 oligonucleotide variant with SEQ ID NO: 1359 increased STMN-FL levels by 96% (rescued to 47%). A 500 nM treatment of a STMN2 oligonucleotide variant with SEQ ID NO: 1360 increased STMN-FL levels by 125% (rescued to 54%).

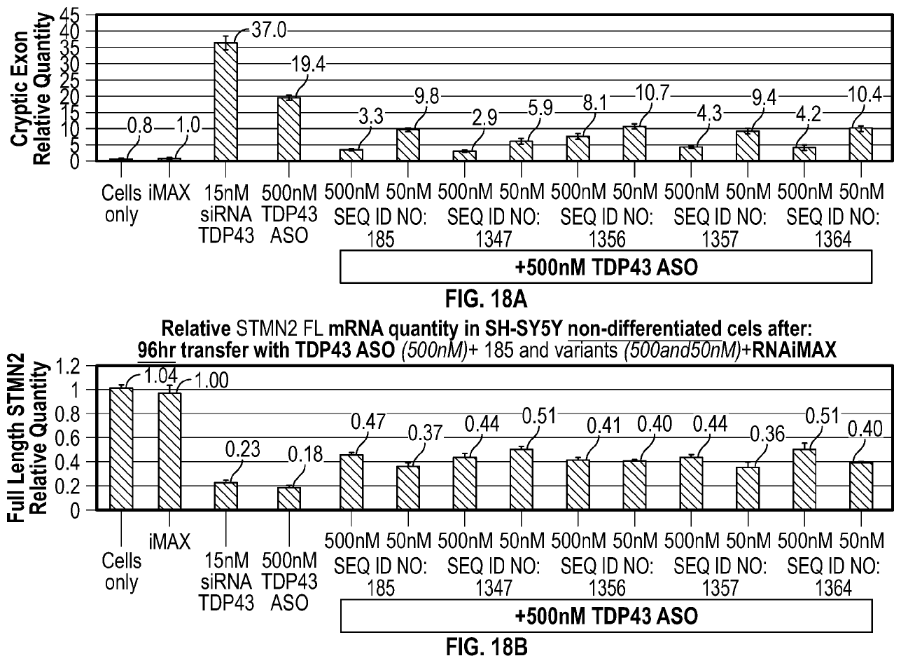

Referring to FIG. 18A, the quantity of STMN2 transcript with cryptic exon was increased more than 19-fold when treated with 500 nM TDP43 AON. A 500 nM treatment of a STMN2 parent oligonucleotide with SEQ ID NO: 185 reduced STMN2 transcript with cryptic exon levels by 83%. A 500 nM treatment of a STMN2 oligonucleotide variant with SEQ ID NO: 1347 reduced STMN2 transcript with cryptic exon levels by 85%. A 500 nM treatment of a STMN2 oligonucleotide variant with SEQ ID NO: 1356 reduced STMN2 transcript with cryptic exon levels by 56%. A 500 nM treatment of a STMN2 oligonucleotide variant with SEQ ID NO: 1357 reduced STMN2 transcript with cryptic exon levels by 78%. A 500 nM treatment of a STMN2 oligonucleotide variant with SEQ ID NO: 1364 reduced STMN2 transcript with cryptic exon levels by 78%.

Referring to FIG. 18B, STMN2-FL was decreased by 82% when treated with 500 nM TDP43 AON. A 500 nM treatment of a STMN2 parent oligonucleotide with SEQ ID NO: 185 increased STMN-FL levels by 161% (rescued to 47%). A 500 nM treatment of a STMN2 oligonucleotide variant with SEQ ID NO: 1347 increased STMN-FL levels by 144% (rescued to 44%). A 500 nM treatment of a STMN2 oligonucleotide variant with SEQ ID NO: 1356 increased STMN-FL levels by 128% (rescued to 41%). A 500 nM treatment of a STMN2 oligonucleotide variant with SEQ ID NO: 1357 increased STMN-FL levels by 144% (rescued to 44%). A 500 nM treatment of a STMN2 oligonucleotide variant with SEQ ID NO: 1364 increased STMN-FL levels by 183% (rescued to 51%).

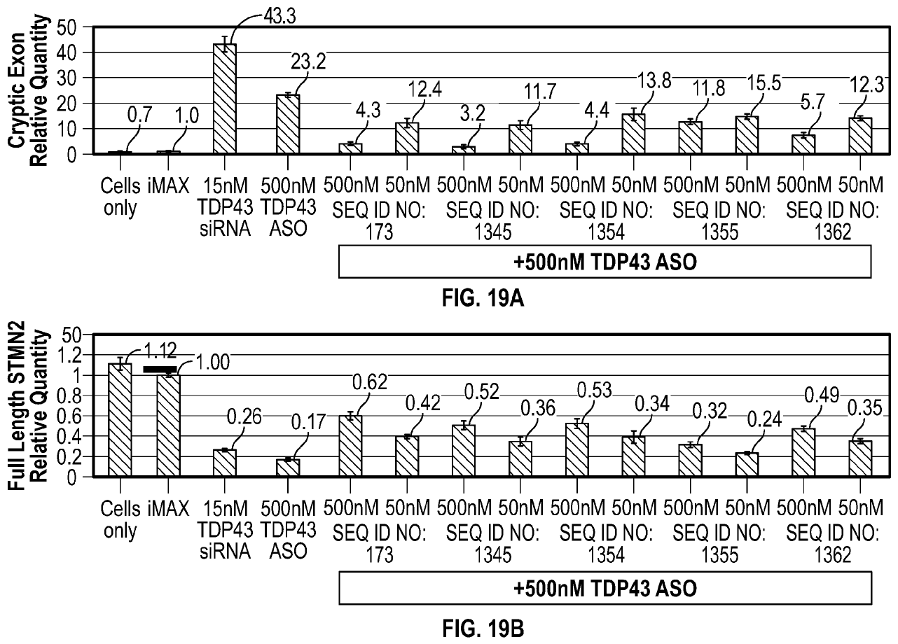

Referring to FIG. 19A, the quantity of STMN2 transcript with cryptic exon was increased more than 23-fold when treated with 500 nM TDP43 AON. A 500 nM treatment of a STMN2 parent oligonucleotide with SEQ ID NO: 173 reduced STMN2 transcript with cryptic exon levels by 81%. A 500 nM treatment of a STMN2 oligonucleotide variant with SEQ ID NO: 1345 reduced STMN2 transcript with cryptic exon levels by 86%. A 500 nM treatment of a STMN2 oligonucleotide variant with SEQ ID NO: 1354 reduced STMN2 transcript with cryptic exon levels by 81%. A 500 nM treatment of a STMN2 oligonucleotide variant with SEQ ID NO: 1355 reduced STMN2 transcript with cryptic exon levels by 47%. A 500 nM treatment of a STMN2 oligonucleotide variant with SEQ ID NO: 1362 reduced STMN2 transcript with cryptic exon levels by 75%.

Referring to FIG. 19B, STMN2-FL was decreased by 83% when treated with 500 nM TDP43 AON. A 500 nM treatment of a STMN2 parent oligonucleotide with SEQ ID NO: 173 increased STMN-FL levels by 265% (rescued to 62%). A 500 nM treatment of a STMN2 oligonucleotide variant with SEQ ID NO: 1345 increased STMN-FL levels by 206% (rescued to 52%). A 500 nM treatment of a STMN2 oligonucleotide variant with SEQ ID NO: 1354 increased STMN-FL levels by 212% (rescued to 53%). A 500 nM treatment of a STMN2 oligonucleotide variant with SEQ ID NO: 1355 increased STMN-FL levels by 88% (rescued to 32%). A 500 nM treatment of a STMN2 oligonucleotide variant with SEQ ID NO: 1362 increased STMN-FL levels by 188% (rescued to 49%).

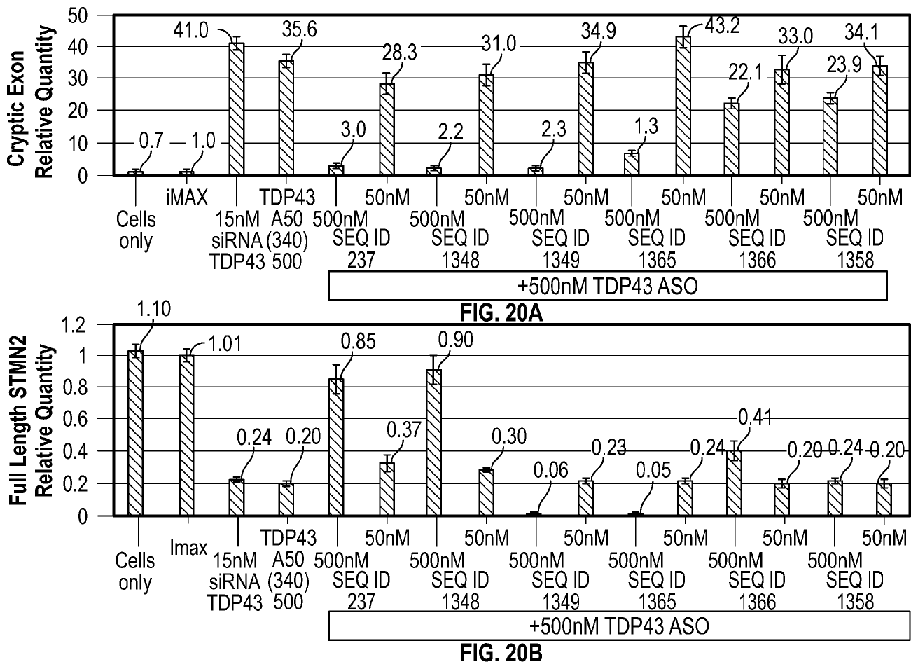

Referring to FIG. 20A, the quantity of STMN2 transcript with cryptic exon was increased more than 35-fold when treated with 500 nM TDP43 AON. A 500 nM treatment of a STMN2 parent oligonucleotide with SEQ ID NO: 237 reduced STMN2 transcript with cryptic exon levels by 91%. A 500 nM treatment of a STMN2 oligonucleotide variant with SEQ ID NO: 1348 reduced STMN2 transcript with cryptic exon levels by 94%. A 500 nM treatment of a STMN2 oligonucleotide variant with SEQ ID NO: 1349 reduced STMN2 transcript with cryptic exon levels by 96%. A 500 nM treatment of a STMN2 oligonucleotide variant with SEQ ID NO: 1365 reduced STMN2 transcript with cryptic exon levels by 82%. A 500 nM treatment of a STMN2 oligonucleotide variant with SEQ ID NO: 1366 reduced STMN2 transcript with cryptic exon levels by 38%. A 500 nM treatment of a STMN2 oligonucleotide variant with SEQ ID NO: 1358 reduced STMN2 transcript with cryptic exon levels by 33%.

Referring to FIG. 20B, STMN2-FL was decreased by 80% when treated with 500 nM TDP43 AON. A 500 nM treatment of a STMN2 parent oligonucleotide with SEQ ID NO: 237 increased STMN-FL levels by 325% (rescued to 85%). A 500 nM treatment of a STMN2 oligonucleotide variant with SEQ ID NO: 1348 increased STMN-FL levels by 350% (rescued to 90%). A 500 nM treatment of a STMN2 oligonucleotide variant with SEQ ID NO: 1366 increased STMN-FL levels by 105% (rescued to 41%). A 500 nM treatment of a STMN2 oligonucleotide variant with SEQ ID NO: 1358 increased STMN-FL levels by 20% (rescued to 24%).

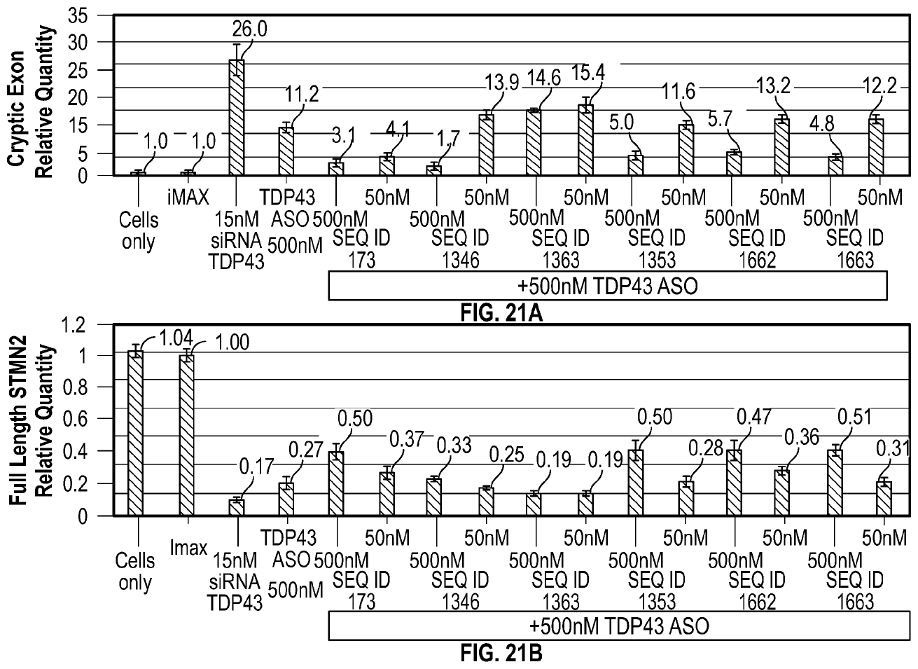

Referring to FIG. 21A, the quantity of STMN2 transcript with cryptic exon was increased more than 11-fold when treated with 500 nM TDP43 AON. A 500 nM treatment of a STMN2 parent oligonucleotide with SEQ ID NO: 173 reduced STMN2 transcript with cryptic exon levels by 72%. A 500 nM treatment of a STMN2 oligonucleotide variant with SEQ ID NO: 1346 reduced STMN2 transcript with cryptic exon levels by 85%. A 500 nM treatment of a STMN2 oligonucleotide variant with SEQ ID NO: 1353 reduced STMN2 transcript with cryptic exon levels by 55%. A 500 nM treatment of a STMN2 oligonucleotide variant with SEQ ID NO: 1662 (G*A*G*TCCTGCAATATGAATATA*AT*T*T, where * indicates phosphodiester linkage) reduced STMN2 transcript with cryptic exon levels by 49%. A 500 nM treatment of a STMN2 oligonucleotide variant with SEQ ID NO: 1663 (GAGTCCTG*C*A*A*T*A*TGAATATAATTT, where * indicates phosphodiester linkage) reduced STMN2 transcript with cryptic exon levels by 57%.

Referring to FIG. 21B, STMN2-FL was decreased by 73% when treated with 500 nM TDP43 AON. A 500 nM treatment of a STMN2 parent oligonucleotide with SEQ ID NO: 173 increased STMN-FL levels by 85% (rescued to 50%). A 500 nM treatment of a STMN2 oligonucleotide variant with SEQ ID NO: 1353 increased STMN-FL levels by 85% (rescued to 50%). A 500 nM treatment of a STMN2 oligonucleotide variant with SEQ ID NO: 1662 increased STMN-FL levels by 74% (rescued to 47%). A 500 nM treatment of a STMN2 oligonucleotide variant SEQ ID NO: 1663 increased STMN-FL levels by 89% (rescued to 51%).

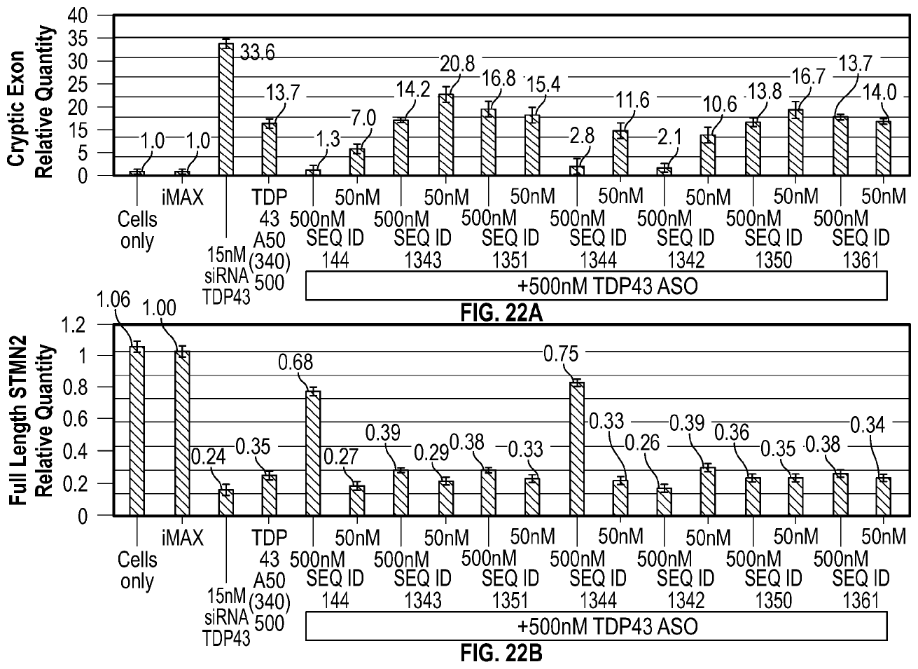

Referring to FIG. 22A, the quantity of STMN2 transcript with cryptic exon was increased more than 13-fold when treated with 500 nM TDP43 AON. A 500 nM treatment of a STMN2 parent oligonucleotide with SEQ ID NO: 144 reduced STMN2 transcript with cryptic exon levels by 91%. A 500 nM treatment of a STMN2 oligonucleotide variant with SEQ ID NO: 1344 reduced STMN2 transcript with cryptic exon levels by 80%. A 500 nM treatment of a STMN2 oligonucleotide variant with SEQ ID NO: 1342 reduced STMN2 transcript with cryptic exon levels by 85%.

Referring to FIG. 22B, STMN2-FL was decreased by 65% when treated with 500 nM TDP43 AON. A 500 nM treatment of a STMN2 parent oligonucleotide with SEQ ID NO: 144 increased STMN-FL levels by 94% (rescued to 68%). A 500 nM treatment of a STMN2 parent oligonucleotide with SEQ ID NO: 1343 increased STMN-FL levels by 11% (rescued to 39%). A 500 nM treatment of a STMN2 parent oligonucleotide with SEQ ID NO: 1351 increased STMN-FL levels by 9% (rescued to 38%). A 500 nM treatment of a STMN2 oligonucleotide variant with SEQ ID NO: 1344 increased STMN-FL levels by 114% (rescued to 75%). A 500 nM treatment of a STMN2 parent oligonucleotide with SEQ ID NO: 1350 increased STMN-FL levels by 3% (rescued to 36%). A 500 nM treatment of a STMN2 parent oligonucleotide with SEQ ID NO: 1361 increased STMN-FL levels by 9% (rescued to 38%).

### Example 4: Neuropathy as an Indication that can be Targeted by a Stathmin-2 Cryptic Splicing Modulator

Experimentally, iCell human motor neurons (Cellular Dynamics International) were plated at 19,000 cells/well in a 96-well plate according to manufacturer's instructions. Neurons were treated with SEQ ID NO: 237 and endoporter (GeneTools, LLC.) or treated with endoporter alone in triplicate wells at day 7 post-plating. After 72 hours, SEQ ID NO: 237 STMN2 parent oligonucleotide and endoporter were washed out and MG132 added. After 18 hours, RNA was isolated, cDNA generated and multiplexed QPCR assay performed for STMN2 cryptic exon and reference GAPDH quantification.

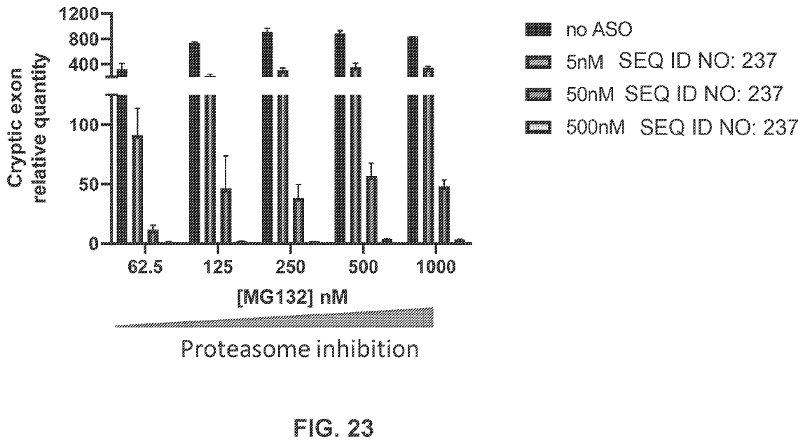

Referring to FIG. 23, it illustrates a bar graph showing reversal of cryptic exon induction using SEQ ID NO: 237 STMN2 parent oligonucleotide even in view of increasing proteasome inhibition. As a control, cells that were treated with endoporter alone (no AON) and then subsequently treated with MG132 (across all concentrations of MG132) demonstrated high levels of cryptic exon. This is indicative of TDP-43 pathology induced by proteasome inhibition in human motor neurons. Mislocalization of TDP-43 leads to STMN2 mis-splicing and increased cryptic exon expression. The addition of SEQ ID NO: 237 parent oligonucleotide reverses cryptic exon induction with high potency (IC50<5 nM). As shown in FIG. 23, increasing concentrations of SEQ ID NO: 237 (ranging from 5 nM up to 500 nM) significantly reduces the cryptic exon relative quantity.

In totality, this data establishes that the SEQ ID NO: 237 parent oligonucleotide protects against proteotoxic stress induction of cryptic exon expression. This is applicable in settings where neurons are to be protected from proteotoxic stress present in neurodegenerative disorders.

### Example 5: Dose Response Restoration of Full Length STMN2 mRNA and STMN2 Protein Using Stathmin-2 Cryptic Splicing Modulator

The experiment was performed as previously described in human neuroblastoma SY5Y cells. The cells were plated in 6-well or 96-well plates and cultured to 80% confluency. TDP-43 expression in cells were knocked down using an AON to TDP43 to express the cryptic exon, thus preventing transcription of full-length STMN2 (STMN2-FL) product. Cells were additionally co-transfected with a STMN2 oligonucleotide variant (specifically, SEQ ID NO: 1348) at varying doses (5 nM, 50 nM, 100 nM, 200 nM, and 500 nM). RNA and protein were isolated for QPCR and western blot assays.

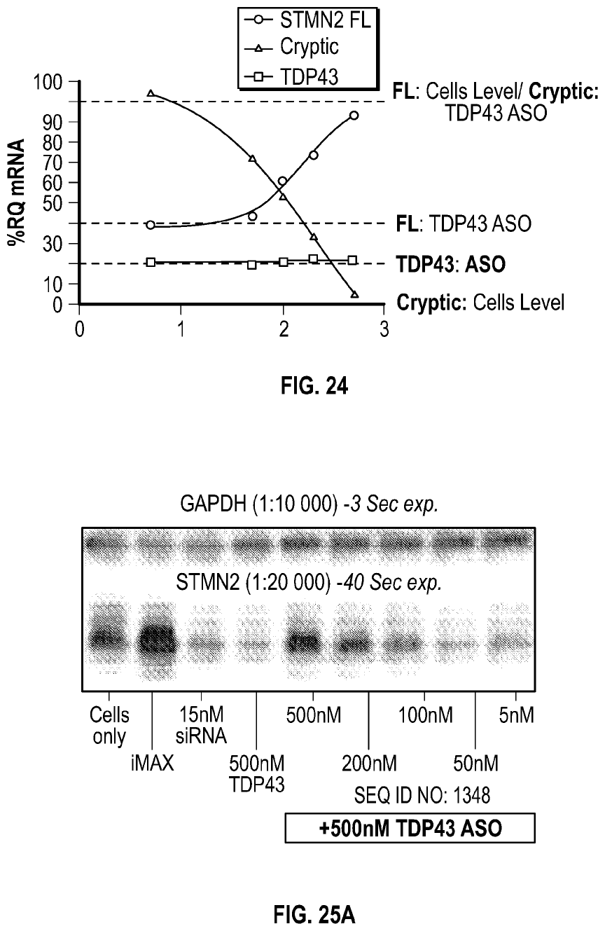

FIG. 24 shows the dose response curve illustrating increasing restoration of full length STMN2 transcript with increasing concentrations of STMN2 oligonucleotide variant with SEQ ID NO: 1348. Generally, increasing concentrations of the oligonucleotide increased full length STMN2 mRNA, decreased cryptic exon levels. Specifically, a 5 nM treatment of the STMN2 oligonucleotide variant resulted in −40% restoration of full length STMN2 transcript. A 500 nM treatment of the STMN2 oligonucleotide variant resulted in nearly 100% restoration of full length STMN2 transcript. Additionally, the 500 nM treatment of the STMN2 oligonucleotide variant resulted in the significant reduction (close to 0%) of cryptic exon.

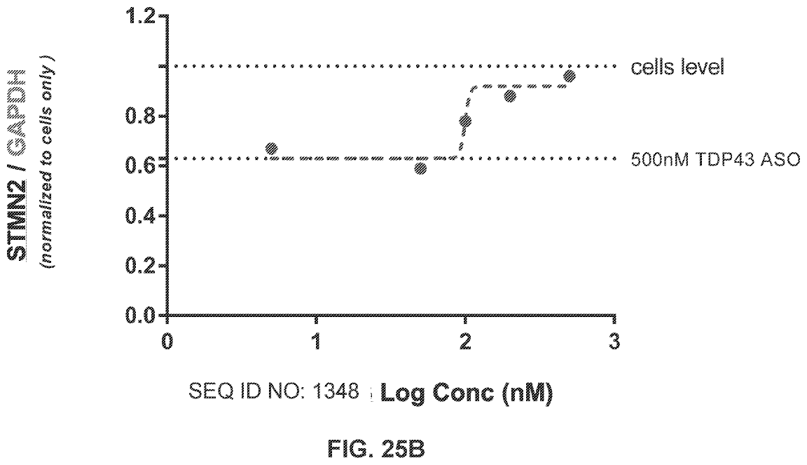

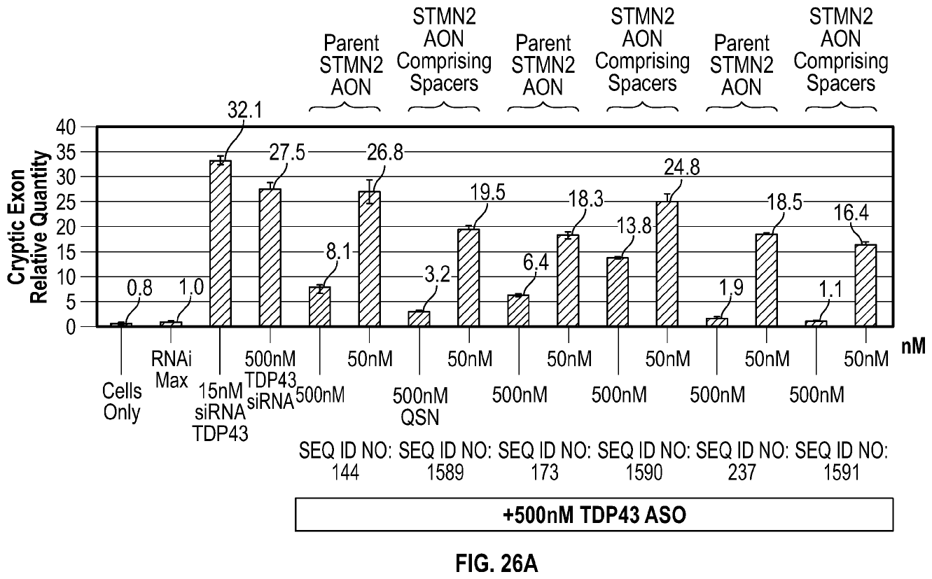

FIG. 25A shows a protein blot assay demonstrating the qualitative increase of full length STMN2 protein in response to higher concentrations of STMN2 oligonucleotide variant with SEQ ID NO: 1348. FIG. 25B shows the quantitated levels of full length STMN2 protein normalized to GAPDH in response to different concentrations of STMN2 oligonucleotide variant. Generally, both FIGS. 25A and 25B show that increasing concentrations of the STMN2 oligonucleotide variant resulted in increasing concentrations of full length STMN2 protein. Specifically, as shown in FIG. 25B, lower concentrations (5 nM and 50 nM) of the STMN2 oligonucleotide variant resulted in full length STMN2 protein concentrations that were −60% of the control group (cell only). Notably, the 500 nM treatment of the STMN2 oligonucleotide variant resulted in nearly 100% restoration of the full length STMN2 protein (in comparison to the cell only control group).

### Example 6: STMN2 AONs with Spacer Technology Restore Full Length STMN2 and Reduces STMN2 Transcripts with a Cryptic Exon

STMN2 AONs with two or three spacers were developed. Here, a spacer is represented by Formula (I), wherein:

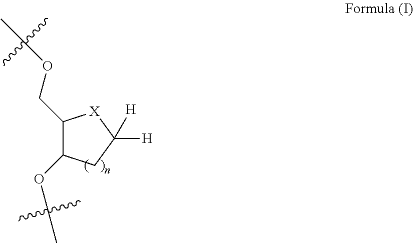

X is —O—; and

n is 1.

STMN2 AONs (e.g., STMN2 oligonucleotides each with two spacers) were tested in human motor neurons (hMN) for their ability to increase or restore full-length STMN2 mRNA (i.e., mRNA from which full-length STMN2 is translated) levels in TDP43 silenced cells. In some cases, STMN2 oligonucleotides are tested for their ability to reduce STMN2 transcripts with a cryptic exon. As described further below, the quantified percentage increase/restoration of STMN2-FL and/or percentage reduction of STMN2 transcripts with cryptic exon is described in reference to levels of STMN-FL and/or STMN2 transcripts with cryptic exon in a control group (e.g., cells treated with 500 nM TDP43 AON).

Three different STMN2 oligonucleotides with two spacers were generated. These three example STMN2 oligonucleotides are named 1) SEQ ID NO: 1589 (a 25mer with a first spacer at position 11 and a second spacer at position 22), 2) SEQ ID NO: 1590 (a 25mer with a first spacer at position 7 and a second spacer at position 14), and 3) SEQ ID NO: 1591 (a 25mer with a first spacer at position 8 and a second spacer at position 19). The STMN2 AONs are shown in Table 11.

TABLE 11
STMN2 AONs (including STMN2 parent
oligonucleotides and STMN2
oligonucleotides with two spacers)

| Sequence | |
|---|---|
| ID | |
| Number | Sequence (where S indicates |
| (SEQ ID | presence of a Spacer) |
| NO) | (5′ → 3′) |
| 144 | AATCCAATTAAGAGAGAGTGATGGG |
| 1589 | AATCCAATTASGAGAGAGTGASGGG |
| 173 | GAGTCCTGCAATATGAATATAATTT |
| 1590 | GAGTCCSGCAATASGAATATAATTT |
| 237 | GCACACATGCTCACACAGAGAGCCA |
| 1591 | GCACACASGCTCACACAGSGAGCCA |

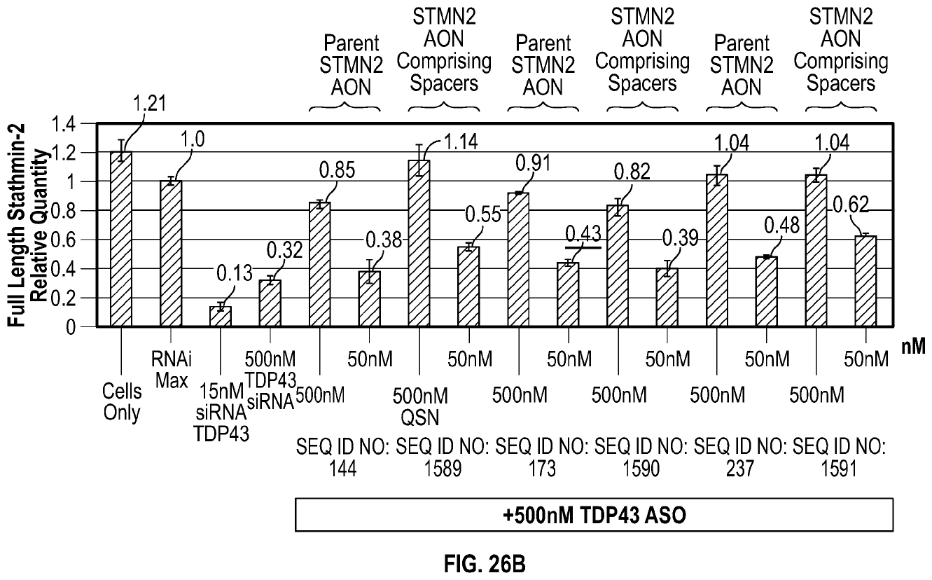

Referring to FIG. 26A, the quantity of STMN2 transcript with cryptic exon was increased more than 27-fold when treated with 500 nM TDP43 AON. A 500 nM treatment of a STMN2 parent oligonucleotide with SEQ ID NO: 144 reduced STMN2 transcript with cryptic exon levels by 71%. A 500 nM treatment of a STMN2 AON with SEQ ID NO: 1589 reduced STMN2 transcript with cryptic exon levels by 88%. Here, SEQ ID NO: 1589 exhibited further reduction of STMN2 transcripts with cryptic exon in comparison to SEQ ID NO: 144 (without two spacers.) A 500 nM treatment of a STMN2 parent oligonucleotide with SEQ ID NO: 173 reduced STMN2 transcript with cryptic exon levels by 77%. A 500 nM treatment of a STMN2 AON with SEQ ID NO: 1590 reduced STMN2 transcript with cryptic exon levels by 48%. A 500 nM treatment of a STMN2 parent oligonucleotide with SEQ ID NO: 237 reduced STMN2 transcript with cryptic exon levels by 93%. A 500 nM treatment of a STMN2 AON with SEQ ID NO: 1591 reduced STMN2 transcript with cryptic exon levels by 96%. Here, SEQ ID NO: 1591 exhibited similar reduction of STMN2 transcripts with cryptic exon in comparison to SEQ ID NO: 237 (without two spacers.)

Referring to FIG. 26B, STMN2-FL was decreased by 68% when treated with 500 nM TDP43 AON. A 500 nM treatment of a STMN2 parent oligonucleotide with SEQ ID NO: 144 increased STMN-FL levels by 165% (rescued to 85%). A 500 nM treatment of a STMN2 AON with SEQ ID NO: 1589 increased STMN-FL levels by 256% (rescued to 114%). Here, SEQ ID NO: 1589 exhibited improved restoration of STMN2 FL mRNA in comparison to SEQ ID NO: 144 (without two spacers.) A 500 nM treatment of a STMN2 parent oligonucleotide with SEQ ID NO: 173 increased STMN-FL levels by 184% (rescued to 91%). A 500 nM treatment of a STMN2 AON with SEQ ID NO: 1590 increased STMN-FL levels by 156% (rescued to 82%). Here, SEQ ID NO: 1590 exhibited similar restoration of STMN2 FL mRNA in comparison to SEQ ID NO: 173 (without two spacers.) A 500 nM treatment of a STMN2 parent oligonucleotide with SEQ ID NO: 237 increased STMN-FL levels by 225% (rescued to 104%). A 500 nM treatment of a STMN2 AON with SEQ ID NO: 1591 increased STMN-FL levels by 225% (rescued to 104%). Here, SEQ ID NO: 1591 exhibited similar restoration of STMN2 FL mRNA in comparison to SEQ ID NO: 237 (without two spacers.).

Additional example STMN2 AONs (including STMN2 AONs described above in Table 11) are shown below in Table 12. Specifically, Table 12 includes example STMN2 AONs with two spacers and STMN2 AONs with three spacers. Furthermore, Table 12 includes example STMN2 AON variants with one or more spacers that are shorter in length (e.g., 23mer, 21mer or 19mer) in comparison to STMN2 parent oligonucleotides described above in Table 11.

TABLE 12
STMN2 AONs with two or three spacers and
STMN2 AON variants with two spacers.

| Sequence | |
|---|---|
| ID | |
| Number | Sequence (where S indicates |
| (SEQ ID | presence of a |
| NO). | Spacer) (5’ → 3’) |
| 144 | AATCCAATTAAGAGAGAGTGATGGG |
| 1589 | AATCCAATTASGAGAGAGTGASGGG |
| 1592 | AATCCAASTAAGAGASAGTGATGSG |
| 1593 | AATCCAASTAAGAGASAGTGATSGG |
| 1594 | ASTCCAATTSAGAGAGASTGATGGG |
| 1417 | AATCCSATTASGAGAGAGSGATGGG |
| 1595 | TCCAATTSAGAGAGASTGATGGG |
| 173 | GAGTCCTGCAATATGAATATAATTT |
| 1590 | GAGTCCSGCAATASGAATATAATTT |
| 1596 | GAGTCCTSCAATATGSATATAATST |
| 1597 | GAGSCCTGCAASATGAATSTAATTT |
| 1418 | GAGTCCSGCAATASGAATATASTTT |
| 1598 | GTCCTGCSATATGAASATAAT |
| 1599 | GTCCTSCAATATGSATATAAT |
| 1419 | GTCCSGCAATASGAATATA |
| 237 | GCACACATGCTCACACAGAGAGCCA |
| 1591 | GCACACASGCTCACACAGSGAGCCA |
| 1600 | GCACACASGCTCACASAGAGAGSCA |
| 1601 | GCSCACATGSTCACACASAGAGCCA |
| 1420 | GCACACASGCTCACASAGAGSGCCA |
| 1602 | GCACACASGCTCACASAGAGAGC |
| 1603 | AATSCAATTAAGAGSGAGTGATGGG |
| 1604 | AATCCAATTASGAGAGAGTGSTGGG |
| 1605 | AATCCASTTAAGAGAGAGSGATGGG |
| 1606 | AATCCASTTAAGAGAGASTGATGGG |
| 1607 | AATCCAASTAAGSGAGASTGATGGG |
| 1608 | GAGSCCTGCAATATSAATATAATTT |
| 1609 | GAGTCCTGCASTATGAATATSATTT |
| 1610 | GAGTCCSGCAATATGAATSTAATTT |
| 1611 | GAGTCCSGCAATATGAASATAATTT |
| 1612 | GAGTCCTSCAATSTGAASATAATTT |
| 1613 | GAGTCCSGCAATSTGAATSTAATTT |
| 1614 | GAGTCSTGCAATSTGAATASAATTT |
| 1615 | GAGTSCTGCAATSTGAATATSATTT |
| 1616 | GTCCTGCSATATGSATATAAT |
| 1617 | CCTTTCTCSCGAAGGTCTTCSGCCG |
| 1618 | CTTTCTCSCGAAGGTSTTCTGCC |
| 1619 | TTTCTCTSGAAGGTCSTCTGCCG |
| 1664 | GCACACASGCSCACACAGSGAGCCA |
| 1621 | GCACACASGCTCSCACASAGAGCCA |

Table 13 depicts the performance of STMN2 AONs, including STMN2 AONs with two or three spacers.

STMN2 AONs that included two spacers increased levels of STMN2-FL. For example, at a dose of 200 nM ASO, SEQ ID NO: 1608 and SEQ ID NO: 1609 increased levels of STMN-FL to 0.65 and 0.78, respectively. Additionally, at a dose of 200 nM ASO, SEQ ID NO: 1610 and SEQ ID NO: 1611 increased levels of STMN-FL to 0.95 and 1.09, respectively. Notably, a number of STMN2 AONs increased levels of STMN-FL to a lesser extent. Specifically, at a 200 nM dose of STMN2 AON, SEQ ID NO: 1612, SEQ ID NO: 1613, SEQ ID NO: 1614, and SEQ ID NO: 1615 increased levels of STMN-FL to between 0.10 and 0.20.

At a dose of 200 nM AON, all STMN2 AON derived from SEQ ID NO: 197 significantly increased levels of STMN-FL. Specifically, SEQ ID NO: 1617, SEQ ID NO: 1618, and SEQ ID NO: 1619 increased levels of STMN-FL to 0.99, 0.94, and 1.00, respectively.

Altogether, these results demonstrate that different STMN2 AONs including two spacers are capable of increasing STMN-FL to levels that are close or comparable to their non-spacer counterparts (e.g., SEQ ID NO: 173 or SEQ ID NO: 197).

The differences in performance between STMN2 AONs derived from SEQ ID NO: 173, including SEQ ID NO: 1612, SEQ ID NO: 1613, SEQ ID NO: 1614, and SEQ ID NO: 1615 and STMN2 AONs derived from SEQ ID NO: 197 including SEQ ID NO: 1617, SEQ ID NO: 1618, and SEQ ID NO: 1619 may be attributable to GC content in the respective STMN2 AONs. Specifically, as shown in Table 13, STMN2 AONs derived from SEQ ID NO: 173 had below 30% GC content, which may lead to their reduced performance. In contrast, as shown in Table 13, STMN2 AONs derived from SEQ ID NO: 197 had above 40% GC content. Thus, including two or more spacers in a higher GC content AON may be preferable.

In addition to GC content, the location of spacers relative to guanine and cytosine nucleobases can also impact the performance of the STMN2 AON. For example, at a 200 nM AON dose, SEQ ID NO: 1615, SEQ ID NO: 1596, and SEQ ID NO: 1597 increased levels of STMN2-FL to 0.12, 0.26, and 0.29. Each of these STMN2 AONs have three spacers. In comparison, at a 200 nM AON dose, SEQ ID NO: 1418 increased levels of STMN2-FL to 0.73. SEQ ID NO: 1418 includes spacers that are positioned to maximize the number of spacers that are immediately preceding a guanine base. Specifically, the first and second spacers of SEQ ID NO: 1418 each respectively precede a guanine base. Thus, maximizing the number of spacers in a STMN2 AON that immediately precede a guanine base can improve the performance of the STMN2 AON.

TABLE 13
Performance of varying STMN2 AONs, including
STMN2 AONs with two or three spacers.

| | | Rela- | Rela- | |
|---|---|---|---|---|
| | | tive | tive | |
| | | Quan- | Quan- | |
| | | tity | tity | |
| | | of | of | |
| | | STMN- | STMN- | |
| Se- | | FL in | FL in | |
| quence | | re- | re- | |
| ID | | sponse | sponse | |
| No. | Sequence (where | to 200 | to 50 | |
| (SEQ | S indicates | nM ASO | nM ASO | GC |
| ID | presence of a | Treat- | treat- | con- |
| NO) | Spacer) (5′ → 3′) | ment | ment | tent |
| 169 | CCTGCAATATGAATATAATTTTAAA | 0.73 | 0.45 | 20% |
| 1421 | CCTGCAATATGAATATAATTTTA | 1.19 | 0.48 | 22% |
| 1422 | TGCAATATGAATATAATTTTAAA | 0.85 | 0.63 | 13% |
| 1423 | CTGCAATATGAATATAATTTTAA | 0.93 | 0.69 | 17% |
| 1424 | TGCAATATGAATATAATTTTA | 0.8 | 0.44 | 14% |
| 170 | TCCTGCAATATGAATATAATTTTAA | 1.01 | 0.46 | 20% |
| 1425 | TCCTGCAATATGAATATAATTTT | 0.83 | 0.49 | 22% |
| 1426 | CTGCAATATGAATATAATTTT | 0.83 | 0.57 | 19% |
| 171 | GTCCTGCAATATGAATATAATTTTA | 0.89 | 0.41 | 24% |
| 1346 | GTCCTGCAATATGAATATAATTT | 1.1 | 1.13 | 26% |
| 1355 | CCTGCAATATGAATATAATTT | 0.82 | 0.44 | 24% |
| 172 | AGTCCTGCAATATGAATATAATTTT | 0.79 | 0.45 | 24% |
| 1427 | AGTCCTGCAATATGAATATAATT | 0.89 | 0.52 | 26% |
| 1428 | TCCTGCAATATGAATATAATT | 1.18 | 0.66 | 24% |
| 252 | CTCTCTCGCACACACGCACACATGC | 0.67 | 0.43 | 60% |
| 1432 | CTCTCGCACACACGCACACATGC | 0.67 | 0.52 | 61% |
| 1433 | CTCTCTCGCACACACGCACACAT | 0.63 | 0.24 | 57% |
| 1434 | TCTCTCGCACACACGCACACATG | 0.73 | 0.45 | 57% |
| 1435 | CTCTCGCACACACGCACACAT | 0.84 | 0.36 | 57% |
| 173 | GAGTCCTGCAATATGAATATAATTT | 1.12 | 0.6 | 28% |
| 1608 | GAGSCCTGCAATATSAATATAATTT | 0.65 | 0.19 | 24% |
| 1609 | GAGTCCTGCASTATGAATATSATTT | 0.78 | 0.25 | 28% |
| 1610 | GAGTCCSGCAATATGAATSTAATTT | 0.95 | 0.43 | 28% |
| 1611 | GAGTCCSGCAATATGAASATAATTT | 1.09 | 0.32 | 28% |
| 1612 | GAGTCCTSCAATSTGAASATAATTT | 0.15 | 0.08 | 24% |
| 1613 | GAGTCCSGCAATSTGAATSTAATTT | 0.2 | 0.13 | 28% |
| 1614 | GAGTCSTGCAATSTGAATASAATTT | 0.13 | 0.18 | 24% |
| 1615 | GAGTSCTGCAATSTGAATATSATTT | 0.12 | 0.12 | 24% |
| 1596 | GAGTCCTSCAATATGSATATAATST | 0.26 | 0.13 | 24% |
| 1597 | GAGSCCTGCAASATGAATSTAATTT | 0.29 | 0.17 | 28% |
| 1418 | GAGTCCSGCAATASGAATATASTTT | 0.73 | 0.24 | 28% |
| 1598 | GTCCTGCSATATGAASATAAT | 0.72 | 0.31 | 29% |
| 1599 | GTCCTSCAATATGSATATAAT | 0.1 | 0.16 | 24% |
| 1616 | GTCCTGCSATATGSATATAAT | 0.77 | 0.23 | 29% |
| 197 | CCTTTCTCTCGAAGGTCTTCTGCCG | 1.04 | 0.44 | 56% |
| 1429 | TTTCTCTCGAAGGTCTTCTGCCG | 1.35 | 1.06 | 48% |
| 1430 | CCTTTCTCTCGAAGGTCTTCTGC | 0.98 | 0.44 | 48% |
| 1431 | CTTTCTCTCGAAGGTCTTCTGCC | 1.33 | 0.55 | 48% |
| 1617 | CCTTTCTCSCGAAGGTCTTCSGCCG | 0.99 | 0.69 | 56% |
| 1618 | CTTTCTCSCGAAGGTSTTCTGCC | 0.94 | 0.58 | 48% |
| 1619 | TTTCTCTSGAAGGTCSTCTGCCG | 1 | 0.54 | 48% |

### Example 7: Additional Experiments Demonstrate STMN2 AONs with Spacer Technology Restore Full Length STMN2 and Reduces STMN2 Transcripts with a Cryptic Exon

STMN2 AONs with one, two, or three spacers were developed. Generally, in this Example, except for SEQ ID NO: 1649 described below, a spacer is represented by Formula (I), wherein:

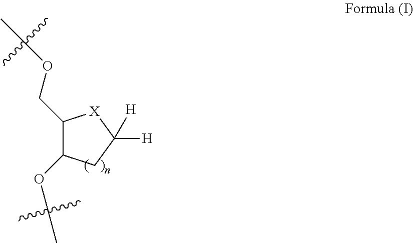

X is —O—, and

n is 1.

For SEQ ID NO: 1649, each spacer included in the ASO is represented by Formula (I), wherein:

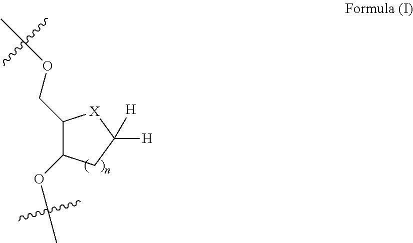

X is —O—, and

n is 2.

STMN2 AONs with spacers were characterized and compared to STMN2 AON without spacer counterparts. Specifically, the melting temperature of STMN2 AON with and without spacers were determined to demonstrate the structural differences of the STMN2 AONs. Table 14 shows the different melting temperatures of STMN2 AONs across two different replicates. STMN2 AONs with two spacers exhibited a lower melting temperature (approximately 10° C. lower) in comparison to STMN2 AONs without spacers.

TABLE 14
Melting temperatures of STMN2 AONs with and without spacers.

| ASO + RNA | Tm (° C.) | Tm (° C.) | ΔTm ° C. | ΔTm ° C. | |
|---|---|---|---|---|---|
| target (25bases) | Replicate 1 | Replicate 2 | Replicate 1 | Replicate 2 | % GC |
| SEQ ID NO: 237 | 86.6 | 86.5 | 11.6 | 11.4 | 56 |
| (no spacer) | | | | | |
| SEQ ID NO: 1591 | 75.0 | 75.1 | | | |
| (2 spacers) | | | | | |
| SEQ ID NO: 144 | 75.5 | 75.5 | 9.5 | 9.7 | 40 |
| (no spacer) | | | | | |
| SEQ ID NO: 1589 | 66.0 | 65.8 | | | |
| (2 spacers) | | | | | |
| SEQ ID NO: 173 | 71.2 | 71.1 | 13.5 | 13.5 | 28 |
| (no spacer) | | | | | |
| SEQ ID NO: 1590 | 57.7 | 57.6 | | | |
| (2 spacers) | | | | | |

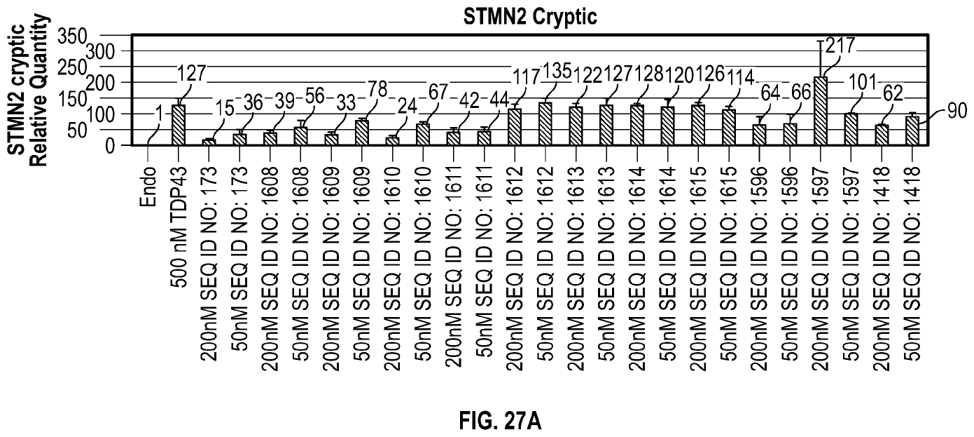

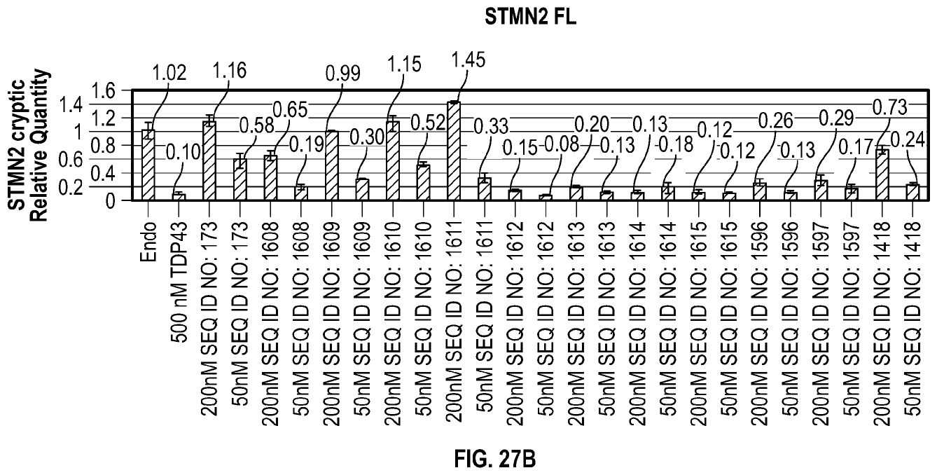

STMN2 AONs (e.g., STMN2 oligonucleotides with one, two, or three spacers) were tested for their ability to increase or restore full-length STMN2 mRNA (i.e., mRNA from which full-length STMN2 is translated) levels in TDP43 silenced cells. In some cases, STMN2 oligonucleotides are tested for their ability to reduce STMN2 transcripts with a cryptic exon. FIGS. 27-35 show effects of STMN2 AONs with spacers in increasing full-length STMN2 mRNA (“STMN2 FL”) and/or in reducing STMN2 transcripts with a cryptic exon (“STMN2 cryptic”). Furthermore, Table 15 identifies the respective STMN2 AONs as well as their respective performances. Treatment groups are identified on the X-axis of FIGS. 27-35 and include the concentration of specific AON sequences. Here, specific AON sequences are labeled according to their corresponding SEQ ID NO.

FIG. 27A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418. FIG. 27B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418. Generally, FIGS. 27A and 27B demonstrate the performance of STMN2 AONs with spacers (e.g., SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418) in comparison to STMN2 AON without spacers (SEQ ID NO: 173). Here, a number of STMN2 AON with spacers perform as well, or outperform the STMN2 AON without spacers (SEQ ID NO: 173). Specifically, 200 nM of SEQ ID NO: 1609, SEQ ID NO: 1610, and SEQ ID NO: 1611 achieve comparable levels of STMN2 transcript with cryptic exon mRNA levels and STMN2 full-length mRNA levels in the presence of TDP43 in comparison to STMN2 AON without spacers (SEQ ID NO: 173).

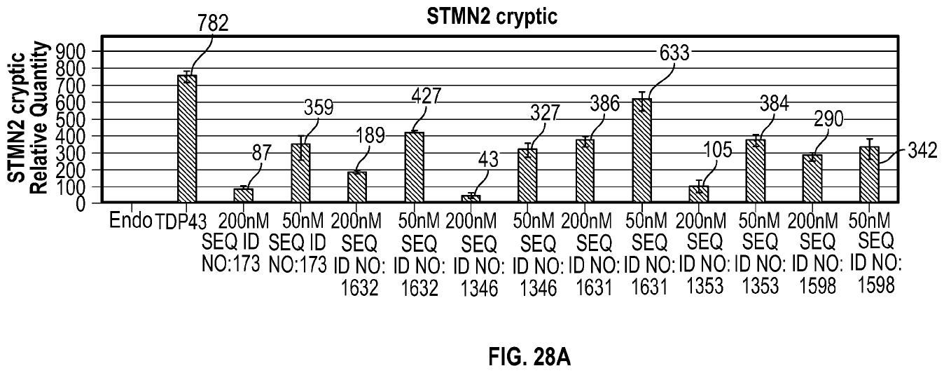

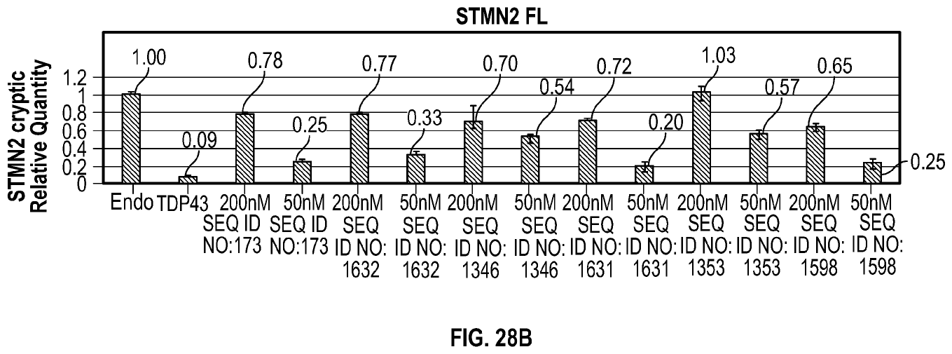

FIG. 28A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598. FIG. 28B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598. Generally, FIGS. 28A and 28B demonstrate the performance of STMN2 AONs with spacers (e.g., SEQ ID NO: 1632, SEQ ID NO: 1631, and SEQ ID NO: 1598) in comparison to their STMN2 AON counterparts without spacer (e.g., SEQ ID NO: 173, SEQ ID NO: 1346, and SEQ ID NO: 1353). Here, a 50 nM or 200 nM dose of SEQ ID NO: 1632 achieves comparable levels of STMN2 transcript with cryptic exon mRNA levels and STMN2 full-length mRNA levels in the presence of TDP43 in comparison to a 50 nM or 200 nM dose of the STMN2 AON counterpart without spacers (SEQ ID NO: 173). A 200 nM dose of SEQ ID NO: 1631 achieves comparable levels of STMN2 full-length mRNA levels in the presence of TDP43 in comparison to 200 nM dose of the STMN2 AON counterpart without spacers (SEQ ID NO: 1346).

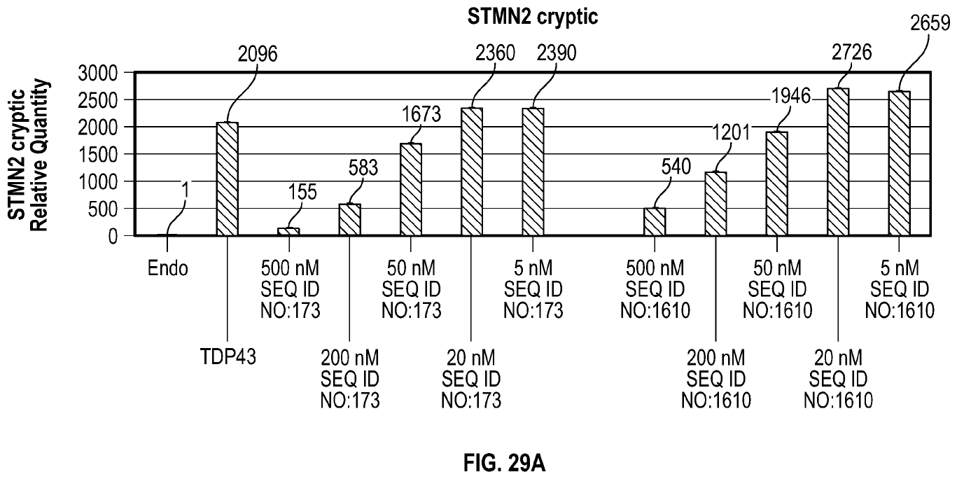

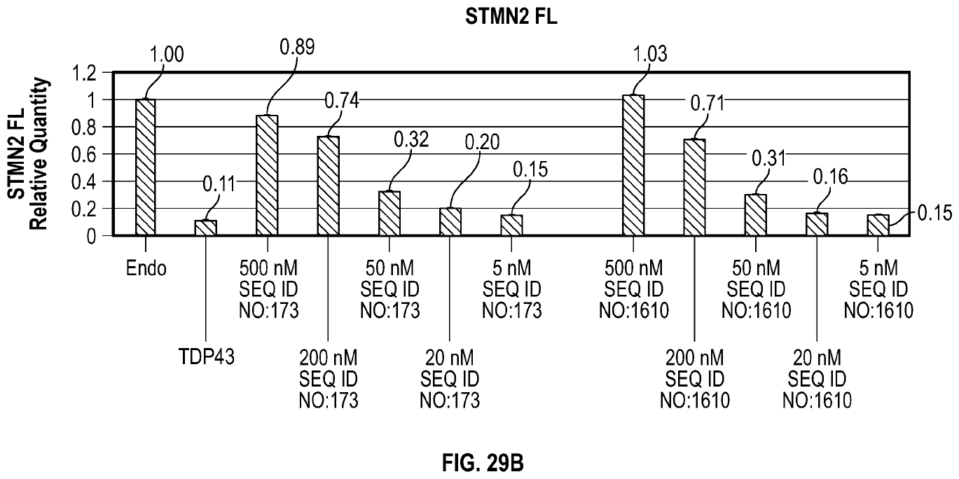

FIG. 29A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173 and SEQ ID NO: 1610. FIG. 29B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173 and SEQ ID NO: 1610. Generally, FIGS. 29A and 29B demonstrate the performance of STMN2 AONs with spacers (e.g., SEQ ID NO: 1610) in comparison to the STMN2 AON counterpart without spacers (e.g., SEQ ID NO: 173). Across the different doses (e.g., 500 nM, 200 nM, 50 nM, 20 nM, and 5 nM), SEQ ID NO: 1610 achieves comparable levels of STMN2 transcript with cryptic exon mRNA levels and STMN2 full-length mRNA levels in the presence of TDP43 in comparison to the STMN2 AON counterpart without spacers (SEQ ID NO: 173).

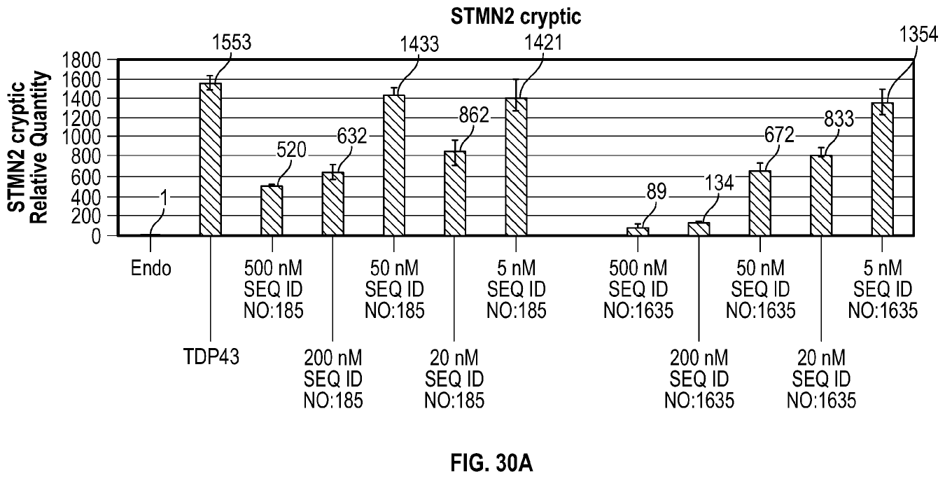

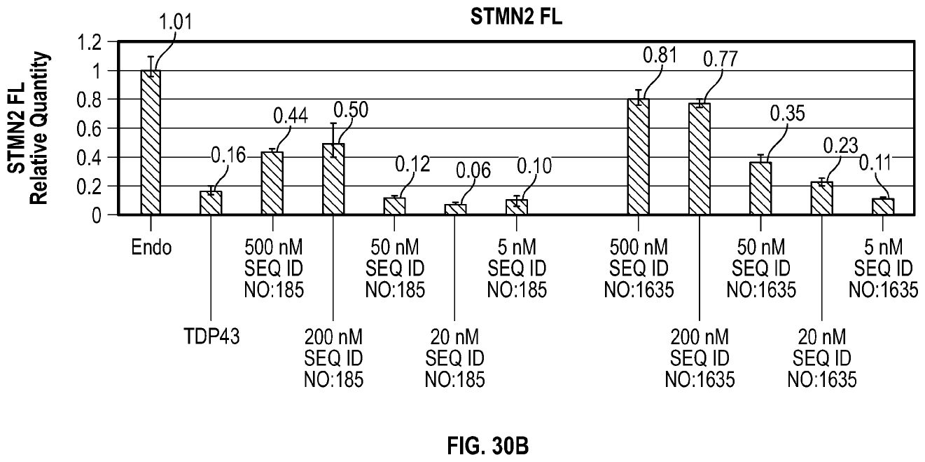

FIG. 30A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 185 and SEQ ID NO: 1635. FIG. 30B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 185 and SEQ ID NO: 1635. Generally, FIGS. 30A and 30B demonstrate the performance of STMN2 AONs with spacers (e.g., SEQ ID NO: 1635) in comparison to the STMN2 AON counterpart without spacers (e.g., SEQ ID NO: 185). Across the different doses (e.g., 500 nM, 200 nM, 50 nM, 20 nM, and 5 nM), SEQ ID NO: 1610 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 185).

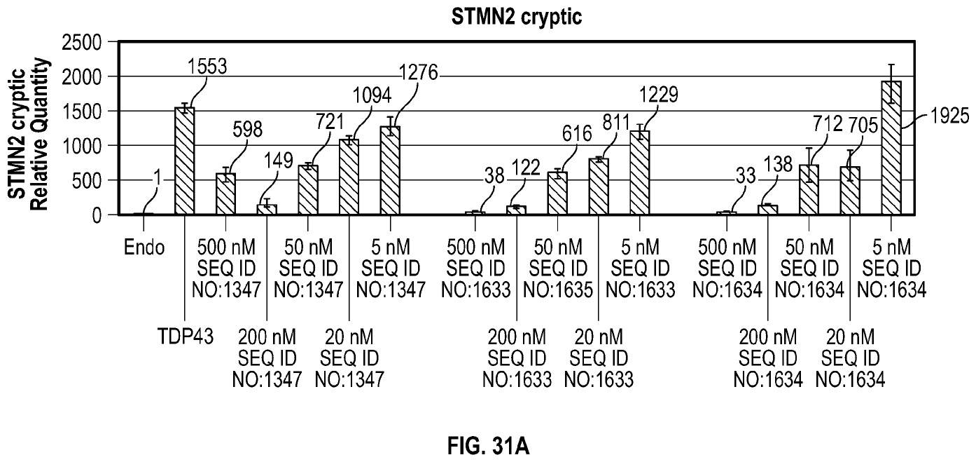

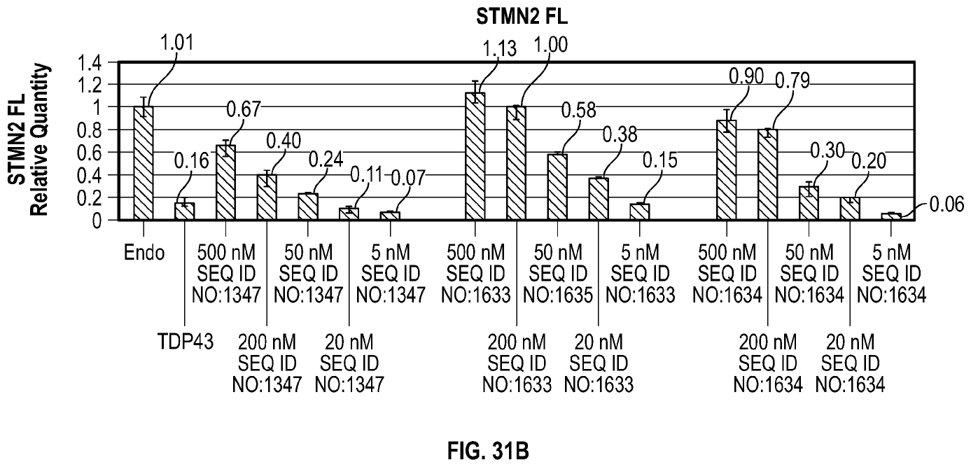

FIG. 31A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1347, SEQ ID NO: 1633, and SEQ ID NO: 1634. FIG. 31B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 1347, SEQ ID NO: 1633, and SEQ ID NO: 1634. Generally, FIGS. 31A and 31B demonstrate the performance of STMN2 AONs with spacers (e.g., SEQ ID NO: 1633 and SEQ ID NO: 1634) in comparison to the STMN2 AON counterpart without spacers (e.g., SEQ ID NO: 1347). Across the different doses (e.g., 500 nM, 200 nM, 50 nM, 20 nM, and 5 nM), SEQ ID NO: 1633 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 1347). Similarly, across the different doses (e.g., 500 nM, 200 nM, 50 nM, 20 nM, and 5 nM), SEQ ID NO: 1634 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 1347).

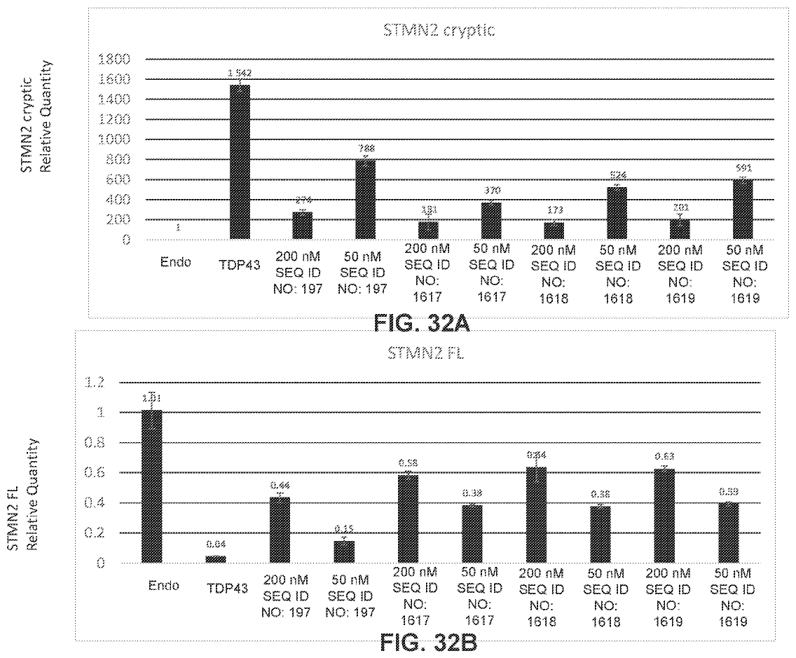

FIG. 32A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 197, SEQ ID NO: 1617, SEQ ID NO: 1618, and SEQ ID NO: 1619. FIG. 32B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 197, SEQ ID NO: 1617, SEQ ID NO: 1618, and SEQ ID NO: 1619. Generally, FIGS. 32A and 32B demonstrate the performance of STMN2 AONs with spacers (e.g., SEQ ID NO: 1617, SEQ ID NO: 1618, and SEQ ID NO: 1619) in comparison to the STMN2 AON counterpart without spacers (e.g., SEQ ID NO: 197). At a 50 nM or 200 nM dose, SEQ ID NO: 1617 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197). At a 50 nM or 200 nM dose, SEQ ID NO: 1618 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197). At a 50 nM or 200 nM dose, SEQ ID NO: 1619 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197).

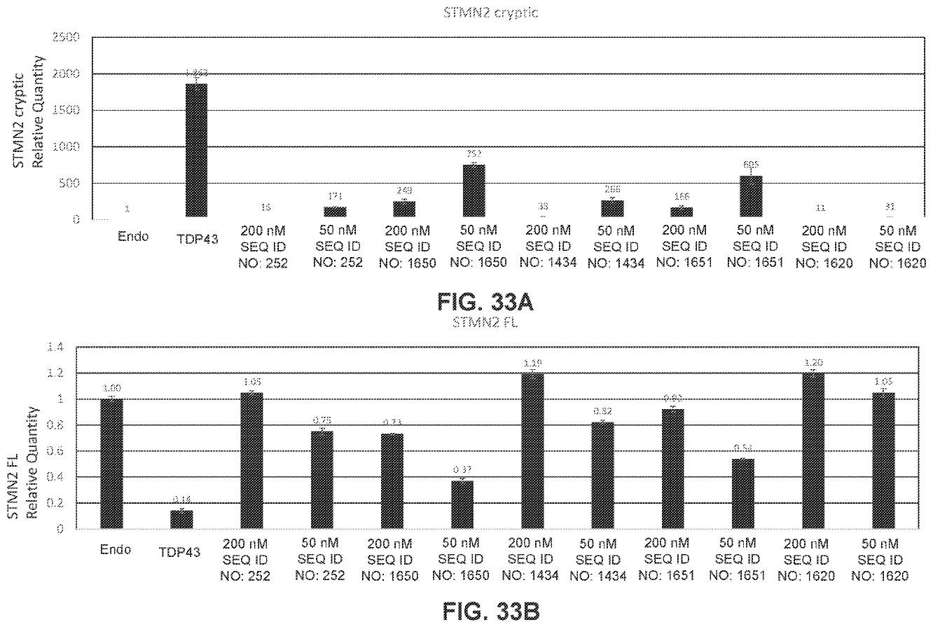

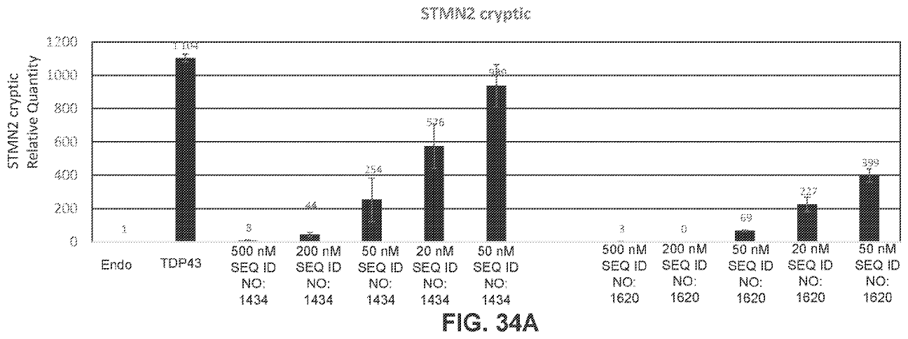

FIG. 33A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 252, SEQ ID NO: 1650, SEQ ID NO: 1434, SEQ ID NO: 1651, and SEQ ID NO: 1620. FIG. 33B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 252, SEQ ID NO: 1650, SEQ ID NO: 1434, SEQ ID NO: 1651, and SEQ ID NO: 1620. Generally, FIGS. 33A and 33B demonstrate the performance of STMN2 AONs with spacers (e.g., SEQ ID NO: 1620) in comparison to the STMN2 AON counterpart without spacers (e.g., SEQ ID NO: 252, SEQ ID NO: 1650, SEQ ID NO: 1434, and SEQ ID NO: 1651). Ata 50 nM or 200 nM dose, SEQ ID NO: 1620 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterparts without spacers (SEQ ID NO: 252, SEQ ID NO: 1650, SEQ ID NO: 1434, and SEQ ID NO: 1651).

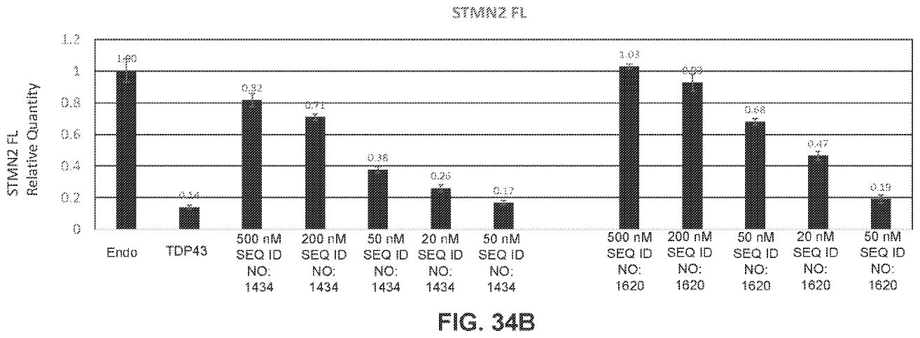

FIG. 34A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1434 and SEQ ID NO: 1620. FIG. 34B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 1434 and SEQ ID NO: 1620. Generally, FIGS. 34A and 34B demonstrate the performance of STMN2 AONs with spacers (e.g., SEQ ID NO: 1620) in comparison to the STMN2 AON counterpart without spacers (e.g., SEQ ID NO: 1434). Across the different doses (e.g., 500 nM, 200 nM, 50 nM, 20 nM, and 5 nM), SEQ ID NO: 1620 achieves reduced levels of STMN2 transcript with cryptic exon mRNA levels and increased STMN2 full-length mRNA levels in the presence of TDP43 in comparison to the STMN2 AON counterpart without spacers (SEQ ID NO: 1434).

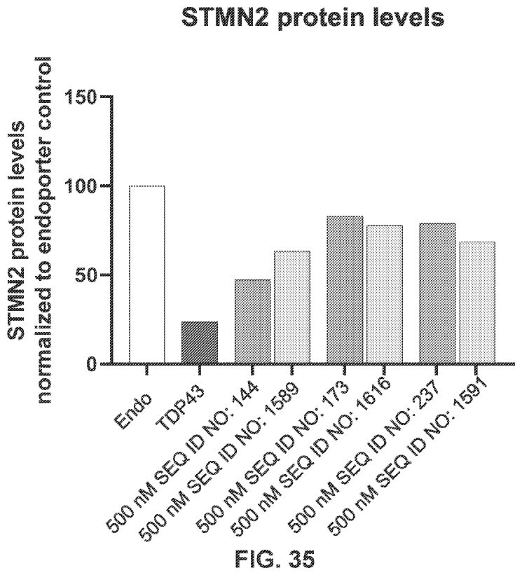

FIG. 35 is a bar graph showing normalized STMN2 protein levels following treatment with TDP43 antisense and restoration using 500 nM STMN2 AONs including SEQ ID NO: 144, SEQ ID NO: 1589, SEQ ID NO: 173, SEQ ID NO: 1616, SEQ ID NO: 237, and SEQ ID NO: 1591. Generally, FIG. 35 demonstrates the performance of STMN2 AONs with spacers (e.g., SEQ ID NO: 1589, SEQ ID NO: 1616, and SEQ ID NO: 1591) in comparison to their STMN2 AON counterparts without spacers (e.g., SEQ ID NO: 144, SEQ ID NO: 173, SEQ ID NO: 237). Generally, STMN2 AONs with spacers are able to achieve comparable levels of STMN2 protein levels in comparison to their STMN2 AON counterparts. Specifically, SEQ ID NO: 1589 achieves comparable levels of STMN2 protein levels in comparison to SEQ ID NO: 144. SEQ ID NO: 1616 achieves comparable levels of STMN2 protein levels in comparison to SEQ ID NO: 173. SEQ ID NO: 1591 achieves comparable levels of STMN2 protein levels in comparison to SEQ ID NO: 237.

Referring to Tables 15 and 17, they show the performance of STMN2 AONs with spacers (e.g., Table 15) and performance of STMN2 AONs without spacers (e.g., Table 16) in human motor neurons. RT-qPCR results for STMN2 full-length transcript provided in Tables 15 and 17 are normalized values using the equation ((RQASO-RQTDP43)/(Rqendo-RQTDP43))*100 where RQ refers to Relative Quantity described above. RT-qPCR results for STMN2 transcript with a cryptic exon provided in Tables 15 and 17 are normalized values using the equation (1-((RQASO-RQTDP43)/(Rqendo-RQTDP43)))*100 where RQ refers to Relative Quantity described above. Each RT-qPCR experiment was run in triplicate wells and performed N number of independent replicate runs. Standard deviation or SD is calculated as the SD between each run. Where N=1, SD was reported as the standard deviation between the triplicate well results in the single experiment. Notably, as shown in Table 15, a 200 nM dose of SEQ ID NO: 1631 (GTCCTGCSATATGAASATAATTT with two spacers) rescued full length STMN2 mRNA to 69% and reduced STMN2 transcript with cryptic exon levels to 49% (reduced by 51%).

Additionally, as shown in Table 15, a 200 nM dose of SEQ ID NO: 1633 (GTCTTCTSCCGAGTCSTGCAATA with two spacers) rescued full length STMN2 mRNA to 83% and reduced STMN2 transcript with cryptic exon levels to 10% (reduced by 90%). Comparatively, as shown in Table 16, a 200 nM dose of SEQ ID NO: 1347 (GTCTTCTGCCGAGTCCTGCAATA with no spacers) rescued full length STMN2 mRNA to 40.2% and reduced STMN2 transcript with cryptic exon levels to 20.8% (reduced by 80.2%). This indicates that the addition of spacers improves the performance of SEQ ID NO: 1633 in comparison to the STMN2 AON counterpart without spacers (e.g., SEQ ID NO: 1347).

Additionally, as shown in Table 15, a 200 nM dose of SEQ ID NO: 1618 (CTTTCTCSCGAAGGTSTTCTGCC with two spacers) rescued full length STMN2 mRNA to 82% and reduced STMN2 transcript with cryptic exon levels to 11% (reduced by 89%). A 200 nM dose of SEQ ID NO: 1619 (TTTCTCTSGAAGGTCSTCTGCCG with two spacers) rescued full length STMN2 mRNA to 80% and reduced STMN2 transcript with cryptic exon levels to 12% (reduced by 88%). Comparatively, as shown in Table 16, a 200 nM dose of SEQ ID NO: 197 (CCTTTCTCTCGAAGGTCTTCTGCCG with no spacers) rescued full length STMN2 mRNA to 79.3% and reduced STMN2 transcript with cryptic exon levels to 12.1% (reduced by 87.9%). Here, at 200 nM dose, the performance of STMN2 AONs with two spacers (e.g., SEQ ID NO: 1618 and SEQ ID NO: 1619) is comparable to the STMN2 AON counterpart without spacers (e.g., SEQ ID NO: 197). Notably, at a 50 nM dose, the performance of STMN2 AONs with two spacers (e.g., SEQ ID NO: 1618 and SEQ ID NO: 1619) is improve din comparison to the STMN2 AON counterpart without spacers (e.g., SEQ ID NO: 197). Specifically, at the 50 nM dose, SEQ ID NO: 1618 rescued full length STMN2 mRNA to 46% and SEQ ID NO: 1619 rescued full length STMN2 mRNA to 42% whereas SEQ ID NO: 197 (without spacers) rescued full length STMN2 mRNA to 26.7%.

Additionally, as shown in Table 15, a 200 nM dose of SEQ ID NO: 1620 (TCTCTCGSACACACGSACACATG with two spacers) rescued full length STMN2 mRNA to 103% and reduced STMN2 transcript with cryptic exon levels to 1% (reduced by 99%). A 50 nM dose of SEQ ID NO: 1620 rescued full length STMN2 mRNA to 74% and reduced STMN2 transcript with cryptic exon levels to 5% (reduced by 95%). Comparatively, as shown in Table 16, a 200 nM dose and 50 nM dose of SEQ ID NO: 1434 (TCTCTCGCACACACGCACACATG with no spacers) rescued full length STMN2 mRNA to 77.5% and 16.6%, respectively and reduced STMN2 transcript with cryptic exon levels to 2.7% (reduced by 97.3%) and 18.3% (reduced by 81.7%), respectively. This indicates that the addition of spacers improves the performance of SEQ ID NO: 1620 in comparison to the STMN2 AON counterpart without spacers (e.g., SEQ ID NO: 1434).

TABLE 15
Performance of STMN2 AONs (STMN2 oligonucleotides with one,
two, or three spacers).

| | | QPCR potency in | | | | | | QPCR potency in | | | | | |
|---|---|---|---|---|---|---|---|---|---|---|---|---|---|
| SEQ | Sequence (where S indi- | hMN STMN2 FL | | | | | | hMN STMN2 cryptic | | | | | |
| ID | cates presence of a | 50 nM | | | 200 nM | | | 50 nM | | | 200 nM | | |
| NO: | Spacer) (5′ □ 3′) | N | Mean | SD | N | Mean | SD | N | Mean | SD | N | Mean | SD |
| 1622 | TGCAATASGAATATASTTTTAAA | 1 | 1 | 1 | 1 | 47 | 12 | 1 | 76 | 24 | 1 | 340 | 92 |
| 1623 | TCCTGCASTATGAATSTAATTTT | 1 | 6 | 3 | 1 | 31 | 16 | 1 | 82 | 17 | 1 | 100 | 44 |
| 1624 | CTGCAATATGSATATAATTTT | 1 | 5 | 7 | 1 | 11 | 5 | 1 | 104 | 25 | 1 | 61 | 10 |
| 1625 | CTGCAATSTGAATATSATTTTAA | 1 | 2 | 8 | 1 | -4 | 2 | 1 | 116 | 15 | 1 | 147 | 9 |
| 1626 | CCTGCAATATSAATATAATTT | 1 | 2 | 2 | 1 | 42 | 7 | 1 | 65 | 5 | 1 | 59 | 14 |
| 1627 | TCCTGCAATASGAATATAATT | 1 | 19 | 5 | 1 | 65 | 1 | 1 | 85 | 17 | 1 | 36 | 2 |
| 1628 | GTCCTGCSATATGAASATAAT | 5 | 20 | 6 | 5 | 65 | 9 | 5 | 79 | 28 | 5 | 45 | 5 |
| 1629 | GTCCTSCAATATGSATATAAT | 4 | 5 | 6 | 4 | 13 | 22 | 4 | 119 | 31 | 4 | 133 | 53 |
| 1630 | GTCCTGCSATATGSATATAAT | 4 | 16 | 9 | 4 | 71 | 23 | 4 | 97 | 23 | 4 | 51 | 17 |
| 1631 | GTCCTGCSATATGAASATAATTT | 1 | 13 | 9 | 1 | 69 | 3 | 1 | 81 | 10 | 1 | 49 | 4 |
| 1596 | GAGTCCTSCAATATGSATATAATST | 1 | 3 | 4 | 1 | 18 | 9 | 1 | 52 | 41 | 1 | 50 | 41 |
| 1597 | GAGSCCTGCAASATGAATSTAATTT | 1 | 7 | 11 | 1 | 20 | 15 | 1 | 79 | 1 | 1 | 82 | 24 |
| 1418 | GAGTCCSGCAATASGAATATASTTT | 1 | 15 | 2 | 1 | 69 | 13 | 1 | 70 | 23 | 1 | 48 | 8 |
| 1632 | GAGTCCTGCAATATSAATATAATTT | 1 | 27 | 5 | 1 | 75 | 6 | 1 | 55 | 1 | 1 | 24 | 2 |
| 1608 | GAGSCCTGCAATATSAATATAATTT | 1 | 10 | 8 | 1 | 60 | 15 | 1 | 44 | 34 | 1 | 30 | 14 |
| 1609 | GAGTCCTGCASTATGAATATSATTT | 3 | 17 | 7 | 3 | 70 | 25 | 3 | 67 | 16 | 3 | 42 | 15 |
| 1610 | GAGTCCSGCAATATGAATSTAATTT | 4 | 29 | 11 | 4 | 83 | 21 | 4 | 76 | 20 | 4 | 40 | 17 |
| 1611 | GAGTCCSGCAATATGAASATAATTT | 3 | 23 | 3 | 3 | 95 | 46 | 3 | 60 | 22 | 3 | 41 | 8 |
| 1612 | GAGTCCTSCAATSTGAASATAATTT | 1 | -2 | 2 | 1 | 5 | 3 | 1 | 106 | 26 | 1 | 92 | 22 |
| 1613 | GAGTCCSGCAATSTGAATSTAATTT | 1 | 3 | 2 | 1 | 11 | 3 | 1 | 100 | 37 | 1 | 96 | 18 |
| 1614 | GAGTCSTGCAATSTGAATASAATTT | 1 | 8 | 1 | 1 | 2 | 4 | 1 | 94 | 38 | 1 | 101 | 4 |
| 1615 | GAGTSCTGCAATSTGAATATSATTT | 1 | 1 | 2 | 1 | 2 | 5 | 1 | 90 | 10 | 1 | 99 | 19 |
| 1633 | GTCTTCTSCCGAGTCSTGCAATA | 2 | 53 | 3 | 2 | 83 | 23 | 2 | 45 | 8 | 2 | 10 | 2 |
| 1634 | GTCTTCTGCCGSGTCCTGCAATA | 2 | 31 | 21 | 2 | 74 | 0 | 2 | 41 | 6 | 2 | 12 | 5 |
| 1635 | AGGTCTTCSGCCGAGTCCSGCAATA | 1 | 23 | 2 | 0 | N/A | N/A | 1 | 43 | 6 | 0 | N/A | N/A |
| 1617 | CCTTTCTCSCGAAGGTCTTCSGCCG | 5 | 49 | 17 | 5 | 89 | 28 | 5 | 24 | 5 | 5 | 9 | 5 |
| 1618 | CTTTCTCSCGAAGGTSTTCTGCC | 3 | 46 | 13 | 3 | 82 | 22 | 3 | 35 | 15 | 3 | 11 | 3 |
| 1619 | TTTCTCTSGAAGGTCSTCTGCCG | 2 | 42 | 8 | 2 | 80 | 28 | 2 | 40 | 3 | 2 | 12 | 1 |
| 1620 | TCTCTCGSACACACGSACACATG | 4 | 74 | 22 | 4 | 103 | 15 | 4 | 5 | 3 | 3 | 1 | 1 |
| 1589 | AATCCAATTASGAGAGAGTGASGGG | 1 | 7 | 1 | 1 | 32 | 1 | 1 | 107 | 14 | 1 | 47 | 22 |
| 1590 | GAGTCCSGCAATASGAATATAATTT | 1 | 23 | 2 | 1 | 63 | 1 | 1 | 76 | 4 | 1 | 47 | 4 |
| 1591 | GCACACASGCTCACACAGSGAGCCA | 1 | 45 | 5 | 1 | 86 | 6 | 1 | 11 | 5 | 1 | 2 | 1 |
| 1636 | GT*C*C*TGCSATATGAASATAAT | 1 | 18 | 7 | 1 | 53 | 3 | 1 | 75 | 13 | 1 | 74 | 9 |
| 1637 | GT*C*C*TSCAATATGSATATAAT | 1 | 4 | 7 | 1 | 2 | 3 | 1 | 130 | 12 | 1 | 105 | 34 |
| 1638 | GT*C*C*TGCSATATGSATATAAT | 1 | 24 | 19 | 1 | 41 | 5 | 1 | 75 | 1 | 1 | 68 | 9 |
| 1639 | GTCTTCTSCCGAGT*C*S*T*GCAATA | 1 | 26 | 7 | 1 | 67 | 15 | 1 | 60 | 33 | 1 | 30 | 4 |
| 1640 | GT*CT*TC*TGCCGSGTCCTGCAATA | 1 | 33 | 8 | 1 | 63 | 11 | 1 | 36 | 9 | 1 | 17 | 6 |
| 1641 | GTCTTCTGCC*G*S*G*TCCTGCAATA | 1 | 21 | 11 | 1 | 91 | 11 | 1 | 34 | 23 | 1 | 23 | 4 |
| 1642 | CCTTTCTCSCGAAGGTCT*T*C*SGCCG | 2 | 40 | 11 | 2 | 77 | 21 | 2 | 28 | 13 | 2 | 14 | 4 |
| 1643 | CCTTTCTCSCGAAGGTCTT* | 2 | 40 | 13 | 2 | 77 | 6 | 2 | 21 | 4 | 2 | 15 | 1 |
| | C*S*G*CCG | | | | | | | | | | | | |
| 1644 | CTTTCTCSCGAAGG*T*S*T*TCTGCC | 1 | 28 | 5 | 1 | 46 | 17 | 1 | 60 | 7 | 1 | 36 | 13 |
| 1645 | GC*A*CA*C*ASGCTCACASAGAGAGC | 1 | 30 | 1 | 1 | 73 | 7 | 1 | 22 | 6 | 1 | 4 | 1 |
| 1646 | GCACAC*A*S*G*CTCACASAGAGAGC | 1 | 12 | 9 | 1 | 37 | 8 | 1 | 29 | 1 | 1 | 11 | 4 |
| 1647 | TC*TC*TC*GSACACACGSACACATG | 2 | 40 | 1 | 2 | 90 | 7 | 2 | 15 | 1 | 2 | 3 | 2 |
| 1648 | TCTCTCGSACACACGSA*CA*CA*TG | 2 | 58 | 5 | 2 | 108 | 7 | 2 | 19 | 2 | 2 | 7 | 4 |
| 1649 | GTCTTCTS^CCGAGTCS^TGCAATA | 3 | 26 | 9 | 3 | 71 | 9 | 3 | 19 | 5 | 3 | 21 | 9 |

indicates presence of phosphodiester linkage. All other linkages are phosphorothioate linkages.
:^indicates a spacer at the indicated position of the ASO, where the spacer is in accordance with Formula (I), where X is -O-; and n is 2.

TABLE 16
Performance of STMN2 AONs (STMN2 oligonucleotides without spacers).

| | | | | | | | | QPCR potency in hMN | | | | | |
|---|---|---|---|---|---|---|---|---|---|---|---|---|---|
| SEQ | | QPCR potency in hMN STMN2 FL | | | | | | STMN2 cryptic | | | | | |
| ID | | 50 nM | | | 200 nM | | | 50 nM | | | 200 nM | | |
| NO: | Sequence (5′ □ 3′) | N | Mean | SD | N | Mean | SD | N | Mean | SD | N | Mean | SD |
| 144 | AATCCAATTAAGAGAGAGTGATG | 1 | 2 | 3 | 1 | 23 | 5 | 1 | 71 | 21 | 1 | 49 | 8 |
| | GG | | | | | | | | | | | | |
| 146 | AAAATCCAATTAAGAGAGAGTGA | 1 | 11 | 4 | 1 | 19 | 5 | 1 | 45 | 5 | 1 | 36 | 4 |
| | TG | | | | | | | | | | | | |
| 150 | TTTAAAAATCCAATTAAGAGAGA | 3 | 43.7 | 39.4 | 3 | 46.7 | 13.7 | 3 | 38.7 | 20.6 | 3 | 17.7 | 7.1 |
| | GT | | | | | | | | | | | | |
| 169 | CCTGCAATATGAATATAATTTTA | 3 | 36.3 | 5.1 | 3 | 72.3 | 0.6 | 3 | 45.3 | 13.8 | 1 | 11.7 | 2.3 |
| | AA | | | | | | | | | | | | |
| 170 | TCCTGCAATATGAATATAATTTT | 3 | 28.3 | 13.1 | 3 | 86.3 | 12.3 | 3 | 69.3 | 34.7 | 3 | 25.3 | 10.1 |
| | AA | | | | | | | | | | | | |
| 171 | GTCCTGCAATATGAATATAATTT | 3 | 30.7 | 6.5 | 3 | 85.0 | 8.9 | 3 | 56.3 | 10.5 | 3 | 12.3 | 2.5 |
| | TA | | | | | | | | | | | | |
| 172 | AGTCCTGCAATATGAATATAATT | 3 | 33.0 | 8.2 | 3 | 79.3 | 5.1 | 3 | 54.7 | 12.7 | 3 | 15.7 | 5.1 |
| | TT | | | | | | | | | | | | |
| 173 | GAGTCCTGCAATATGAATATAAT | 6 | 29.0 | 13.3 | 6 | 81.5 | 16.1 | 6 | 61.3 | 14.2 | 6 | 21.0 | 7.3 |
| | TT | | | | | | | | | | | | |
| 197 | CCTTTCTCTCGAAGGTCTTCTGC | 8 | 26.7 | 14.5 | 8 | 79.3 | 31.3 | 8 | 44.4 | 15.4 | 8 | 12.1 | 7.2 |
| | CG | | | | | | | | | | | | |
| 237 | GCACACATGCTCACACAGAGAGC | 1 | 46 | 4 | 1 | 80 | 3 | 1 | 7 | 1 | 1 | 1 | 0 |
| | CA | | | | | | | | | | | | |
| 252 | CTCTCTCGCACACACGCACACAT | 5 | 37.6 | 20.0 | 5 | 69.6 | 31.1 | 5 | 19.0 | 9.6 | 5 | 2.3 | 1.5 |
| | gc | | | | | | | | | | | | |
| 1343 | AATCCAATTAAGAGAGAGTGATG | 1 | 7 | 1 | 1 | 15 | 6 | 1 | 56 | 8 | 1 | 33 | 11 |
| 1346 | GTCCTGCAATATGAATATAATTT | 3 | 67.3 | 40.4 | 3 | 98.0 | 10.4 | 3 | 49.3 | 31.0 | 3 | 10.3 | 1.2 |
| 1347 | GTCTTCTGCCGAGTCCTGCAATA | 2 | 12.5 | 3.6 | 2 | 40.2 | 16.7 | 2 | 55.7 | 13.2 | 2 | 20.8 | 15.9 |
| 1348 | GCACACATGCTCACACAGAGAGC | 2 | 45.6 | 13.6 | 2 | 89.5 | 2.1 | 2 | 11.6 | 7.6 | 2 | 0.7 | 0.4 |
| 1351 | AATCCAATTAAGAGAGAGTGA | 1.0 | 10.0 | 2.0 | 1.0 | 12.0 | 2.0 | 1.0 | 69.0 | 5.0 | 1.0 | 35.0 | 9.0 |
| 1353 | GTCCTGCAATATGAATATAAT | 5 | 48.2 | 12.9 | 5 | 100.5 | 18.8 | 5 | 47.2 | 11.4 | 5 | 18.3 | 6.0 |
| 1353 | GT*CC*TG*CAATATGAA*TA*T | 1 | 36.4 | 7.0 | 1 | 84.3 | 7.0 | 1 | 64.0 | 5.0 | 1 | 32.8 | 12.0 |
| | A*AT | | | | | | | | | | | | |
| 1355 | CCTGCAATATGAATATAATTT | 4 | 50.0 | 9.3 | 4 | 79.0 | 19.5 | 4 | 21.5 | 7.2 | 4 | 7.0 | 2.2 |
| 1421 | CCTGCAATATGAATATAATTTTA | 1.0 | 44.0 | 18.0 | 1.0 | 120.0 | 39.0 | 1.0 | 32.0 | 1.0 | 1.0 | 8.0 | 1.0 |
| 1422 | TGCAATATGAATATAATTTTAAA | 4 | 43.9 | 14.5 | 4 | 80.7 | 3.9 | 4 | 40.5 | 8.8 | 4 | 24.0 | 16.3 |
| 1423 | CTGCAATATGAATATAATTTTAA | 3 | 48.0 | 17.6 | 3 | 88.7 | 9.5 | 3 | 38.3 | 13.2 | 3 | 10.7 | 4.9 |
| 1424 | TGCAATATGAATATAATTTTA | 1.0 | 40.0 | 5.0 | 1.0 | 79.0 | 13.0 | 1.0 | 33.0 | 5.0 | 1.0 | 15.0 | 0.0 |
| 1425 | TCCTGCAATATGAATATAATTTT | 4 | 39.0 | 5.1 | 4 | 95.8 | 9.8 | 4 | 40.6 | 14.9 | 4 | 12.0 | 2.9 |
| 1426 | CTGCAATATGAATATAATTTT | 4 | 45.5 | 9.3 | 4 | 85.2 | 6.5 | 4 | 39.4 | 16.7 | 4 | 12.6 | 3.2 |
| 1427 | AGTCCTGCAATATGAATATAATT | 3 | 39.7 | 9.0 | 3 | 76.0 | 18.2 | 3 | 42.3 | 5.7 | 3 | 13.3 | 5.0 |
| 1428 | TCCTGCAATATGAATATAATT | 4 | 43.0 | 14.0 | 4 | 91.5 | 18.6 | 4 | 42.8 | 14.2 | 4 | 10.0 | 2.1 |
| 1429 | TTTCTCTCGAAGGTCTTCTGCCG | 3 | 49.5 | 49.5 | 3 | 85.5 | 44.5 | 3 | 40.9 | 22.8 | 3 | 9.5 | 6.6 |
| 1430 | CCTTTCTCTCGAAGGTCTTCTGC | 1.0 | 41.5 | 5.0 | 1.0 | 98.2 | 10.0 | 1.0 | 27.5 | 8.0 | 1.0 | 5.9 | 1.0 |
| 1431 | CTTTCTCTCGAAGGTCTTCTGCC | 4 | 32.6 | 17.9 | 4 | 83.3 | 37.7 | 4 | 40.6 | 27.1 | 4 | 12.6 | 9.7 |
| 1432 | CTCTCGCACACACGCACACATGC | 4 | 34.0 | 12.7 | 4 | 51.8 | 10.5 | 4 | 25.5 | 8.0 | 4 | 3.1 | 2.1 |
| 1433 | CTCTCTCGCACACACGCACACAT | 1.0 | 20.2 | 2.0 | 1.0 | 60.8 | 6.0 | 1.0 | 6.5 | 7.0 | 1.0 | 2.9 | 2.0 |
| 1434 | TCTCTCGCACACACGCACACATG | 8 | 43.3 | 16.6 | 8 | 77.5 | 19.8 | 8 | 18.3 | 8.0 | 8 | 2.7 | 2.1 |
| 1435 | CTCTCGCACACACGCACACAT | 1.0 | 33.0 | 32.0 | 1.0 | 83.4 | 25.0 | 1.0 | 22.6 | 9.0 | 1.0 | 3.7 | 2.0 |
| 1650 | CT*C*TC*T*CGCACACACGCAC | 1.0 | 26.6 | 4.0 | 1.0 | 68.8 | 1.0 | 1.0 | 40.3 | 3.0 | 1.0 | 13.3 | 3.0 |
| | ACATGC | | | | | | | | | | | | |
| 1651 | TC*TC*TC*GCACACACGCACAC | 1.0 | 46.1 | 7.0 | 1.0 | 91.0 | 6.0 | 1.0 | 32.4 | 1.0 | 1.0 | 8.9 | 1.0 |
| | ATG | | | | | | | | | | | | |
| 1652 | TTTCTCTCGAAGGTCTTCTGC | 2 | 26.0 | 2.4 | 2 | 75.9 | 6.0 | 2 | 49.4 | 2.7 | 2 | 8.9 | 0.8 |
| 1653 | AAAATCCAATTAAGAGAGAGTGA | 1.0 | 15.0 | 2.0 | 1.0 | 19.0 | 2.0 | 1.0 | 49.0 | 3.0 | 1.0 | 29.0 | 5.0 |
| 1654 | AAATCCAATTAAGAGAGAGTGAT | 1.0 | 12.0 | 1.0 | 1.0 | 18.0 | 2.0 | 1.0 | 55.0 | 2.0 | 1.0 | 31.0 | 4.0 |
| 1655 | TAAAAATCCAATTAAGAGAGAGT | 1.0 | 32.0 | 4.0 | 1.0 | 42.0 | 6.0 | 1.0 | 37.0 | 5.0 | 1.0 | 24.0 | 5.0 |
| 1656 | TTTAAAAATCCAATTAAGAGAGA | 1.0 | 25.0 | 1.0 | 1.0 | 32.0 | 1.0 | 1.0 | 37.0 | 4.0 | 1.0 | 29.0 | 2.0 |
| 1657 | TTAAAAATCCAATTAAGAGAGAG | 1.0 | 18.0 | 4.0 | 1.0 | 20.0 | 8.0 | 1.0 | 33.0 | 25.0 | 1.0 | 19.0 | 2.0 |
| 1658 | TAAAAATCCAATTAAGAGAGA | 3 | 21.7 | 7.5 | 3 | 52.0 | 29.1 | 3 | 60.0 | 29.0 | 3 | 42.0 | 18.1 |
| 1659 | CC*T*T*TCTCTCGAAGGTCTTC | 1.0 | 40.0 | 1.0 | 1.0 | 99.7 | 2.0 | 1.0 | 35.5 | 5.0 | 1.0 | 13.8 | 2.0 |
| | TGCCG | | | | | | | | | | | | |
| 1660 | GCACACATGCTCACACA*GA*GA | 1 | 40.8 | 4.0 | 1 | 85.1 | 6.0 | 1 | 12.9 | 2.0 | 1 | 3.4 | 0.0 |
| | *GC | | | | | | | | | | | | |
| 1661 | GC*A*CA*C*ATGCTCACACAGA | 1 | 38.2 | 6.0 | 1 | 81.0 | 3.0 | 1 | 26.0 | 1.0 | 1 | 4.4 | 2.0 |
| | GAGC | | | | | | | | | | | | |

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A compound comprising a modified oligonucleotide comprising a sequence that is between 85 and 98% complementary to an equal length portion of any one of SEQ ID NO: 1339 or SEQ ID NO: 1341, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

2. An oligonucleotide comprising a sequence that is between 85 and 98% complementary to an equal length portion of any one of SEQ ID NO: 1339 or SEQ ID NO: 1341, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

3. The compound of claim 1 or oligonucleotide of claim 2, wherein the oligonucleotide comprises a segment with at most 11 linked nucleosides.

4. The compound of claim 1 or 3, or oligonucleotide of claim 2 or 3, wherein the oligonucleotide comprises a segment with at most 10, 9, or 8 linked nucleosides.

5. The compound of any one of claim 1 or 3-4 or oligonucleotide of any one of claims 2-4, wherein the oligonucleotide comprises a segment with at most 7 linked nucleosides.

6. The compound of any one of claim 1 or 3-5 or oligonucleotide of any one of claims 1-5, wherein the oligonucleotide comprises a segment with at most 6, 5, 4, 3, or 2 linked nucleosides.

7. The compound of any one of claim 1 or 3-6 or oligonucleotide of any one of claims 1-6, wherein every segment of the oligonucleotide comprises at most 7 linked nucleosides.

8. The compound or oligonucleotide of any one of claims 3-7, wherein the oligonucleotide comprises a sequence that shares at least 85% identity with an equal length portion of any one of SEQ ID NOs: 1-466, SEQ ID NOs: 893-1338, SEQ ID NOs: 1342-1366, or SEQ ID NOs: 1392-1664.

9. The compound or oligonucleotide of any one of claims 3-8, wherein the oligonucleotide comprises a sequence that shares at least 90% identity with an equal length portion of any one of SEQ ID NOs: 1-466, SEQ ID NOs: 893-1338, SEQ ID NOs: 1342-1366, or SEQ ID NOs: 1392-1664.

10. The compound or oligonucleotide of any one of claims 3-9, wherein the oligonucleotide comprises a sequence that shares at least 95% identity with an equal length portion of any one of SEQ ID NOs: 1451-1664.

11. The compound or oligonucleotide of any one of claims 3-9, wherein the oligonucleotide comprises a sequence that shares 100% identity with an equal length portion of any one of SEQ ID NOs: 1451-1664.

12. The compound of claim 1 or oligonucleotide of claim 2, wherein the oligonucleotide comprises a segment with at most 11 linked nucleosides or at most 7 linked nucleosides, and wherein the oligonucleotide comprises a sequence that is between 85 and 98% complementary to an equal length portion within any one of positions 144-168, 173-197, 185-209, or 237-261 of SEQ ID NO: 1339.

13. The compound or oligonucleotide of claim 12, wherein the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 144-164, 144-166, 145-167, 146-166, 146-168, 147-165, or 148-168 of SEQ ID NO: 1339.

14. The compound or oligonucleotide of claim 12, wherein the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 173-191, 173-193, 173-195, 173-197, 175-195, 175-197, 177-197, or 179-197 of SEQ ID NO: 1339.

15. The compound or oligonucleotide of claim 12, wherein the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 185-205, 187-209, 189-209, or 191-209 of SEQ ID NO: 1339.

16. The compound or oligonucleotide of claim 12, wherein the oligonucleotide comprises a segment with at most 11 linked nucleosides or at most 7 linked nucleosides, and wherein the oligonucleotide comprises a sequence that is between 85 and 98% complementary to an equal length portion of nucleobases within any one of positions 237-255, 237-259, 239-259, 239-261, 241-261, or 243-261 of SEQ ID NO: 1339.

17. The compound or oligonucleotide of claim 12, wherein the oligonucleotide comprises a segment with at most 6, 5, 4, 3, or 2 linked nucleosides, and wherein the oligonucleotide comprises a sequence that is between 85 and 98% complementary to an equal length portion of nucleobases within any one of positions 144-168, 173-197, 185-209, or 237-261 of SEQ ID NO: 1339.

18. The compound or oligonucleotide of claim 12, wherein the oligonucleotide comprises a segment with at most 6, 5, 4, 3, or 2 linked nucleosides, and wherein the oligonucleotide comprises a sequence that is between 85 and 98% complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 187-209, 189-209, 191-209, 237-255, 237-259, 239-259, 239-261, 241-261, or 243-261 of SEQ ID NO: 1339.

19. The compound or oligonucleotide of claim 12, wherein the oligonucleotide comprises a segment with at most 11 linked nucleosides or at most 7 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, or SEQ ID NOs: 1392-1664.

20. The compound or oligonucleotide of claim 19, wherein the oligonucleotide comprises a segment with at most 6, 5, 4, 3, or 2 linked nucleosides, and wherein the oligonucleotide comprises a sequence that shares at least 85% identity with an equal length portion of any one of SEQ ID NOs: 36, 55, 144, 173, 177, 181, 185, 197, 203, 209, 215, 237, 244, 252, 380, 385, 390, 395, 400, 928, 947, 1036, 1065, 1069, 1073, 1077, 1089, 1095, 1101, 1107, 1129, 1136, 1144, 1272, 1277, 1282, 1287, or 1292.

21. The compound or oligonucleotide of claim 19 or 20, wherein the oligonucleotide comprises a segment with at most 6, 5, 4, 3, or 2 linked nucleosides, and wherein the oligonucleotide comprises a sequence that shares at least 90% identity with an equal length portion of any one of SEQ ID NOs: 36, 55, 144, 173, 177, 181, 185, 197, 203, 209, 215, 237, 244, 252, 380, 385, 390, 395, 400, 928, 947, 1036, 1065, 1069, 1073, 1077, 1089, 1095, 1101, 1107, 1129, 1136, 1144, 1272, 1277, 1282, 1287, or 1292.

22. The compound of any one of claims 1 and 3-21 or oligonucleotide of any one of claims 2-21, wherein the oligonucleotide is at least 18, at least 19, at least 20, at least 21, at least 22, at least 23, at least 24, or at least 25 oligonucleotide units in length.

23. The compound of claim 21 or oligonucleotide of claim 21, wherein the oligonucleotide is at least 19 oligonucleotide units in length.

24. The compound of any one of claims 1 and 3-23 or oligonucleotide of any one of claims 2-23, wherein the spacer is a nucleoside-replacement group comprising a non-sugar substitute that is incapable of linking to a nucleotide base.

25. The compound or oligonucleotide of claim 24, wherein the spacer is located between positions 10 and 15 of the oligonucleotide.

26. The compound or oligonucleotide of claim 24, wherein the spacer is located between positions 7 and 11 of the oligonucleotide.

27. The compound or oligonucleotide of claim 24 or 26, wherein the oligonucleotide further comprises a second spacer, wherein the second spacer is located between positions 14 and 22 of the oligonucleotide.

28. The compound or oligonucleotide of claim 27, wherein the spacer and the second spacer are separated by at least 5 nucleobases, at least 6 nucleobases, or at least 7 nucleobases in the oligonucleotide.

29. The compound or oligonucleotide of claim 27 or 28, wherein the spacer is located between positions 7 and 9 of the oligonucleotide, and wherein the second spacer is located between positions 15 and 18 of the oligonucleotide.

30. The compound or oligonucleotide of any one of claims 27-29, wherein the spacer is located at position 8 of the oligonucleotide, and wherein the second spacer is located at position 16 of the oligonucleotide.

31. The compound or oligonucleotide of any one of claims 27-30, wherein the oligonucleotide further comprises a third spacer, wherein the third spacer is located between positions 21 and 24 of the oligonucleotide.

32. The compound or oligonucleotide of claim 24, wherein the spacer is located between positions 2 and 5 of the oligonucleotide.

33. The compound or oligonucleotide of claim 32, wherein the oligonucleotide further comprises a second spacer, wherein the second spacer is located between positions 8 and 12 of the oligonucleotide.

34. The compound or oligonucleotide of claim 33, wherein the oligonucleotide further comprises a third spacer, wherein the third spacer is located between positions 18 and 22 of the oligonucleotide.

35. The compound or oligonucleotide of claim 24, wherein the oligonucleotide further comprises a second spacer and a third spacer, wherein the three spacers are located at positions in the oligonucleotide such that each segment of the oligonucleotide has at most 7 linked nucleosides.

36. The compound or oligonucleotide of claim 35, wherein at least two of the three spacers are adjacent to a guanine nucleobase.

37. The compound or oligonucleotide of claim 36, wherein each of the at least two of the three spacers immediately precede a guanine nucleobase.

38. The compound or oligonucleotide of any one of claims 24-37, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

39. The compound or oligonucleotide of any one of claims 24-37, wherein each of the first, second or third spacers is independently represented by Formula (X), wherein: 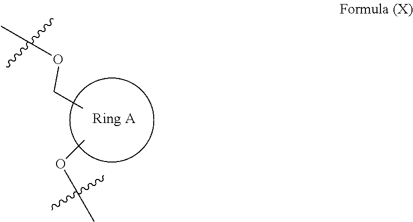
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 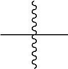
symbol represents the point of connection to an internucleoside linkage.

40. The compound or oligonucleotide of claim 39, wherein each of the first, second or third spacers is independently represented by Formula (Xa), wherein:

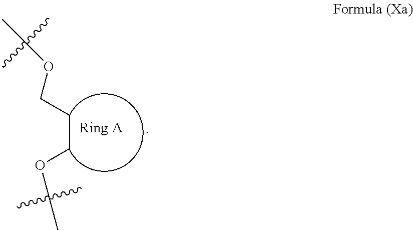 [SMILES: *OCCCO*]

41. The compound or nucleotide of claim 39 or 40, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, 216yrrolidinyl, piperidinyl, piperazinyl, morpholinyl and azepanyl.

42. The compound or nucleotide of claim 41 wherein ring A is tetrahydrofuranyl.

43. The compound or nucleotide of claim 41 wherein ring A is tetrahydropyranyl.

44. The compound or oligonucleotide of any one of claims 24-37, wherein each of the first, second or third spacers is independently represented by Formula I, wherein: 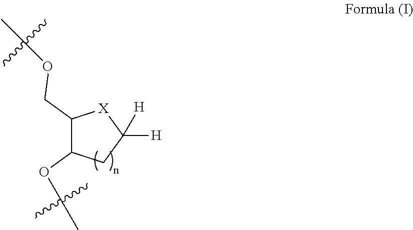
X is selected from —CH₂— and —O—; and
n is 0, 1, 2 or 3.

45. The compound or oligonucleotide of any one of claims 24-37, wherein each of the first, second or third spacers is independently represented by Formula I′, wherein: 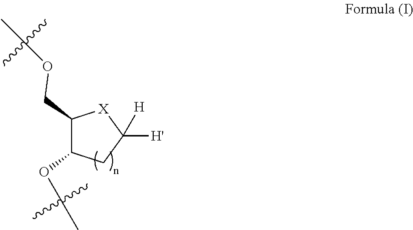
X is selected from —CH₂— and —O—; and
n is 0, 1, 2 or 3.

46. The compound or oligonucleotide of any one of claims 24-37, wherein each of the first, second or third spacers is independently represented by Formula (Ia), wherein: 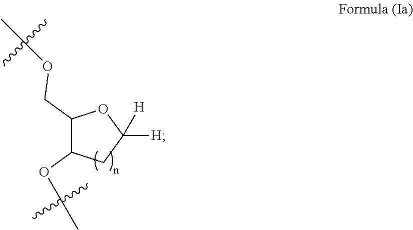
and
n is 0, 1, 2 or 3.

47. The compound or oligonucleotide of any one of claims 24-37, wherein each of the first, second or third spacers is independently represented by Formula (Ia′), wherein: 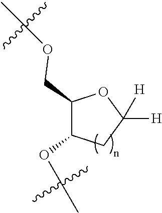
and
n is 0, 1, 2 or 3.

48. The compound or oligonucleotide of any one of claims 24-37, wherein each of the first, second or third spacers is independently represented by Formula II, wherein: 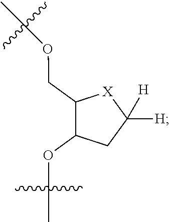
and
X is selected from —CH₂— and —O—.

49. The compound or oligonucleotide of any one of claims 24-37, wherein each of the first, second or third spacers is independently represented by Formula II′, wherein: 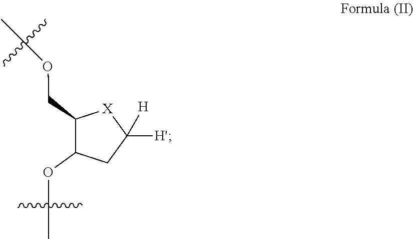
and
X is selected from —CH₂— and —O—.

50. The compound or oligonucleotide of any one of claims 24-37, wherein each of the first, second or third spacers is independently represented by Formula (Iia), wherein:

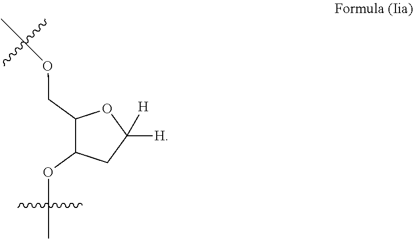

51. The compound or oligonucleotide of any one of claims 24-37, wherein each of the first, second or third spacers is independently represented by Formula (Iia′), wherein:

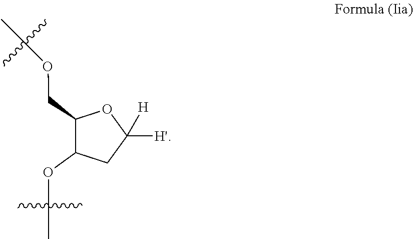

52. The compound or oligonucleotide of any one of claims 24-37, wherein each of the first, second or third spacers is independently represented by Formula III, wherein: 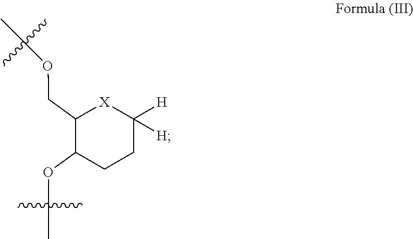
and
X is selected from —CH₂— and —O—.

53. The compound or oligonucleotide of any one of claims 24-37, wherein each of the first, second or third spacers is independently represented by Formula III′, wherein: 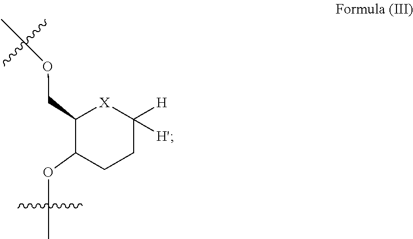
and
X is selected from —CH₂— and —O—.

54. The compound or oligonucleotide of any one of claims 24-37, wherein each of the first, second or third spacers is independently represented by Formula (IIIa), wherein:

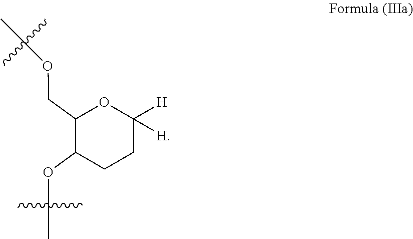

55. The compound or oligonucleotide of any one of claims 24-37, wherein each of the first, second or third spacers is independently represented by Formula (IIIa′), wherein:

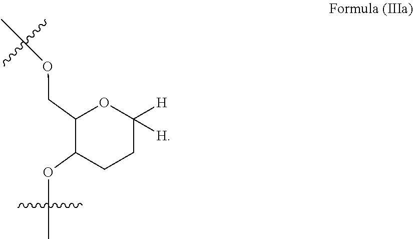

56. The compound or oligonucleotide of any one of the above claims, wherein the oligonucleotide comprising the spacer has a GC content of at least 10%.

57. The compound or oligonucleotide of any one of the above claims, wherein the oligonucleotide comprising the spacer has a GC content of at least 20%.

58. The compound or oligonucleotide of any one of the above claims, wherein the oligonucleotide comprising the spacer has a GC content of at least 25%.

59. The compound or oligonucleotide of any one of the above claims, wherein the oligonucleotide comprising the spacer has a GC content of at least 30%.

60. The compound or oligonucleotide of any one of the above claims, wherein the oligonucleotide comprising the spacer has a GC content of at least 40%.

61. The compound or oligonucleotide of any one of the above claims, wherein the oligonucleotide comprising the spacer has a GC content of at least 50%.

62. The compound or oligonucleotide of any one of the above claims, wherein the oligonucleotide is between 12 and 40 oligonucleotide units in length.

63. The compound or oligonucleotide of any one of the above claims, wherein at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

64. The compound or oligonucleotide of any one of claims 1-63, wherein one or more nucleoside linkages that link a base at position 3 or position 4 of the oligonucleotide are phosphodiester linkages.

65. The compound or oligonucleotide of claim 64, wherein only one nucleoside linkage that links a base at position 3 or position 4 of the oligonucleotide is a phosphodiester linkage.

66. The compound or oligonucleotide of any one of claims 1-63, wherein nucleoside linkages that link bases at both position 3 and position 4 of the oligonucleotide are phosphodiester linkages.

67. The compound or oligonucleotide of any one of claims 1-63, wherein one or more bases immediately preceding a spacer in the oligonucleotide are linked through phosphodiester bonds.

68. The compound or oligonucleotide of claim 67, wherein only the base immediately preceding the spacer in the oligonucleotide is linked to the spacer through a phosphodiester bond.

69. The compound or oligonucleotide of claim 68, wherein the base immediately preceding the spacer in the oligonucleotide is further linked to a further preceding base through a phosphodiester bond.

70. The compound or oligonucleotide of claim 68, wherein the oligonucleotide comprises a second spacer, wherein a base immediately preceding the second spacer is linked to a further preceding base through a phosphodiester bond.

71. The compound or oligonucleotide of any one of claims 1-63, wherein one or more bases immediately succeeding a spacer in the oligonucleotide are linked through phosphodiester bonds.

72. The compound or oligonucleotide of claim 71, wherein only the base immediately succeeding the spacer in the oligonucleotide is linked to the spacer through a phosphodiester bond.

73. The compound or oligonucleotide of claim 67, wherein two bases immediately preceding the spacer in the oligonucleotide are linked through phosphodiester bonds.

74. The compound or oligonucleotide of any one of claims 1-63, wherein one or more bases immediately preceding a spacer in the oligonucleotide are linked through phosphodiester bonds and wherein one or more bases immediately succeeding the spacer in the oligonucleotide are linked through phosphodiester bonds.

75. The compound or oligonucleotide of claim 74, wherein one base immediately preceding the spacer and one base immediately succeeding the spacer are linked through phosphodiester bonds.

76. The compound or oligonucleotide of claim 74 or 75, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

77. The compound or oligonucleotide of claim 76, wherein one base immediately preceding the second spacer and one base immediately succeeding the second spacer are linked through phosphodiester bonds.

78. The compound or oligonucleotide of any one of claims 1-63, wherein the oligonucleotide comprises a range of bases that are linked through phosphodiester bonds, the range of bases comprising at least two bases.

79. The compound or oligonucleotide of any one of claims 1-63, wherein the oligonucleotide comprises a range of bases that are linked through phosphodiester bonds, the range of bases comprising at least five bases.

80. The compound or oligonucleotide of claim 78 or 79, wherein the oligonucleotide comprises two or more spacers, and wherein the range of bases are positioned between the at least two spacers.

81. A compound comprising an oligonucleotide comprising a nucleobase sequence that shares at least 90% identity to an equal length portion of any one of SEQ ID NOs: 1-466, SEQ ID NOs: 893-1338, SEQ ID NOs: 1342-1366, or SEQ ID NOs: 1392-1664.

82. An oligonucleotide comprising a nucleobase sequence that shares at least 90% identity to an equal length portion of any one of SEQ ID NOs: 1-466, SEQ ID NOs: 893-1338, SEQ ID NOs: 1342-1366, or SEQ ID NOs: 1392-1664.

83. The compound of claim 81 or the oligonucleotide of claim 81 or 82, wherein the nucleobase sequence shares at least 95% identity to an equal length portion of any one of SEQ ID NOs: 1-466, SEQ ID NOs: 893-1338, SEQ ID NOs: 1342-1366, or SEQ ID NOs: 1392-1664.

84. The compound of claim 81 or the oligonucleotide of claim 81 or 82, wherein the nucleobase sequence shares at least 100% identity to an equal length portion of any one of SEQ ID NOs: 1-466, SEQ ID NOs: 893-1338, SEQ ID NOs: 1342-1366, or SEQ ID NOs: 1392-1664.

85. The compound or oligonucleotide of any of claims 64-84, wherein the oligonucleotide is any one of a 19mer, 21mer, 23mer, or 25mer.

86. The compound or oligonucleotide of any one of the above claims, wherein one or more internucleoside linkage of the oligonucleotide is a modified internucleoside linkage.

87. The compound or oligonucleotide of claim 86, wherein the modified internucleoside linkage of the oligonucleotide is a phosphorothioate linkage.

88. The compound or oligonucleotide of claim 86 or 87, wherein all internucleoside linkages of the oligonucleotide are phosphorothioate linkages.

89. The compound or oligonucleotide of claim 87, wherein the phosphorothioate linkage is in one of a Rp configuration or a Sp configuration.

90. The compound or oligonucleotide of any one of the preceding claims, wherein the oligonucleotide comprises at least one modified sugar moiety.

91. The compound or oligonucleotide of claim 90, wherein the modified sugar moiety is one of a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

92. The compound or oligonucleotide of any one of the above claims, wherein the oligonucleotide exhibits at least a 30%, 40%, 50%, 60%, 70%, 80%, or 90% increase of full length STMN2 protein.

93. The compound or oligonucleotide of any one of the above claims, wherein the oligonucleotide exhibits at least a 100% increase of full length STMN2 protein.

94. The compound or oligonucleotide of any one of the above claims, wherein the oligonucleotide exhibits at least a 200% increase of full length STMN2 protein.

95. The compound or oligonucleotide of any one of the above claims, wherein the oligonucleotide exhibits at least a 300% increase of full length STMN2 protein.

96. The compound or oligonucleotide of any one of the above claims, wherein the oligonucleotide exhibits at least a 400% increase of full length STMN2 protein.

97. The compound or oligonucleotide of any one of claims 92-96, wherein increase of the full length STMN2 protein is measured in comparison to a reduced level of full length STMN2 protein achieved using a TDP43 antisense oligonucleotide.

98. The compound or oligonucleotide of any one of the above claims, wherein the oligonucleotide exhibits at least a 10%, 20%, 30%, 40%, 50%, 60%, 70%, 80%, 90%, or 100% rescue of full length STMN2 protein.

99. The compound or oligonucleotide of any one of the above claims, wherein the oligonucleotide exhibits at least a 50%, 60%, 70%, 80%, or 90% reduction of a STMN2 transcript with a cryptic exon.

100. A method of treating a neurological disease and/or a neuropathy in a patient in need thereof, the method comprising administering to the patient a compound or an oligonucleotide of any one of claims 1-99.

101. The method of claim 100, wherein the neurological disease selected from the group consisting of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

102. The method of claim 101, wherein the neurological disease is ALS.

103. The method of claim 101, wherein the neurological disease is FTD.

104. The method of claim 101, wherein the neurological disease is ALS with FTD.

105. The method of claim 100, wherein the neuropathy is chemotherapy induced neuropathy.

106. A method of restoring axonal outgrowth and/or regeneration of a neuron, the method comprising exposing the neuron to a compound or an oligonucleotide of any one of claims 1-99.

107. A method of increasing, promoting, stabilizing, or maintaining STMN2 expression and/or function in a neuron, the method comprising exposing the cell to a compound or an oligonucleotide of any one of claims 1-99.

108. The method of claim 106 or 107, wherein the neuron is a motor neuron.

109. The method of claim 106 or 107, wherein the neuron is a spinal cord neuron.

110. The method of any one of claims 106-109, wherein the neuron is a neuron of a patient in need of treatment of a neurological disease and/or a neuropathy.

111. The method of claim 110, wherein the neuropathy is chemotherapy induced neuropathy.

112. The method of any one of claims 106-111, wherein the exposing is performed in vivo or ex vivo.

113. The method of any one of claims 106-111, wherein the exposing comprises administering the oligonucleotide to a patient in need thereof.

114. The method of any one of claims 106-113, wherein the oligonucleotide is administered topically, parenterally, intrathecally, intrathalamically, intracisternally, orally, rectally, buccally, sublingually, vaginally, pulmonarily, intratracheally, intranasally, transdermally, or intraduodenally.

115. The method of claim 114, wherein the oligonucleotide is administered orally.

116. The method of any one of claims 106-114, wherein a therapeutically effective amount of the oligonucleotide is administered intrathecally, intrathalamically or intracisternally.

117. The method of any one of claims 106-116, wherein the patient is a human.

118. A pharmaceutical composition comprising the oligonucleotide of any one of claims 1-99, or a pharmaceutically acceptable salt thereof, and a pharmaceutically acceptable excipient.

119. The pharmaceutical composition of claim 118, wherein the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

120. A method of treating a neurological disease or a neuropathy in a patient in need thereof, the method comprising administering to a patient in need thereof a therapeutically effective amount of a pharmaceutical composition of claim 118 or 119.

121. The method of claim 120, wherein the neurological disease is selected from the group consisting of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)).

122. The method of claim 121, wherein the neurological disease is ALS.

123. The method of claim 121, wherein the neurological disease is FTD.

124. The method of claim 121, wherein the neurological disease is ALS with FTD.

125. The method of claim 120, wherein the neuropathy is chemotherapy induced neuropathy.

126. The method of any one of claims 120-125, wherein the pharmaceutical composition is administered topically, parenterally, orally, pulmonarily, rectally, buccally, sublingually, vaginally, intratracheally, intranasally, intracisternally, intrathecally, intrathalamically, transdermally, or intraduodenally.

127. The method of any one of claims 120-125, wherein the pharmaceutical composition is administered intrathecally, intrathalamically, or intracisternally.

128. The method of any one of claims 120-127, wherein a therapeutically effective amount of the oligonucleotide is administered intrathecally, intrathalamically or intracisternally.

129. The method of any one of claims 120-128, wherein the patient is human.

130. A method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA) optionally, wherein the oligonucleotide further comprises a spacer.

131. A method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

132. A method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

133. A method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, or SEQ ID NOs: 1392-1664, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

134. The method of any one of claims 130-133, wherein nucleoside linkages that link a base at position 3 or position 4 of the oligonucleotide are phosphodiester linkages.

135. The method of claim 134, wherein only one nucleoside linkage that links a base at position 3 or position 4 of the oligonucleotide is a phosphodiester linkage.

136. The method of any one of claims 130-133, wherein nucleoside linkages that link bases at both position 3 and position 4 of the oligonucleotide are phosphodiester linkages.

137. The method of any one of claims 130-133, wherein one or more bases immediately preceding a spacer in the oligonucleotide are linked through phosphodiester bonds.

138. The method of claim 137, wherein only the base immediately preceding the spacer in the oligonucleotide is linked to the spacer through a phosphodiester bond.

139. The method of claim 138, wherein the base immediately preceding the spacer in the oligonucleotide is further linked to a further preceding base through a phosphodiester bond.

140. The method of claim 138, wherein the oligonucleotide comprises a second spacer, wherein a base immediately preceding the second spacer is linked to a further preceding base through a phosphodiester bond.

141. The method of any one of claims 130-133, wherein one or more bases immediately succeeding a spacer in the oligonucleotide are linked through phosphodiester bonds.

142. The method of claim 141, wherein only the base immediately succeeding the spacer in the oligonucleotide is linked to the spacer through a phosphodiester bond.

143. The method of any one of claims 130-133, wherein two bases immediately preceding the spacer in the oligonucleotide are linked through phosphodiester bonds.

144. The method of any one of claims 130-133, wherein one or more bases immediately preceding a spacer in the oligonucleotide are linked through phosphodiester bonds and wherein one or more bases immediately succeeding the spacer in the oligonucleotide are linked through phosphodiester bonds.

145. The method of claim 144, wherein one base immediately preceding the spacer and one base immediately succeeding the spacer are linked through phosphodiester bonds.

146. The method of claim 144 or 145, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

147. The compound or oligonucleotide of claim 146, wherein one base immediately preceding the second spacer and one base immediately succeeding the second spacer are linked through phosphodiester bonds.

148. The method of any one of claims 130-133, wherein the oligonucleotide comprises a range of bases that are linked through phosphodiester bonds, the range of bases comprising at least two bases.

149. The method of any one of claims 130-133, wherein the oligonucleotide comprises a range of bases that are linked through phosphodiester bonds, the range of bases comprising at least five bases.

150. The method of claim 148 or 149, wherein the oligonucleotide comprises two or more spacers, and wherein the range of bases are positioned between the at least two spacers.

151. The method of any of claims 134-150, wherein the oligonucleotide is any one of a 19mer, 21mer, 23mer, or 25mer.

152. The method of any one of claims 130-133, wherein at least one (i.e., one or more) internucleoside linkage of the oligonucleotide is a phosphorothioate linkage.

153. The method of any one of claims 130-133, wherein all internucleoside linkages of the oligonucleotide are phosphorothioate linkages.

154. An oligonucleotide and a pharmaceutically acceptable excipient, the oligonucleotide comprising a sequence that is between 85 and 98% complementary to an equal length portion of any one of SEQ ID NO: 1339 or SEQ ID NO: 1341, a sequence having 90% identity thereof, or to a 15 to 50 contiguous nucleobase portion thereof, optionally wherein the oligonucleotide comprises a spacer and wherein the oligonucleotide is capable of increasing, restoring, or stabilizing expression of the STMN2 mRNA capable of translation of a functional STMN2 and/or activity and/or function of STMN2 protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

155. The method of any one of claim 100-117 or 120-153, the pharmaceutical composition of claim 118 or 119, or the oligonucleotide of any one of claim 1-99 or 154, wherein the oligonucleotide comprises one or more chiral centers and/or double bonds.

156. The method of any one of claim 100-117, 120-153, or 155, the pharmaceutical composition of claim 118, 119, or 155, or the oligonucleotide of any one of claim 1-99 or 154-155, wherein the oligonucleotide exist as stereoisomers selected from geometric isomers, enantiomers, and diastereomers.

157. A method of treating a neurological disease and/or a neuropathy in a patient in need thereof, the method comprising administering to a patient in need thereof a therapeutically effective amount of a pharmaceutical composition of claim 118 or 119, in combination with a second therapeutic agent.

158. The method of claim 157, wherein the second therapeutic agent is selected from Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g. BIIB067, BIIB078, and BIIB105), BIIB100, levodopa/carbidopa, dopaminergic agents (e.g., ropinirole, pramipexole, rotigotine), medroxyprogesetrone, KCNQ2/KCNQ3 openers (e.g., retigabine, XEN1101, or QRL-101), anticonvulsants and psychostimulant agents, and/or a therapy (e.g., selected from breathing care, physical therapy, occupational therapy, speech therapy, nutritional support), for treating said neurologic disease.

159. A method of treating a neurological disease and/or a neuropathy in a patient in need thereof, the method comprising administering to a patient in need thereof a therapeutically effective amount of a pharmaceutical composition of claim 118 or 119, wherein at least one nucleoside linkage of the oligonucleotide is a non-natural linkage, optionally wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

160. The method of any one of claim 100-117, 120-153, or 155-159, wherein the spacer is a nucleoside-replacement group comprising a non-sugar substitute that is incapable of linking to a nucleotide base.

161. The method of claim 160, wherein the spacer is located between positions 10 and 15 of the oligonucleotide.

162. The method of claim 160, wherein the spacer is located between positions 7 and 11 of the oligonucleotide.

163. The method of claim 160 or 162, wherein the oligonucleotide further comprises a second spacer, wherein the second spacer is located between positions 14 and 22 of the oligonucleotide.

164. The method of claim 163, wherein the spacer and the second spacer are separated by at least 5 nucleobases, at least 6 nucleobases, or at least 7 nucleobases in the oligonucleotide.

165. The method of claim 163 or 164, wherein the spacer is located between positions 7 and 9 of the oligonucleotide, and wherein the second spacer is located between positions 15 and 18 of the oligonucleotide.

166. The method of any one of claims 163-165, wherein the spacer is located at position 8 of the oligonucleotide, and wherein the second spacer is located at position 16 of the oligonucleotide.

167. The method of any one of claims 163-166, wherein the oligonucleotide further comprises a third spacer, wherein the third spacer is located between positions 21 and 24 of the oligonucleotide.

168. The method of claim 160, wherein the spacer is located between positions 2 and 5 of the oligonucleotide.

169. The method of claim 168, wherein the oligonucleotide further comprises a second spacer, wherein the second spacer is located between positions 8 and 12 of the oligonucleotide.

170. The method of claim 169, wherein the oligonucleotide further comprises a third spacer, wherein the third spacer is located between positions 18 and 22 of the oligonucleotide.

171. The method of claim 160, wherein the oligonucleotide further comprises a second spacer and a third spacer, wherein the three spacers are located at positions in the oligonucleotide such that each segment of the oligonucleotide has at most 7 linked nucleosides.

172. The method of claim 171, wherein at least two of the three spacers are adjacent to a guanine nucleobase.

173. The method of claim 172, wherein each of the at least two of the three spacers immediately precede a guanine nucleobase.

174. The method of any one of claims 160-173, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

175. The method of any one of claims 160-173, wherein each of the first, second or third spacers is independently represented by Formula (X), wherein: 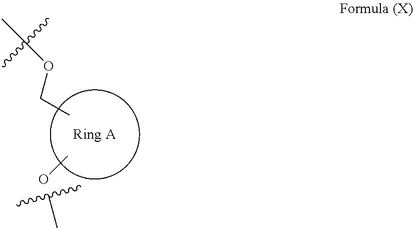
Ring A is is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 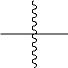
symbol represents the point of connection to an internucleoside linkage.

176. The method of claim 175, wherein each of the first, second or third spacers is independently represented by Formula (Xa), wherein:

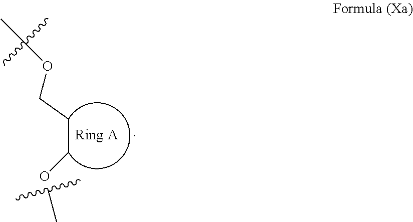

177. The method of claim 175 or 176, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

178. The method of claim 177, wherein ring A is tetrahydrofuranyl.

179. The method of claim 177, wherein ring A is tetrahydropyranyl.

180. The method of any one of claims 160-173 wherein each of the first, second or third spacers is independently represented by Formula (I), wherein: 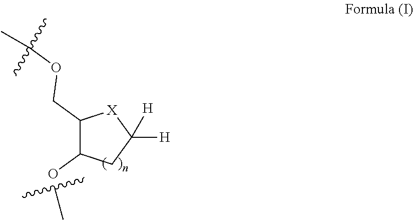
X is selected from —CH₂— and —O—; and
n is 0, 1, 2 or 3.

181. The method of any one of claims 160-173, wherein each of the first, second or third spacers is independently represented by Formula (I′), wherein:

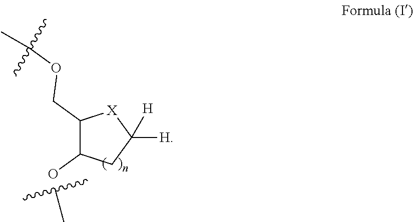

182. The method of any one of claims 160-173, wherein each of the first, second or third spacers is independently represented by Formula (Ia), wherein:

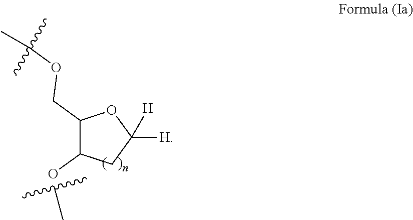

183. The method of any one of claims 160-173, wherein each of the first, second or third spacers is independently represented by Formula (Ia′), wherein:

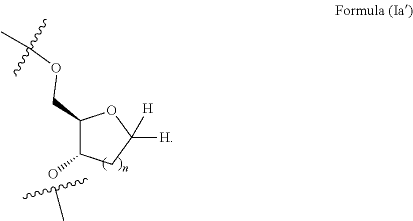

184. The method of any one of claims 160-173, wherein each of the first, second or third spacers is independently represented by Formula II, wherein: 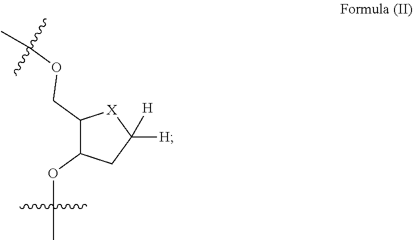
and
X is selected from —CH₂— and —O—.

185. The method of any one of claims 160-173, wherein each of the first, second or third spacers is independently represented by Formula II′, wherein: 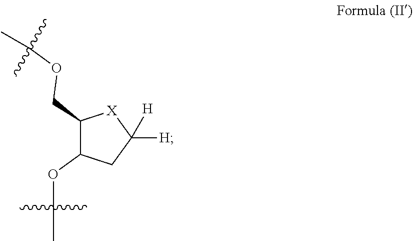
and
X is selected from —CH₂— and —O—.

186. The method of any one of claims 160-173, wherein each of the first, second or third spacers is independently represented by Formula (Ha), wherein:

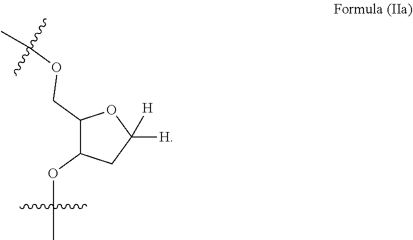

187. The method of any one of claims 160-173, wherein each of the first, second or third spacers is independently represented by Formula (IIa′), wherein:

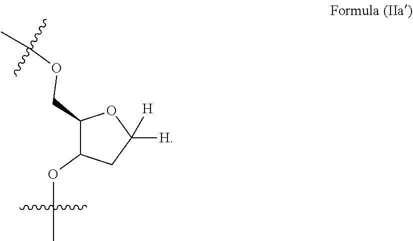

188. The method of any one of claims 160-173, wherein each of the first, second or third spacers is independently represented by Formula III, wherein: 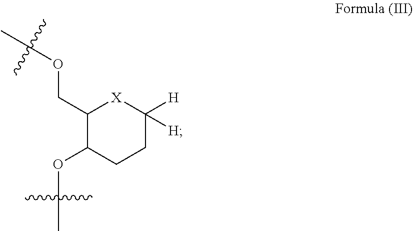
and
X is selected from —CH₂— and —O—.

189. The method of any one of claims 160-173, wherein each of the first, second or third spacers is independently represented by Formula III′, wherein: 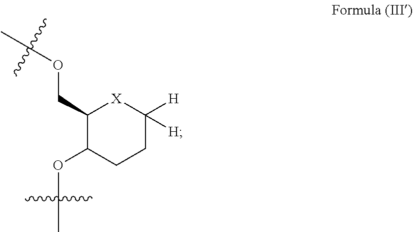
and
X is selected from —CH₂— and —O—.

190. The method of any one of claims 160-173, wherein each of the first, second or third spacers is independently represented by Formula (IIIa), wherein:

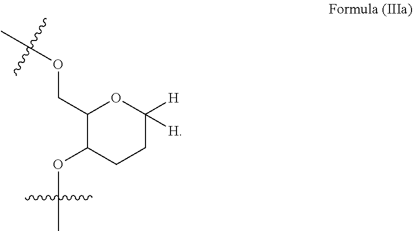

191. The method of any one of claims 160-173, wherein each of the first, second or third spacers is independently represented by Formula (IIIa′), wherein:

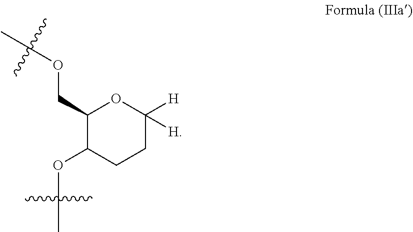

192. The method of any one of claims 160-191, wherein the oligonucleotide comprising the spacer has a GC content of at least 10%.

193. The method of any one of claims 160-192, wherein the oligonucleotide comprising the spacer has a GC content of at least 20%.

194. The method of any one of claims 160-193, wherein the oligonucleotide comprising the spacer has a GC content of at least 25%.

195. The method of any one of claims 160-194, wherein the oligonucleotide comprising the spacer has a GC content of at least 30%.

196. The method of any one of claims 160-195, wherein the oligonucleotide comprising the spacer has a GC content of at least 40%.

197. The method of any one of claims 160-196, wherein the oligonucleotide comprising the spacer has a GC content of at least 50%.
